SEQUENCE LISTING (5 sequences)

SEQ ID NO: 1 - DNA, 46 bp, Artificial Sequence
  Synthetic oligonucleotide probe sequence probe 1
ggatcctgcagcggccgctagcatgaccatgattacgccaagcttg

SEQ ID NO: 2 - DNA, 46 bp, Artificial Sequence
  Synthetic oligonucleotide probe sequence probe 2A
gtaccgagctcgaattcactggccgctagcggccgctgcaggatcc

SEQ ID NO: 3 - DNA, 54 bp, Artificial Sequence
  Synthetic oligonucleotide probe sequence probe 2B
gtaccgagctcgaattcactggccgctagcggcccgccggcccgccaacatggc

SEQ ID NO: 4 - DNA, 81 bp, Artificial Sequence
  Synthetic oligonucleotide probe sequence target probe
ggccagtgaattcgagctcggtacccggggatcctctagagtcgacctgcagccatgcaagcttggcgtaatcatggtcat

SEQ ID NO: 5 - DNA, 30 bp, Artificial Sequence
  Synthetic oligonucleotide probe sequence displacement probe
gccatgttggcgggccggcgggccgctagc

(19) United States
(12) Patent Application Publication
(10) Pub. No.: US 2004/0248093 A1
(43) Pub. Date: Dec. 9, 2004

(54) Magneto-optical bio-discs and systems including related methods

(76) Inventors: James Howard Coombs, Irvine, CA (US); Brigitte Chau Phan, Irvine, CA (US); Ramoncito Magpantay Valencia, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/307,263

(22) Filed: Nov. 27, 2002

Related U.S. Application Data
(63) Continuation-in-part of application No. 10/099,266, filed on Mar. 14, 2002.
Application No. 10/099,266 is a continuation-in-part of application No. 09/997,741, filed on Nov. 27, 2001.
(60) Provisional application No. 60/253,283, filed on Nov. 27, 2000.
(60) Provisional application No. 60/253,958, filed on Nov. 28, 2000.
(60) Provisional application No. 60/272,525, filed on Mar. 1, 2001.
(60) Provisional application No. 60/355,644, filed on Feb. 5, 2002.
(60) Provisional application No. 60/356,982, filed on Feb. 13, 2002.
(60) Provisional application No. 60/358,479, filed on Feb. 19, 2002.
(60) Provisional application No. 60/372,007, filed on Apr. 11, 2002.
(60) Provisional application No. 60/388,132, filed on Jun. 12, 2002.
(60) Provisional application No. 60/408,227, filed on Sep. 4, 2002.

(51) Int. Cl. C12Q 1/68; C12M 1/34

(52) U.S. Cl. 435/6; 435/287.2

(57) ABSTRACT

The present invention relates in general to molecular and cellular biomagnetic assays and, in particular, to molecular and cellular biomagnetic assays conducted on magneto-optical bio-discs. The invention further relates to magneto-optical bio-disc systems including the magneto-optical bio-discs and MO drives. More specifically, but without restriction to the particular embodiments hereinafter described in accordance with the best mode of practice, this invention relates to biomagnetic methods, including immunomagnetic methods, for detection and selective manipulation of specific target cells in cell populations and solutions of cell populations, using magnetic particles or beads, and to magnetically guided neurite growth, nerve regeneration, and magnetically formed neural networks using the MOBDS.

### CROSS-REFERENCE TO RELATED APPLICATIONS

This application is a continuation-in-part of U.S. application Ser. No. 10/099,266 filed Mar. 14, 2002 which is a continuation-in-part of U.S. application Ser. No. 09/997,741 filed Nov. 27, 2001 which claimed the benefit of priority from U.S. Provisional Application Ser. No. 60/253,283 filed Nov. 27, 2000; U.S. Provisional Application Ser. No. 60/253,958 filed Nov. 28, 2000; and U.S. Provisional Application Ser. No. 60/272,525 filed Mar. 1, 2001.

This application also claims the benefit of priority from U.S. Provisional Application Ser. No. 60/355,644 filed Feb. 5, 2002; U.S. Provisional Application Ser. No. 60/356,982 filed Feb. 13, 2002; U.S. Provisional Application Ser. No. 60/358,479 filed Feb. 19, 2002; U.S. Provisional Application Ser. No. 60/372,007 filed Apr. 11, 2002; U.S. Provisional Application Ser. No. 60/388,132 filed Jun. 12, 2002; and U.S. Provisional Application Ser. No. 60/408,227 filed Sep. 4, 2002.

Each of the above utility and provisional applications is herein incorporated by reference in its entirety.

### BACKGROUND OF THE INVENTION

1. Field of the Invention

This invention relates in general to molecular and cellular biomagnetic assays and, in particular, to molecular and cellular biomagnetic assays conducted on optical bio-discs. The invention further relates to magneto-optical (MO) analysis discs and MO drive systems, hereinafter referred to as magneto-optical bio-disc systems (MOBDS). More specifically, but without restriction to the particular embodiments hereinafter described in accordance with the best mode of practice, this invention relates to biomagnetic methods, including immunomagnetic methods, for detection and selective manipulation of specific target cells in cell populations and solutions of cell populations, using magnetic particles or beads, and to magnetically guided neurite growth and nerve regeneration.

2. Discussion of the Related Art

There is a significant need to make diagnostic assays and forensic assays of all types faster and more local to the end-user. Ideally, clinicians, patients, investigators, the military, other health care personnel, and consumers should be able to test themselves for the presence of certain factors or indicators in their systems, and for the presence of certain biological material at a crime scene or on a battlefield. At present, there are a number of silicon-based chips with nucleic acids and/or proteins attached thereto, which are commercially available or under development. These chips are not for use by the end-user, or for use by persons or entities lacking very specialized expertise and expensive equipment.

### SUMMARY OF THE INVENTION

The present invention relates to performing biomagnetic assays and laboratory analysis, and particularly to using magnetic, paramagnetic, or superparamagnetics particles, herein referred to as bio-magnetic or magnetic particles or beads, on optical bio-discs, including, but not limited to, CD, CD-R, DVD, DVD-R, and MO discs. The biomagnetic assays of the present invention may include, for example, immunomagnetic assays, and molecumagnetic assays such as assays using DNA and RNA, implemented on non-magnetic and magnetic platforms. The non-magnetic platforms may include, for example, microtiterplates and non-magneto-optical bio-discs systems. The magnetic platform includes, for example, the magneto-optical bio-disc system (MOBDS). Assays conducted using the MOBDS are herein referred to as MO bio-magnetic assays (MOBMA) and assays using non-magneto-optical bio-discs are herein referred to as optical disc bio-magnetic assays (ODBMA). The ODBMA may be carried out, for example, using a modified optical disc drive having a controllable electromagnet associated therewith. The invention includes methods for preparing assays, methods for performing assays, methods for performing laboratory or clinical analysis, discs for performing assays or analysis, and related detection systems.

The biological sample can include blood, serum, plasma, cerebrospinal fluid, breast aspirate, synovial fluid, pleural fluid, perintoneal fluid, pericardial fluid, urine, saliva, amniotic fluid, semen, mucus, a hair, feces, a biological particulate suspension, a single-stranded or double-stranded nucleic acid molecule, a cell, an organ, a tissue, or a tissue extract, or any other sample that includes a target that may be bound to a magnetic particle through chemical or biological processes. Further details relating to other aspects associated with the selection and detection of various targets is disclosed in, for example, commonly assigned co-pending U.S. Provisional Patent Application Ser. No. 60/278,697 entitled “Dual Bead Assays for Detecting Medical Targets” filed Mar. 26, 2001, which is incorporated herein by reference in its entirety.

The target of interest can include tumor cells, bacteria, virus, or a target agent molecule such as a nucleic acid characteristic of a disease, or a nucleotide sequence specific for a person, or a nucleotide sequence or an antigenic determinant specific for an organism or cell type, which may be a bacterium, a virus, a mycoplasm, a fungus, a plant, or an animal. The target agent can include a nucleic acid molecule or antigenic determinant associated with cancer. The target nucleic acid molecule can include a nucleic acid, which is at least a portion of a gene selected from the group consisting of HER2neu, p52, p53, p21, and bcl-2. The target agent can be an antibody that is present only in a subject infected with HIV-1, a viral protein antigen, or a protein characteristic of a disease state in a subject. The methods and apparatus of the present invention can be used for determining whether a subject is infected by a virus, whether nucleic acid obtained from a subject exhibits a single nucleotide mutation (SNM) relative to corresponding wild-type nucleic acid sequence, or whether a subject expresses a protein of interest, such as a bacterial protein, a fungal protein, a viral protein, an HIV protein, a hepatitis C protein, a hepatitis B protein, or a protein known to be specifically associated with a disease. An example of a dual bead experiment detecting a nucleic acid target is presented below in Example 1.

According to another aspect of the invention, there is provided multiplexing methods wherein more than one target agent (e.g., tens, hundreds, or even thousands of different target agents) can be identified on one optical analysis disc. Multiple capture agents can be provided in a single chamber together in capture fields, or separately in separate capture fields. Different reporter beads can be used to be distinguishable from each other, such as beads that fluoresce at different wavelengths or different size reporter beads. Experiments were performed to identify two different targets using the multiplexing technique. An example of one such assay is discussed below in Example 2.

In accordance with yet another aspect, the invention includes an optical disc with a substrate, a capture layer associated with the substrate, and a capture agent bound to the capture layer, such that the capture agent binds to a dual bead complex. Multiple different capture agents can be used for different types of dual bead complexes. The disc can be designed to allow for some dual bead processing on the disc with appropriate chambers and fluidic structures, and can be pre-loaded with reporter and capture beads so that only a sample needs to be added to form the dual bead complex structures.

ODBMA Cell Analysis

According to one aspect of the current invention, there is provided a disc and disc drive system for performing bead or bead-cell assays. The disc drive can include an electromagnet for performing the isolation process, and may include appropriate light source control and detection for the type of reporter beads used. The disc drive can be optical or magneto-optical.

For processing performed on the disc, the drive may advantageously include an electromagnet, and the disc preferably has a mixing chamber, a waste chamber, and capture area. In this embodiment, the sample is mixed with beads in the mixing chamber, a magnetic field is applied adjacent the mixing chamber, and the sample not held by the magnet is directed to the waste chamber so that all magnetic beads, whether bound into a dual bead complex or unbound, remain in the mixing chamber. The magnetic beads are then directed to the capture area. One of a number of different valving arrangements can be used to control the flow.

The Magneto-Optical Bio-Disc System (MOBDS)

In another aspect of the present invention, a MOBMA is performed using MO discs made for use with biological samples and used in conjunction with a disc drive, such as a magneto-optical (MO) disc drive, that can selectively form magnetic domains or regions on a disc. In this MOBMA aspect of the present invention, magnetic domains can be formed in the MO disc in a highly controllable and precise manner. These domains may be employed advantageously to magnetically bind bio-magnetic particles like magnetic beads, including unbound magnetic capture beads, and magnetic complexes including dual bead complexes with magnetic capture beads, magnetic bead-cell complexes, or any biological or chemical complex having at least one magnetic particle or a magnetic property associated therewith so that these complexes may be bound using a magnet or magnetic domains. The MO disc drive can write to selected locations on the disc, and then use an optical reader to detect features located at those domains or regions. The domains can be selectively erased, thereby allowing individual beads and complexes to be selectively released. Biomedical applications related to using the MOBDS for analysis is described below.

In still another aspect of the invention, there is provided a method of using a bio-disc and drive including forming magnetic domains or regions on the optical bio-disc or medical CD. This method includes providing magnetic beads to the discs so that the beads bind at the magnetic domains. The method preferably further includes detecting at the locations where the magnetic beads bind biological samples, preferably using reporter beads that are detectable, such as by fluorescence or optical event detection. The method can be formed in multiple stages in terms of time or in terms of location through the use of multiple chambers. The domains are selectively written at pre-determined locations on the MO bio-disc and a sample is moved over the magnetic domains in order to capture magnetic beads or magnetic complexes. The regions can then be selectively erased and the magnetic beads or magnetic complexes released individually and relocated if desired. This method allows many different tests to be performed at one time, and can allow a level of interactivity between the user and the disc drives such that additional tests can be created during the testing process. Further details relating to magneto-optical recording, precise creation of magnetic regions on a magneto-optical disc, and magneto-optical detection methods are disclosed in, for example, U.S. Pat. No. 6,212,136 to Maeda at al., U.S. Pat. No. 5,329,503 to Ohmori et al., U.S. Pat. No. 4,985,881 to Saito et al., U.S. Pat. No. 4,843,604 to Fujiwara et al., and U.S. Pat. No. 4,748,606 to Naito et al. All of which are hereby incorporated by reference in their entireties.

The MO bio-disc may also be optimized fort different types of MOBMA applications including, for example, optimization of the optical properties of the magneto-optic stack of the MO bio-disc so that an incident tracking and detection beam of electromagnetic radiation is allowed to partially pass through the reflective layer and a transmitted beam detected using a separate detector and components of the operative layer of the MO bio-disc may be modified such that the magnetic field and strength of the magnetic field generated on the MO bio-disc is large and strong enough to capture and distinguish between magnetic complexes having different types of magnetic beads attached thereto. Furthermore, different types of electromagnetic radiation sources may be used in conjunction with the MOBDS including, but not limited to, infra-red, red, blue, and fluorescent type optical sources. The magnetic beads and magnetic complexes may be detected, for example, optically by analysis of the characteristics of the reflected and/or transmitted beam, as discussed below, for example, in conjunction with FIGS. 28A, 28B, 29A and 29B, or by fluorescence using a fluorescent type optical source in conjunction with a fluorescent detector and fluorescent labelled target molecules or cells.

The bio-magnetic bead assays according to the present invention may be implemented, in a genetic assay, herein referred to as the molecumagnetic assay, with magnetic capture beads and fluorescent reporter beads. These bio-magnetic beads or particles are coated with capture probes and reporter probes respectively. The capture probes and reporter probes are complementary to a target sequence but not to each other. The capture beads are mixed with varying quantities of target DNA. Unbound target is removed from the solution by magnetic concentration of the magnetic beads. Fluorescent reporter beads are then allowed to bind to the captured target DNA. Unbound reporter beads are removed by magnetic concentration of the magnetic beads. Thus, only in the presence of the target sequence, the magnetic capture beads bind to fluorescent reporter beads, resulting in a dual bead assay.

Capture Probe Binding

A number of different surface chemistries and different methods for binding the probes to the beads were investigated including covalently conjugating the capture or reporter probe onto carboxylated capture beads and reporter beads, respectively, via EDC conjugation. One observed result using the EDC conjugation method of attaching the probes on the beads was non-covalent attachment of the probes. This limitation was overcome by the development of a method for attaching the probes using partially double stranded DNA probes and by selection of an appropriate bead type with high conjugation efficiency. The use of double stranded probes in the conjugation process reduces the non-covalent attachment of probes to beads significantly. By using appropriate bead type and conjugation conditions, the covalent conjugation efficiency was as high as 95%. Details relating to DNA probe conjugation onto solid surfaces is disclosed in, for example, commonly assigned U.S. patent application Ser. No. 10/087,547, entitled “Methods for Decreasing Non-Specific Binding of Beads in Dual Bead Assays Including Related Optical Bio-discs and Disc Drive Systems”, filed Feb. 28, 2002, which is herein incorporated by reference in its entirety.

The use of magnetic beads in the capture of target DNA speeds up the washing steps and facilitates the separation steps between bound and unbound significantly. Furthermore, when the target concentration is limiting, each target molecule may hybridize to one reporter bead. Due to its size, a single target molecule is not detectable by any existing technologies. However, a 1 μm or larger reporter bead can be easily detected and quantified by various methods. Therefore, the bead assay increases the sensitivity of the target capture tremendously.

Bio-Disc Drive and Related Signal Processing Systems

In yet another principal aspect, the present invention also involves implementing the methods recited above on an analysis disc, modified optical disc, MOBDS, or a bio-disc. A bio-disc drive assembly may be employed to rotate the disc, read and process any encoded information stored on the disc, and analyze the test samples in a flow channel of the bio-disc. The bio-disc drive is thus provided with a motor for rotating the bio-disc, a controller for controlling the rate of rotation of the disc, a processor for processing return and/or transmitted signals form the disc, and an analyzer for analyzing the processed signals. The rotation rate of the motor is controlled to achieve the desired rotation of the disc. The bio-disc drive assembly may also be utilized to write information to the bio-disc either before or after the test material in the flow channel and target zones is interrogated by a read beam of the drive and analyzed by an analyzer. The bio-disc may include encoded information for controlling the rotation rate of the disc, providing processing information specific to the type of test to be conducted, and for displaying the results on a monitor associated with the bio-drive.

The various embodiments of the apparatus and methods of the present invention can be designed for use by an end-user, inexpensively, without specialized expertise and expensive equipment. The system can be made portable, and thus usable in remote locations where traditional diagnostic equipment may not generally be available. Other related aspects applicable to components of this assay system and signal acquisition methods are disclosed in commonly assigned and co-pending U.S. patent application Ser. No. 10/038,297 entitled “Dual Bead Assays Including Covalent Linkages For Improved Specificity And Related Optical Analysis Discs” filed Jan. 4, 2002; U.S. Provisional Application Ser. No. 60/272,525 entitled “Biological Assays Using Dual Bead Multiplexing Including Optical Bio-Disc and Related Methods” filed Mar. 1, 2001; and U.S. Provisional Application Ser. Nos. 60/275,643, 60/314,906, and 60/352,270 each entitled “Surface Assembly for Immobilizing Capture Agents and Dual Bead Assays Including Optical Bio-Disc and Methods Relating Thereto” respectively filed Mar. 14, 2001, Aug. 24, 2001, and Jan. 30, 2002. All of these applications are herein incorporated by reference in their entirety.

Other features and advantages will become apparent from the following detailed description, drawing figures, and technical examples.

### BRIEF DESCRIPTION OF THE DRAWING FIGURES

Further objects of the present invention together with additional features contributing thereto and advantages accruing therefrom will be apparent from the following description of preferred embodiments of the present invention which are shown in the accompanying drawing figures with like reference numerals indicating like components throughout, wherein:

FIG. 1 is a perspective view of an optical disc system according to the present invention;

FIG. 2 is a block and pictorial diagram of an optical reading system according to embodiments of the present invention;

FIGS. 3A, 3B, and 3C are respectively exploded, top, and perspective views of a reflective disc according to embodiments of the present invention;

FIGS. 4A, 4B, and 4C are respectively exploded, top, and perspective views of a transmissive disc according to embodiments of the present invention;

FIG. 5A is a partial longitudinal cross sectional view of the reflective optical bio-disc shown in FIGS. 3A, 3B, and 3C illustrating a wobble groove formed therein;

FIG. 5B is a partial longitudinal cross sectional view of the transmissive optical bio-disc illustrated in FIGS. 4A, 4B, and 4C showing a wobble groove formed therein and a top detector;

FIG. 6A is a partial radial cross-sectional view of the disc illustrated in FIG. 5A;

FIG. 6B is a partial radial cross-sectional view of the disc illustrated in FIG. 5B;

FIGS. 7A, 8A, 9A, and 10A are schematic representations of a capture bead, a reporter bead, and a dual bead complex as utilized in conjunction with genetic assays;

FIGS. 7B, 8B, 9B, and 10B are schematic representations of a capture bead, a reporter bead, and a dual bead complex as employed in conjunction with immunochemical assays;

FIG. 11A is a pictorial representation of one embodiment of a method for producing genetic dual bead complex solutions;

FIG. 11B is a pictorial representation of one embodiment of a method for producing immunochemical dual bead complex solutions;

FIG. 12A is a pictorial representation of another embodiment of a method for producing genetic dual bead complex solutions;

FIG. 12B is a pictorial representation of another embodiment of a method for producing immunochemical dual bead complex solutions;

FIG. 13 is a longitudinal cross sectional view illustrating the disc layers in combination with a mixing or loading chamber;

FIG. 14 is a view similar to FIG. 13 showing the mixing chamber loaded with dual bead complex solution;

FIGS. 15A and 15B are radial cross sectional views of the disc and target zone illustrating one embodiment for binding of reporter beads to capture agents in a genetic assay;

FIGS. 16A and 16B are radial cross sectional views of the disc and target zone showing another embodiment for binding of reporter beads to capture agents in a genetic assay;

FIG. 17 is radial cross sectional view of the disc and target zone illustrating one embodiment for binding of capture beads to capture agents in a genetic assay;

FIG. 18 is radial cross sectional view of the disc and target zone depicting another embodiment for binding of capture beads to capture agents in a genetic assay;

FIGS. 19A, 19B, and 19C are partial cross sectional views illustrating one embodiment of a method according to this invention for binding the reporter bead of a dual bead complex to a capture layer in a genetic assay;

FIGS. 20A, 20B, and 20C are partial cross sectional views showing one embodiment of a method according to the present invention for binding the reporter bead of a dual bead complex to a capture layer in a immunochemical assay;

FIGS. 21A, 21B, and 21C are partial cross sectional views illustrating another embodiment of a method according to this invention for binding the reporter bead of a dual bead complex to a capture layer in a genetic assay;

FIGS. 22A, 22B, and 22C are partial cross sectional views presenting another embodiment of a method according to the invention for binding the reporter bead of a dual bead complex to a capture layer in a immunochemical assay;

FIGS. 23A and 23B are partial cross sectional views depicting one embodiment of a method according to the present invention for binding the capture bead of a dual bead complex to a capture layer in a genetic assay;

FIGS. 24A and 24B are partial cross sectional views showing another embodiment of a method according to this invention for binding the capture bead of a dual bead complex to a capture layer in a genetic assay;

FIGS. 25A-25D illustrate a method according to the present invention for detecting the presence of target DNA or RNA in a genetic sample utilizing an optical bio-disc;

FIGS. 26A-26D illustrate another method according to this invention for detecting the presence of target DNA or RNA in a genetic sample utilizing an optical bio-disc;

FIGS. 27A-27D illustrate a method according to the present invention for detecting the presence of a target antigen in a biological test sample utilizing an optical bio-disc;

FIG. 28A is a graphical representation of an individual 2.1 um reporter bead and a 3 um capture bead positioned relative to the tracks of an optical bio-disc according to the present invention;

FIG. 28B is a series of signature traces derived from the beads of FIG. 28A utilizing a detected signal from the optical drive according to the present invention;

FIG. 29A is a graphical representation of a 2.1 um reporter bead and a 3 um capture bead linked together in a dual bead complex positioned relative to the tracks of an optical bio-disc according to the present invention;

FIG. 29B is a series of signature traces derived from the dual bead complex of FIG. 29A utilizing a detected signal from the optical drive according to this invention;

FIG. 30A is a bar graph showing results from a dual bead assay according to the present invention;

FIG. 30B is a graph showing a standard curve demonstrating the detection limit for fluorescent beads detected with a flourimeter;

FIG. 30C is a pictorial representation demonstrating the formation of the dual bead complex;

FIG. 31 is a bar graph showing the sensitivity of disc drive detection using a dual bead complex;

FIG. 32 is a schematic representation of combining beads for dual bead assay multiplexing according to embodiments of the present invention;

FIG. 33A is a schematic representation of a fluidic circuit according to the present invention utilized in conjunction with a magnetic field generator to control movement of magnetic beads;

FIGS. 33B-33D are schematics of a first fluidic circuit that implements the valving structure of FIG. 33A according to one embodiment of fluid transport aspects of the present invention;

FIGS. 34A-34C are schematics of a second fluidic circuit that implements the valving structure of FIG. 33A according to another embodiment of the fluid transport aspects of this invention;

FIG. 35 is a perspective view of the magnetic field generator and a disc including one embodiment of a fluidic circuit employed in conjunction with magnetic beads according to this invention;

FIGS. 36A, 36B, and 36C are plan views illustrating a method of separation and detection for dual bead assays using the fluidic circuit shown in FIG. 35;

FIG. 37 is a perspective view of a magneto-optical bio-disc showing magnetic domains or regions, magnetically bound capture beads, and the formation of dual bead complexes according to another aspect of the present invention;

FIG. 38 shows the use of ligation to form a covalent bond between the capture and reporter probes;

FIG. 39 is a bar graph showing the results from a genetic test detected by an enzyme assay in a ligation experiment;

FIG. 40 is a bar graph comparing the number of beads bound as a function of target concentration using 2.1 μm reporter beads with and without ligation;

FIG. 41 is a bar graph comparing the number of beads bound as a function of target concentration using a 39mer bridge with and without ligation;

FIG. 42A is schematic representation of various probe structures including DNA sequences for use in a dual bead complex employing cleavable or displaceable spacers according to the present invention;

FIG. 42B is pictorial diagrammatic representation showing a cleavable spacer connecting a dual bead complex prior to binding of a target;

FIG. 42C is a view similar or FIG. 42B illustrating the cleavable spacer including a NotI connecting the dual bead complex after target binding;

FIG. 42D is a view similar to FIG. 42C depicting the dual bead complex after target binding and after cleavage by NotI;

FIG. 43A is pictorial diagrammatic representation showing a displaceable spacer connecting a dual bead complex prior to binding of a target;

FIG. 43B is a view similar to FIG. 43A illustrating initial binding of a displacement probe to the displaceable spacer connecting the dual bead complex after target binding;

FIG. 43C is a view similar to FIG. 43B depicting complete displacement of the displacement probe connecting the dual bead complex in the presence of target mediated binding;

FIG. 44 is a pictorial representation of cleavable spacers covalently attached to a capture bead according to the present invention;

FIG. 45 is a view similar to FIG. 44 showing thiol groups attached to the cleavable spacers binding covalently to a metallic reporter bead;

FIG. 46A is a pictorial representation of a pair of dual bead complexes bound together by a cleavable spacer before target binding;

FIG. 46B is a view similar to FIG. 46A showing the dual bead complexes bound together by the cleavable spacer after target binding and without target binding;

FIG. 46C is a view similar to FIG. 46B showing one of the dual bead complexes dissociated after enzyme cleavage and the other held together by the presence of the target;

FIG. 47A is a pictorial presentation of a dual bead complex formed by a pair of cleavable spacers and use of a bridge bound to a target;

FIG. 47B is a view similar to FIG. 47A after target binding including the bridge resulting in a double helix containing two breaks;

FIG. 47C is a view similar to FIG. 47B after restriction digestion of the cleavable spacers and ligation of the breaks in the double helix;

FIG. 48A is a pictorial representation of two dual bead complexes each joined together by a pair of cleavable spacers as implemented in an immunochemical assay prior to target antigen binding;

FIG. 48B is a view similar to FIG. 48A showing the dual bead complexes bound together by the cleavable spacer with and without target binding;

FIG. 48C is a view similar to FIG. 48B illustrating one of the dual bead complexes dissociated after enzyme digestion and the other held together by the presence of the target;

FIG. 49 is a schematic presenting a method for evaluating a solid phase for covalent conjugation of a probe;

FIG. 50 is a schematic detailing various steps in the quantification of covalently-bound and non-covalently bound probes to a solid substrate;

FIG. 51A is a graphic presentation of experimental results of various tests of magnetic bead carriers for covalent linkage of a probe;

FIG. 51B is a graphic presentation of experimental results of various tests of fluorescent bead carriers for covalent linkage of a probe;

FIG. 52A is a pictorial representation illustrating the structural differences between single-stranded and double-stranded DNA that are relevant to their use as probes;

FIG. 52B is a graphic presentation of results of an experiment designed to evaluate the binding properties of single-stranded and double-stranded DNA to a solid phase;

FIG. 53A is graphic presentation of enzyme assay results of a screen of two different capture beads for use in a dual bead assay, these results indicating that both of the tested beads bind a similar amount of target regardless of whether the probe is bound covalently or non-covalently;

FIG. 53B is a graphic presentation of results of a dual bead assay designed to examine the number of reporter beads captured by two different capture beads, these results indicate that covalent bonding of the probe to the capture bead greatly improves assay sensitivity;

FIG. 54 is a graphic presentation demonstrating that the introduction of PEG linkers into probes significantly improves target mediated binding;

FIG. 55 is a bar graph presentation illustrating probe density determination employing 3 μm beads;

FIG. 56 is a bar graph presentation demonstrating the pretreatment of the beads with various detergents including salmon sperm DNA which reduced nonspecific binding by over 10 fold;

FIG. 57 is a bar graph presentation showing the range of detection of the dual bead assay;

FIG. 58 is a bar graph illustrating the use of NaCl in varying concentrations and the related non-specific binding;

FIG. 59 is a bar graph presentation showing increasing EDTA concentration and the related non-specific binding;

FIG. 60 is a bar graph presentation depicting an increasing NaCl concentration and the related non-specific binding;

FIGS. 61A and 61B are bar graph presentations illustrating an increasing concentration of MgCl₂ and related non-specific binding;

FIG. 62 is a pictorial schematic representation showing the use of probe blocking agents to increase the sensitivity of the bead assay;

FIG. 63 is a bar graph presentation illustrating the effect of incubation time during a hybridization reaction;

FIG. 64 is a bar graph presentation showing a mixing method directed to increasing efficiency in dual bead binding;

FIGS. 65A and 65B together comprise a pictorial representation of another embodiment of a method for producing genetic dual bead complex solutions related to the method discussed in connection with FIG. 11A;

FIGS. 66A and 66B taken together form a pictorial representation of another embodiment of a method for producing immunochemical dual bead complex solutions similar to that shown in FIG. 11B;

FIGS. 67A and 67B together show a pictorial representation of yet another embodiment of a method for producing genetic dual bead complex solutions, this method being related to the method illustrated in FIG. 12A;

FIGS. 68A and 68B taken together illustrate a pictorial representation of still another embodiment of a method for producing immunochemical dual bead complex solutions which is similar to that shown in FIG. 12B;

FIG. 69 is a bar graph presentation demonstrating the effect of DNAseI digestion in absence of reporter beads;

FIG. 70 is a bar graph presentation showing the efficiency of dual bead assay by the effect of DNAseI enzymes digestion;

FIG. 71 is a schematic representation of separation of reporter beads from capture beads by enzyme digestion and physical or chemical treatments;

FIG. 72 is a bar graph presentation showing dual bead complexes prior to and after washing in a basic solution;

FIG. 73A is a bar graph presentation illustrating dual bead complexes prior to and after washing in a 7M urea solution;

FIG. 73B is a bar graph presentation representing dual bead complexes prior to and after washing in a 7M urea solution including the detection of dissociated reporter beads after the urea wash;

FIG. 74 is a bar graph presentation demonstrating the use of 1.5M guanidine isothiocyanate as a denaturing agent during dual bead assay; and

FIG. 75 is a bar graph presentation showing the varying concentrations of guanidine isothiocyanate employed as a denaturing agent during dual bead assay;

FIG. 76 is a top plan view of a portion of a magneto-optical bio-disc having fluidic circuits; and

FIGS. 77A-77E are plan views illustrating a method of separating and testing cells using the fluidic circuit shown in FIG. 76.

### DETAILED DESCRIPTION OF THE PREFERRED EMBODIMENTS

The following description of the present invention relates to optical analysis discs, disc drive systems, optical disc biomagnetic assays, and assay chemistries and techniques. The invention further relates to alternate magneto-optical drive systems, MO bio-discs, MO bio-disc systems, MO biomagnetic assays, and related processing methods.

Disc Drive System and Related Optical Analysis Discs

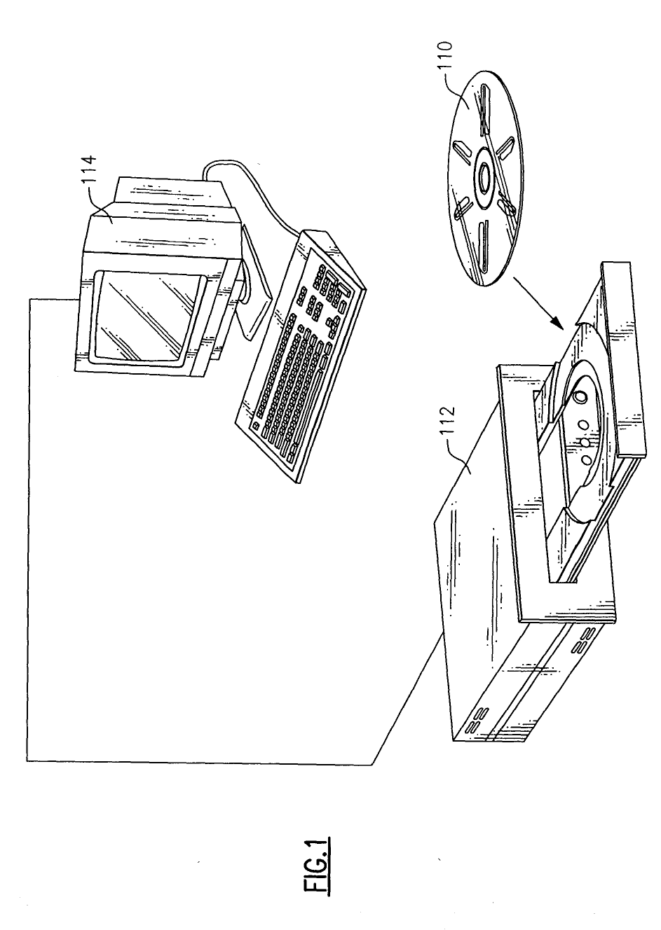

With reference now to FIG. 1, there is shown a perspective view of an optical analysis disc, optical bio-disc, or medical CD 110 for use in an optical disc drive 112. Drive 112, in conjunction with software in the drive or associated with a separate computer, can cause images, graphs, or output data to be displayed on display monitor 114. As indicated below, there are different types of discs and drives that can be used including, but not limited to, magneto-optical discs and magneto-optical disc drives. The disc drive can be in a unit separate from a controlling computer, or provided in a bay within a computer. The device can be made as portable as a laptop computer, and thus usable with battery power and in remote locations not generally served by advanced diagnostic equipment. The drive is preferably a conventional drive with minimal or no hardware modification, but can be a dedicated bio-disc or medical CD drive. Further details regarding these types of drive systems and related signal processing methods are disclosed in, for example, commonly assigned and co-pending U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S; and U.S. patent application Ser. No. 10/043,688 entitled “Optical Disc Analysis System Including Related Methods For Biological and Medical Imaging” filed Jan. 10, 2002. These applications are herein incorporated by reference in their entirety.

Optical bio-disc 110 for use with embodiments of the present invention may have any suitable shape, diameter, or thickness, but preferably is implemented on a round disc with a diameter and a thickness similar to those of a compact disc (CD), a recordable CD (CD-R), CD-RW, a digital versatile disc (DVD), DVD-R, DVD-RW, a magneto-optical disc, or other standard optical disc format. The disc may include encoded information, preferably in a known format, for performing, controlling, and post-processing a test or assay, such as information for controlling the rotation rate and direction of the disc, timing for rotation, stopping and starting, delay periods, locations of samples, position of the light source, and power of the light source. Such encoded information is referred to generally here as operational information.

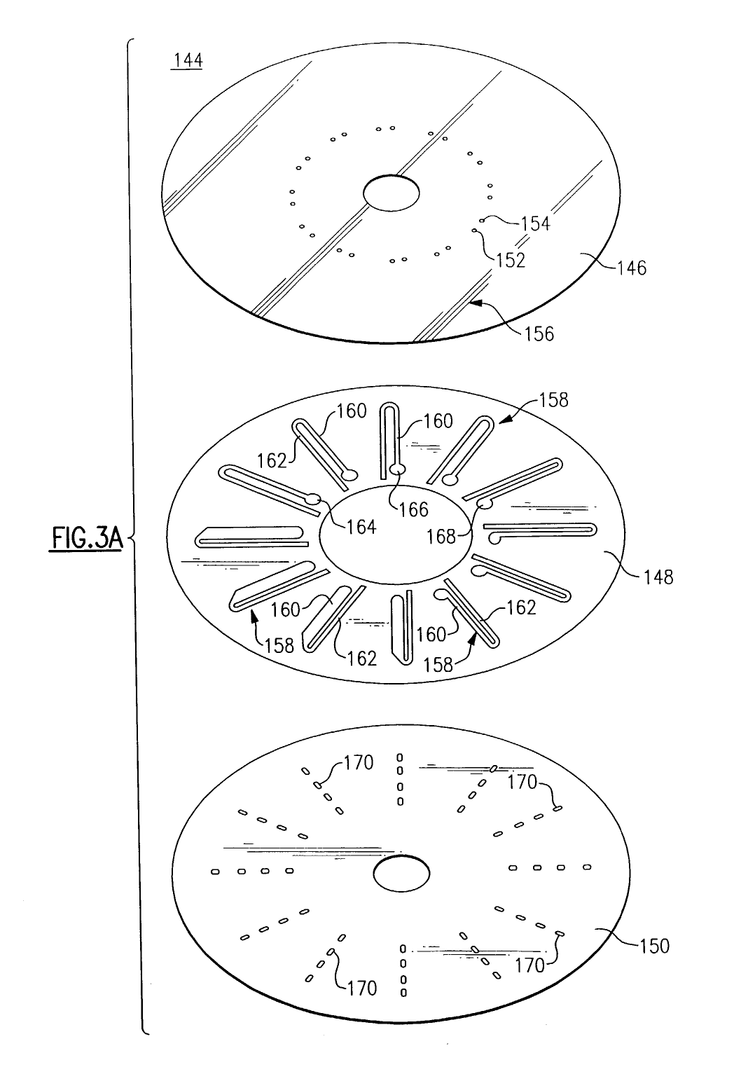

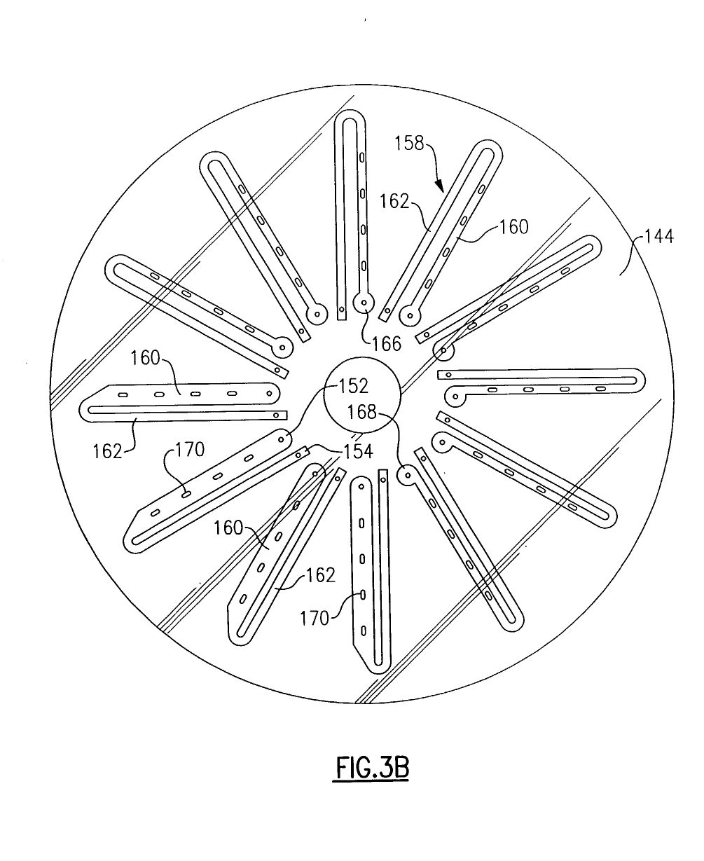

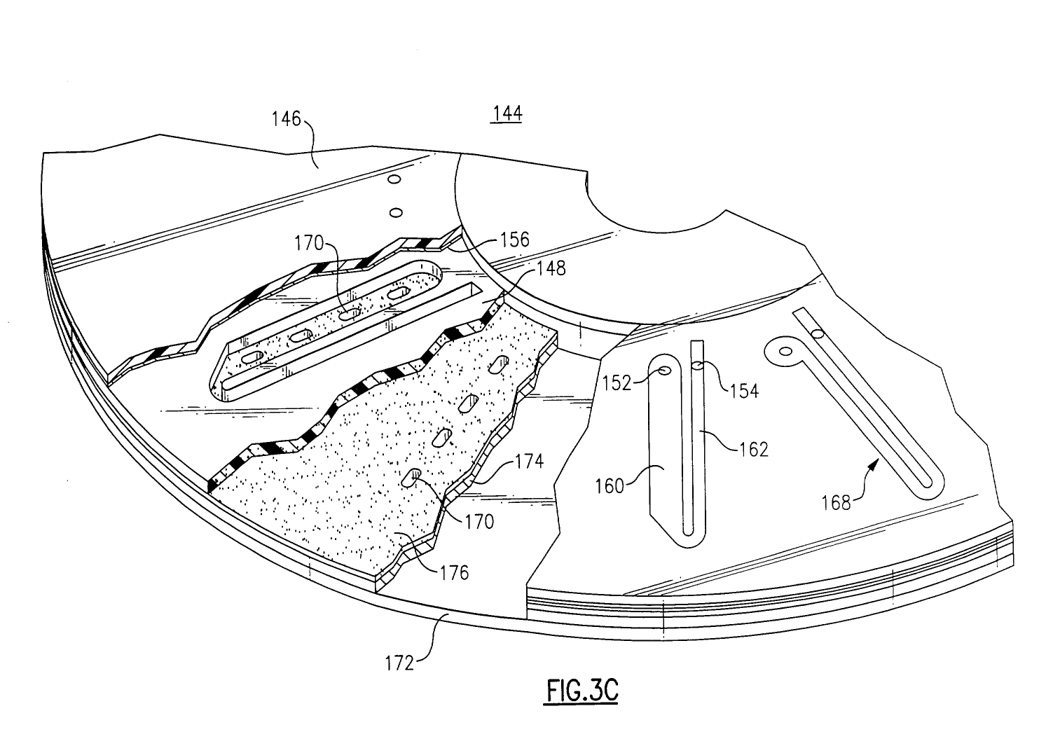

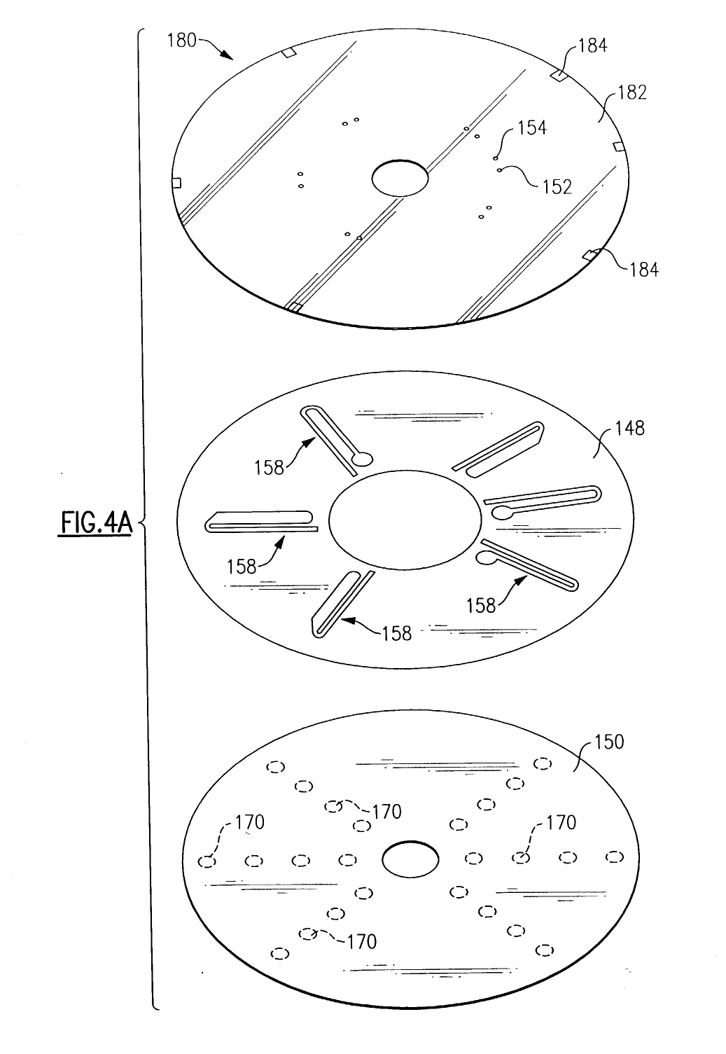

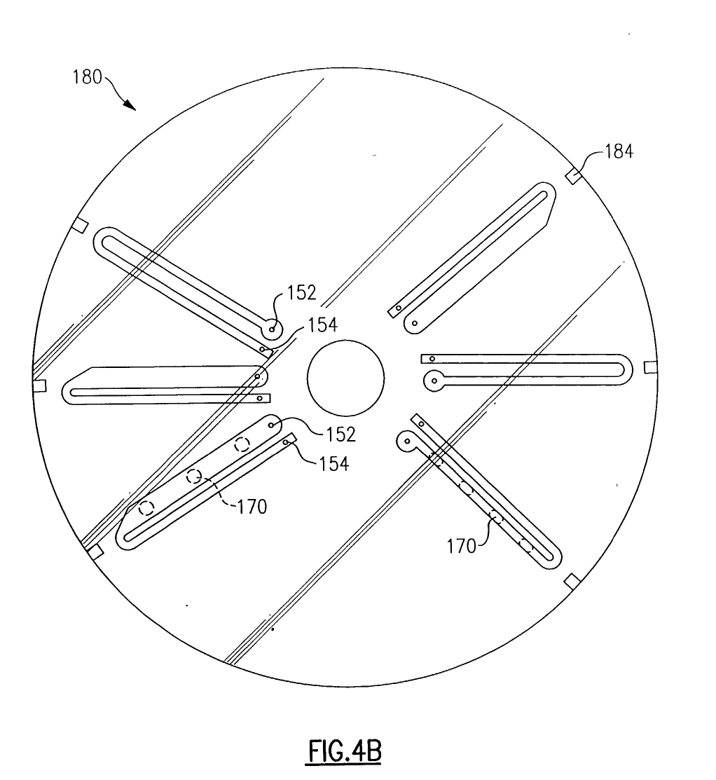

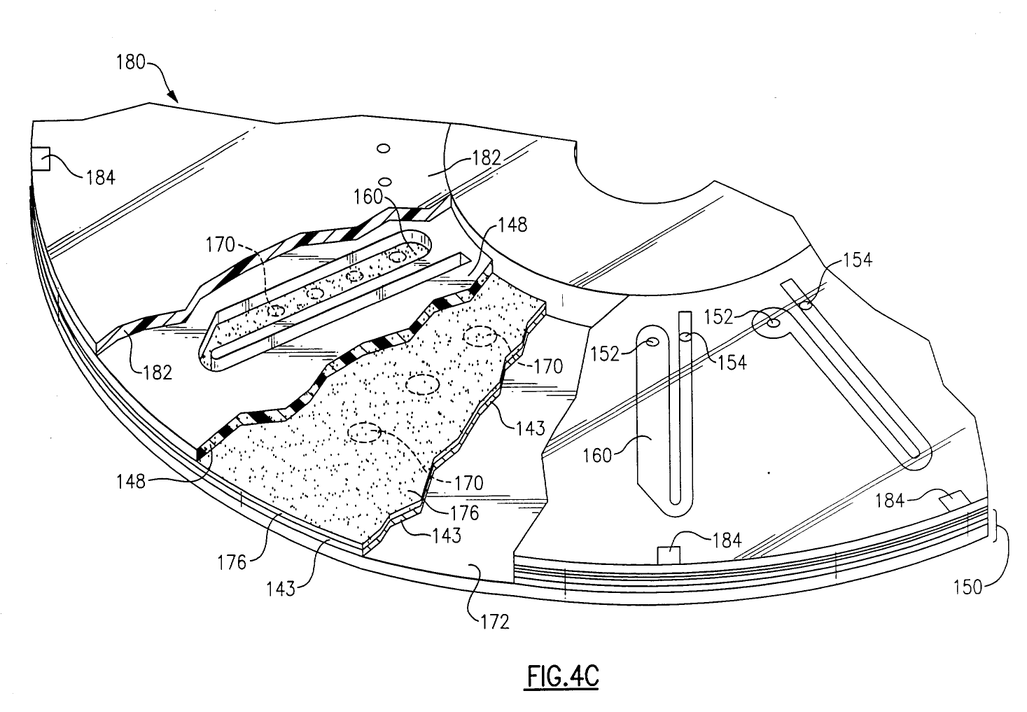

The disc may be a reflective disc, as shown in FIGS. 3A-3C, a transmissive disc, FIGS. 4A-4C, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light.

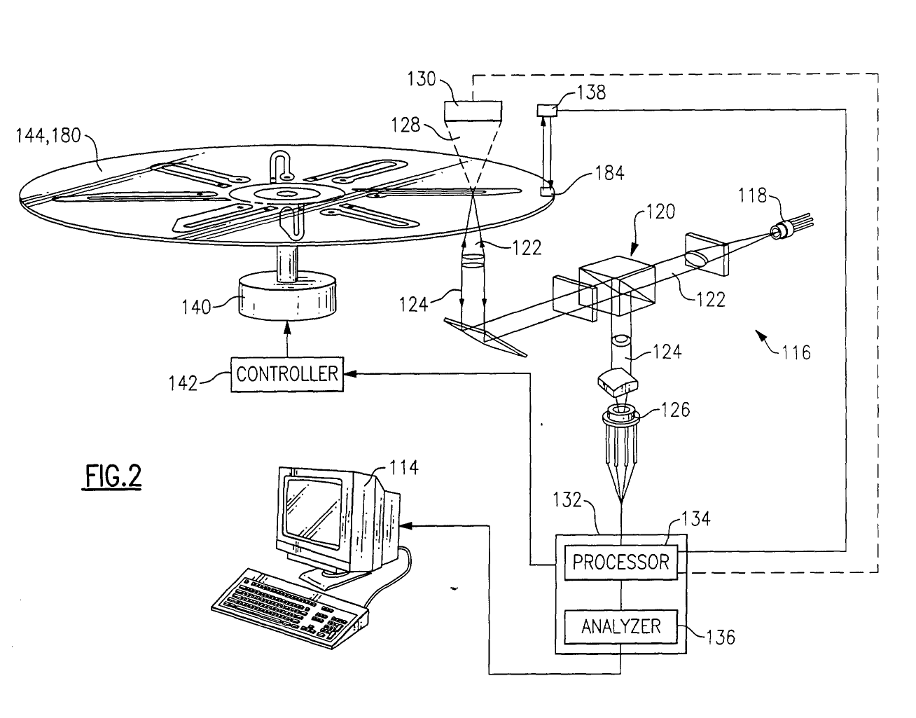

FIG. 2 shows an optical disc reader system 116. This system may be a conventional reader for CD, CD-R, DVD, MO, or other known comparable format, a modified version of such a drive, or a completely distinct dedicated device. The basic components are a motor for rotating the disc, a light system for providing light, and a detection system for detecting light.

With reference now generally to FIGS. 2-4C, a light source 118 provides light to optical components 120 to produce an incident light beam 122. In the case of reflective disc 144, FIGS. 3A-3C, a return beam 124 is reflected from either reflective surface 156, 174, or 186, FIGS. 3C and 4C. Return beam 124 is provided back to optical components 120, and then to a bottom detector 126. In this type of disc, the return beam may carry operational information or other encoded data as well as characteristic information about the investigational feature or test sample under study.

For transmissive disc 180, FIGS. 4A-4C, some of the energy from the incident beam 122 will undergo a light/matter interaction with an investigational feature or test sample and then proceed through the disc as a transmitted beam 128 that is detected by a top detector 130. For a transmissive disc including a semi-reflective layer 186 (FIG. 4C) as the operational layer, some of the energy from the incident beam 122 will also reflect from the operational layer as return beam 124, which carries operational information or stored data. Optical components 120 can include a lens, a beam splitter, and a quarter wave plate that changes the polarization of the light beam so that the beam splitter directs a reflected beam through the lens to focus the reflected beam onto the detector. An astigmatic element, such as a cylindrical lens, may be provided between the beam splitter and detector to introduce astigmatism in the reflected light beam. The light source can be controllable to provide variable wavelengths and power levels over a desired range in response to data introduced by the user or read from the disc. This controllability is especially useful when it is desired to detect multiple different structures that fluoresce at different wavelengths.

Now with continuing reference to FIG. 2, it is shown that data from detector 126 and/or detector 130 is provided to a computer 132 including a processor 134 and an analyzer 136. An image or output results can then be provided to a monitor 114. Computer 132 can represent a desktop computer, programmable logic, or some other processing device, and also can include a connection (such as over the Internet) to other processing and/or storage devices. A drive motor 140 and a controller 142 are provided for controlling the rotation rate and direction or rotation of disc the 144 or 180. Controller 142 and the computer 132 with processor 134 can be in remote communication or implemented in the same computer. Methods and systems for reading such a disc are also shown in Gordon, U.S. Pat. No. 5,892,577, which is incorporated herein by reference.

The detector can be designed to detect all light that reaches the detector, or light only at specific wavelengths, through its design or an external filter. By making the detector controllable in terms of the detectable wavelength, beads or other structures that fluoresce at different wavelengths can be separately detected.

A hardware trigger sensor 138 may be used with either a reflective disc 144 or transmissive disc 180. Triggering sensor 138 provides a signal to computer 132 (or to some other electronics) to allow for the collection of data by processor 134 only when incident beam 122 is on a target zone or inspection area. Alternatively, software read from a disc can be used to control data collection by processor 134 independent of any physical marks on the disc. Such software or logical triggering is discussed in further detail in commonly assigned and co-pending U.S. Provisional Application Ser. No. 60/352,625 entitled “Logical Triggering Methods And Apparatus For Use With Optical Analysis Discs And Related Disc Drive Systems” filed Jan. 28, 2002, which is herein incorporated by reference in its entirety.

The substrate layer of the optical analysis disc may be impressed with a spiral track that starts at an innermost readable portion of the disc and then spirals out to an outermost readable portion of the disc. In a non-recordable CD, this track is made up of a series of embossed pits with varying length, each typically having a depth of approximately one-quarter the wavelength of the light that is used to read the disc. The varying lengths and spacing between the pits encode the operational data. The spiral groove of a recordable CD-like disc has a detectable dye rather than pits. Furthermore, in the MO disc, the data is stored in magnetic domains created on the MO disc. This is where the operation information, such as the rotation rate, is recorded. Depending on the test, assay, or investigational protocol, the rotation rate may be variable with intervening or consecutive periods of acceleration, constant speed, and deceleration. These periods may be closely controlled both as to speed and time of rotation to provide, for example, mixing, agitation, or separation of fluids and suspensions with agents, reagents, antibodies, or other materials. Different optical analysis disc, medical CD, and bio-disc designs that may be utilized with the present invention, or readily adapted thereto, are disclosed, for example, in commonly assigned, co-pending U.S. patent application Ser. No. 09/999,274 entitled “Optical Bio-discs with Reflective Layers” filed on Nov. 15, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; and U.S. patent application Ser. No. 10/006,619 entitled “Optical Disc Assemblies for Performing Assays” filed Dec. 10, 2001, which are all herein incorporated by reference in their entirety.

Numerous designs and configurations of an optical pickup and associated electronics may be used in the context of the embodiments of the present invention. Further details and alternative designs for compact discs and readers are described in Compact Disc Technology, by Nakajima and Ogawa, IOS Press, Inc. (1992); The Compact Disc Handbook, Digital Audio and Compact Disc Technology, by Baert et al. (eds.), Books Britain (1995); and CD-Rom Professional's CD-Recordable Handbook: The Complete Guide to Practical Desktop CD, Starrett et al. (eds.), ISBN:0910965188 (1996); all of which are incorporated herein in their entirety by reference.

The disc drive assembly is thus employed to rotate the disc, read and process any encoded operational information stored on the disc, and analyze the liquid, chemical, biological, or biochemical investigational features in an assay region of the disc. The disc drive assembly may be further utilized to write information to the disc either before, during, or after the material in the assay zone is analyzed by the read beam of the drive. In alternate embodiments, the disc drive assembly is implemented to deliver assay information through various possible interfaces such as via Ethernet to a user, over the Internet, to remote databases, or anywhere such information could be advantageously utilized. Further details relating to this type of disc drive interfacing are disclosed in commonly assigned co-pending U.S. patent application Ser. No. 09/986,078 entitled “Interactive System For Analyzing Biological Samples And Processing Related Information And The Use Thereof” filed Nov. 7, 2001, which is incorporated herein by reference in its entirety.

Referring now specifically to FIGS. 3A, 3B, and 3C, the reflective disc 144 is shown with a cap 146, a channel layer 148, and a substrate 150. The channel layer 148 may be formed by a thin-film adhesive member. Cap 146 has inlet ports 152 for receiving samples and vent ports 154. Cap 146 may be formed primarily from polycarbonate, and may be coated with a cap reflective layer 156 on the bottom thereof. Reflective layer 156 is preferably made from a metal such as aluminum or gold.

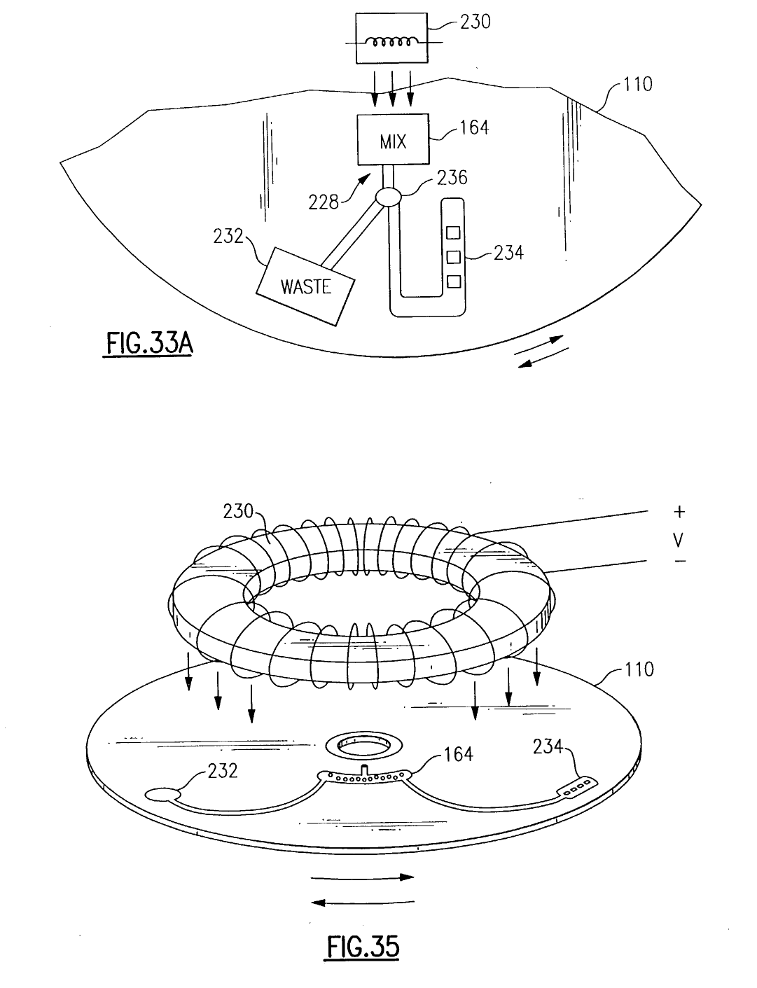

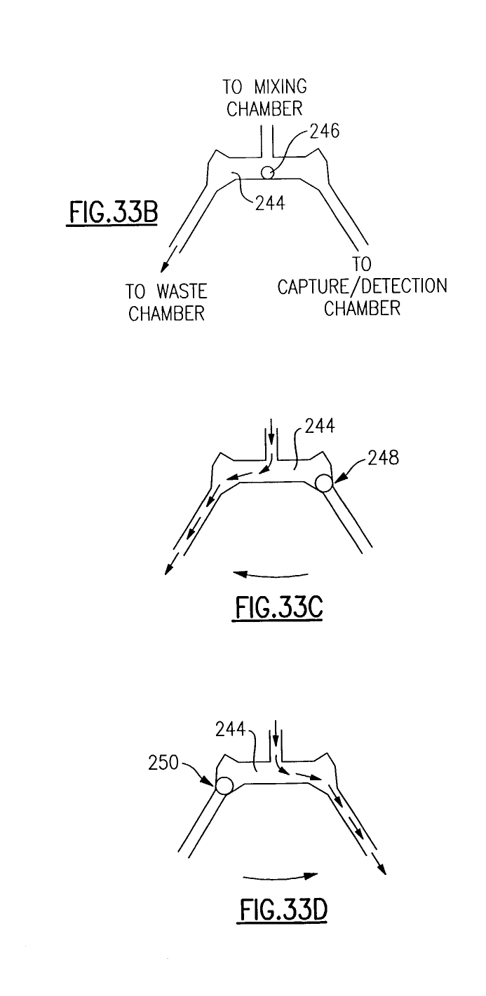

Channel layer 148 defines fluidic circuits 158 by having desired shapes cut out from channel layer 148. Each fluidic circuit 158 preferably has a flow channel 160 and a return channel 162, and some have a mixing chamber 164. A mixing chamber 166 can be symmetrically formed relative to the flow channel 160, while an off-set mixing chamber 168 is formed to one side of the flow channel 160. Fluidic circuits 158 are rather simple in construction, but a fluidic circuit can include other channels and chambers, such as preparatory regions or a waste region, as shown, for example, in U.S. Pat. No. 6,030,581 entitled “Laboratory in a Disc” which is incorporated herein by reference. These fluidic circuits can include valves and other fluid control structures such as those alternatively employed herein and discussed in further detail in connection with FIGS. 33A-33D, 34A-34C, 35, and 36A-36C. Channel layer 148 can include adhesives for bonding to the substrate and to the cap.

Substrate 150 has a plastic layer 172, and has target zones 170 formed as openings in a substrate reflective layer 174 deposited on the top of layer 172. In this embodiment, reflective layer 174, best illustrated in FIG. 3C, is used to encode operational information. The reflective layer 174 is not limited to a single layer but may also be several stacks of reflective layers on the substrate 150 such as optical stacks on an MO disc, for example. Plastic layer 172 is preferably formed from polycarbonate. Target zones 170 may be formed by removing portions of the substrate reflective layer 174 in any desired shape, or by masking target zone areas before applying substrate reflective layer 174. The substrate reflective layer 174 is preferably formed from a metal, such as aluminum, gold, or magnetic alloys, and can be configured with the rest of the substrate to encode operational information that is read with incident light, such as through a wobble groove or through an arrangement of pits. Light incident from under substrate 150 thus is reflected by layer 174, except at target zones 170, where it is reflected by layer 156. Target zones are where investigational features are detected. If the target zone is a location where an antibody, strand of DNA, or other material that can bind to a target is located, the target zone can be referred to as a capture zone.

With reference now particularly to FIG. 3C, optical disc 144 is cut away to illustrate a partial cross-sectional perspective view. An active layer 176 is formed over substrate reflective layer 174. Active layer 176 may generally be formed from nitrocellulose, polystyrene, polycarbonate, gold, activated glass, modified glass, or a modified polystyrene such as, for example, polystyrene-co-maleic anhydride. In this embodiment, channel layer 148 is situated over active layer 174.

In operation, samples can be introduced through inlet ports 152 of cap 146. When rotated, the sample moves outwardly from inlet port 152 along active layer 176. Through one of a number of biological or chemical reactions or processes, detectable features, referred to as investigational features, may be present in the target zones. Examples of such processes are shown in the incorporated U.S. Pat. No. 6,030,581 and in commonly assigned, co-pending U.S. patent application Ser. No. 09/988,728 entitled “Methods And Apparatus For Detecting And Quantifying Lymphocytes With Optical Biodiscs” filed Nov. 16, 2001; and U.S. patent application Ser. No. 10/035,836 entitled “Surface Assembly For Immobilizing DNA Capture Probes And Bead-Based Assay Including Optical Bio-Discs And Methods Relating Thereto” filed Dec. 21, 2001, both of which are herein incorporated by reference in their entireties.

The investigational features captured within the target zones, by the capture layer with a capture agent, may be designed to be located in the focal plane coplanar with reflective layer 174, where an incident beam is typically focused in conventional readers. Alternatively, the investigational features may be captured in a plane spaced away from the focal plane. The former configuration is referred to as a “proximal” type disc, and the latter a “distal” type disc.

Referring to FIGS. 4A, 4B, and 4C, it is shown that one particular embodiment of the transmissive optical disc 180 includes a clear cap 182, a channel layer 148, and a substrate 150. The clear cap 182 includes inlet ports 152 and vent ports 154 and is preferably formed mainly from polycarbonate. Trigger marks 184 may be included on the cap 182. Channel layer 148 has fluidic circuits 158, which can have structure and use similar to those described in conjunction with FIGS. 3A, 3B, and 3C. Substrate 150 may include target zones 170, and preferably includes a polycarbonate layer 172. Substrate 150 may, but need not, have a thin semi-reflective layer 186 deposited on top of layer 172. Semi-reflective layer 186 is preferably significantly thinner than substrate reflective layer 174 on substrate 150 of reflective disc 144 (FIGS. 3A-3C). The semi-reflective layer 186 is not limited to a single layer but may also be several stacks of semi-reflective layers on the substrate 150 such as optical stacks on an MO disc, for example. Semi-reflective layer 186 is preferably formed from a metal, such as aluminum, gold, or magnetic alloys, but is sufficiently thin to allow a portion of an incident light beam to penetrate and pass through layer 186, while some of the incident light is reflected back. A gold film layer, for example, is 95% reflective at a thickness greater than about 700 Å, while the transmission of light through the gold film is about 50% transmissive at approximately 100 Å.

FIG. 4C is a cut-away perspective view of transmissive disc 180. The semi-reflective nature of layer 186 makes its entire surface potentially available for target zones, including virtual zones defined by trigger marks or encoded data patterns on the disc. Target zones 170 may also be formed by marking the designated area in the indicated shape or alternatively in any desired shape. Markings to indicate target zone 170 may be made on semi-reflective layer 186 or on a bottom portion of substrate 150 (under the disc). Target zones 170 may be created by silk screening ink onto semi-reflective layer 186.

An active layer 176 is applied over semi-reflective layer 186. Active layer 176 may be formed from the same materials as described above in conjunction with layer 176 (FIG. 3C) and serves substantially the same purpose when a sample is provided through an opening in disc 180 and the disc is rotated. In transmissive disc 180, there is no reflective layer, on the clear cap 182, comparable to reflective layer 156 in reflective disc 144 (FIG. 3C).

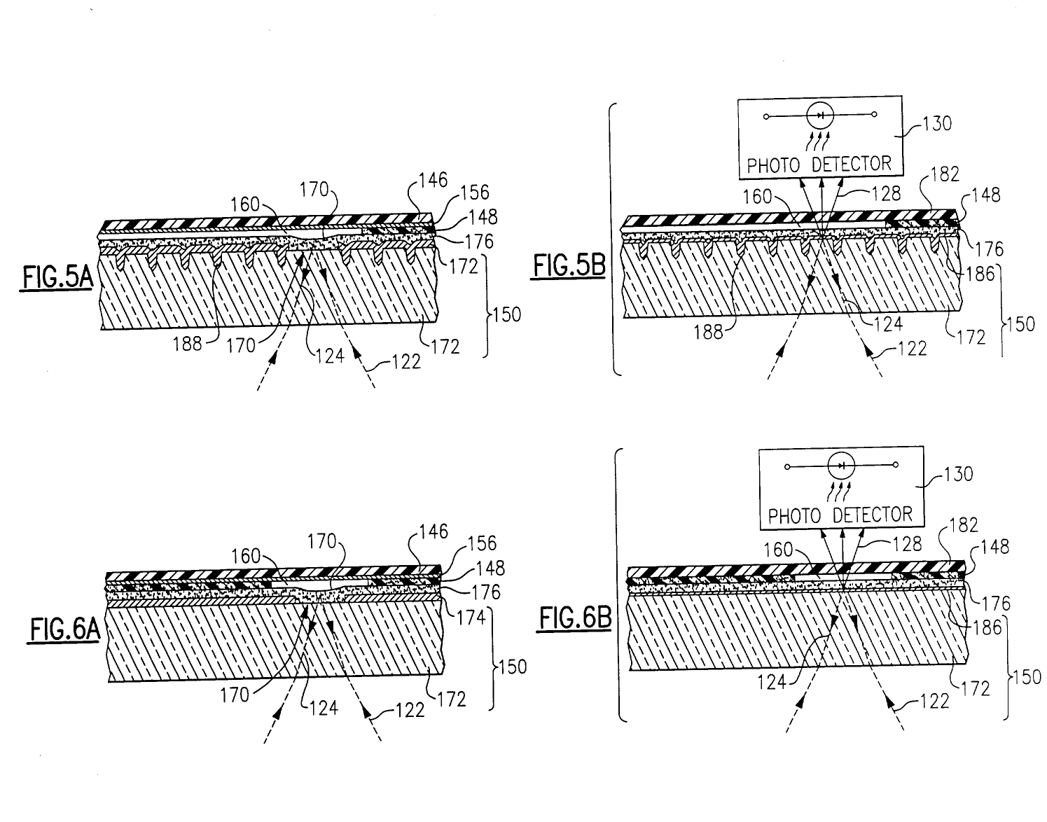

Referring now to FIG. 5A, there is shown a cross sectional view taken across the tracks of the reflective disc embodiment 144 (FIGS. 3A-3C) of the bio-disc 110 (FIG. 1) according to the present invention. As illustrated, this view is taken longitudinally along a radius and flow channel of the disc. FIG. 5A includes the substrate 150 that is composed of a plastic layer 172 and a substrate reflective layer 174. In this embodiment, the substrate 150 includes a series of grooves 188. The grooves 188 are in the form of a spiral extending from near the center of the disc toward the outer edge. The grooves 188 are implemented so that the interrogation or incident beam 122 may track along the spiral grooves 188 on the disc. This type of groove 188 is known as a “wobble groove”. The groove 188 is formed by a bottom portion having undulating or wavy side walls. A raised or elevated portion separates adjacent grooves 188 in the spiral. The reflective layer 174 applied over the grooves 188 in this embodiment is, as illustrated, conformal in nature. FIG. 5A also shows the active layer 176 applied over the reflective layer 174. As shown in FIG. 5A, the target zone 170 is formed by removing an area or portion of the reflective layer 174 at a desired location or, alternatively, by masking the desired area prior to applying the reflective layer 174. As further illustrated in FIG. 5A, the plastic adhesive member or channel layer 148 is applied over the active layer 176. FIG. 5A also shows the cap portion 146 and the reflective surface 156 associated therewith. Thus, when the cap portion 146 is applied to the plastic adhesive member 148 including the desired cut-out shapes, the flow channel 160 is thereby formed.

FIG. 5B is a cross sectional view, similar to that illustrated in FIG. 5A, taken across the tracks of the transmissive disc embodiment 180 (FIGS. 4A-4C) of the bio-disc 110 (FIG. 1) according to the present invention. This view is taken longitudinally along a radius and flow channel of the disc. FIG. 5B illustrates the substrate 150 that includes the thin semi-reflective layer 186. This thin semi-reflective layer 186 allows the incident or interrogation beam 122, from the light source 118 (FIG. 2), to penetrate and pass through the disc to be detected by the top detector 130, while some of the light is reflected back in the form of the return beam 124. The thickness of the thin semi-reflective layer 186 is determined by the minimum amount of reflected light required by the disc reader to maintain its tracking ability. The substrate 150 in this embodiment, like that discussed in FIG. 5A, includes the series of grooves 188. The grooves 188 in this embodiment are also preferably in the form of a spiral extending from near the center of the disc toward the outer edge. The grooves 188 are implemented so that the interrogation beam 122 may track along the spiral. FIG. 5B also shows the active layer 176 applied over the thin semi-reflective layer 186. As further illustrated in FIG. 5B, the plastic adhesive member or channel layer 148 is applied over the active layer 176. FIG. 5B also shows the clear cap 182. Thus, when the clear cap 182 is applied to the plastic adhesive member 148 including the desired cut-out shapes, the flow channel 160 is thereby formed and a part of the incident beam 122 is allowed to pass therethrough substantially unreflected. The amount of light that passes through can then be detected by the top detector 130.

FIG. 6A is a view similar to FIG. 5A but taken perpendicularly to a radius of the disc to illustrate the reflective disc and the initial refractive property thereof when observing the flow channel 160 from a radial perspective. In a parallel comparison manner, FIG. 6B is a similar view to FIG. 5B but taken perpendicularly to a radius of the disc to represent the transmissive disc and the initial refractive property thereof when observing the flow channel 160 from a radial perspective. Grooves 188 are not seen in FIGS. 5A and 5B since the sections are cut along the grooves 188. FIGS. 6A and 6B show the presence of the narrow flow channel 160 that is situated perpendicular to the grooves 188 in these embodiments. FIGS. 5A, 5B, 6A, and 6B show the entire thickness of the respective reflective and transmissive discs. In these views, the incident beam 122 is illustrated initially interacting with the substrate 150 which has refractive properties that change the path of the incident beam as shown to provide focusing of the beam 122 on the reflective layer 174 or the thin semi-reflective layer 186.

The fluidic circuits 158 may also be configured in the equi-radial or “e-rad” format disclosed in commonly assigned and copending U.S. Provisional Application Ser. No. 60/353,014 entitled “Optical Discs Including Equi-Radial and/or Spiral Analysis Zones and Related Disc Drive Systems and Methods” filed Jan. 29, 2002, which is hereby incorporated by reference in its entirety.

Assay Chemistries and Dual Bead Formation

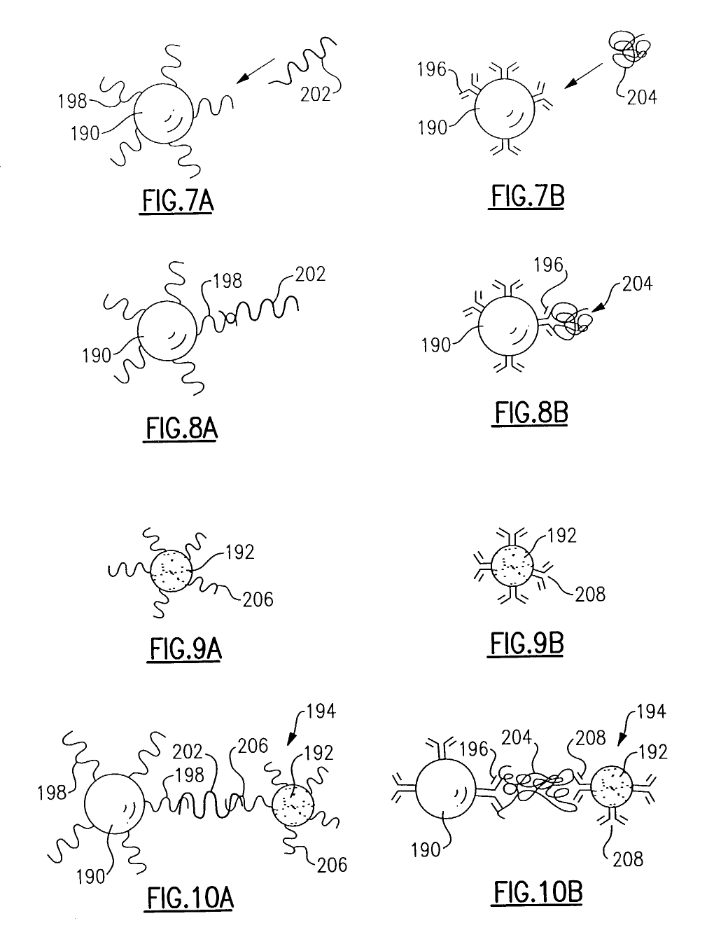

Referring now to FIGS. 7A-10A and 7B-10B, there is shown a capture bead 190, a reporter bead 192, and the formation of a dual bead complex 194. Capture bead 190 can be used in conjunction with a variety of different assays including biological assays such as immunoassays (FIGS. 7B-10B), molecular assays, and more specifically genetic assays (FIGS. 7A-10A). In the case of immunoassays, antibody transport probes 196 are conjugated onto the beads. Antibody transport probes 196 include proteins, such as antigens or antibodies, implemented to capture protein targets. In the case of molecular assays, oligonucleotide transport probes 198 would be conjugated onto the beads. Oligonucleotide transport probes 198 include nucleic acids such as DNA or RNA implemented to capture genetic targets.

As shown in FIG. 7A, a target agent such as target DNA or RNA 202, obtained from a test sample, is added to a capture bead 190 coated with oligonucleotide transport probes 198. In this implementation, transport probes 198 are formed from desired sequences of nucleic acids. Aspects relating to DNA probe conjugation onto solid phase of this system of assays are discussed in further detail in commonly assigned and co-pending U.S. Provisional Application Ser. No. 60/278,685 entitled “Use of Double Stranded DNA for Attachment to Solid Phase to Reduce Non-Covalent Binding” filed Mar. 26, 2001. This application is herein incorporated by reference in its entirety.

As shown in FIG. 7B, a target agent such as target antigen 204 from a test sample is added to a capture bead 190 coated with antibody transport probes 196. In this alternate implementation, the transport probes 196 are formed from proteins such as antibodies.

Capture bead 190 has a characteristic that allows it to be isolated from a material suspension or solution. The capture bead may be selected based upon a desired size, and a preferred way to make it isolatable is for it to be magnetic.

FIG. 8A illustrates the binding of target DNA or RNA 202 to complementary transport probes 198 on capture bead 190 in the genetic assay implementation of the present invention. FIG. 8B shows an immunoassay version of FIG. 8A, transport probes 196 can alternatively include antibodies or antigens for binding to a target protein 204.

FIG. 9A shows a reporter bead 192 coated with oligonucleotide signal probes 206 complementary to target agent 202 (see FIG. 8A). Reporter bead 192 is selected based upon a desired size and the material properties for detection and reporting purposes. In one specific embodiment a 2.1 um polystyrene bead is employed. Signal probes 206 can be strands of DNA or RNA to capture target DNA or RNA.

FIG. 9B illustrates a reporter bead 192 coated with antibody signal probes 208 that bind to the target agent 204 as shown in FIG. 8B. Reporter bead 192 is selected based upon a desired size and the material properties for detection and reporting purposes. This may also preferably include a 2.1 um polystyrene bead. Signal probes 208 can be antigens or antibodies implemented to capture protein or glycoprotein targets.

FIG. 10A is a pictorial representation of a dual bead complex 194 that can be formed from capture bead 190 with probe 198, target agent 202, and reporter bead 192 with probe 206. Probes 198 and 206 conjugated on capture bead 190 and reporter bead 192, respectively, have sequences complementary to the target agent 202, but not to each other. Further details regarding target agent detection and methods of reducing non-specific binding of target agents to beads are discussed in commonly assigned and co-pending U.S. Provisional Application Ser. No. 60/278,106 entitled “Dual Bead Assays Including Use of Restriction Enzymes to Reduce Non-Specific Binding” filed Mar. 23, 2001; and U.S. Provisional Application Ser. No. 60/278,110 entitled “Dual Bead Assays Including Use of Chemical Methods to Reduce Non-Specific Binding” also filed Mar. 23, 2001, which are both incorporated herein by reference in their entirety.

FIG. 10B is a pictorial representation of the immunoassay version of a dual bead complex 194 that can be formed from capture bead 190 with probe 196, target agent 204, and reporter bead 192 with probe 208. Probes 196 and 208 conjugated on capture bead 190 and reporter bead 192, respectively, only bind to the target agent 202, and not to each other.

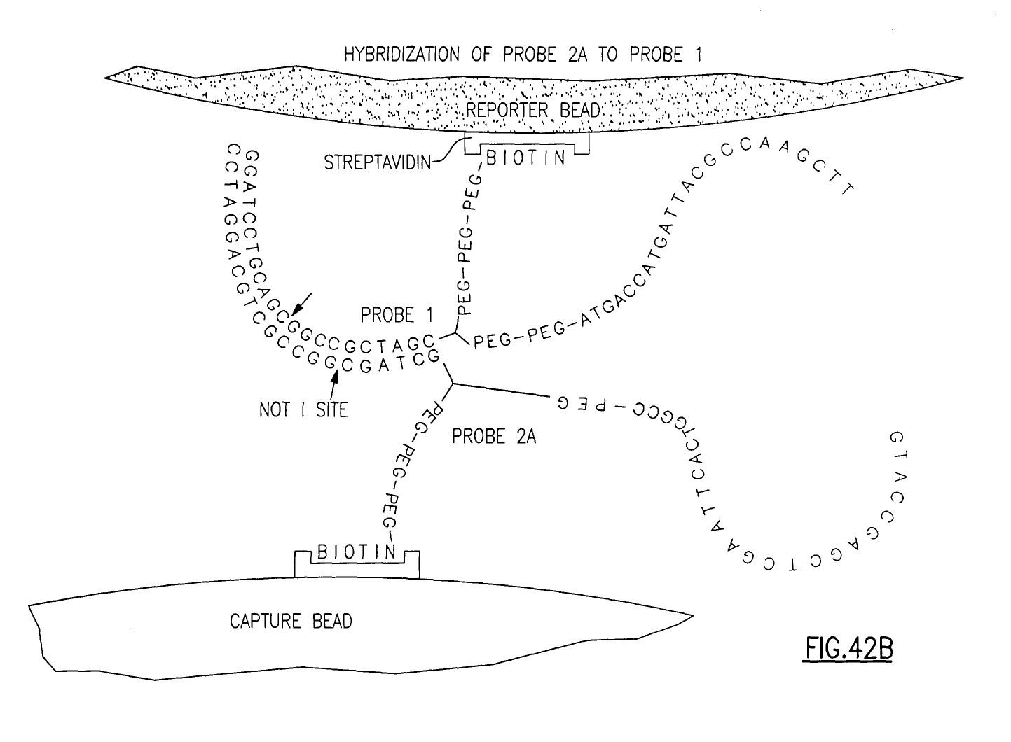

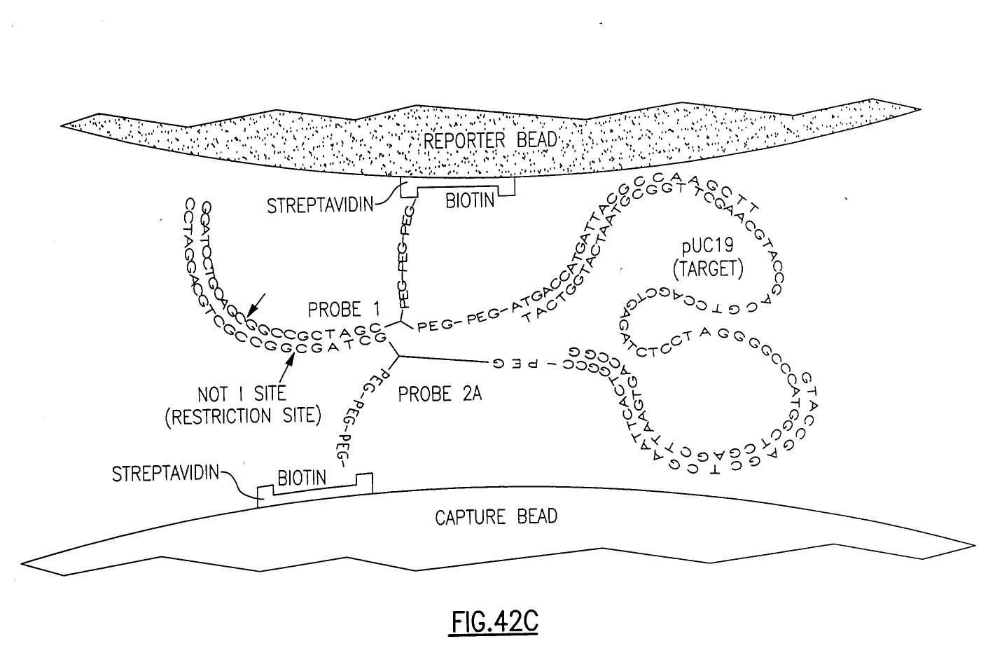

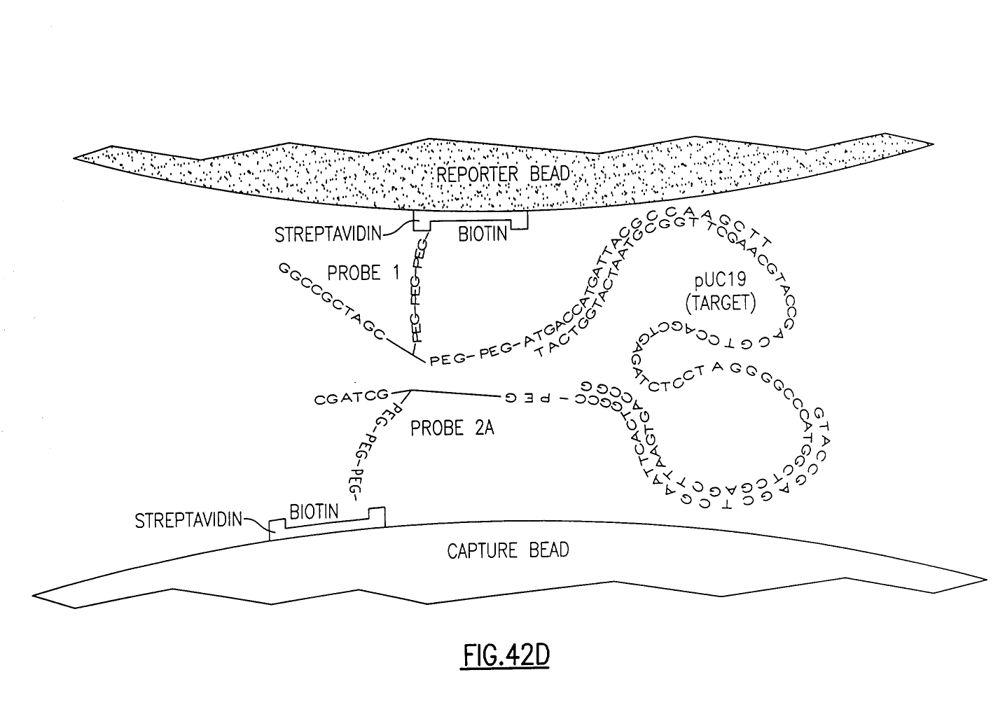

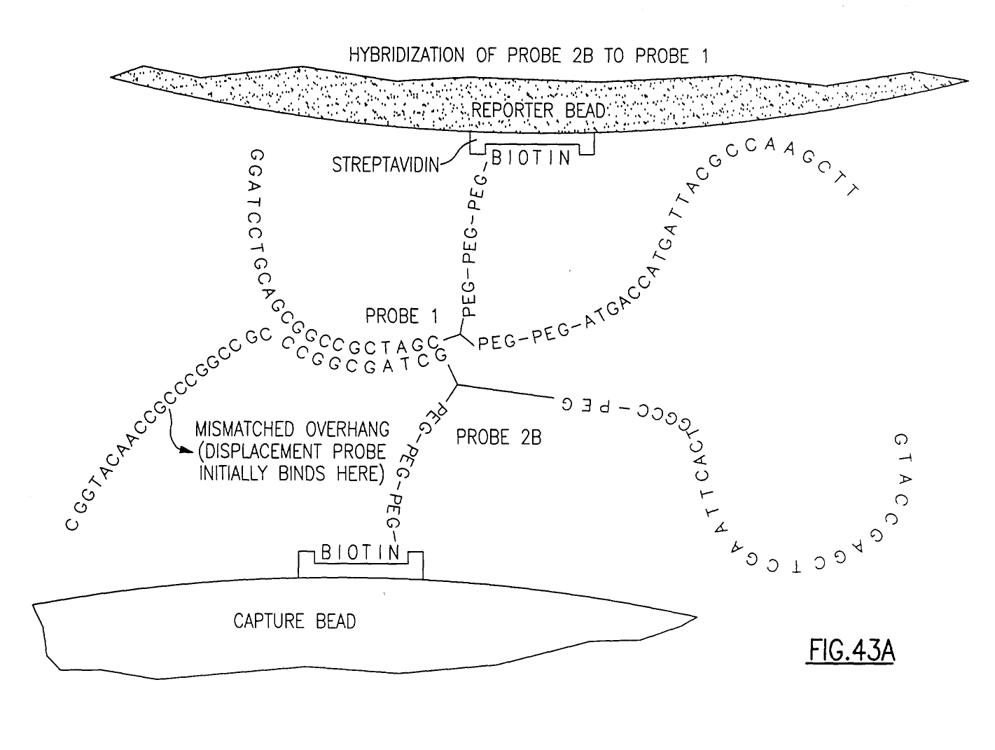

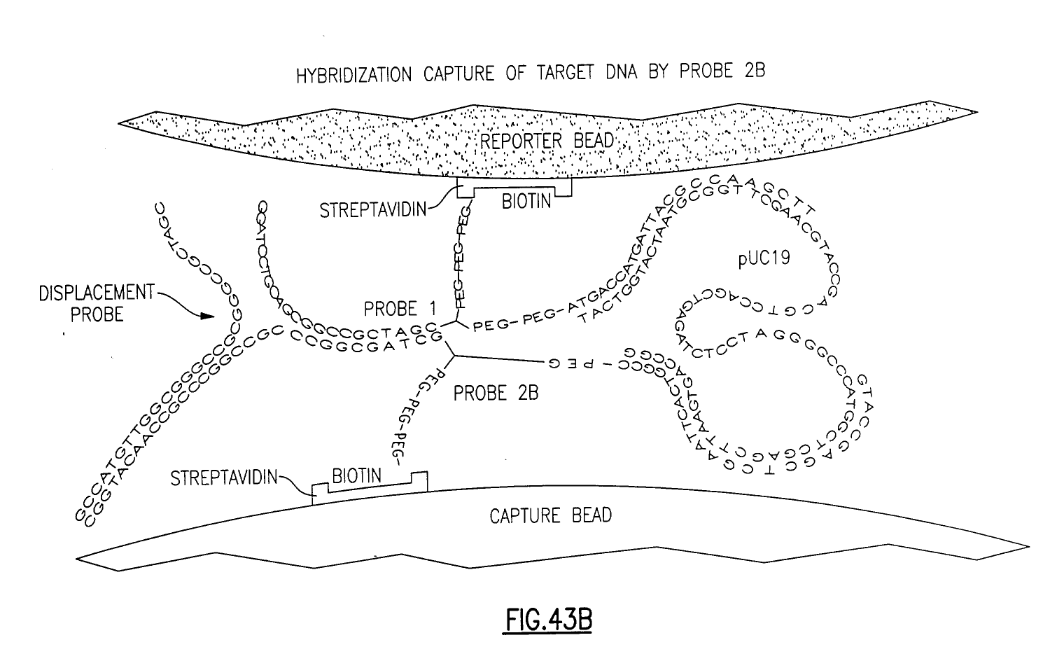

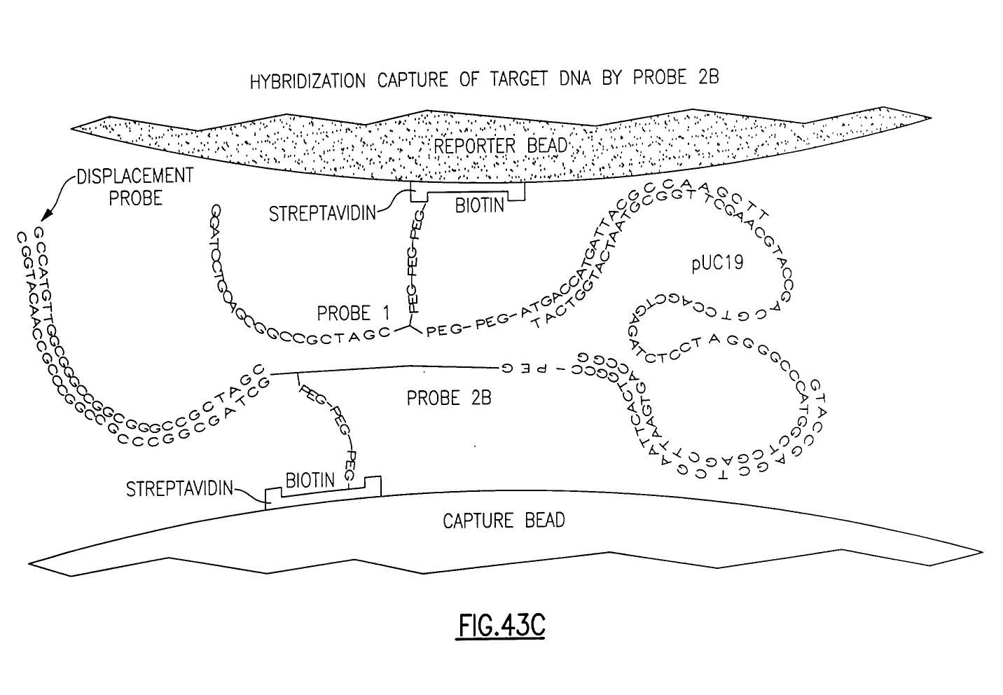

In an alternative embodiment of the current system of assays, the efficiency and specificity of target agent binding may be enhanced by using a cleavable spacer or a displaceable spacer that temporarily links the reporter bead 192 and capture bead 190. The dual bead complex formed by the cleavable spacer or displaceable spacer essentially places the transport probe and the signal probe in close proximity to each other thus allowing more efficient target binding to both probes. Once the target agent is bound to the probes, the spacer may then be cleaved permitting the bound target agent to retain the dual bead structure. The use of cleavable spacers in dual bead assay systems is disclosed in further detail in commonly assigned and co-pending U.S. Provisional Application Ser. No. 60/278,688 entitled “Dual Bead Assays Using Cleavable Spacers to Improve Specificity and Sensitivity” filed Mar. 26, 2001, which is herein incorporated in its entirety by reference. The use of cleavable spacers and displaceable spacers are also described below in conjunction with FIGS. 42A-42D and 43A-43C.

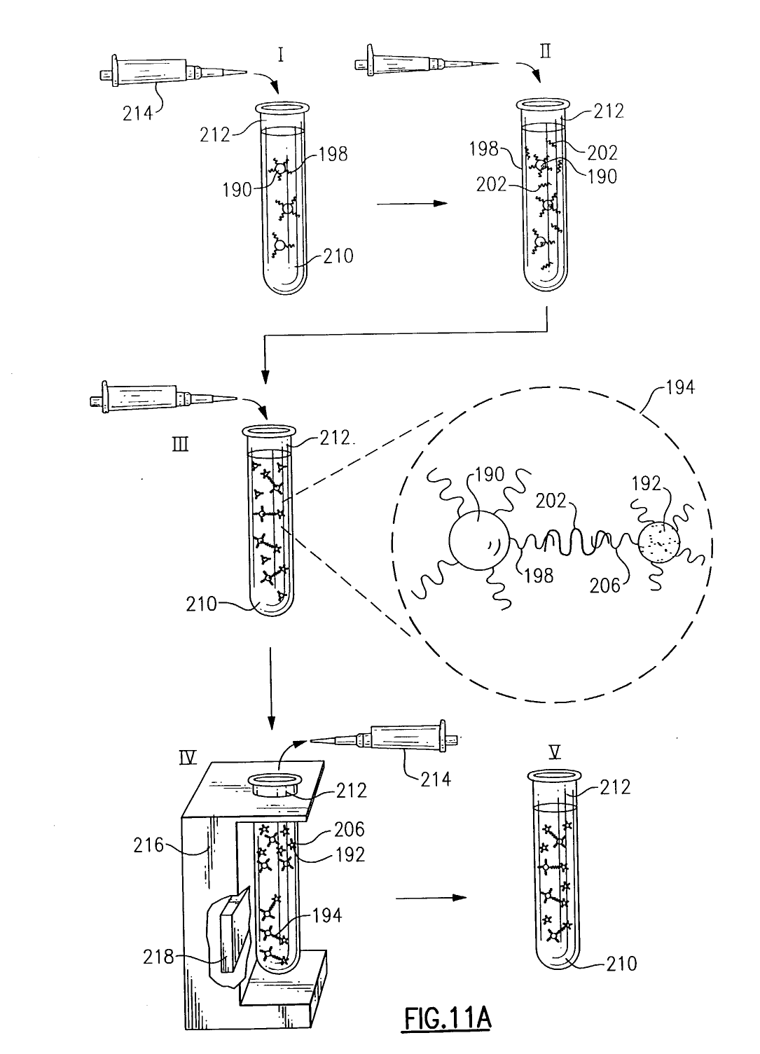

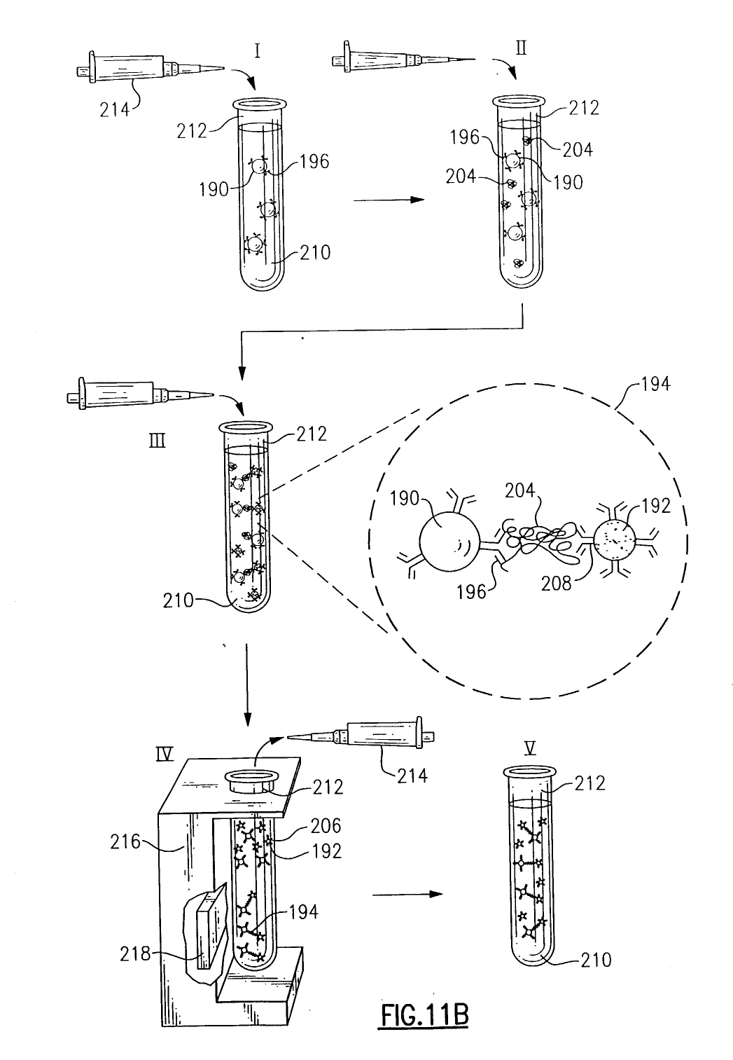

With reference now to FIG. 11A, there is illustrated a method of preparing a molecular assay using a “single-step hybridization” technique to create dual bead complex structures in a solution according to one aspect of the present invention. This method includes 5 principal steps identified consecutively as Steps I, II, III, IV, and V.

In Step I of this method, a number of capture beads 190 coated with oligonucleotide transport probes 198 are deposited into a test tube 212 containing a buffer solution 210. The number of capture beads 190 used in this method may be, for example, on the order of 10E+07 and each on the order of 1 um or greater in diameter. Capture beads 190 are suspended in hybridization solution and are loaded into the test tube 212 by injection with pipette 214. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius. In a preliminary step in this embodiment, transport probes 198 are conjugated to 3 um magnetic capture beads 190 by EDC conjugation. Further details regarding conjugation methods are disclosed in commonly assigned U.S. Provisional Application Ser. No. 60/271,922 entitled, “Methods for Attaching Capture DNA and Reporter DNA to Solid Phase Including Selection of Bead Types as Solid Phase” filed Feb. 27, 2001; and U.S. Provisional Application Ser. No. 60/277,854 entitled “Methods of Conjugation for Attaching Capture DNA and Reporter DNA to Solid Phase” filed Mar. 22, 2001, both of which are herein incorporated by reference in their entirety.

As shown in Step II, target DNA or RNA 202 is added to the solution. Oligonucleotide transport probes 198 are complementary to the DNA or RNA target agent 202. The target DNA or RNA 202 thus binds to the complementary sequences of transport probe 198 attached to the capture bead 190 as shown in FIG. 8A.

With reference now to Step II, there is added to the solution 210 reporter beads 192 coated with oligonucleotide signal probes 206. As also shown in FIGS. 9A and 10A, signal probes 206 are complementary to the target DNA or RNA 202. In one embodiment, signal probes 206, which are complementary to a portion of the target DNA or RNA 202, are conjugated to 2.1 um fluorescent reporter beads 192. Signal probes 206 and transport probes 198 each have sequences that are complementary to the target DNA 202, but not complementary to each other. After adding reporter beads 192, the dual bead complex 194 is formed such that the target DNA 202 links capture bead 190 and reporter beads 192. With specific and thorough washing, there should be minimal non-specific binding between reporter bead 192 and capture bead 190. The target agent 202 and signal probe 206 are preferably allowed to hybridize for three to four hours at 37 degrees Celsius.

In this embodiment and others, it was found that intermittent mixing (i.e., periodically mixing and then stopping) produced greater yield of dual bead complex than continuous mixing during hybridization. Thus when this step is performed on-disc, the disc drive motor 140 and controller 142, FIG. 2, may be advantageously employed to periodically rotate the disc to achieve the desired intermittent mixing. This may be implemented in mixing protocols encoded on the disc that rotate the disc in one direction, then stop the disc, and thereafter rotate the disc again in the same direction in a prescribed manner with a preferred duty cycle of rotation and stop sessions. Alternatively, the encoded mixing protocol may rotate the disc in a first direction, then stop the disc, and thereafter rotate the disc again in the opposite direction with a preferred duty cycle of rotation, stop, and reverse rotation sessions. These features of the present invention are discussed in further detail in connection with FIGS. 33A and 35.

As next shown in Step IV of FIG. 11A, after hybridization, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated. Alternatively, this magnetic removal step may be performed on-disc as shown in FIGS. 33A, 35, and 36A-36C.

The purification process illustrated in Step IV includes the removal of supernatant containing free-floating particles. Wash buffer is added into the test tube and the bead solution is mixed well. The preferred wash buffer for the one step assay consists of 145 mM NaCl, 50 mM Tris, pH 7.5, 0.1% SDS, 0.05% Tween, 0.25% NFDM, and 10 mM EDTA. Most of the unbound reporter beads 182, free-floating DNA, and non-specifically bound particles are agitated and removed from the supernatant. The dual bead complex can form a matrix of capture beads, target sequences, and reporter beads, wherein the wash process can further assist in the extraction of free floating particles trapped in the lattice structure of overlapping dual bead particles. Further details relating to other aspects associated with methods of decreasing non-specific binding of reporter beads to capture beads are disclosed in, for example, commonly assigned U.S. Provisional Application Ser. No. 60/272,134 entitled “Reduction of Non-Specific Binding in Dual Bead Assays by Selection of Bead Type and Bead Treatment” filed Feb. 28, 2001; and U.S. Provisional Application Ser. No. 60/275,006 entitled “Reduction of Non-Specific Binding in Dual Bead Assays by Selection of Buffer Conditions and Wash Conditions” filed Mar. 12, 2001. Both of these applications are herein incorporated by reference in their entirety.

The last principal step shown in FIG. 11A is Step V. In this step, once the dual bead complex has been washed approximately 3-5 times with wash buffer solution, the assay mixture may be loaded into the disc and ready to be analyzed.

FIG. 11B illustrates an immunoassay using a “single-step antigen binding” method, similar to that in FIG. 11A, to create dual bead complex structures in a solution. This method similarly includes 5 principal steps. These steps are respectively identified as Steps I, II, III, IV, and V in FIG. 11A.

As shown in Step I, capture beads 190, e.g., on the order of 10E+07 in number and each on the order of 1 um or above in diameter, which are coated with antibody transport probes 196 are added to a buffer solution 210. This solution may be that same as that employed in the method shown in FIG. 11A or alternatively may be specifically prepared for use with immunochemical assays. The antibody transport probes 196 have a specific affinity for the target antigen 204. The transport probes 196 bind specifically to epitopes within the target antigen 204 as also shown in FIG. 8B. In one embodiment, antibody transport probes 196 that have an affinity for a portion of the target antigen may be conjugated to 3 um magnetic capture beads 190 via EDC conjugation. Alternatively, conjugation of the transport probes 196 to the capture bead 190 may be achieved by passive adsorption.

With reference now to Step II shown in FIG. 11B, the target antigen 204 is added to the solution. The target antigen 204 binds to the antibody transport probe 196 attached to the capture bead 190 as also shown in FIG. 8B.

As illustrated in Step III, reporter beads 192 coated with antibody signal probes 208 are added to the solution. Antibody signal probes 208 specifically binds to the epitopes on target antigen 204 as also represented in FIGS. 9B and 10B. In one embodiment, signal probes 208 are conjugated to 2.1 um fluorescent reporter beads 192. Signal probes 208 and transport probes 196 each bind to specific epitopes on the target antigen, but not to each other. After adding reporter beads 192, the dual bead complex 194 is formed such that the target antigen 204 links capture bead 190 and reporter bead 192. With specific and thorough washing, there should be minimal non-specific binding between reporter bead 192 and capture bead 190.

In Step IV, after the binding in Step III, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated. Alternatively, as indicated above, this magnetic removal step may also be performed on-disc as shown in FIGS. 33A, 35, and 36A-36C.

The purification process of Step IV includes the removal of supernatant containing free-floating particles. Wash buffer is added into the test tube and the bead solution is mixed well. Most of the unbound reporter beads 182, free-floating protein samples, and non-specifically bound particles are agitated and removed from the supernatant. The dual bead complex can form a matrix of capture beads, target antigen, and reporter beads, wherein the wash process can further assist in the extraction of free floating particles trapped in the lattice structure of overlapping dual bead particles.

The last principal step in FIG. 11B is Step V. In this step, once the dual bead complex has been washed approximately 3-5 times with wash buffer solution, the assay mixture is loaded into the disc and is thereby in condition to be analyzed.

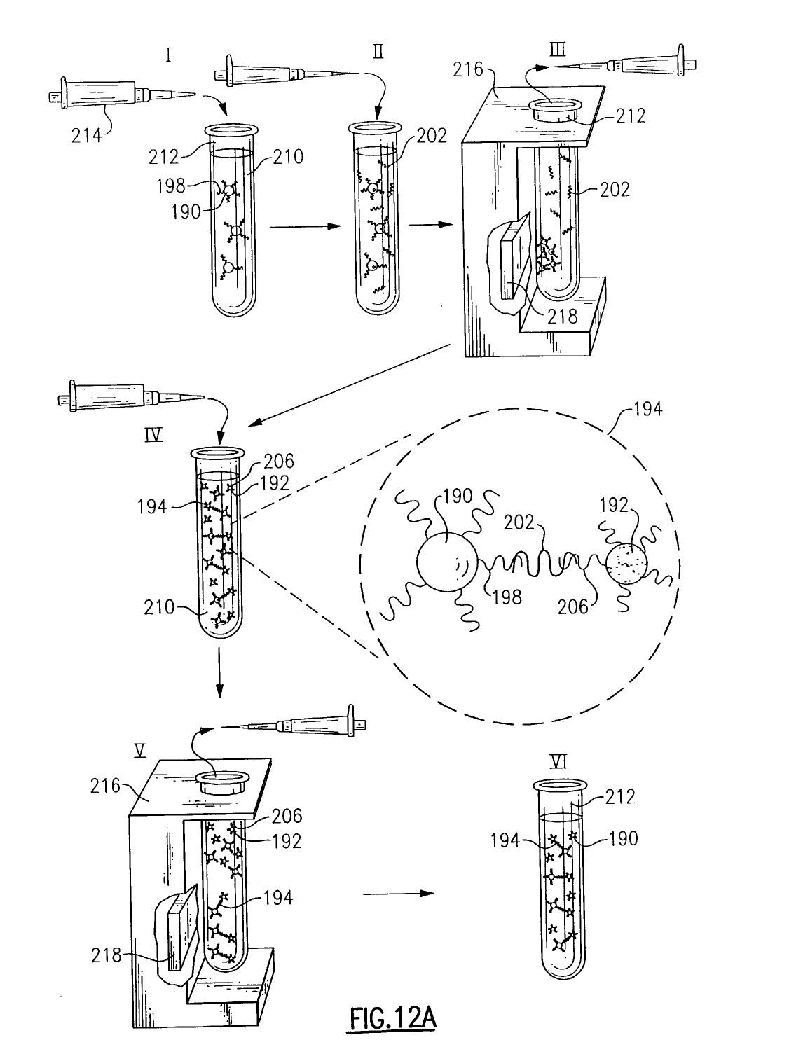

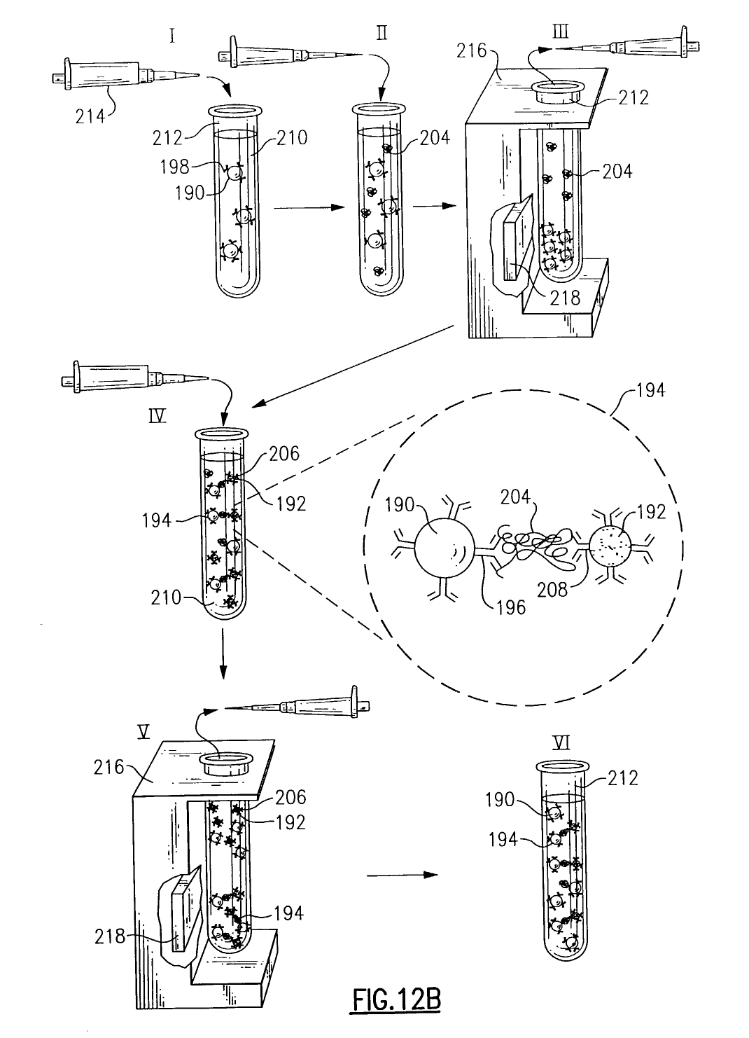

FIG. 12A shows an alternative genetic assay method referred to here as a “two-step hybridization” to create the dual bead complex which has 6 principal steps. Generally, capture beads are coated with oligonucleotide transport probes 198 complementary to DNA or RNA target agent and placed into a buffer solution. In this embodiment, transport probes that are complementary to a portion of target agent are conjugated to 3 um magnetic capture beads via EDC conjugation. Other types of conjugation of the oligonucleotide transport probes to a solid phase may be utilized. These include, for example, passive adsorption or use of streptavidin-biotin interactions. These 6 main steps according to this method of the present invention are consecutively identified as Steps I, II, III, IV, V, and VI in FIG. 12A.

More specifically now with reference to Step I shown in FIG. 12A, capture beads 190, suspended in hybridization solution, are loaded from the pipette 214 into the test tube 212. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius.

In Step II, target DNA or RNA 202 is added to the solution and binds to the complementary sequences of transport probe 198 attached to capture bead 190. In one specific embodiment of this method, target agent 202 and the transport probe 198 are allowed to hybridize for 2 to 3 hours at 37 degrees Celsius. Sufficient hybridization, however, may be achieved within 30 minutes at room temperature. At higher temperatures, hybridization may be achieved substantially instantaneously.

As next shown in Step III, target agents 202 bound to the capture beads are separated from unbound species in solution by exposing the solution to a magnetic field to isolate bound target sequences by using the magnetic properties of the capture bead 190. The magnetic field can be enclosed in a magnetic test tube rack 216 with a built-in magnet permanent 218 or electromagnet to draw out the magnetic beads and remove any unbound target DNA 202 free-floating in the suspension via pipette extraction of the solution. As with the above methods, in the on-disc counterpart hereto, this magnetic removal step may be performed as shown in FIGS. 33A, 35, and 36A-36C. A wash buffer is added and the separation process can be repeated. The preferred wash buffer after the transport probes 198 and target DNA 202 hybridize, consists of 145 mM NaCl, 50 mM Tris, pH 7.5, and 0.05% Tween. Hybridization methods and techniques for decreasing non-specific binding of target agents to beads are further disclosed in commonly assigned and co-pending U.S. Provisional Application Ser. No. 60/278,691 entitled “Reduction of Non-Specific Binding of Dual Bead Assays by Use of Blocking Agents” filed Mar. 26, 2001. This application is herein incorporated by reference in its entirety.

Referring now to Step IV illustrated in FIG. 12A, reporter beads 192 are added to the solution as discussed in conjunction with the method shown in FIG. 11A. Reporter beads 192 are coated with signal probes 206 that are complementary to target agent 202. In one particular embodiment of this method, signal probes 206, which are complementary to a portion of target agent 202, are conjugated to 2.1 um fluorescent reporter beads 192. Signal probes 206 and transport probes 198 each have sequences that are complementary to target agent 202, but not complementary to each other. After the addition of reporter beads 192, the dual bead complex structures 190 are formed. As would be readily apparent to one of skill in the art, the dual bead complex structures are formed only if the target agent of interest is present. In this formation, target agent 202 links magnetic capture bead 190 and reporter bead 192. Using the preferred buffer solution, with specific and thorough washing, there is minimal non-specific binding between the reporter beads and the capture beads. Target agent 202 and signal probe 206 are preferably allowed to hybridize for 2-3 hours at 37 degrees Celsius. As with Step II discussed above, sufficient hybridization may be achieved within 30 minutes at room temperature. At higher temperatures, the hybridization in this step may also be achieved substantially instantaneously.

With reference now to Step V shown in FIG. 12A, after the hybridization in Step IV, the dual bead complex 194 is separated from unbound species in solution. The solution is again exposed to a magnetic field to isolate the dual bead complex 194 using the magnetic properties of the capture bead 190. Note again that the isolate will include capture beads not bound to reporter beads. As with Step III above in the on-disc counterpart hereto, this magnetic separation step may be performed as shown in FIGS. 33A, 35, and 36A-36C.

A purification process to remove supernatant containing free-floating particles includes adding wash buffer into the test tube and mixing the bead solution well. The preferred wash buffer for the two-step assay consists of 145 mM NaCl, 50 mM Tris, pH 7.5, 0.1% SDS, 0.05% Tween, 0.25% NFDM, and 10 mM EDTA. Most unbound reporter beads, free-floating DNA, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles. Other related aspects directed to reduction of non-specific binding between reporter bead, target agent, and capture bead are disclosed in, for example, commonly assigned U.S. Provisional Application Ser. No. 60/272,243 entitled “Mixing Methods to Reduce Non-Specific Binding in Dual Bead Assays” filed Feb. 28, 2001; and U.S. Provisional Application Ser. No. 60/272,485 entitled “Dual Bead Assays Including Linkers to Reduce Non-Specific Binding” filed Mar. 1, 2001, which are incorporated herein in their entirety.

The final principal step shown in FIG. 12A is Step VI. In this step, once the dual bead complex 194 has been washed approximately 3-5 times with wash buffer solution, the assay mixture is loaded into the disc and analyzed. Alternatively, during this step, the oligonucleotide signal and transport probes may be ligated to prevent breakdown of the dual bead complex during the disc analysis and signal detection processes. Further details regarding probe ligation methods are disclosed in commonly assigned and co-pending U.S. Provisional Application Ser. No. 60/278,694 entitled “Improved Dual Bead Assays Using Ligation” filed Mar. 26, 2001, which is herein incorporated in its entirety by reference.

In accordance with another aspect of this invention, FIG. 12B shows an immunoassay method, similar to those discussed in connection with the immunoassay method of FIG. 11B and following the steps of the genetic assay of FIG. 12A. This method is also referred to here as a “two-step binding” to create the dual bead complex in an immunochemical assay. As with the method shown in FIG. 12A, this method includes 6 main steps. In general, capture beads coated with antibody transport probes that specifically bind to epitopes on target antigens are placed into a buffer solution. In one specific embodiment, antibody transport probes are conjugated to 3 um magnetic capture beads. Different sized magnetic capture beads may be employed depending on the type of disc drive and disc assembly utilized to perform the assay. These 6 main steps according to this alternative method of the invention are respectively identified as Steps I, II, III, IV, V and VI in FIG. 12B.

With specific reference now to Step I shown in FIG. 12B, capture beads 190, suspended in buffer solution 210, are loaded into a test tube 212 via injection from pipette 214.

In Step II, target antigen 204 is added to the solution and binds to the antibody transport probe 196 attached to capture bead 190. Target antigen 204 and the transport probe 196 are preferably allowed to bind for 2 to 3 hours at 37 degrees Celsius. Shorter binding times are also possible.

As shown in Step III, target antigen 204 bound to the capture beads 190 is separated from unbound species in solution by exposing the solution to a magnetic field to isolate bound target proteins or glycoproteins by using the magnetic properties of capture bead 190. The magnetic field can be enclosed in a magnetic test tube rack 216 with a built-in magnet permanent 218 or electromagnet to draw out the magnetic beads and remove any unbound target antigen 204 free-floating in the suspension via pipette extraction of the solution. A wash buffer is added and the separation process can be repeated.

As next illustrated in Step IV, reporter beads 192 are added to the solution as discussed in conjunction with the method shown in FIG. 11B. Reporter beads 192 are coated with signal probes 208 that have an affinity for the target antigen 204. In one particular embodiment of this two-step immunochemical assay, signal probes 208, which bind specifically to a portion of target agent 204, are conjugated to 2.1 um fluorescent reporter beads 192. Signal probes 208 and transport probes 196 each bind to specific epitopes on the target agent 204, but do not bind to each other. After the addition of reporter beads 192, the dual bead complex structures 190 are formed. As would be readily apparent to those skilled in the art, these dual bead complex structures are formed only if the target antigen of interest is present. In this formation, target antigen 204 links magnetic capture bead 190 and reporter bead 192. Using the preferred buffer solution, with specific and thorough washing, there is minimal non-specific binding between the reporter beads and the capture beads. Target antigen 204 and signal probe 208 are allowed to hybridize for 2-3 hours at 37 degrees Celsius. As with Step II discussed above, sufficient binding may be achieved within 30 minutes at room temperature. In the case of immunoassays temperatures higher than 37 degrees Celsius are not preferred because the proteins will denature.

Turning next to Step V as illustrated in FIG. 12B, after the binding shown in Step IV, the dual bead complex 194 is separated from unbound species in solution. This is achieved by exposing the solution to a magnetic field to isolate the dual bead complex 194 using the magnetic properties of the capture bead 190 as shown. Note again that the isolate will include capture beads not bound to reporter beads.

A purification process to remove supernatant containing free-floating particles includes adding wash buffer into the test tube and mixing the bead solution well. Most unbound reporter beads, free-floating proteins, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles.

The final main step shown in FIG. 12B is Step VI. In this step, once the dual bead complex 194 has been washed approximately 3-5 times with wash buffer solution, the assay mixture is loaded into the disc and analyzed.

As with any of the other methods discussed above, the magnetic removal or separation steps in the method shown in FIG. 12B may be alternatively performed on-disc using the disc, fluidic circuits, and apparatus illustrated in FIGS. 33A-33D, 34A-34C, 35, and 36A-36C.

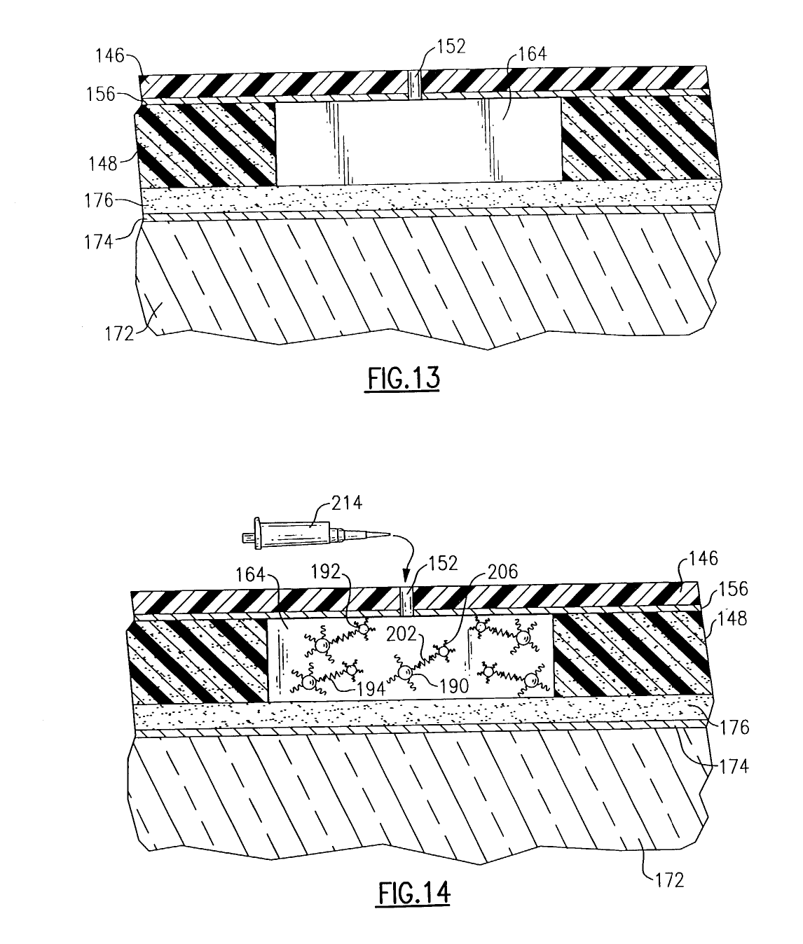

With reference now to FIG. 13, there is shown a cross sectional view illustrating the disc layers (similar to FIG. 6) of the mixing or loading chamber 164. Access to the loading chamber 164 is achieved by an inlet port 152 where the dual bead assay preparation is loaded into the disc system.

FIG. 14 is a view similar to FIG. 13 showing the mixing or loading chamber 164 with the pipette 214 injection of the dual bead complex 194 onto the disc. In this example, the complex includes reporters 192 and capture bead 190 linked together by the target DNA or RNA 202. The signal DNA 206 is illustrated as single stranded DNA complementary to the capture agent. The discs illustrated in FIGS. 13 and 14 may be readily adapted to other assays including the immunoassays and general molecular assays discussed above which employ, alternatively, proteins such as antigens or antibodies implemented as the transport probes, target agents, and signal probes accordingly.

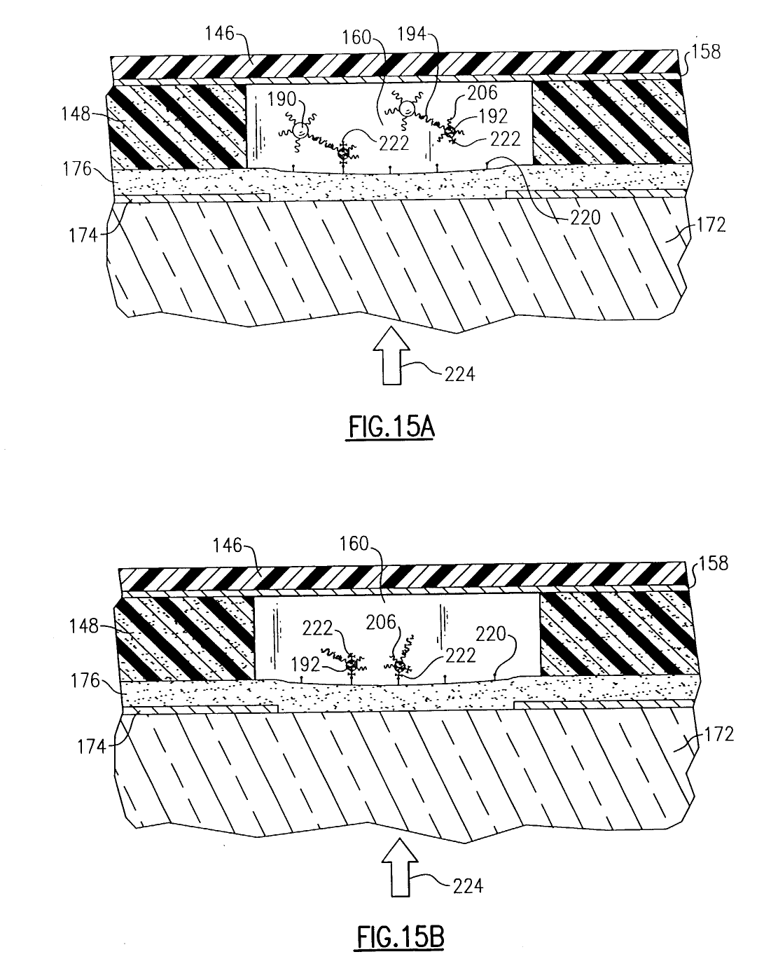

FIG. 15A shows the flow channel 160 and the target or capture zone 170 after anchoring of dual bead complex 194 to a capture agent 220. The capture agent 220 in this embodiment is attached to the active layer 176 by applying a small volume of capture agent solution to the active layer 176 to form clusters of capture agents within the area of the target zone 170. In this embodiment, the capture agent includes biotin or BSA-biotin. FIG. 15A also shows reporters 192 and capture beads 190 as components of a dual bead complex 194 as employed in the present invention. In this embodiment, anchor agents 222 are attached to the reporter beads 192. The anchor agent 222, in this embodiment, may include streptavidin or Neutravidin. So when the reporter beads 192 come in close proximity to the capture agents 220, binding occurs between the anchor probe 222/206 and the capture agent 220, via biotin-streptavidin interactions, thereby retaining the dual bead complex 194 within the target zone 170. At this point, an interrogation beam 224 directed to the target zone 170 can be used to detect the dual bead complex 194 within the target zone 170.

The embodiment of the present invention illustrated in FIGS. 15A and 15B, may alternatively be implemented on the transmissive disc shown in FIGS. 4A-4C, 5B, and 6B.

FIG. 15B is a cross sectional view similar to FIG. 15A illustrating the entrapment of the reporter bead 192 within the target zone 170 after a subsequent change in disc rotational speed. The change in rotational speed removes the capture beads 190 from the dual bead complex 194, ultimately isolating the reporter bead 192 in the target zone 170 to be detected by the interrogation or read beam 224.

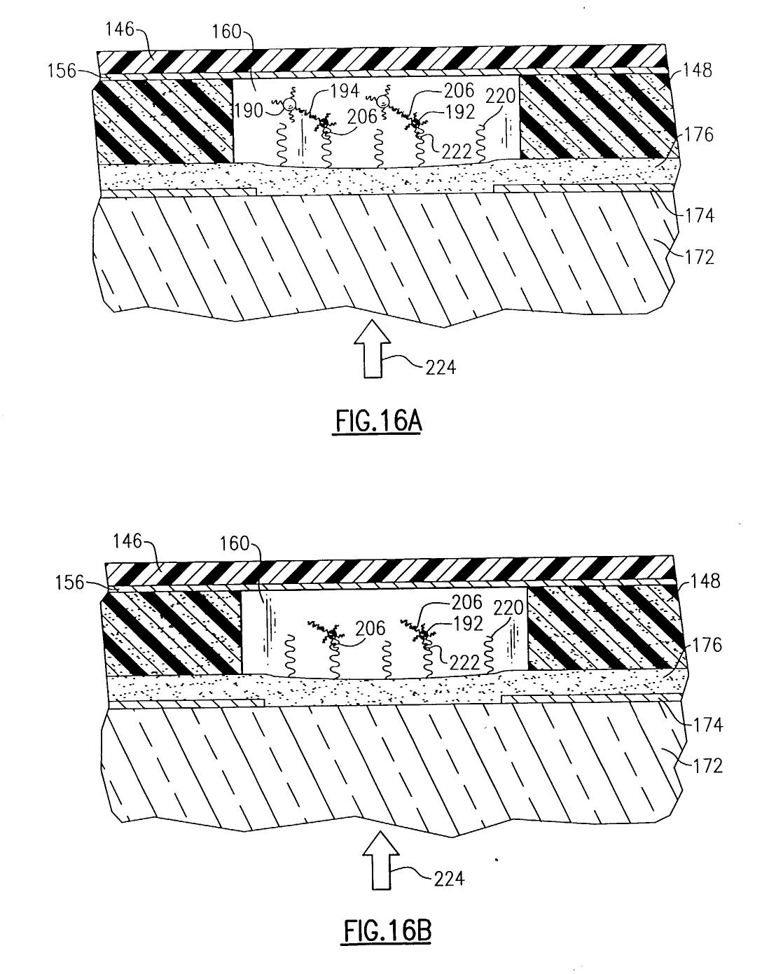

FIG. 16A is a cross sectional view, similar to FIG. 15A, that illustrates an alternative embodiment to FIG. 15A wherein the signal probes 206 or anchor agents 222, on the reporter beads 192, directly hybridize to the capture agent 220. FIG. 16A shows the flow channel 160 and the target or capture zone 170 after anchoring of dual bead complex 194 with the capture agent 220. The capture agent 220 in this embodiment is attached to the active layer 176 by applying a small volume of capture agent solution to the active layer 176 to form clusters of capture agents within the area of the target zone 170. Alternatively, the capture agent 220 may be attached to the active layer using an amino group that covalently binds to the active layer 176. In this embodiment, the capture agent includes DNA. FIG. 16A also shows reporters 192 and capture beads 190 as components of a dual bead complex 194 as employed in the present invention. In this embodiment, anchor agents 222 are attached to the reporter beads 192. The anchor agent 222 in this embodiment may be a specific sequence of nucleic acids that are complimentary to the capture agent 220 or the oligonucleotide signal probe 206 itself. So when the reporter beads 192 come in close proximity to the capture agents 220, hybridization occurs between the anchor agent 222 and the capture agent 220 thereby retaining the dual bead complex 194 within the target zone 170. In an alternate embodiment, the signal probe 206 serves the function of anchor agent 222. At this point, an interrogation beam 224 directed to the target zone 170 may be used to detect the dual bead complex 194 within the target zone 170.

FIG. 16B illustrates the embodiment in FIG. 16A after a subsequent change in disc rotational speed. The change in rational speed removes the capture bead 190 from the dual bead complex 194, ultimately isolating the reporter bead 192 and the target DNA sequence 202 in the target zone 170 to be detected by an interrogation beam 224.

The embodiment of the present invention depicted in FIGS. 16A and 16B, may alternatively be implemented on the transmissive disc illustrated in FIGS. 4A-4C, 5B, and 6B.

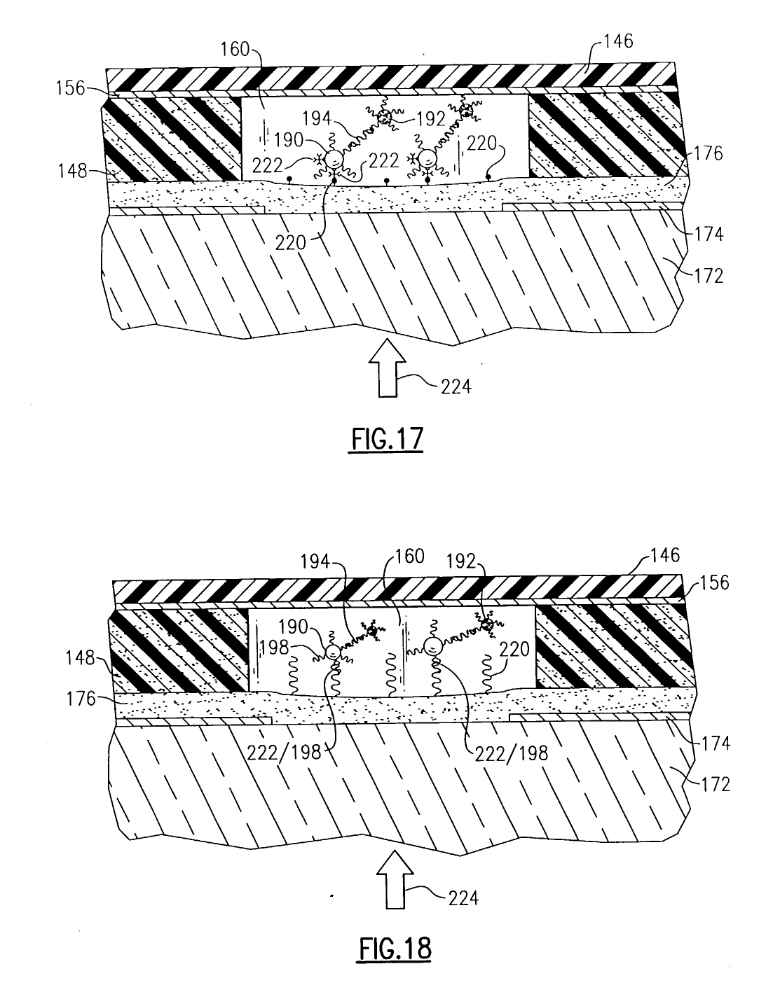

Referring now to FIG. 17, there is shown an alternative to the embodiment illustrated in FIG. 15A. In this embodiment, anchor agents 222 are attached to the capture beads 190 instead of the reporter beads. The anchor agent 222 in this embodiment may include streptavidin or Neutravidin. As in FIG. 15A, the target zone 170 is coated with a capture agent 220. The capture agent may include biotin or BSA-biotin. FIG. 17 also shows reporters 192 and capture beads 190 as components of a dual bead complex 194 as employed in the present invention. When the capture beads 190 come in close proximity to the capture agents 220, binding occurs between the anchor probe 222 and the capture agent 220, via biotin-streptavidin interactions, thereby retaining the dual bead complex 194 within the target zone 170. At this point, an interrogation beam 224 directed to the target zone 170 can be used to detect the dual bead complex 194 within the target zone 170. The embodiment of the present invention shown in FIG. 17, may alternatively be implemented on the transmissive disc illustrated in FIGS. 4A-4C, 5B, and 6B.

FIG. 18 is an alternative to the embodiment illustrated in FIG. 16A. In this embodiment, anchor agents 222 are attached to the capture beads 190 instead of the reporter beads. In this embodiment the transport probes 198, or an anchor agent 222 on the capture bead 190, directly hybridizes to the capture agent 220. In this embodiment, the capture agent 220 includes specific sequences of nucleic acid. The anchor agent 222 in this embodiment may be a specific sequence of nucleic acids that are complimentary to the capture agent 220 or the oligonucleotide signal transport probe 198 itself. So when the capture beads 190 come in close proximity to the capture agents 220, hybridization occurs between the anchor agent 222 and the capture agent 220 thereby retaining the dual bead complex 194 within the target zone 170. At this point, an interrogation beam 224 directed to the target zone 170 can be used to detect the dual bead complex 194 within the target zone 170. The embodiment of the present invention illustrated in FIG. 18, may alternatively be implemented on the transmissive disc shown in FIGS. 4A-4C, 5B, and 6B.

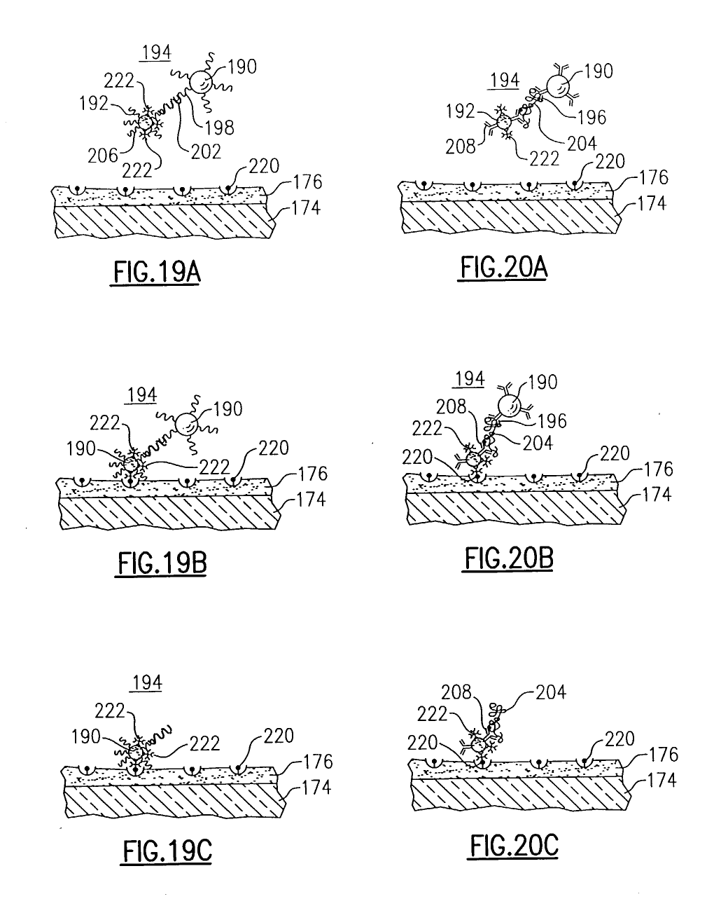

FIGS. 19A-19C are detailed partial cross sectional views showing the active layer 176 and the substrate 174 of the present bio-disc 110 as implemented in conjunction with the genetic assays discussed herein. FIGS. 19A-19C illustrates the capture agent 220 attached to the active layer 176 by applying a small volume of capture agent solution to the active layer 176 to form clusters of capture agents within the area of the target zone. The bond between capture agent 220 and the active layer 176 is sufficient so that the capture agent 220 remains attached to the active layer 176 within the target zone when the disc is rotated. FIGS. 19A and 19B also depict the capture bead 190 from the dual bead complex 194 binding to the capture agent 220 in the capture zone. These dual bead complexes are prepared according to the methods such as those discussed in FIGS. 11A and 12A. The capture agent 220 includes biotin and BSA-biotin. In this embodiment, the reporter bead 192 anchors the dual bead complex 194 in the target zone via biotin/streptavidin interactions. Alternatively, the target zone may be coated with streptavidin and may bind biotinylated reporter beads. FIG. 19C illustrates an alternative embodiment which includes an additional step to those discussed in connection with FIGS. 19A and 19B. In this preferred embodiment, a variance in the disc rotations per minute may create a centrifugal force great enough to break the capture beads 190 away from the dual bead complex 194 based on the differential size and/or mass of the bead. Although there is a shift in the rotation speed of the disc, the reporter bead 192 remains anchored to the target zone. Thus, the reporter beads 192 are maintained within the target zone and detected using an optical bio-disc or medical CD reader.

The embodiment of the present invention discussed in connection FIGS. 19A-19C, may be implemented on the reflective disc illustrated in FIGS. 3A-3C, 5A, and 6A or on the transmissive disc shown in FIGS. 4A-4C, 5B, and 6B.

FIGS. 20A, 20B, and 20C illustrate an alternative embodiment to the embodiment discussed in FIGS. 19A-19C. FIGS. 20A-20C show detailed partial cross sectional views of, a target zone implemented in conjunction with immunochemical assays. FIGS. 20A and 20B also depict the capture bead 190 from the dual bead complex 194 binding to the capture agent 220 in the capture zone. The capture agent 220 includes biotin and BSA-biotin. These dual bead complexes may be prepared according to methods such as those discussed in FIGS. 11B and 12B. In this embodiment, the reporter bead 192 anchors the dual bead complex 194 in the target zone via biotin/streptavidin interactions. The embodiment of the present invention discussed with reference to FIGS. 20A-20C, may be implemented on the reflective disc depicted in FIGS. 3A-3C, 5A, and 6A or on the transmissive disc shown in FIGS. 4A-4C, 5B, and 6B.

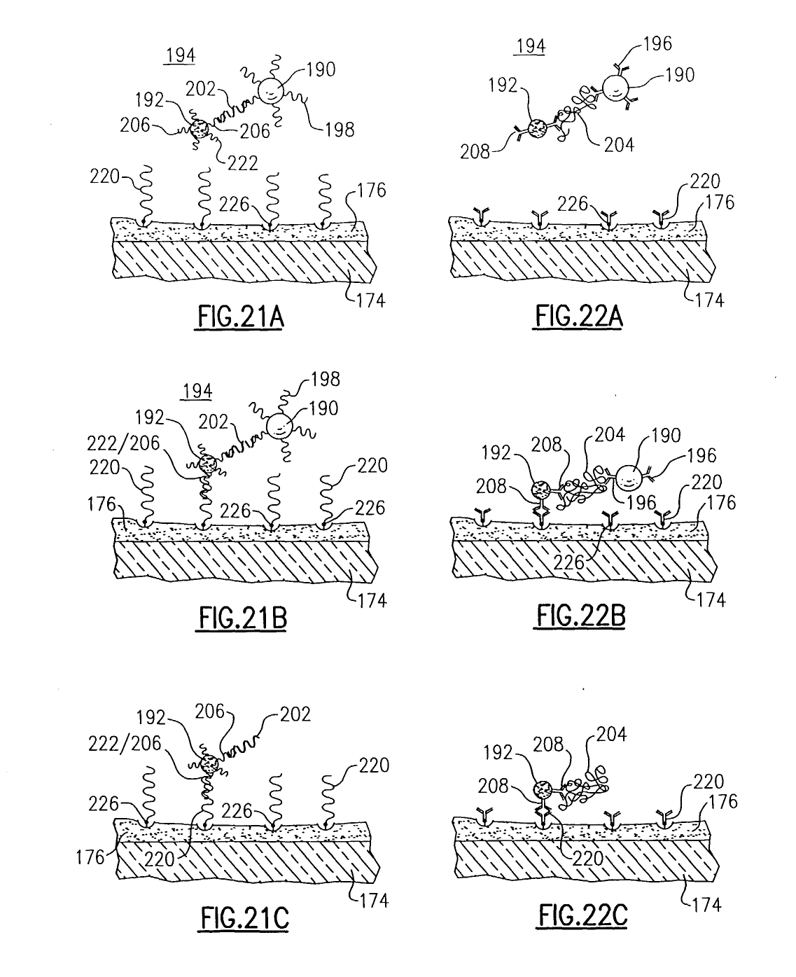

Referring now to FIGS. 21A, 21B, and 21C, there is shown detailed partial cross sectional views of a target zone including the active layer 176 and the substrate 174 of the present bio-disc 110 as implemented in conjunction with the genetic assays discussed herein. FIGS. 21A-21C illustrate the capture agent 220 attached to the active layer 176 by use of an amino group 226 that is an integral part of the capture agent 220. As indicated, the capture agent 220 is situated within the target zone. The bond between the amino group 226 and the capture agent 220, and the amino group 226 and the active layer 176 is sufficient so that the capture agent 220 remains attached to the active layer 176 within the target zone when the disc is rotated. The preferred amino group 226 is NH₂. A thiol group may alternatively be employed in place of the amino group 226. In this embodiment of the present invention, the capture agent 220 includes the specific sequences of amino acids that are complimentary to anchor agent 222 or oligonucleotide signal probe 206 which are attached to the reporter bead 192.

FIG. 21B depicts the reporter bead 192 of the dual bead complex 194, prepared according to methods such as those discussed in FIGS. 11A and 12A, binding to the capture agent 220 in the target zone. As the dual bead complex 194 flows towards the capture agent 220 and is in sufficient proximity thereto, hybridization occurs between the anchor agent 222, or oligonucleotide signal probe 206, and the capture agent 220. Thus, the reporter bead 192 anchors the dual bead complex 192 within the target zone.

FIG. 21C illustrates an alternative embodiment that includes an additional step to those discussed in connection with FIGS. 21A-21B. In this preferred embodiment, a variance in the disc rotations per minute may create enough centrifugal force to break the capture beads 190 away from the dual bead complex 194 based on the differential size and/or mass of the bead. Although there is a shift in the rotation speed of the disc, the reporter bead 192 with the target DNA sequence 202 remains anchored to the target zone. In either case, the reporter beads 192 are maintained within the target zone as desired.

The embodiment of the present invention discussed with reference to FIGS. 21A-21C, may be implemented on the reflective disc shown in FIGS. 3A-3C, 5A, and 6A or on the transmissive disc illustrated in FIGS. 4A-4C, 5B, and 6B.

FIGS. 22A, 22B, and 22C illustrate an alternative embodiment to the embodiment discussed in FIGS. 21A-21C. FIGS. 22A-22C show detailed partial cross sectional views of a target zone implemented in conjunction with immunochemical assays. FIGS. 22A and 22B also depict the reporter bead 192 from the dual bead complex 194, prepared according to methods such as those discussed in FIGS. 11B and 12B, binding to the capture agent 220 in the capture zone. In this embodiment, the capture agent 220 includes antibodies bound to the target zone by use of an amino group 226 that is made an integral part of the capture agent 220. Alternatively, the capture agents 220 may be bound to the active layer 176 by passive absorption, and hydrophobic or ionic interactions. In this embodiment, the reporter bead 192 anchors the dual bead complex 194 in the target zone via specific antibody binding. As with the embodiment illustrated in FIG. 21C, FIG. 22C shows an alternative embodiment that includes an additional step to those discussed in connection with FIGS. 22A-22B. In this alternative embodiment, a variance in the disc rotations per minute may create enough centrifugal force to break the capture beads 190 away from the dual bead complex 194 based on the differential size and/or mass of the bead. Although there is a shift in the rotation speed of the disc, the reporter bead 192 with the target antigen 204 remains anchored to the target zone. In either case, the reporter beads 192 are maintained within the target zone as desired. The embodiment of the present invention described in conjunction with FIGS. 22A-22C, may be implemented on the reflective disc illustrated in FIGS. 3A-3C, 5A, and 6A or on the transmissive disc shown in FIGS. 4A-4C, 5B, and 6B.

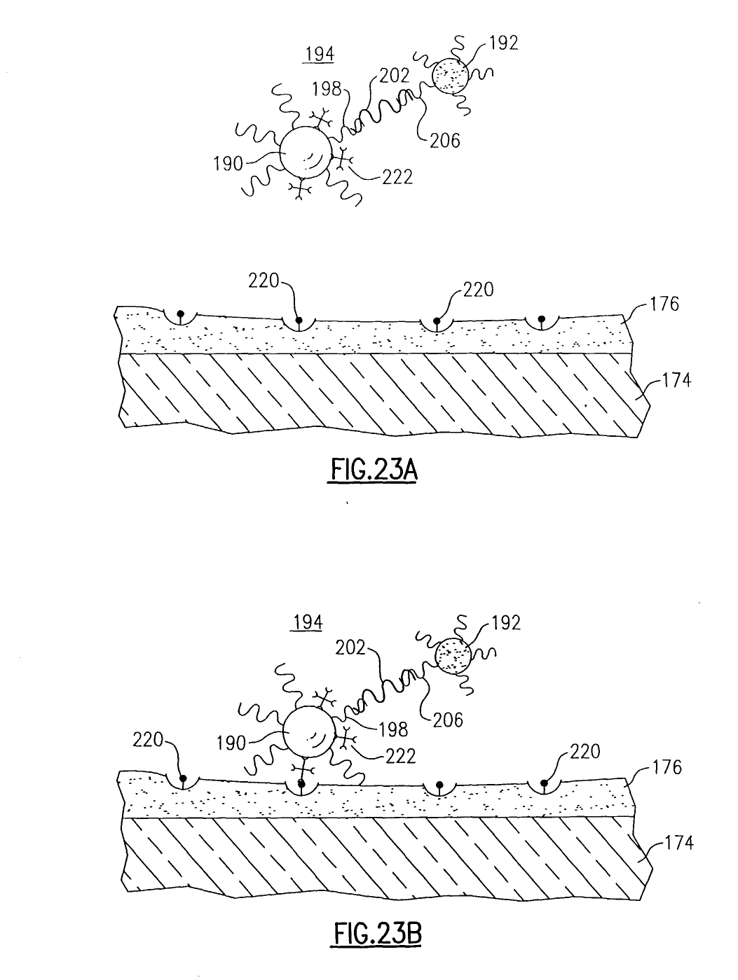

FIGS. 23A and 23B are detailed partial cross sectional views showing the active layer 176 and the substrate 174 of the present bio-disc 110 as implemented in conjunction with the genetic assays. FIGS. 23A and 23B illustrate an alternative embodiment to that discussed in FIGS. 19A and 19B above. In contrast to the embodiment in FIGS. 19A and 19B, in the present embodiment, the anchor agent 222 is attached to the capture bead 190 instead of the reporter bead 192. FIG. 23B illustrates the capture bead 190, from the dual bead complex 194, binding to the capture agent 220 in the capture zone. The capture agent 220 includes biotin and BSA-biotin. In this embodiment, the capture bead 190 anchors the dual bead complex 194 in the target zone via biotin/streptavidin interactions.

The embodiment of the present invention discussed with reference to FIGS. 23A and 23B, may be implemented on the reflective disc illustrated in FIGS. 3A-3C, 5A, and 6A or on the transmissive disc shown in FIGS. 4A-4C, 5B, and 6B.

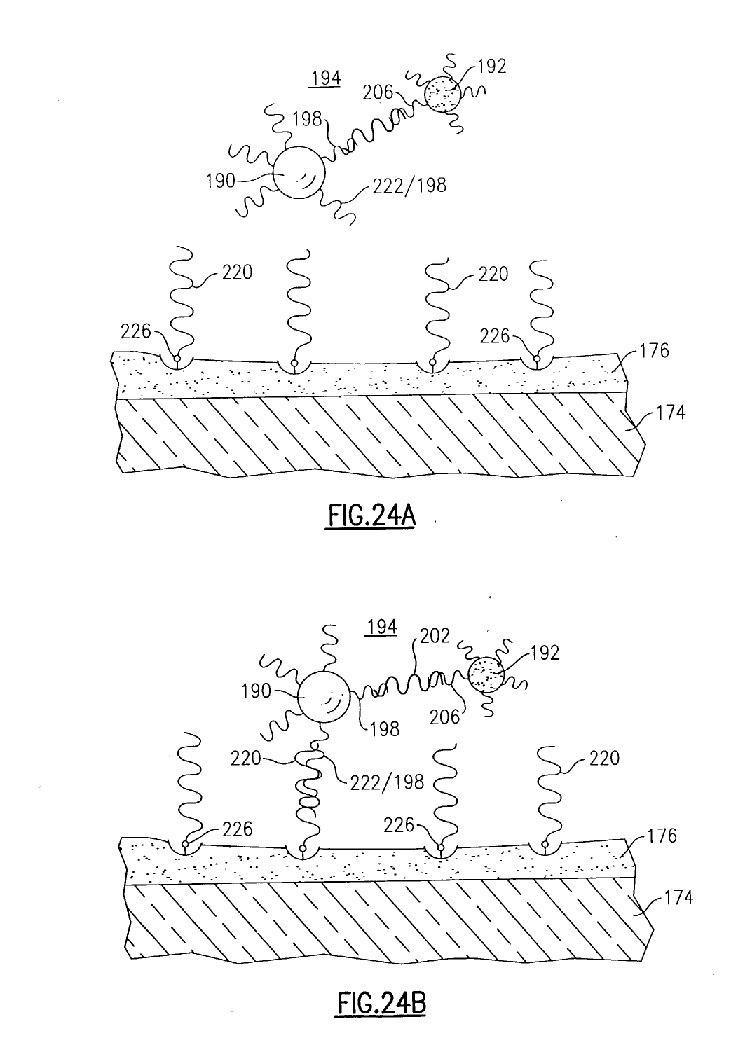

With reference now to FIGS. 24A and 24B, there is presented detailed partial cross sectional views showing the active layer 176 and the substrate 174 of the present bio-disc 110 as implemented in conjunction with the genetic assays. FIGS. 23A and 23B illustrate an alternative embodiment to that discussed in FIGS. 21A and 21B above. In contrast to the embodiment in FIGS. 21A and 21B, in the present embodiment, the anchor agent 222 is attached to the capture bead 190 instead of the reporter bead 192. FIG. 23B illustrates the capture bead 190, from the dual bead complex 194, binding to the capture agent 220 in the capture zone. The capture agent 220 is attached to the active layer 176 by use of an amino group 226 that is made an integral part of the capture agent 220. As indicated, the capture agent 220 is situated within the target zone. The bond between the amino group 226 and the capture agent 220, and the amino group 226 and the active layer 176 is sufficient so that the capture agent 220 remains attached to the active layer 176 within the target zone when the disc is rotated. In this embodiment of the present invention, the capture agent 220 includes the specific sequences of amino acids that are complimentary to the anchor agent 222 or oligonucleotide transport probe 198 which are attached to the capture bead 190. In this embodiment, the capture bead 190 anchors the dual bead complex 194 in the target zone via hybridization between the capture agent 220 and the anchor agent or the transport probe 198.

The embodiment of the present invention shown in FIGS. 24A and 24B, may be implemented on the reflective disc illustrated in FIGS. 3A-3C, 5A, and 6A or on the transmissive disc depicted in FIGS. 4A-4C, 5B, and 6B.

Disc Processing Methods

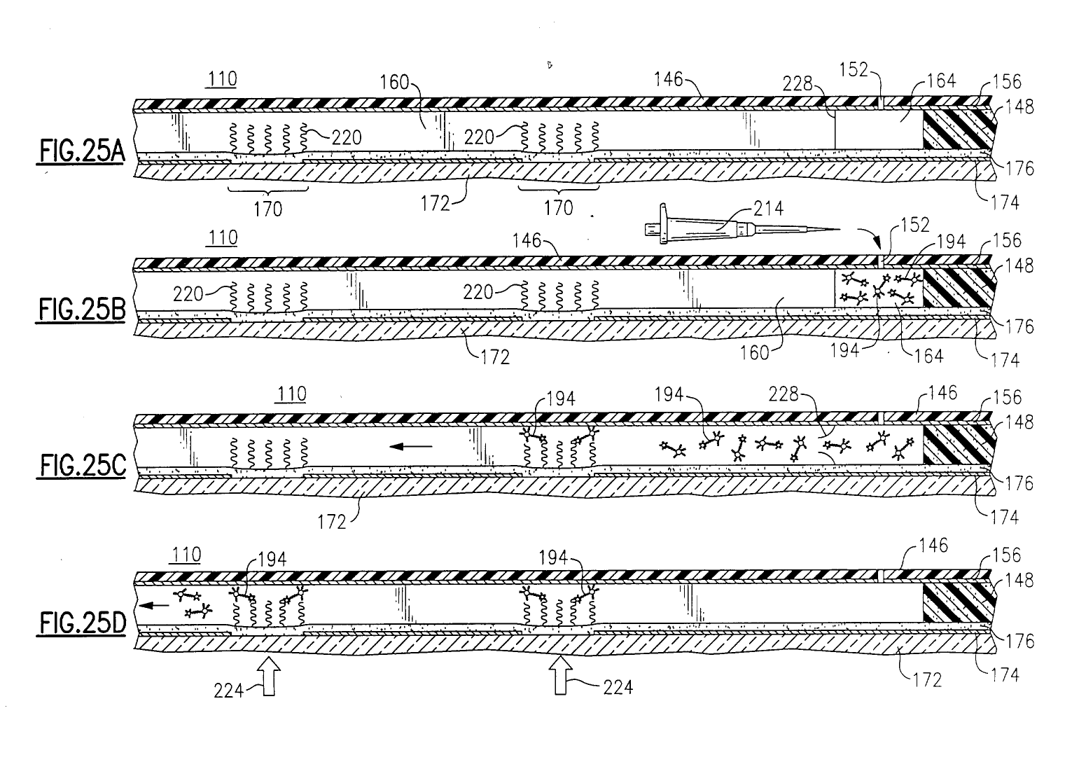

Turning now to FIGS. 25A-25D, there is shown the target zones 170 set out in FIGS. 21A-21C and FIGS. 24A-24B in the context of a disc, using as an input the solution created according to methods such as those shown in FIGS. 11A and 12A.

FIG. 25A shows a mixing/loading chamber 164, accessible through an inlet port 152, and leading to a flow channel 160. Flow channel 160 is pre-loaded with capture agents 220 situated in clusters. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include specific sequences of nucleic acids that are complimentary to anchor agents 222 on either the reporter 192 or capture bead 190.

In FIG. 25B, a pipette 214 is loaded with a test sample of DNA or RNA that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 can include a break-away retaining wall 228 so that complex 194 moves down the flow channel at one time.

In this embodiment, anchor agents 222, attached to reporter beads 192, bind to the capture agents 220 by hybridization, as illustrated in FIG. 25C. In this manner, reporter beads 192 are retained within target zone 170. Binding can be further facilitated by rotating the disc so that the dual bead complex 194 can slowly move or tumble down the flow channel. Slow movement allows ample time for additional hybridization. After hybridization, the disc can be rotated further at the same speed or faster to clear target zone 170 of any unattached dual bead complex 194, as illustrated in FIG. 25D.

An interrogation beam 224 can then be directed through target zones 170 to determine the presence of reporters, capture beads, and dual bead complex, as illustrated in FIG. 25D. In the event no target DNA or RNA is present in the test sample, there will be no dual bead complex structures, reporters, or capture beads bound to the target zones 170, but a small amount of background signal may be detected in the target zones from non-specific binding. In this case, when the interrogation beam 224 is directed into the target zone 170, a zero or low reading results, thereby indicating that no target DNA or RNA was present in the sample.

The speed, direction, and stages of rotation, such as one speed for one period followed by another speed for another period, can all be encoded in the operational information on the disc. The method discussed in connection with FIGS. 25A-25D may also be performed on the transmissive disc illustrated in FIGS. 4A-4C, 5B, and 6B using a system with the top detector 130.

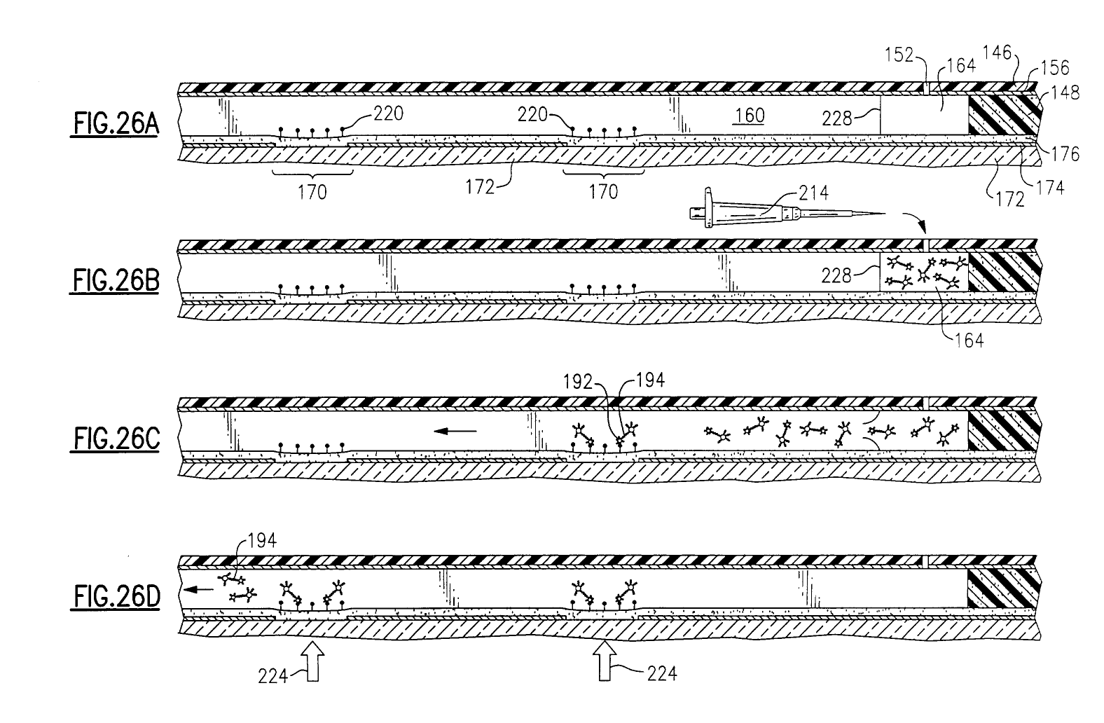

FIGS. 26A-26D show the target zones 170 including the capture chemistries discussed in FIGS. 19A-19C and FIGS. 23A-23B. This method uses as an input the solution created according to methods shown in FIGS. 11A and 12A. FIGS. 26A-26D illustrate an alternative embodiment to that discussed in FIGS. 25A-25D showing a different bead capture method described in further detail below.

FIG. 26A shows a mixing/loading chamber 164, accessible through an inlet port 152, and leading to a flow channel 160. Flow channel 160 is pre-loaded with capture agents 220 situated in clusters. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include specific biotin and BSA-biotin that has affinity to the anchor agents 222 on either the reporter 192 or capture bead 190. The anchor agents may include streptavidin and Neutravidin.

In FIG. 26B, a pipette 214 is loaded with a test sample of DNA or RNA that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 can include a break-away retaining wall 228 so that complex 194 moves down the flow channel at one time.

In this embodiment, anchor agents 222, attached to reporter beads 192, bind to the capture agents 220 by biotin-streptavidin interactions, as illustrated in FIG. 26C. In this manner, reporter beads 192 are retained within target zone 170. Binding can be further facilitated by rotating the disc so that the dual bead complex 194 can slowly move or tumble down the flow channel. Slow movement allows ample time for additional binding between the capture agent 220 and the anchor agent 222. After binding, the disc can be rotated further at the same speed or faster to clear target zone 170 of any unattached dual bead complex 194, as illustrated in FIG. 26D.

An interrogation beam 224 can then be directed through target zones 170 to determine the presence of reporters, capture beads, and dual bead complex, as illustrated in FIG. 26D. In the event no target DNA is present in the test sample, there will be no dual bead complex structures beads bound to the target zones 170. A small amount of background signal may be detected in the target zones from non-specific binding. In this case, when the interrogation beam 224 is directed into the target zone 170, a zero or low reading results, thereby indicating that no target DNA or RNA was present in the sample.

The speed, direction, and stages of rotation, such as one speed for one period followed by another speed for another period, can all be encoded in the operational information on the disc.

The method discussed in conjunction with FIGS. 26A-26D was illustrated on a reflective disc such as the disc shown in FIGS. 3A-3C, 5A, and 6A. This method may also be performed on the transmissive disc shown in FIGS. 4A-4C, 5B, and 6B using a system with the top detector 130.

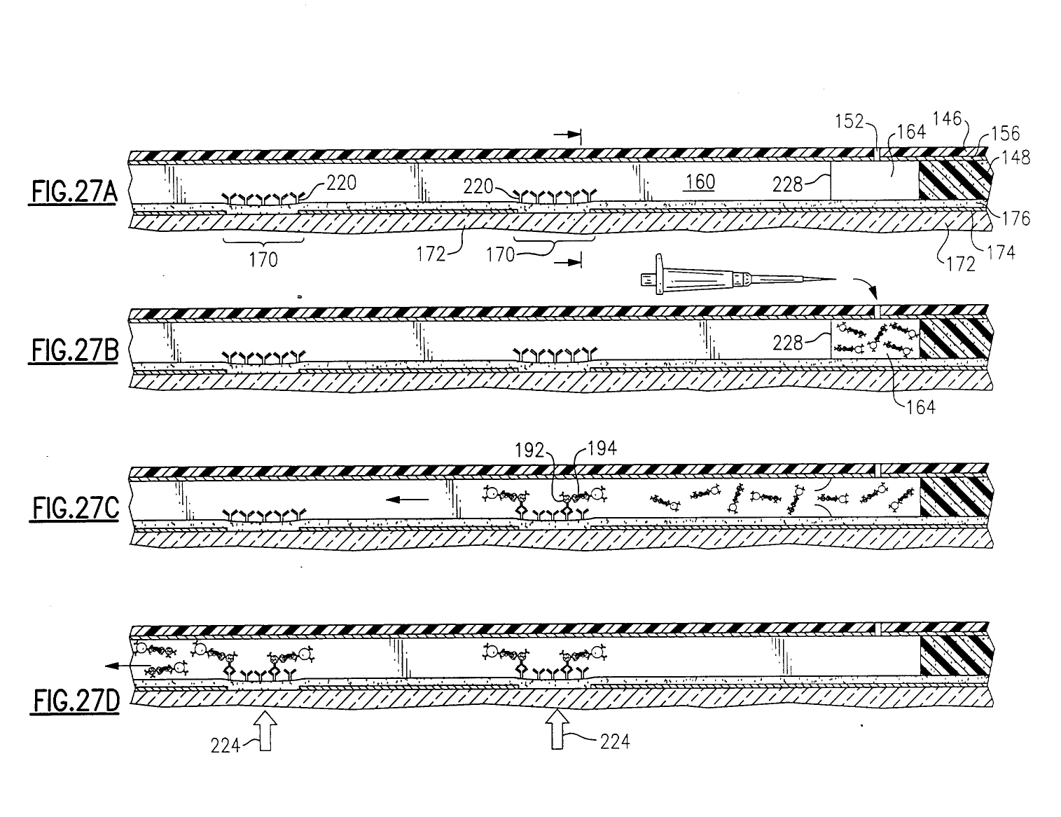

Referring next to FIGS. 27A-27D there is shown a series of cross sectional side views illustrating the steps of yet another alternative method according to the present invention. FIGS. 27A-27D show the target zones 170 including the capture mechanisms discussed in connection with FIGS. 22A-22C. This method uses an input the solution created according to the preparation methods shown in FIGS. 11B and 12B. FIGS. 27A-27D illustrate an immunochemical assay and an alternative bead capture method.

FIG. 27A shows a mixing/loading chamber 164, accessible through an inlet port 152, and leading to a flow channel 160. Flow channel 160 is pre-loaded with capture agents 220 situated in clusters. Each of the clusters of capture agents 220 is situated within a respective target zone 170. Each target zone 170 can have one type of capture agent or multiple types of capture agents, and separate target zones can have one and the same type of capture agent or multiple different capture agents in multiple capture fields. In the present embodiment, the capture agent can include antibodies that specifically bind to epitopes on the anchor agents 222 on either the reporter 192 or capture bead 190. Alternatively, the capture agent can directly bind to epitopes on the target antigen 204 within the dual bead complex 194. The anchor agents 222 can include the target antigen, antibody transport probe 196, the antibody signal probe 208, or any antigen, bound to either the reporter bead 192 or the capture bead 190, that has epitopes than can specifically bind to the capture agent 220.

In FIG. 27B, a pipette 214 is loaded with a test sample of target antigen that has been sequestered in the dual bead complex 194. The dual bead complex is injected into the flow channel 160 through inlet port 152. As flow channel 160 is further filled with the dual bead complex from pipette 214, the dual bead complex 194 begins to move down flow channel 160 as the disc is rotated. The loading chamber 164 may include a break-away retaining wall 228 so that complex 194 moves down the flow channel at one time.

In this embodiment, anchor agents 222, attached to reporter beads 192, bind to the capture agents 220 by antibody-antigen interactions, as illustrated in FIG. 27C. In this manner, reporter beads 192 are retained within target zone 170. Binding can be further facilitated by rotating the disc so that the dual bead complex 194 can slowly move or tumble down the flow channel. Slow movement allows ample time for additional binding between the capture agents 220 and the anchor agent 222. After binding, the disc can be rotated further at the same speed or faster to clear target zone 170 of any unattached dual bead complex 194, as illustrated in FIG. 27D.

An interrogation beam 224 can then be directed through target zones 170 to determine the presence of reporters, capture beads, and dual bead complex, as illustrated in FIG. 27D. In the event no target antigen is present in the test sample, there will be no dual bead complex structures, reporters, or capture beads bound to the target zones 170, but a small amount of background signal may be detected in the target zones from non-specific binding. In this case, when the interrogation beam 224 is directed into the target zone 170, a zero or low reading results, thereby indicating that no target was present in the sample.

The speed, direction, and stages of rotation, such as one speed for one period followed by another speed for another period, can all be encoded in the operational information on the disc.

The methods described in FIGS. 25A-25D, 26A-26D, and 27A-27D are implemented using the reflective disc system 144. As indicated above, it should be understood that these methods and any other bead or sphere detection may also be carried out using the transmissive disc embodiment 180, as described in FIGS. 4A-4C, 5B, and 6B. It should also be understood that the methods described in FIGS. 11A-11B, 12A-12B, 25A-25D, 26A-26D, and 27A-27D are not limited to creating the dual bead complexes outside of the optical bio-discs but may include embodiments that use “in-disc” or “on-disc” formation of the dual bead complexes. In these on-disc implementations the dual bead complex is formed within the fluidic circuits of the optical bio-disc 110. For example, the dual bead formation may be carried out in the loading or mixing chamber 164. In one embodiment, the beads and sample are added to the disc at the same time, or nearly the same time. Alternatively, the beads with the probes can be pre-loaded on the disc for future use with a sample so that only a sample needs to be added.

The beads would typically have a long shelf life, with less shelf life for the probes. The probes can be dried or lyophilized (freeze dried) to extend the period during which the probes can remain in the disc. With the probes dried, the sample essentially reconstitutes the probes and then mixes with the beads to produce dual bead complex structures can be performed.

In either case, the basic process for on-disc processing includes: (1) inserting the sample into a disc with beads with probes; (2) causing the sample and the beads to mix on the disc; (3) isolating, such as by applying a magnetic field, to hold the dual bead complex and move the non-held beads away, such as to a region referred to here as a waste chamber; and (4) directing the dual bead complexes (and any other material not moved to the waste chamber) to the capture fields. The detection process can be the same as one of those described above, such as by event detection or fluorimetry.

In addition to the above, it would be apparent to those of skill in the art that the disc surface capturing techniques and the linking techniques for forming the dual bead complexes illustrated in FIGS. 25A-25D, 26A-26D, and 27A-27D may be interchanged to create alternate variations thereof. For example, the inventors have contemplated that the capture agents 220 as implemented to include specific sequences of nucleic acids may be used to capture dual bead complexes formed by either DNA hybridization as illustrated in FIG. 10A or the antibody-antigen interactions shown in FIG. 10B. Similarly, capture agents 220 as implemented to include antibodies may be employed to capture dual bead complexes formed by either the DNA hybridization method shown in FIG. 10A or the antibody-antigen interactions illustrated in FIG. 10B. And also, capture agents 220 as implemented to include biotin or BSA-biotin may be similarly utilized to capture dual bead complexes formed by either the DNA hybridization techniques illustrated in FIG. 10A or the antibody-antigen interactions depicted in FIG. 10B. Other combinations including different anchor agents to perform the binding function with the capture agent, are readily apparent from the present disclosure and are thus specifically provided for herein.

Detection and Related Signal Processing Methods and Apparatus

The number of reporter beads, target cells, or particles bound in the capture field or zone can be detected in a qualitative manner, and may also be quantified by the optical disc reader.

The test results of any of the test methods described above can be readily displayed on monitor 114 (FIG. 1). The disc according to the present invention preferably includes encoded software that is read to control the controller, the processor, and the analyzer as shown in FIG. 2. This interactive software is implemented to facilitate the methods described herein and the display of results.

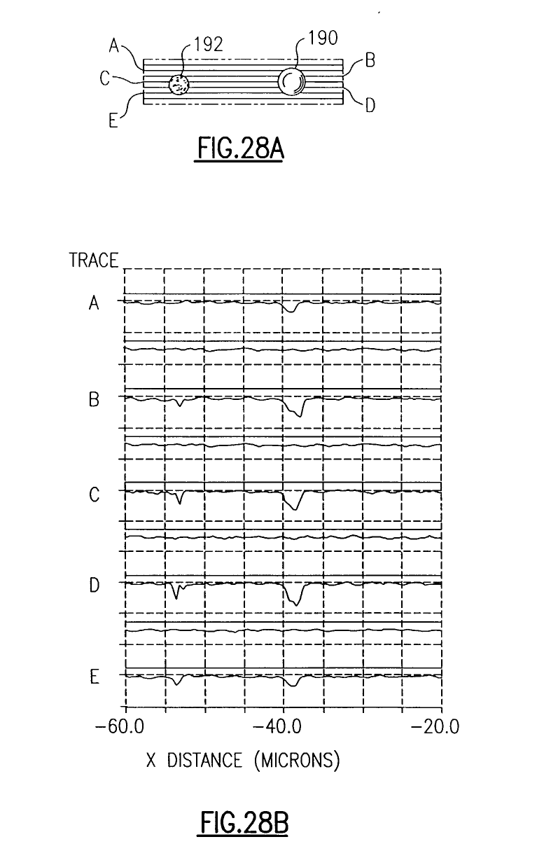

FIG. 28A is a graphical representation of an individual 2.1 um reporter bead 192 and a 3 um capture bead 190 positioned relative to tracks A, B, C, D, and E of an optical bio-disc or medical CD according to the present invention.

FIG. 28B is a series of signature traces, from tracks A, B, C, D, and E, derived from the beads of FIG. 28A utilizing a detected signal from the optical drive according to the present invention. These graphs represent the detected return beam 124 of the reflective disc illustrated in FIGS. 5A and 6A for example, or the transmitted beam 128 of the transmissive disc illustrated in FIGS. 5B and 6B. As shown, the signatures for a 2.1 um reporter bead 190 are sufficiently different from those for a 3 um capture bead 192 such that the two different types of beads can be detected and discriminated. This detection method is not limited to beads or bead complexes but may also be used to detect other objects such as cells or bead-cell complexes. As would be apparent to one of skill in the art, signature traces from a 1 um magnetic bead and an 8 um cell in an MO bio-disc, for example, would be sufficiently different so as to distinguish these particles from one another. A sufficient deflection of the trace signal from the detected return beam as it passes through a bead is referred to as an event.

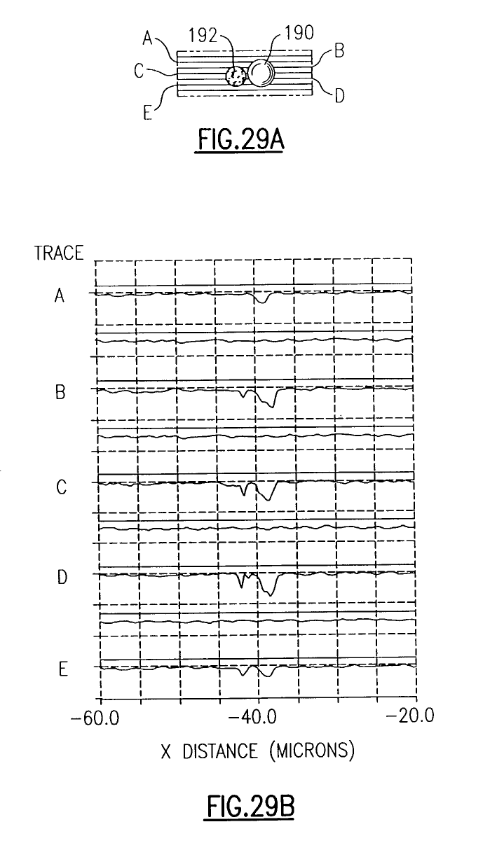

FIG. 29A is a graphical representation of a 2.1 um reporter bead and a 3 um capture bead linked together in a dual bead complex positioned relative to the tracks A, B, C, D, and E of an optical bio-disc or medical CD according to the present invention.

FIG. 29B is a series of signature traces, from tracks A, B, C, D, and E, derived from the beads of FIG. 29A utilizing a detected signal from the optical drive according to the present invention. These graphs represent the detected return beam 124 of a reflective disc 144 or transmitted beam 128 of a transmissive disc 180. As shown, the signatures for a 2.1 um reporter bead 190 are sufficiently different from those for a 3 um capture bead 192 such that the two different types of beads can be detected and discriminated. A sufficient deflection of the trace signal from the detected return beam or transmitted as it passes through a bead is referred to as an event. The relative proximity of the events from the reporter and capture bead indicates the presence or absence the dual bead complex. As shown, the traces for the reporter and the capture bead are right next to each other indicating the beads are joined in a dual bead complex.

Alternatively, other detection methods can be used. For example, reporter beads can be fluorescent or phosphorescent. Detection of these reporters can be carried out in fluorescent or phosphorescent type optical disc readers. Other signal detection methods are described, for example, in commonly assigned co-pending U.S. patent application Ser. No. 10/008,156 entitled “Disc Drive System and Methods for Use with Bio-Discs” filed Nov. 9, 2001, which is expressly incorporated by reference; U.S. Provisional Application Ser. No. 60/270,095 filed Feb. 20, 2001 and 60/292,108, filed May 18, 2001; and the above referenced U.S. patent application Ser. No. 10/043,688 entitled “Optical Disc Analysis System Including Related Methods For Biological and Medical Imaging” filed Jan. 10, 2002.

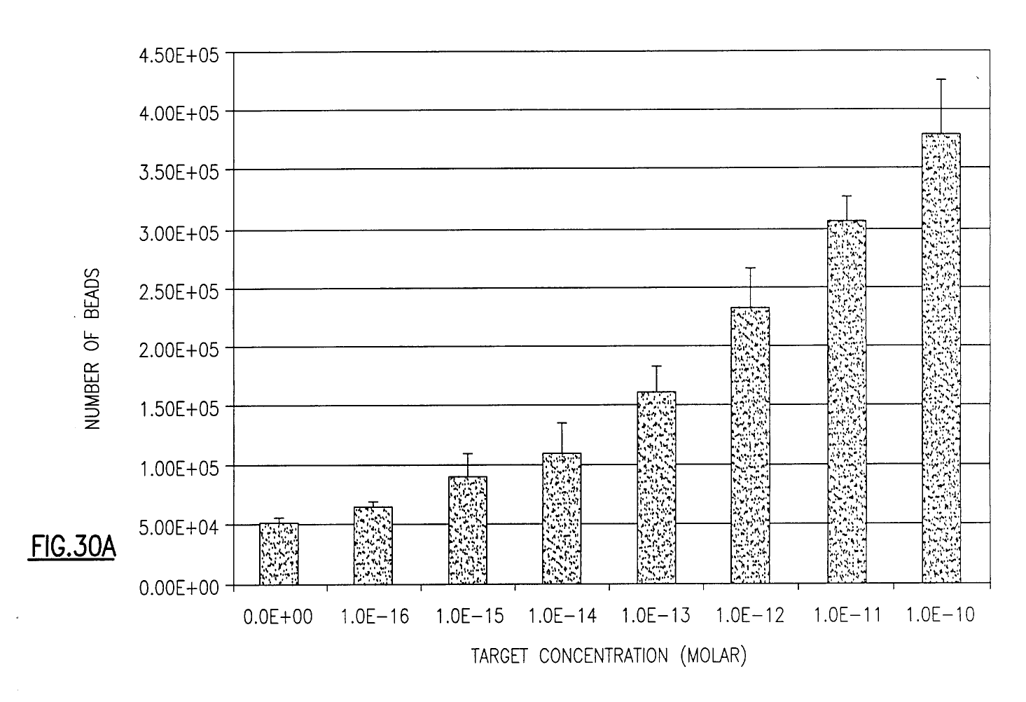

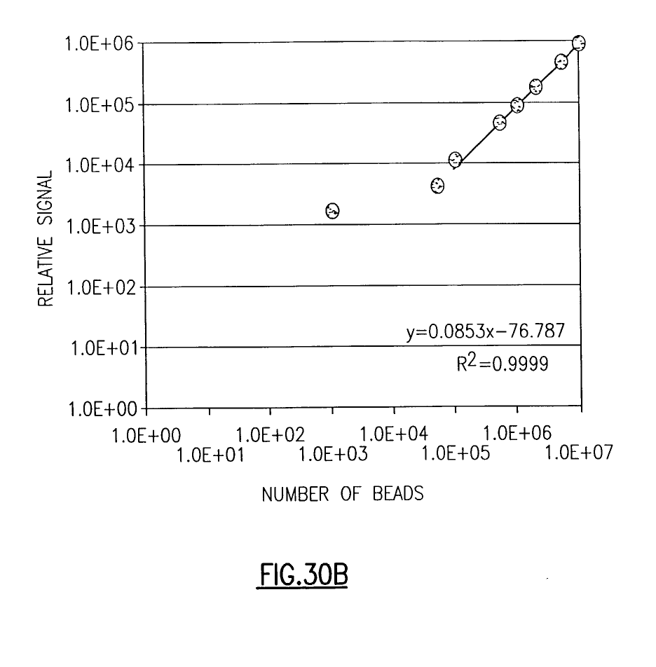

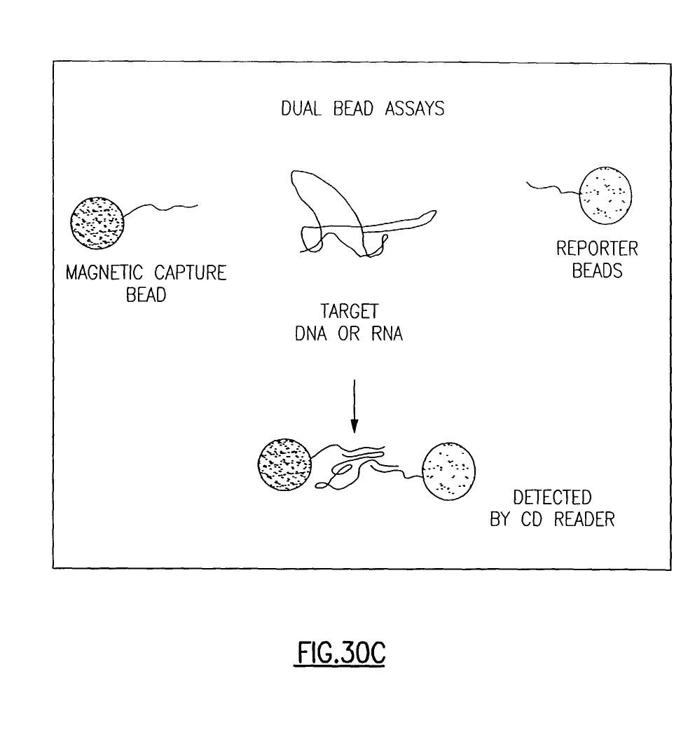

FIG. 30A is a bar graph of data generated using a fluorimeter showing concentration-dependent target detection using fluorescent reporter beads. This graph shows the molar concentration of target DNA versus the number of detected beads. The dynamic range of target detection shown in the graph is 10E-16 to 10E-10 Molar (moles/liter). While the particular graph shown was generated using data from a fluorimeter, the results may also be generated using a fluorescent type optical disc drive.

FIG. 30B presents a standard curve demonstrating that the sensitivity of a fluorimeter is approximately 1000 beads in a fluorescent dual bead assay. The sensitivity of any assay depends on the assay itself and on the sensitivity of the detection system. Referring to FIGS. 30A-30C, various studies were done to examine the sensitivity of the dual bead assay using different detection methods, e.g., a fluorimeter, and bio-disc or medical CD detection according to the present invention.

As stated above and shown in FIG. 30B, the sensitivity of a fluorimeter is approximately 1000 beads in a fluorescent dual bead assay. In contrast, FIG. 30A shows that even at 10E-16 Molar (moles/liter), a sufficient number of beads over zero concentration can be detected to sense the presence of the target. With a sensitivity of 10E-16 Molar, a dual bead assay represents a very sensitive detection method for DNA that does not require DNA amplification (such as through PCR) and can be used to detect even a single bead.

In contrast to conventional detection methods, the use of a medical CD or bio-disc coupled with a CD-reader or optical bio-disc drive (FIG. 1) improves the sensitivity of detection. For example, while detection with a fluorimeter is limited to approximately 1000 beads (FIG. 30B), use of a bio-disc coupled with CD-reader may enable the user to detect a single bead with the interrogation beam as illustrated in FIGS. 29A, 29B, and 30C. Thus, the bioassay system provided herein improves the sensitivity of dual bead assays significantly.

The detection of single beads using an optical bio-disc or medical CD is discussed in detail in conjunction with FIGS. 28A and 28B. FIG. 28B shows the signal traces of each bead as detected by the medical CD or bio-disc reader. Dual bead complexes may also be identified by the bio-disc reader using the unique signature traces collected from the detection of a dual bead complex as shown in FIGS. 29A and 29B. Different optical bio-disc platforms, including but not limited to the reflective and the transmissive disc formats illustrated respectively in FIGS. 3C and 4C, may be used in conjunction with the reader device for detection of beads.

FIG. 30C is a pictorial representation demonstrating the formation of the dual bead complex linked together by the presence of the target in a genetic assay. Sensitivity to within one reporter molecule is possible with the present dual bead assay quantified with a bio-CD reader shown in FIGS. 1 and 2 above. Similarly, the dual bead complex formation may also be implemented in an immunochemical assay format as illustrated above in FIGS. 7B, 8B, 9B, 10B, 11B, and 12B.

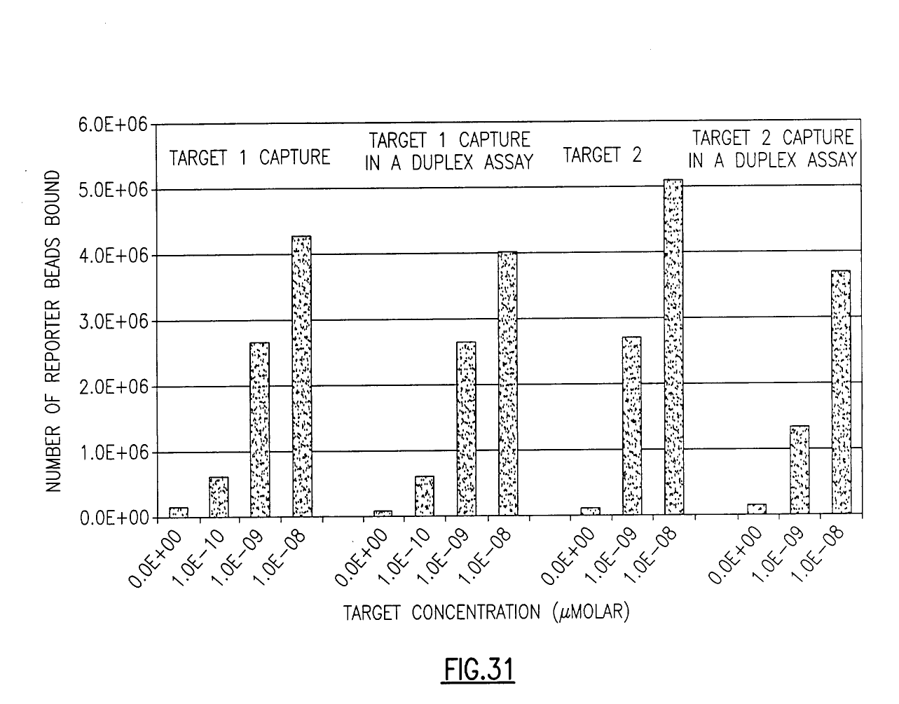

FIG. 31 shows data generated using a fluorimeter illustrating the concentration-dependent detection of two different targets. Target detection was carried out using two different methods (the single and the duplex assays). In the single assay, the capture bead contains a transport probe specific to a single target and a reporter probe coated with a signal probe specific to the same target is mixed in a solution together with the target. In the duplex assay, the capture bead contains two different transport probes specific to two different targets. Experimental details regarding the use of the duplex target detection method are discussed in further detail in Example 2. Mixing different reporter beads (red and green fluorescent or silica and polystyrene beads, for example) containing signal probes specific to one of the two targets, allows the detection of two different targets simultaneously.

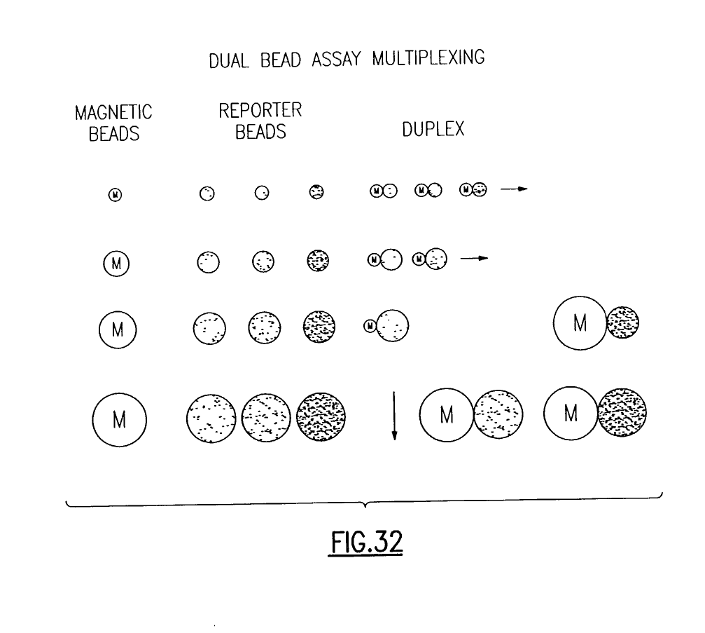

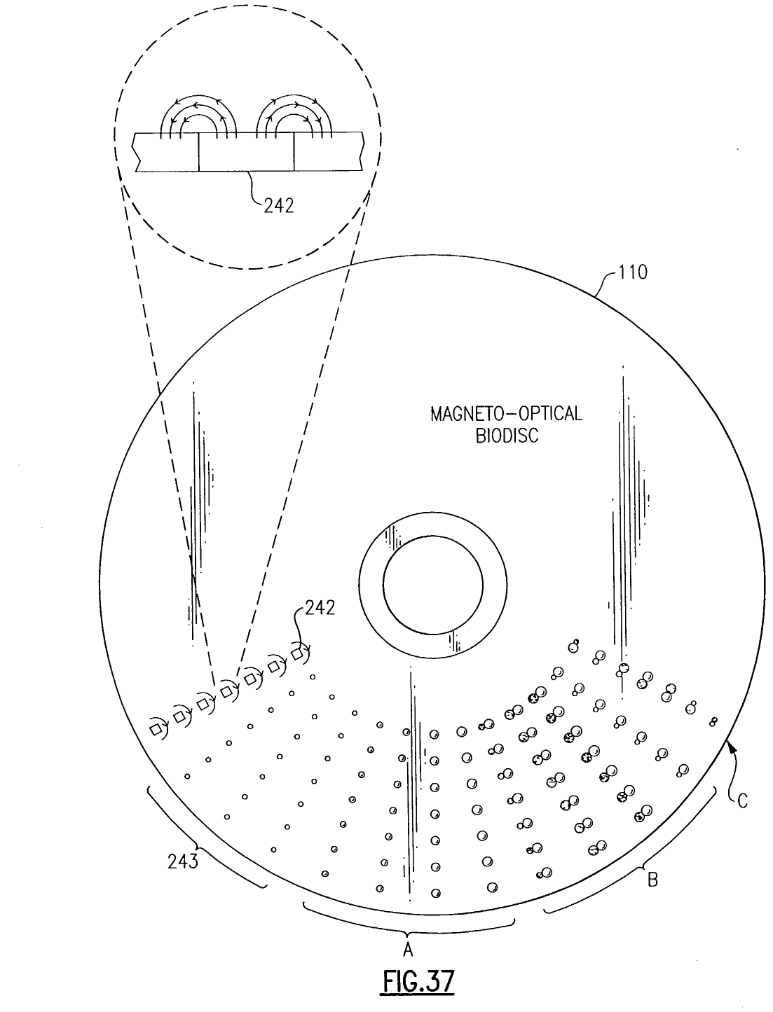

Detection of the dual bead duplex assay may be carried out using a magneto-optical disc system described below. FIGS. 32 and 37 illustrate the formation and binding of various dual bead complexes onto an optical disc which may be detected by an optical bio-disc drive (FIG. 2), a magneto-optical disc system, a fluorescent disc system, or any similar device. Unique signature traces of a dual bead complex collected from an optical disc reader are shown in FIG. 29B above. The traces from FIG. 29B further illustrate that different bead types can be detected by an optical disc reader since different beads show different signature profiles.

Multiplexing, Magneto-Optical, and Magnetic Discs Systems

The use of a dual bead assay in the capture of targets allows for the use in multiplexing assays. This type of multiplexing is achieved by combining different sizes of magnetic beads with different types and sizes of reporter beads. Thus, different target agents can be detected simultaneously. As indicated in FIG. 32, four sizes of magnetic capture beads, and four sizes of three types of reporter beads produce up to 48 different types of dual bead complex. In a multiplexing assay, probes specific to different targets are thus conjugated to capture beads. Reporter beads having different physical and/or optical properties, such as fluorescence at different wavelengths, allow for simultaneous detection of different target agents from the same biological sample. As indicated in FIGS. 28A, 28B, 29A, and 29B, small differences in size can be detected by detecting reflected or transmitted light.

Multiple dual bead complex structures for capturing different target agents can be carried out on or off the disc. The dual bead suspension is loaded into a port on the disc. The port is sealed and the disc is rotated in the disc reader. During spinning, free (unbound) reporter beads are spun off to a periphery of the disc while the magnetic capture beads and magnetic bead complexes or dual beads are captured in a magnetic field, in magnetic domains on the MO disc, for example. The reporter beads detecting various target agents are thus localized in capture regions. In this manner, the presence of a specific target agent can be detected, and the amount of a specific target agent can be quantified by the disc reader.

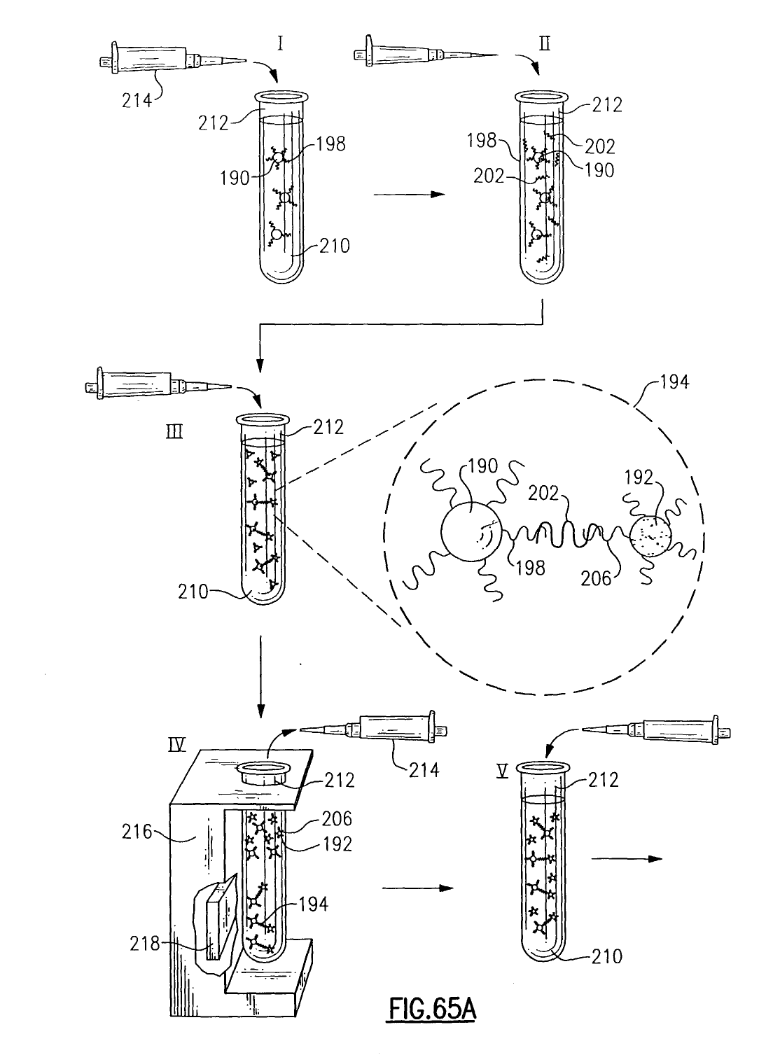

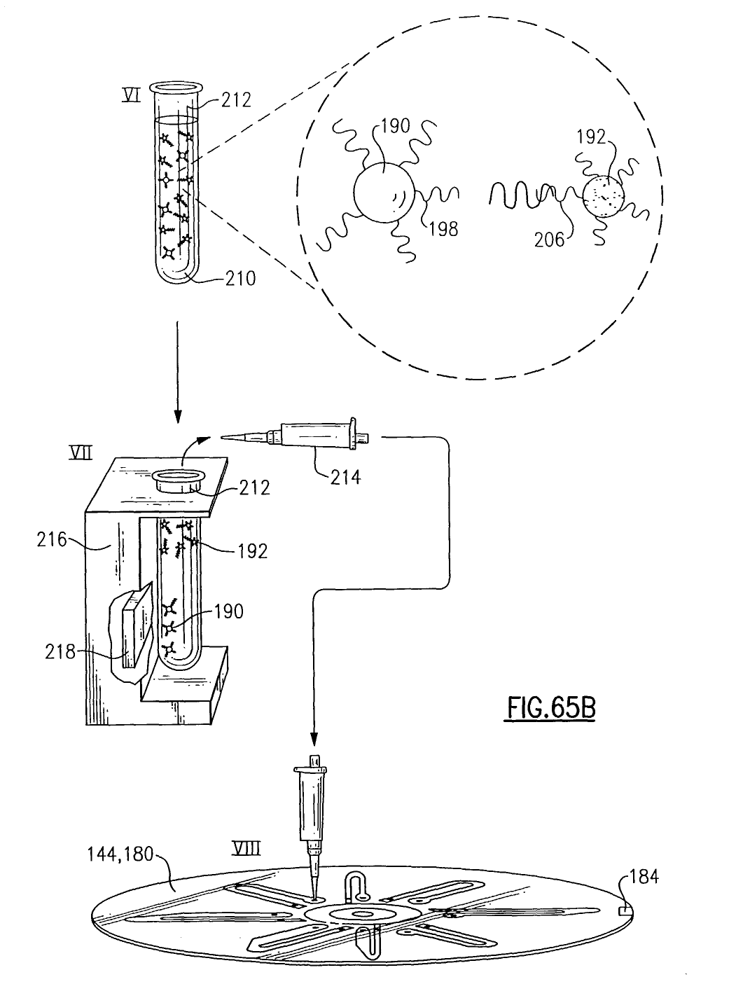

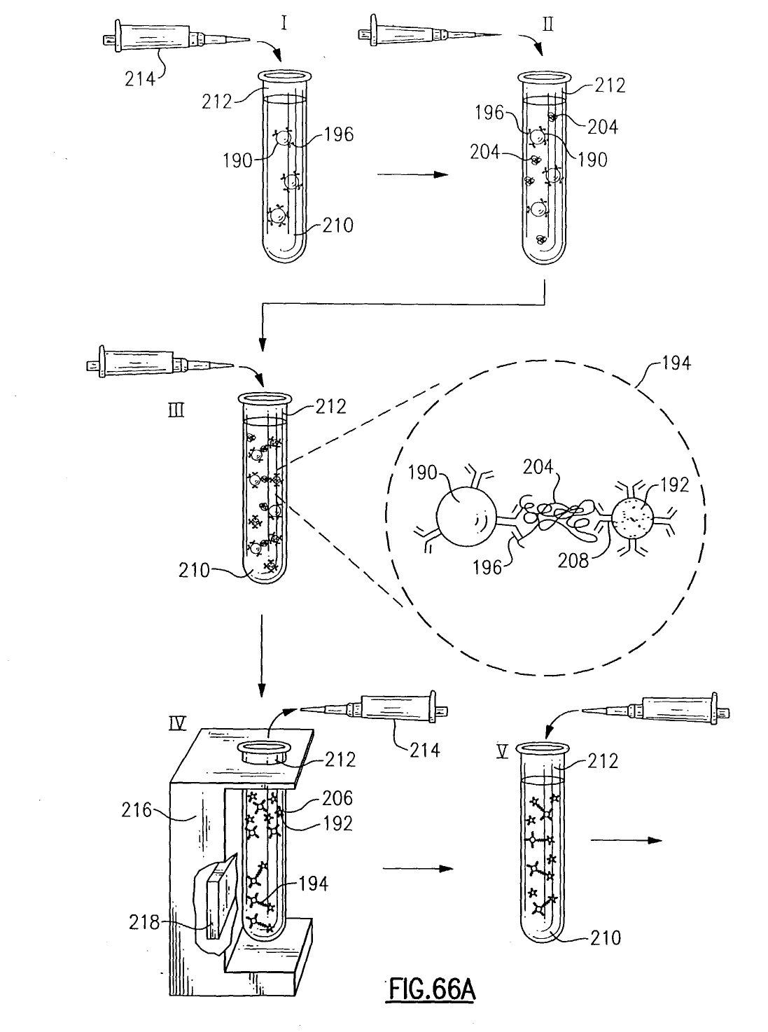

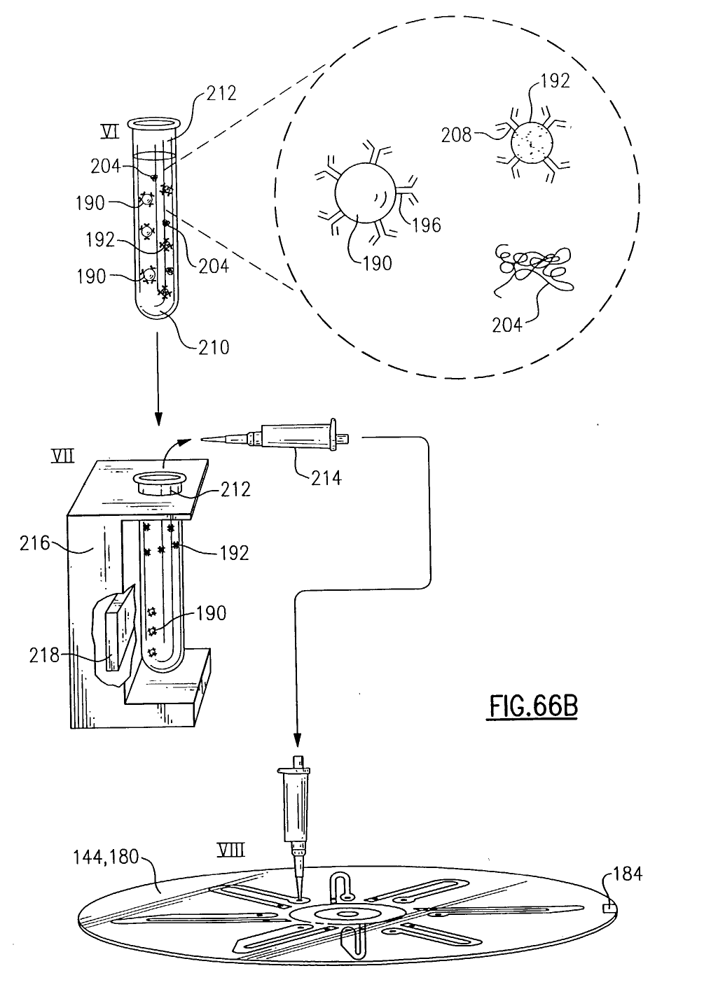

FIG. 33A is a general representation of an optical disc according to another aspect of the present invention. The disc 110 illustrated in FIG. 33A may be employed to practice the methods corresponding generally to the single-step methods of FIGS. 11A and 11B illustrated above or the related single step methods described below in connection with FIGS. 65A-65B and 66A-66B. The sample and beads can be added at one time or successively but closely in time. Alternatively, the beads can be pre-loaded into a portion of the disc. These materials can be provided to a mixing chamber 164 that can have a breakaway wall 228 (see FIG. 25A), which holds in the solution within the mixing chamber 164. Mixing the sample and beads on the disc would be accomplished through rotation at a rate insufficient to cause the wall to break or the capillary forces to be overcome.

The disc can be rotated in one direction, or it can be rotated alternately in opposite directions to agitate the material in a mixing chamber. The mixing chamber is preferably sufficiently large so that circulation and mixing is possible. The mixing can be continuous or intermittent.

FIG. 33B shows one embodiment of a rotationally-directionally-dependent valve arrangement that uses a movable component for a valve. The mixing chamber leads to an intermediate chamber 244 that has a movable component, such as a ball 246. In the non-rotated state, the ball 246 may be kept in a slight recessed portion, or chamber 244 may have a gradual V-shaped tapering in the circumferential direction to keep the ball centered when there is no rotation.

Referring to FIGS. 33C and 33D in addition to FIGS. 33A and 33B, when the disc is rotated clockwise (FIG. 33C), ball 246 moves to a first valve seat 248 to block passage to detection chamber 234 and to allow flow to waste chamber 232, shown in FIG. 33A. When the disc is rotated counter-clockwise (FIG. 33D), ball 246 moves to a second valve seat 250 to block a passage to waste chamber 232 and to allow flow to detection chamber 234.

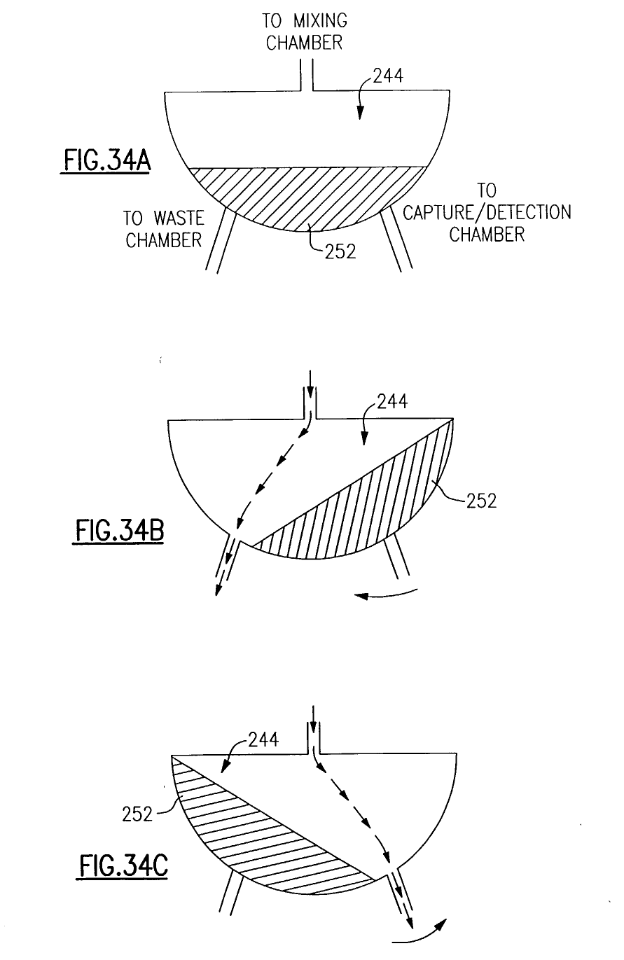

FIGS. 34A-34C show a variation of the prior embodiment in which the ball is replaced by a wedge 252 that moves one way or the other in response to acceleration of the disc. The wedge 252 can have a circular outer shape that conforms to the shape of an intermediate chamber 244. The wedge is preferably made of a heavy dense material relative to chamber 244 to avoid sticking. A coating can be used to promote sliding of the wedge relative to the chamber.

When the disc is initially rotated clockwise as shown in FIG. 34B, the angular acceleration causes wedge 252 to move to block a passage to detection chamber 234 and to allow flow to waste chamber 232. When the disc is initially rotated counter-clockwise, FIG. 34C, the angular acceleration causes wedge 252 to block passage to waste chamber 232 and allows flow to detection chamber 234. During constant rotation after the acceleration, wedge 252 remains in place blocking the appropriate passage.

In another embodiment of the present invention where the capture beads are magnetic, a magnetic field from a magnetic field generator or field coil 230 can be applied over the mixing chamber 164 to hold the dual bead complexes and unbound magnetic beads in place while material without magnetic beads are allowed to flow away to a waste chamber 232. This technique may also be employed to aid in mixing of the assay solution within the fluidic circuits or channels before any unwanted material is washed away. At this stage, only magnetic capture beads, unbound or as part of a dual bead complex, remain. The magnetic field is released, and the dual bead complex with the magnetic beads is directed to a capture and detection chamber 234.

The process of directing non-magnetic beads to waste chamber 232 and then magnetic beads to capture chamber 234 can be accomplished through the microfluidic construction and/or fluidic components. A flow control valve 236 or some other directing arrangement can be used to direct the sample and non-magnetic beads to waste chamber 232 and then to capture chamber 234. A number of embodiments for rotationally dependent flow can be used. Further details relating to the use of flow control mechanisms are disclosed in commonly assigned co-pending U.S. patent application Ser. No. 09/997,741 entitled “Dual Bead Assays Including Optical Biodiscs and Methods Relating Thereto” filed Nov. 27, 2001, which is herein incorporated by reference in its entirety.

FIG. 35 is a perspective view of a disc including one embodiment of a fluidic circuit employed in conjunction with magnetic beads and the magnetic field generator 230 according to the present invention. FIG. 35 also shows the mixing chamber 164, the waste chamber 232, and the capture chamber 234. The magnetic field generator 230 is positioned over disc 110 and has a radius such that as disc 110 rotates, magnetic field generator 230 remains over mixing chamber 164, and is radially spaced from chambers 232 and 234. As with the prior embodiment discussed above, a magnetic field from the magnetic field generator 230 can be applied over the mixing chamber 164 to hold the dual bead complexes and/or unbound magnetic beads in place while additional material is allowed to enter the mixing chamber 164. The method of rotating the disc while holding magnetic beads in place with the magnetic field generator 230 may also be employed to aid in mixing of the assay solution within the mixing chamber 164 before the solution contained therein is directed elsewhere.

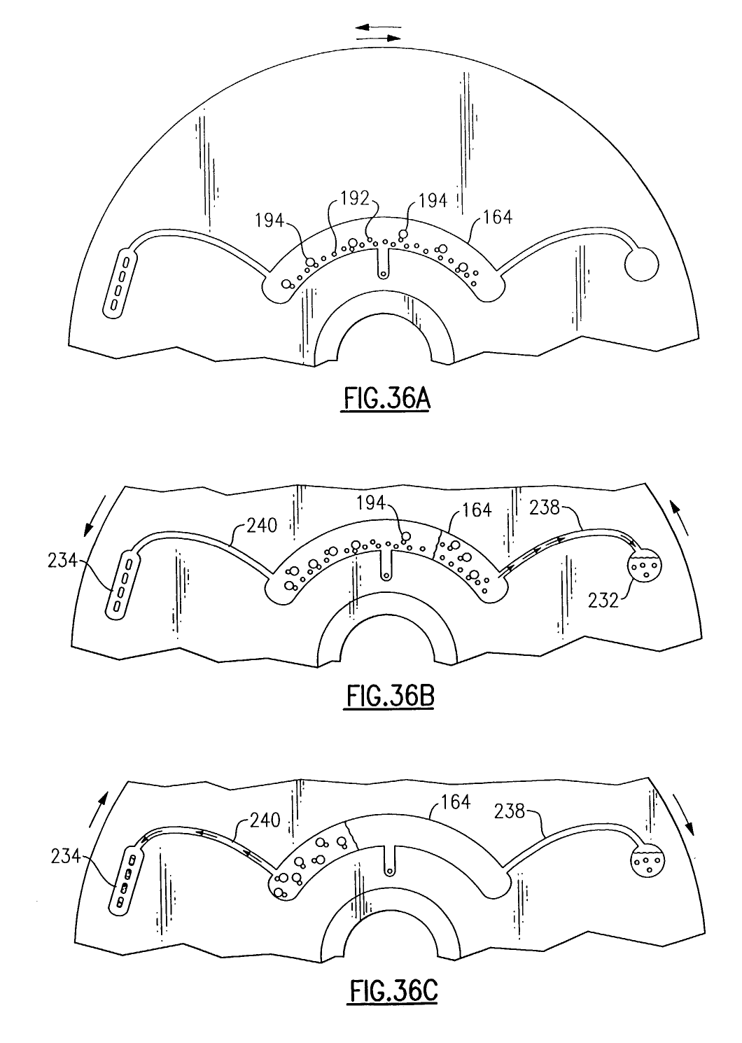

FIGS. 36A-36C are plan views illustrating a method of separation and detection for dual bead assays using the fluidic circuit shown in FIG. 35. FIG. 36A shows an unrotated optical disc with a mixing chamber 164 shaped as an annular sector holding a sample with dual bead complexes 194 and various unbound reporter beads 192. The electromagnet is activated and the disc is rotated counter-clockwise (FIG. 36B), or it can be agitated at a lower rpm, such as 1× or 3×. Dual bead complexes 194, with magnetic capture beads, remain in mixing chamber 164 while the liquid sample and the unbound reporter beads 192 move in response to angular acceleration to a rotationally trailing end of mixing chamber 164. The disc is rotated in the counter-clockwise direction illustrated in FIG. 36B with sufficient speed to overcome capillary forces to allow the unbound reporter beads in the sample to move through a waste fluidic circuit 238 to waste chamber 232. At this stage in the process, the liquid will not move down the capture fluidic circuit 240 because of the physical configuration of the fluidic circuit as illustrated.

As illustrated next in FIG. 36C, the magnet is deactivated and the disc is rotated clockwise. Dual bead complexes 194 move to the opposite trailing end of the mixing chamber 164 in response to angular acceleration and then through a capture fluidic circuit 240 to the capture chamber 234. At this later stage in the process, the dual bead solution will not move down the waste fluidic circuit 238 due to the physical layout of the fluidic circuit, as shown. The embodiment shown in FIGS. 36A-36C thus illustrates directionally-dependent flow as well as rotational speed dependent flow.

In this embodiment and others in which a fluidic circuit is formed in a region of the disc, a plurality of regions can be formed and distributed about the disc, for example, in a regular manner to promote balance. Furthermore, as discussed above, instructions for controlling the rotation can be provided on the disc. Accordingly, by reading the disc, the disc drive can have instructions to rotate for a particular period of time at a particular speed, stop for some period of time, and rotate in the opposite direction for another period of time. In addition, the encoded information can include control instructions such as those relating to, for example, the power and wavelength of the light source. Controlling such system parameters is particularly relevant when fluorescence is used as a detection method.

In yet another embodiment, a passage can have a material or configuration that can seal or dissolve either under influence from a laser in the disc drive, or with a catalyst pre-loaded in the disc, or such a catalyst provided in the test sample. For example, a gel may solidify in the presence of a material over time, in which case the time to close can be set sufficiently long to allow the unbound capture beads to flow to a waste chamber before the passage to the waste chamber closes. Alternatively, the passage to the waste chamber can be open while the passage to the detection chamber is closed. After the unbound beads are directed to the waste chamber, the passage to the direction chamber is opened by energy introduced from the laser to allow flow to the detection chamber.

With reference now generally to FIG. 37, it is understood that magneto-optic recording is an optical storage technique in which magnetic domains or areas are written into a thin film by heating it with a focused laser in the presence of an external magnetic field. The presence of these domains is then detected by the same laser from differences in the polarization of the reflected light between the different magnetic domains in the layer (Kerr rotation). By switching either the magnetic field for constant high laser power, or modulating the laser power with a constant magnetic field, a data pattern can be written into the layer. Many magneto-optic storage systems have been introduced into the market, including both computer data storage systems and audio systems (most notably MiniDisc). Descriptions of the current status of this field can be found in “The Principles of Optical Disc Systems”, Bouwhuis et. al. 1985 (ISBN 0-85274-785-3); “Optical Recording, A Technical Overview” A. B. Marchant 1990 (ISBN 0-201-76247-1); and “The Physical Principles of Magneto-Optical Recording”, M. Mansuripur 1995 (ISBN 0521461243). All of these documents are herein incorporated by reference in their respective entireties.

Moving now specifically to FIG. 37, there is illustrated yet another embodiment of the optical disc 110 for use with a multiplexing dual bead assay. In this case, a disc, such as one used with a magneto-optical drive, has magnetic domains or regions 242 that can be selectively written and erased with a magnetic head. Hereafter this type of disc will generally be referred to as a “magneto-optical bio-disc” or an “MO bio-disc”. A magneto-optical disc drive, for example, can create magnetic domains 242 as small as 1 um by 1 um square. The close-up section of the magnetic domain 242 shows the direction of the magnetic field with respect to adjacent regions.

The ability to selectively write to small areas in a highly controllable manner to make them magnetic allows capture areas to be created in desired locations. These magnetic capture areas or domains can be formed in any desired configuration or location in one fluidic chamber or in multiple fluidic chambers. These areas capture and hold magnetic beads when applied over the disc. The domains can be selectively erased if desired, thereby allowing them to be made non-magnetic and allowing the beads to be released.

In one configuration of a magnetic bead array according to this aspect of the present invention, a set of three radially oriented magnetic capture regions 243 are shown, by way of example, with no beads attached to the magnetic capture regions in the columns illustrated therein. With continuing reference to FIG. 37, there is shown a set of four columns in Section A with individual magnetic beads magnetically attached to the magnetic areas in a magnetic capture region. Another set of four columns arrayed in Section B is shown after binding of reporter beads to form dual bead complexes attached to specific magnetic domains or areas, with different columns having different types of reporter beads. As illustrated in Section B, some of the reporter beads utilized vary in size to thereby achieve the multiplexing aspects of the present invention as implemented on a magneto-optical bio-disc or MO medical disc. In Section C, a single column of various dual bead complexes is shown as another example of multiplexing assays employing various bead sizes individually attached at separate magnetic areas.

In a method of using such a magneto-optical bio-disc, the write head in an MO drive is employed to create magnetic domains, and then a sample can be directed over that domain to capture magnetic beads provided in the sample. After introduction of the first sample set, other magnetic domains may also be created and another sample set can be provided to the newly created magnetic capture region for detection. Thus detection of multiple sample sets may be performed on a single disc at different time periods. The magneto-optical drive also allows the demagnetization of the magnetic domains or capture regions to thereby release and isolate the magnetic beads if desired. Thus this system provides for the controllable capture, detection, isolation, and release of one or more specific target molecules from a variety of different biochemical, chemical, or biological samples.

As described above, a sample can be provided to a fluidic chamber on a disc. Alternatively, a sample could be provided to multiple chambers that have sets of different beads. In addition, a series of chambers can be created such that a sample can be moved by rotational motion from one chamber to the next, and separate tests can then be performed in each chamber.

With such an MO bio-disc, a large number of tests can be performed at one time and can be performed interactively. In this manner, when a test is performed and a result is obtained, the system can be instructed to create a new set of magnetic regions for capturing the dual bead complex. Regions can be created one at a time or in large groups, and can be performed in successive chambers that have different pre-loaded beads. Other processing advantages can be obtained with an MO bio-disc that has writeable magnetic regions. For example, the “capture agent” is essentially the magnetic field created by the magnetic region on the disc and therefore there is no need to add an additional biological or chemical capture agent.

Instructions for controlling the locations for magnetic regions written or erased on the MO bio-disc, and other information such as rotational speeds, stages of rotation, waiting periods, wavelength of the light source, and other parameters can be encoded on and then read from the disc itself. As would be readily apparent to one of ordinary skill in the art given the disclosure provided herein, the MO bio-disc illustrated in FIG. 37 may include any of the fluidic circuits, mixing chambers, flow channels, detection chambers, inlet ports, or vent ports employed in the reflective and transmissive discs discussed above. Illustrative examples of the use of the MO bio-disc according to this aspect of the present are provided below in Examples 5 and 6.

Thus in summary, the following embodiments of the magneto-optical aspects of the present invention have been contemplated by the inventors and are herein described in detail. Firstly, there is provided a method of performing a genetic dual bead assay in association with a magneto-optical bio-disc. This method includes the steps providing a plurality of magnetic capture beads having covalently attached transport probes, providing a plurality of reporter beads having covalently attached specific sequences of DNA, preparing a sample containing target DNA molecules to be tested for DNA sequences complementary to the specific DNA sequences, and loading the capture beads into a magneto-optical bio-disc via an inlet port provided therein. The magneto-optical bio-disc has a magnetic capture layer. This method further includes loading the sample and the plurality of reporter beads into the bio-disc, rotating the bio-disc to facilitate hybridization of any target DNA present in the sample to the specific sequences of DNA on the reporter beads and to the transport probes to form dual bead complexes, interrogating a number of the magnetic capture beads with an incident beam of radiant energy to determine whether each of the number of magnetic capture beads has formed a dual bead complex, magnetizing specific regions of the magnetic capture layer to bind thereto a plurality of the dual bead complexes, and quantitating the plurality of the dual bead complexes.

The method may include the further steps of rotating the disc to direct any unbound beads into a waste chamber and then de-magnetizing the specific regions of the magnetic capture layer to thereby release a number of the plurality of the dual bead complexes. Thereafter the disc may be rotated to direct the released number of dual bead complexes to an analysis area for further processing so that the released number of dual bead complexes are sequestered in the analysis area. The analysis area may be an analysis chamber having agents that react with the sequestered dual bead complexes.

According to a second embodiment of the magneto-optical aspects of the present invention there is provided another method of performing a dual bead assay in association with a magneto-optical bio-disc. This other method includes the steps of providing a plurality of magnetic capture beads having attached transport probes, providing a plurality of reporter beads having attached signal probes, and loading the capture beads into a magneto-optical bio-disc via an inlet port provided therein. The magneto-optical bio-disc has a magnetic capture layer. This second method further includes loading a sample containing a target and the plurality of reporter beads into the bio-disc, rotating the bio-disc to facilitate binding of the target and the reporter beads to the magnetic capture beads to form dual bead complexes, interrogating a number of the magnetic capture beads with an incident beam of radiant energy to determine whether each of the number of magnetic capture beads has formed a dual bead complex, magnetizing specific regions of the magnetic capture layer to bind thereto a plurality of the dual bead complexes, and quantitating the plurality of the dual bead complexes.

This method may similarly include the further step of rotating the disc to direct any unbound beads into a waste chamber and then de-magnetizing the specific regions of the magnetic capture layer to thereby release a number of the plurality of the dual bead complexes. It is also an aspect of this method to then rotate the disc to direct the released number of dual bead complexes to an analysis area for further processing so that the released number of dual bead complexes are sequestered in the analysis area. The analysis area may include a reaction chamber having agents that react with the sequestered dual bead complexes.

In accordance with a third embodiment of the magneto-optical aspects of the present invention there is provided a method of performing a multiplexed dual bead assay in association with a magneto-optical bio-disc. This multiplexing method includes the steps of (1) providing at least two groups of differently sized magnetic capture beads, each group having magnetic capture beads of the same size and having a different specific type of transport probe associated with each group; (2) providing a plurality of reporter beads having attached at least two different types of signal probes; and (3) loading the capture beads into a magneto-optical bio-disc via an inlet port provided therein. As in the above MO bio-disc methods, this magneto-optical bio-disc has a magnetic capture layer. The method also includes (4) loading a sample containing at least one target and the plurality of reporter beads into the bio-disc; (5) rotating the bio-disc to facilitate binding of the target and the reporter beads to the magnetic capture beads to form dual bead complexes; (6) interrogating a number of the magnetic capture beads with an incident beam of radiant energy to determine whether each of the number of magnetic capture beads has formed a dual bead complex; and (7) determining the size of the magnetic bead in the dual bead complex. This particular method concludes with the steps of (8) magnetizing specific regions of the magnetic capture layer to bind thereto a plurality of the dual bead complexes; and (9) quantitating the plurality of the dual bead complexes.

According to one aspect of this specific method, the step of quantitating may advantageously include quantitating the plurality of the dual bead complexes according to the size of the magnetic capture bead. The method may include the further step of rotating the disc to direct any unbound beads into a waste chamber and then de-magnetizing the specific regions of the magnetic capture layer to thereby release a number of the plurality of the dual bead complexes containing same-sized magnetic capture beads. The method may further include rotating the disc to direct the released number of same-sized dual bead complexes to an analysis area for further processing so that the released number of same-sized dual bead complexes are sequestered in the analysis area. The analysis area may include a reaction chamber having agents that react with the sequestered same-sized dual bead complexes. In one particular embodiment hereof, the signal probe is a specific sequence of DNA.

According to yet a fourth embodiment of the magneto-optical aspects of the present invention there is provided another principal method of performing a multiplexed dual bead assay in association with a magneto-optical bio-disc. This additional dual bead multiplexing method includes the steps of (1) providing at least two groups of different types of reporter beads, each group having reporter beads of the same type and having a different specific type of signal probe associated with each group; (2) providing a plurality of magnetic capture beads having different types of transport probes attached thereto; and (3) loading the capture beads into a magneto-optical bio-disc via an inlet port provided therein. As in the above MO bio-diac methods, this particular magneto-optical bio-disc has a magnetic capture layer. The method continues with the additional steps of (4) loading a sample to be tested for at least one target and the plurality of reporter beads into the bio-disc; (5) rotating the bio-disc to facilitate binding of any target present in the sample to the reporter beads and to the magnetic capture beads to form dual bead complexes; and (6) interrogating a number of the reporter beads with an incident beam of radiant energy to determine whether each of the number of reporter beads has formed a dual bead complex. This particular embodiment of the present method then concludes with (7) determining the type of the reporter bead in the dual bead complex; (8) magnetizing specific regions of the magnetic capture layer to bind thereto a plurality of the dual bead complexes; and (9) quantitating the plurality of the dual bead complexes.

In one specific embodiment hereof, the step of quantitating includes quantitating the plurality of the dual bead complexes according to the type of reporter bead. The method may further include the further step of rotating the disc to direct any unbound beads into a waste chamber and then, if desired, de-magnetizing the specific regions of the magnetic capture layer to thereby release a number of the plurality of the dual bead complexes containing same-type reporter beads. The further step of rotating the disc to direct the released number of same-type dual bead complexes to an analysis area for further processing so that the released number of same-type dual bead complexes are sequestered in the analysis area may also be performed.

As with the above methods, the analysis area may include a reaction chamber having agents that react with the sequestered same-type dual bead complexes. The present invention further contemplates an optical bio-disc used to perform any of the above methods, and an optical bio-disc used to analyze any the dual bead complexes prepared according to the methods discussed above in connection with FIGS. 11A, 11B, 12A, 12B or those methods discussed in detail below in conjunction with FIGS. 65A and 65B, 66A and 66B, 67A and 67B as well as 68A and 68B. Furthermore, the MO bio-disc may be implemented in other biomagnetic assays including immunomagnetic and molecumagnetic assays such as cellular capture and analysis methods using the MO biodisc as discussed below.

Genetic Assays Using Ligation to Increase Assay Sensitivity

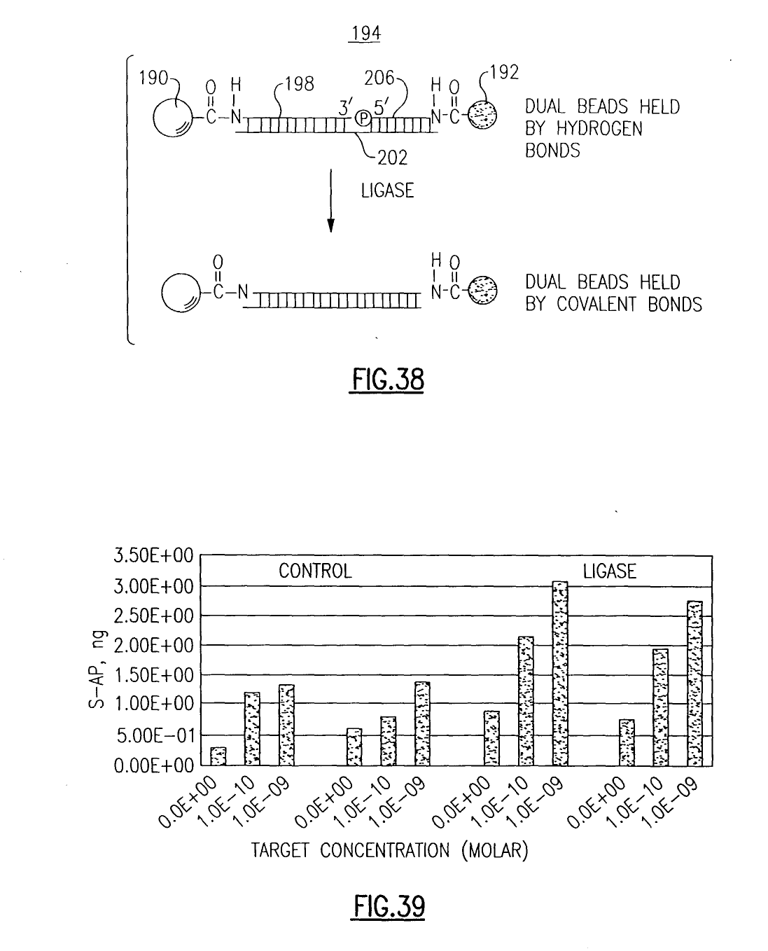

Referring to FIG. 38, there is shown the dual bead complex 194 held together by the target DNA 202 through the covalently bound transport probes 198 and signal probes 206 on the capture bead 190 and the reporter bead 192, respectively. As depicted in this figure, the 5′ end of the signal probe 206 is held right next to the 3′ end of the transport probe 198. This configuration allows the ligation of the 3′ and 5′ ends of the probes upon addition of ligase. Ligation of both probes only occurs in the presence of the target and it enhances the sensitivity of the assay by increasing the bond strength between the reporter and capture beads preventing the dissociation of the dual bead complex.

Referring now to FIG. 39, there is a bar graph illustrating the results from a genetic test detected by an enzyme assay. A 3 μm capture bead bound to transport probes was used to capture the target in this test. Once the target was captured, a biotinylated reporter probe was introduced and allowed to bind to the target. The capture beads were then washed to remove unbound reporter probes. Ligase is then added to the solution to ligate the ends of the reporter and transport probes, as shown in FIG. 38. After a series of wash steps, streptavidinated-alkaline phosphatase is added to the bead solution and allowed to bind with the biotin on the reporter probe. The beads are again washed and a chromagen alkaline phosphatase substrate is added to the bead solution. The intensity of the color formed by the alkaline phosphatase and substrate reaction is then quantified using a spectrophotometer. The results from this quantification are shown in FIG. 39. The data presented in this figure indicates that there is approximately a 50% increase in signal when the probes are ligated. Thus the assay sensitivity is significantly increased by the ligation step in this experiment. Examples 3 and 4 discuss in detail the procedures followed in carrying out a similar experiment.

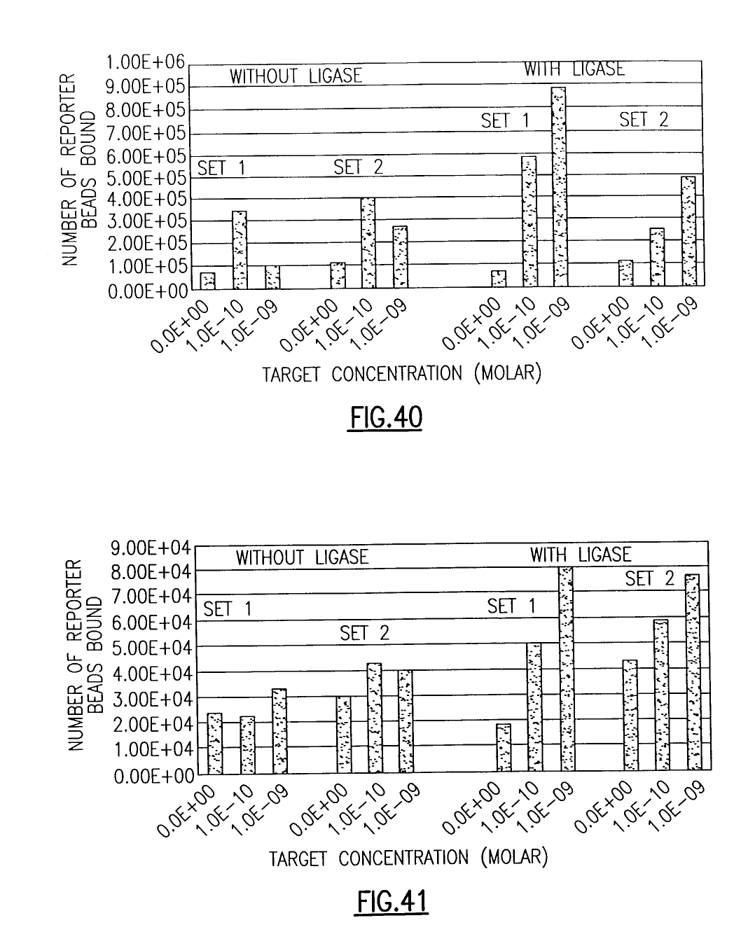

FIG. 40 shows a bar graph from a genetic test using a ligation step implemented in a dual bead assay instead of an enzyme assay. The enzyme assay as discussed in FIG. 39, is used to verify the activity of ligase in a non-dual bead format, which serves as a control in the dual bead experiment. As with the enzyme assay, the same 3 um capture beads bound to transport probes were used in the dual bead assay. The reporter beads used in the dual bead assay were 2.1 um fluorescent beads. The dual beads were formed as discussed in either FIG. 11A or 12A. The ligation step is implemented in Step V in FIG. 11A or Step VI in FIG. 12A where ligase is added to the dual bead complex solution and allowed to ligate the transport probes to the signal probes. The data shown in FIG. 40 indicates that ligation significantly increases the signal and sensitivity of the assay relative to the non-ligated control treatment in Set 1 but not in Set 2.

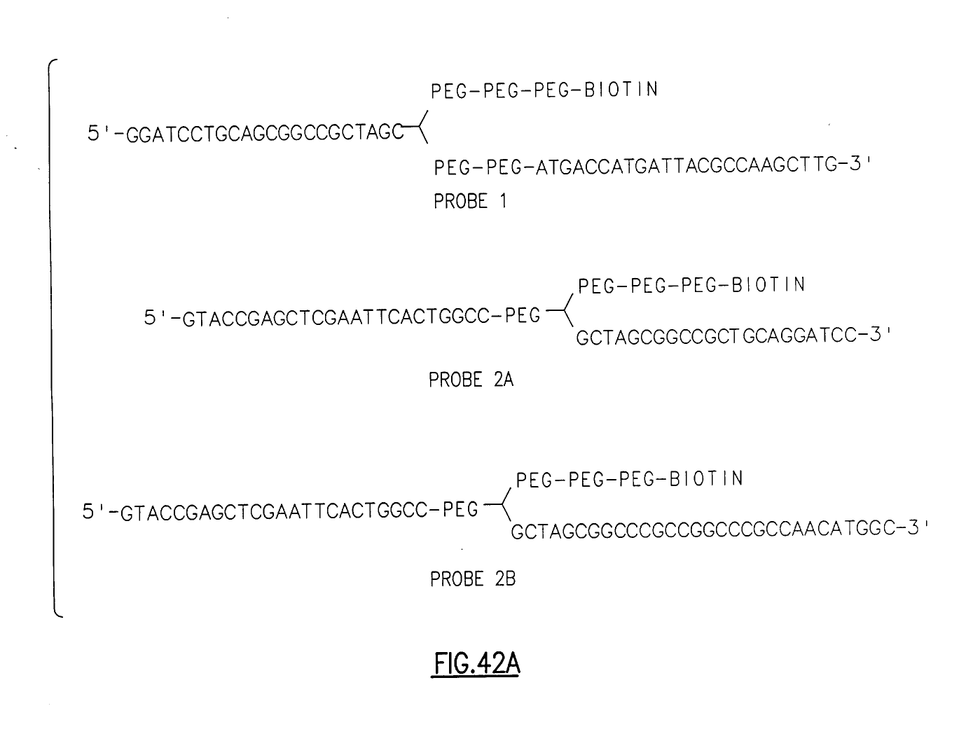

Similarly, FIG. 41 is a bar graph showing the number of reporter beads bound in a dual bead complex using a 39mer bridge employing the same ligation step as discussed in FIG. 40. As in FIG. 40, the data in FIG. 41 indicates that ligation significantly increases the sensitivity of the dual bead assay in both Sets 1 and 2. This data demonstrates that the use of a 39mer bridge aids in the ligation process thus enhancing the signal from both Sets as implemented in the dual bead assay.

Dual Bead Assays Using Cleavable Spacer or Displacement Probes

The use of cleavable spacers in dual bead assay increases the specificity of the assay. Indeed, in addition to complementary sequences to the target DNA, the capture probes and reporter probes contain sequences that are complementary to each other. This additional requirement enhances specificity to target capture. Furthermore, additional bonding between the capture bead and reporter beads via the hydrogen bonds between capture and reporter probes strengthen the interactions between the dual beads.

In this embodiment of the present invention, in the absence of a target, the capture probe hybridizes to the reporter probes, resulting in the formation of the dual bead complexes as shown in 42B and 43A. As illustrated in FIGS. 42B and 42C, the dual bead complexes are subjected to selective restriction enzyme digestion after target capture. The sequence specific digestion will selectively cleave the hydrogen bonds between the capture probes and reporter probes as depicted in FIG. 42D. In the absence of target, with the severance of the hydrogen bonds holding the capture and reporter probes, the dual beads dissociate from each other. In the presence of target, the capture and reporter beads remain bound via the target-mediated hydrogen bonds (FIG. 42D). The amount of target captured therefore is correlated with the number of dual beads remaining after enzyme digestion.

Alternatively, instead of restriction enzyme digestion, the bond holding the capture probes and reporter probes can be unraveled by the use of a displaceable linker. The linker is detached using a displacement probe. In this case, the reporter probe contains a sequence that is partially complementary to the capture probe resulting in a mismatched overhang as depicted in FIG. 43A. To dissociate the capture and reporter probes from each other, the complex is subjected to heat treatment that will initiate the melting of the reporter probe from the capture probe, followed by addition of a large excess of displacement probe. The higher concentration of displacement probe and the tighter interactions between the displacement probe and the mismatched overhang which will result in the unraveling of the reporter probe from the capture probe as illustrated in FIGS. 43B and 43C. This will result in the dissociation of reporter beads from capture beads in the absence of target DNA.

More specifically, the dual bead assay according to the present invention may be implemented using 3 μm magnetic capture beads and 2.1 μm fluorescent reporter beads. These beads are coated with transport probes and signal probes respectively. The transport probes and signal probes, in addition to being complementary to a target sequence, pUC19 for example, contain sequences that are complementary to each other, as illustrated in FIGS. 42A, 42B, 42C, and 43D. The sequences that bind the transport probe and the signal probes together are designed such that they are susceptible to the cleavage of very rare restriction enzymes including Not 1. The use or rare restriction enzymes and restriction sites prevents the accidental cleavage of the target DNA. The capture beads and reporter beads are mixed with varying quantities of target DNA. After target capture, the DNA complex is subjected to restriction digestion by a rare restriction enzyme including Not 1. The restriction digestion by this enzyme will cleave the DNA sequence connecting the reporter beads to the capture beads. In the absence of target DNA, the reporter beads will be dissociated from the capture beads and removed by magnetic concentration of the magnetic beads. Thus, only in the presence of the target sequence, the magnetic capture beads bind to fluorescent reporter beads, resulting in a dual bead assay. The introduction of cleavable spacers into the capture and reporter probes improves the specificity and the sensitivity of the dual bead significantly.

In an alternative embodiment of the present invention, a shorter overlap and a mismatched overhang between the complementary sequences of probes on the reporter bead and the capture bead (probe 1 and probe 2B), resulting in the formation of a displaceable linker, is used in conjunction with a displacement probe as illustrated in FIGS. 43A and 43B. The mismatched overhang on probe 2B is the site for initial binding of the displacement probe as shown in FIG. 42B. Once the displacement probe binds to the overhang, the displacement probe proceeds to displace the overlaping sequences between probe 1 and probe 2B which is depicted in FIG. 43C. In the absence of target DNA, the reporter beads will be dissociated from the capture beads by the actions of the displacement probe and consequently removed by magnetic concentration of the magnetic beads. Thus, only in the presence of the target sequence, the magnetic capture beads bind to fluorescent reporter beads, resulting in the non-dissociation of the dual bead complex.

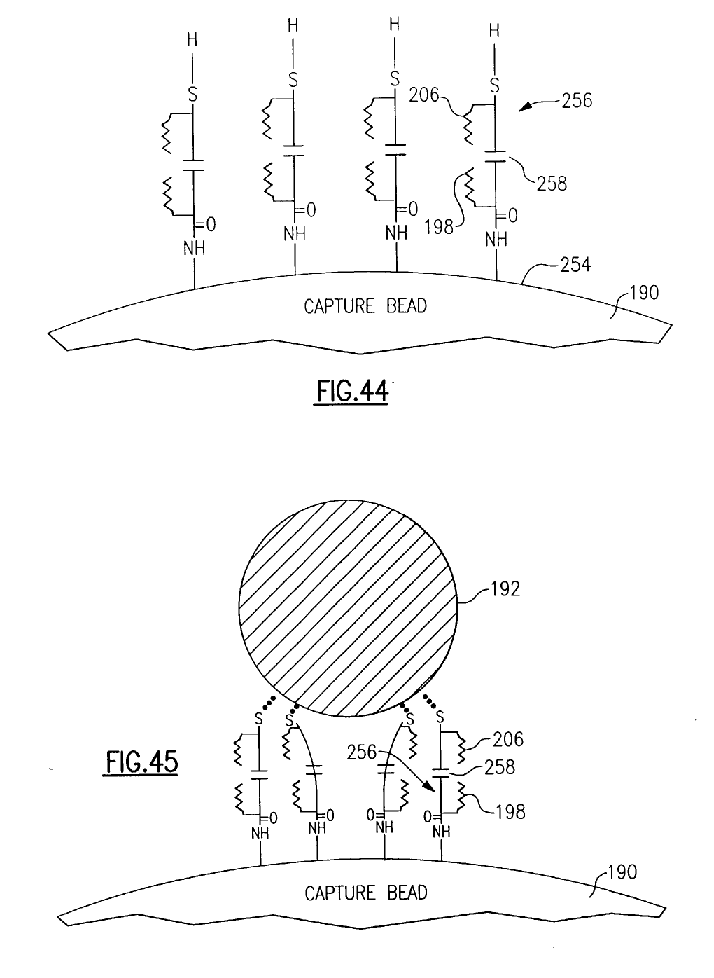

The general operation of the cleavable spacer according to the present invention can be understood more particularly by reference to FIGS. 44, 45, 46A-46C, 47, 48A, 48B, and 49A-49C, which schematize two embodiments of the present invention. With reference to FIG. 44, a capture bead is provided with a derivatized surface to which is attached a plurality of cleavable spacer molecules 256. Each spacer 256 including a cleavage site 258, a signal probe 206, and a transport probe 198. As shown in FIG. 44, the transport probes include a thiol group which reacts to form a covalent bond with metallic elements as discussed in conjunction with FIG. 45. The capture bead, which may be porous or solid, can be selected from a variety of materials such as plastics, glass, mica, silicon, and the like.

The surface of the capture bead 190 or reporter bead 192 can be conveniently derivatized to provide covalent bonding to each of the probes including the cleavable spacer molecule 256. Referring now to FIG. 45, there is shown metallic reporter beads that provide a convenient reflective signal-generating means for detecting the presence of a target. Typical materials used in creating metallic beads are gold, silver, nickel, chromium, platinum, copper, and the like, with gold being presently preferred for its ability readily and tightly to bind e.g. via dative binding to a free SH group at the signal responsive end of the cleavable spacer. The metal beads may be solid metal or may be formed of plastic, or glass beads or the like, on which a coating of metal has been deposited. Also, other reflective materials can be used instead of metal. The presently preferred gold spheres bind directly to the thio group of the signal probe 206.

As depicted in FIGS. 44 and 45, the transport probe 198 is attached covalently at the amino end via an amide linkage. The cleavable spacer molecule includes the cleavage site 258 that is susceptible to cleavage during the assay procedure, by chemical or enzymatic means, heat, light or the like, depending on the nature of the cleavage site. Chemical means are presently preferred with a siloxane cleavage group, and a solution of sodium fluoride or ammonium fluoride, exemplary, respectively, of a chemical cleavage site and chemical cleaving agent. Other groups susceptible to cleaving, such as ester groups or dithio groups, can also be used. Dithio groups are especially advantageous if gold spheres are added after cleaving the spacer. Alternatively, the cleavage site may be a restriction site for cleavage using restriction enzymes. Restriction cleavage is the preferred method when performing genetic or immunochemical assays. Spacers may contain two or more cleavage sites to optimize the complete cleavage of all spacers.

Nucleic Acid Assays Using Cleavable Spacers

In one aspect of the invention, the transport and signal probes are adapted to bind complementary strands of nucleic acids that may be present in a test sample. The complementary oligonucleotides comprise members of a specific binding pair, i.e., one oligonucleotide will bind to a second complementary oligonucleotide.

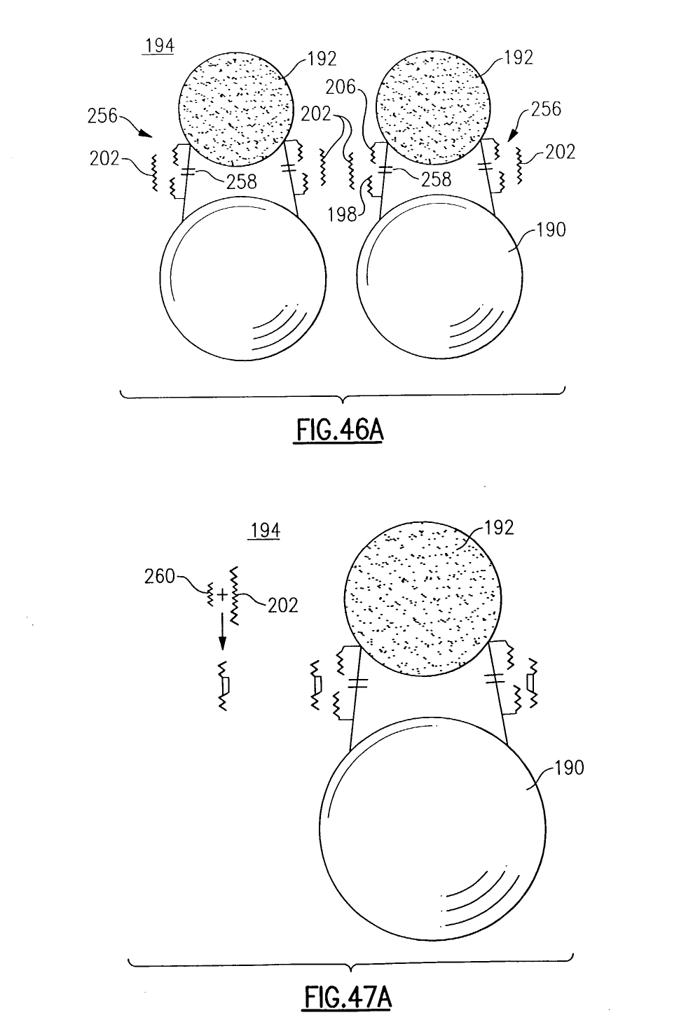

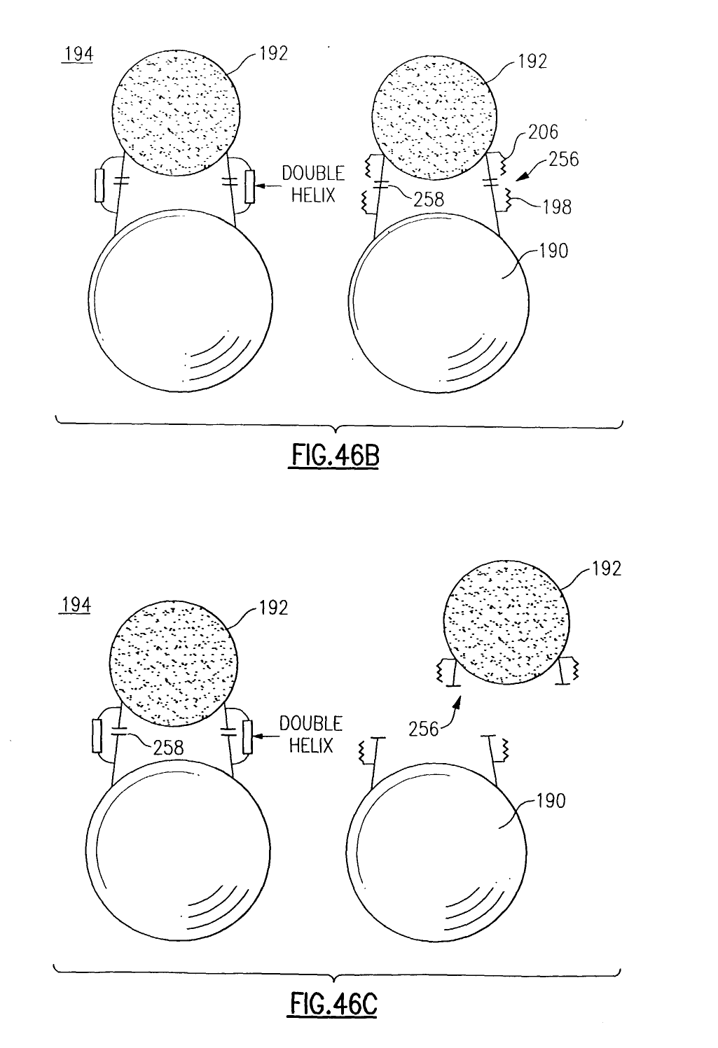

As is shown more particularly in FIGS. 46A through 46C, schematizing one embodiment of the invention, cleavable spacer molecules 256 including the transport probes 198 and signal probes 206 located at different sites on the surface of the capture bead 190 and reporter bead 192. As illustrated in FIG. 46A, oligonucleotide target agents 202 are located in close proximity to the transport probes 198 and signal probes 206. In the event these target agents are complimentary to both probes, hybridization occurs between the target agent 202, transport probe 198, and the reporter probes 206 to form a double helix as is shown in FIG. 46B. If there is no complementarity between the target agent 202 and the probes, there is no binding between those groups as is further illustrated in FIG. 46B where no double helix if formed.

When the cleavage site 258 is cleaved, but for the binding by the double helix-coupled oligonucleotides, the reporter beads 192 will be free of the capture bead 190 and dissociated therefrom. This is illustrated more fully in FIG. 46C. The presence or absence of dual bead complexes 194 may then be detected by an incident light, particularly an incident laser light.

Nucleic Acid Assays Using Cleavable Spacers and Ligation

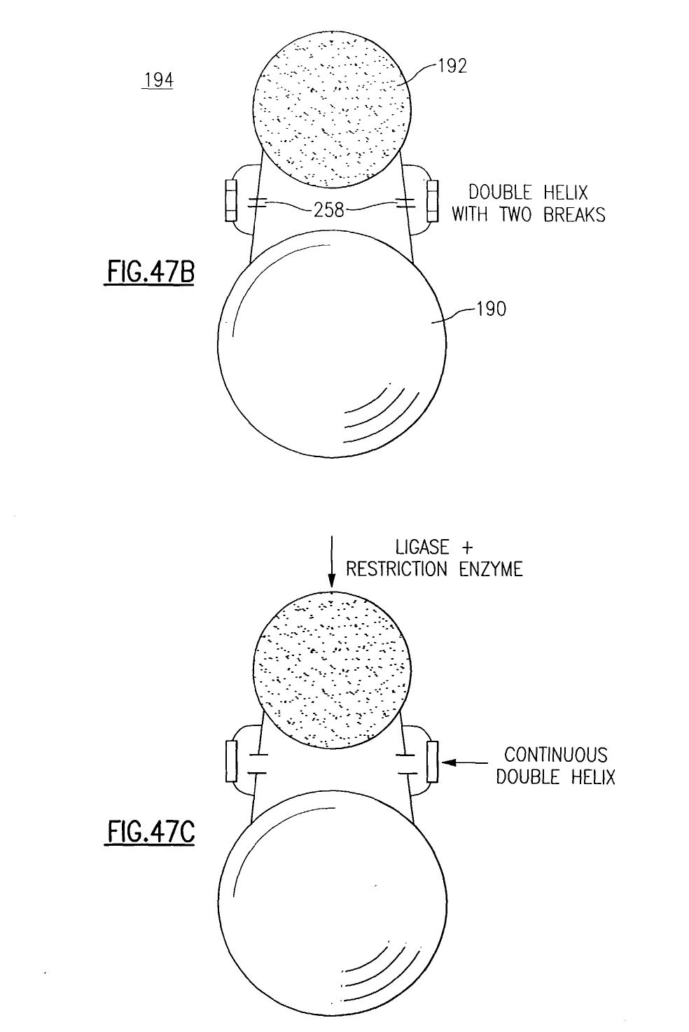

With reference now to FIG. 47A, there is illustrated a schematic representation of an alternative embodiment employing a bridging agent 260. The bridging agent 260 may include a relatively short oligonucleotide sequence for binding to a portion of a target such that when the target binds to the transport 198 and signal probes 206, the bridging agent 260 acts as a bridge between the ends on the transport probe 198 and the signal probe 206. This results in the formation of a double helix with two breaks as depicted in FIG. 47B.

Continuing on to the next step shown in FIG. 47C, there is shown a schematic representation of the use of DNA ligase in conjunction with the cleavable spacer in a further embodiment of the nucleic acid detection embodiment of the present invention. The ligation procedure links the breaks in the double helix covalently. This covalent linkage increases the strength with which analyte-specific binding adheres the dual bead complex thus permitting, in this embodiment, increased stringency of wash affording increased specificity of the assay.

It will be appreciated by those skilled in nucleic acid detection that the cleavable reflective signal elements of the present invention are particularly well suited for detecting amplified nucleic acids of defined size, particularly nucleic acids amplified using the various forms of polymerase chain reaction (PCR), ligase chain reaction (LCR), amplification schemes using T7 and SP6 RNA polymerase, and the like.

Immunoassays Using Cleavable Spacers

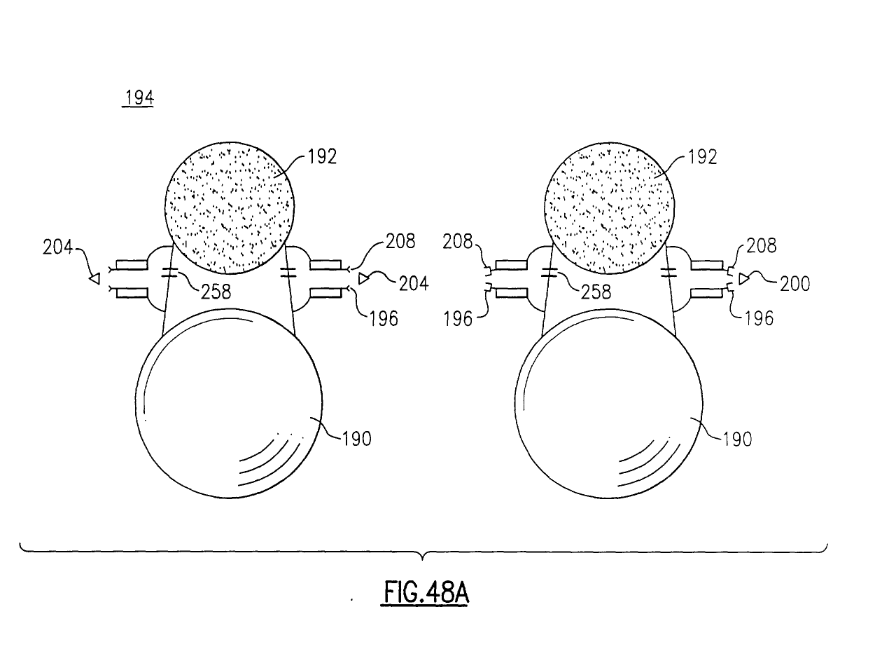

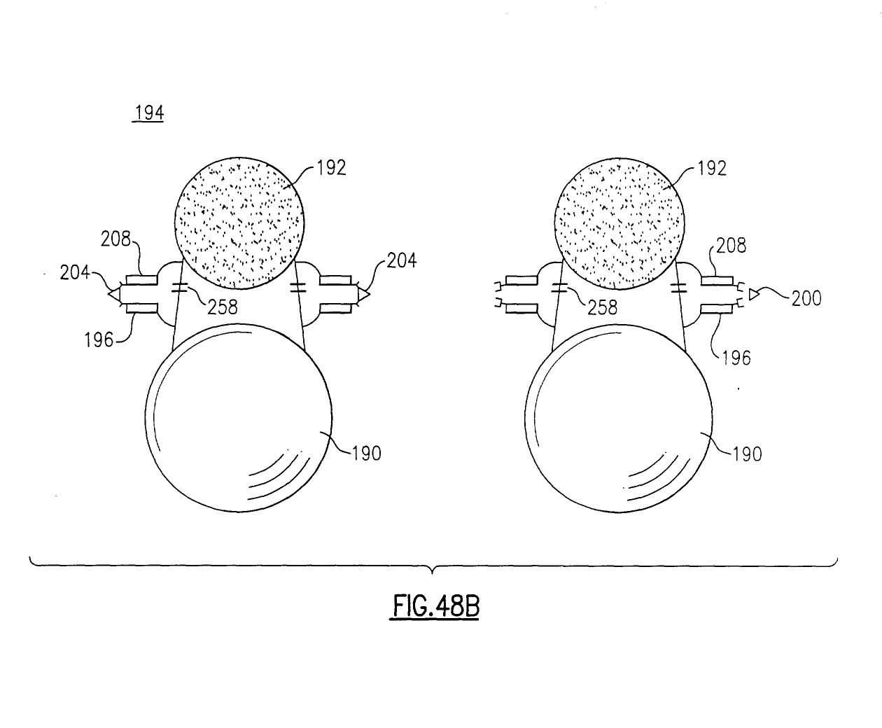

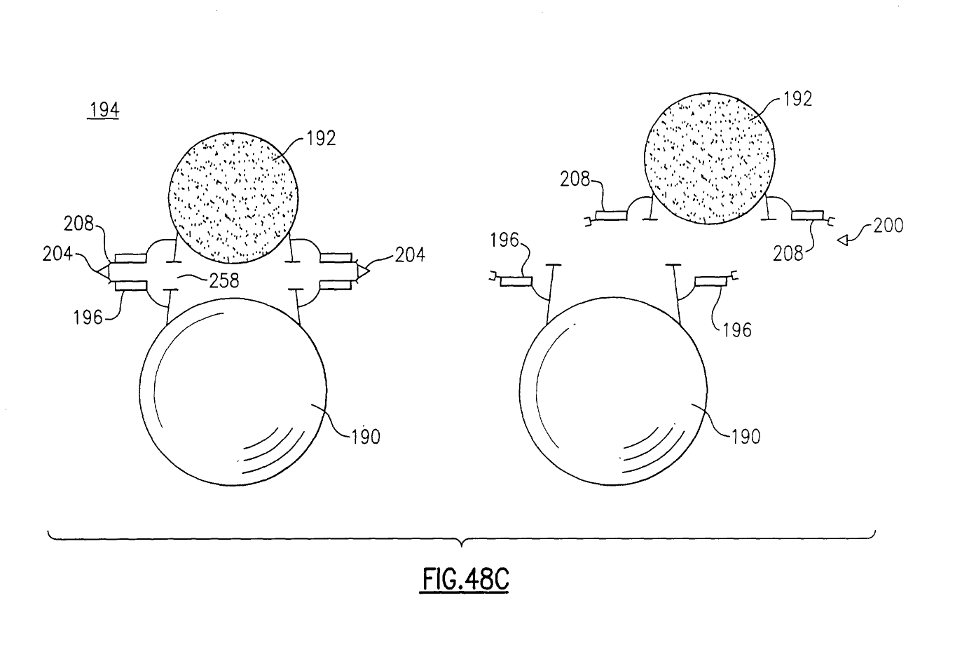

In a further embodiment of the invention shown in FIGS. 48A through 48C, the cleavable spacer 258 includes modified antibodies to permit an immunoassay. The modified antibodies may be attached non-covalently to the cleavable spacer 258 mediated by oligonucleotides that are covalently attached to the antibodies. Use of complementary nucleic acid molecules to effectuate non-covalent, combinatorial assembly of supramolecular structures is described in further detail in co-owned and co-pending U.S. patent application Ser. No. 08/332,514, filed Oct. 31, 1994; Ser. No. 08/424,874, filed Apr. 19, 1995; and Ser. No. 08/627,695, filed Mar. 29, 1996, incorporated herein by reference. In another embodiment, antibodies can be attached covalently to the cleavable spacer using conventional cross-linking agents, either directly or through linkers.

The antibody probes include an antibody transport probe 196 bound to the capture bead 190 and an antibody signal probe 208 bound to the reporter bead 192. Both beads and probes are held together by the cleavable spacer 258. The antibody transport probe 196 and the antibody signal probe 208 have affinity to different epitopic sites of an antigen of interest.

With further reference to the immunoassay schematized in FIGS. 48A-48C, upon application of a test solution containing target antigen 204 or a non-specific target agent 200 to the collection of dual bead complexes 194 as illustrated in FIG. 48A, target antigen 204 binds to the antibody transport probe 196 and the antibody signal probe 208 as shown in FIG. 48B. This binding prevents decoupling of the dual bead complex 194 when the cleavage site 258 is cleaved, such as, for example, by contact with a chemical cleaving agent. In contrast, the second cleavable signal element, which was not bound by the non-specific target agent 200 because the lack of binding affinity of the antibodies to the target agent 200, allow the dual bead complexes to dissociate as illustrated in FIG. 48C.

Presence and absence of the dual bead complex 194 may then be detected as reflectance or absence of reflectance of incident light, particularly incident laser light.

As should be apparent, coupling of antibodies as depicted permits the adaptation of standard immunoassay chemistries and immunoassay geometries for use with the cleavable spacers in the dual bead assay of the present invention. Some of these classical immunoassay geometries are further described in U.S. Pat. No. 5,168,057, issued Dec. 1, 1992, incorporated herein by reference. Other immunoassay geometries and techniques that may usefully be adapted to the present invention are disclosed in Diamandis et al. (eds.), Immunoassay, AACC Press (July 1997); Gosling et al. (eds.), Immunoassay: Laboratory Analysis and Clinical Applications, Butterworth-Heinemann (June 1994); and Law (ed.), Immunoassay: A Practical Guide, Taylor & Francis (October 1996), the disclosures of which are incorporated herein by reference. Thus, it should be apparent that the direct detection of analytes schematized in FIGS. 48A-48C is but one of the immunoassay geometries adaptable to the cleavable spacer type dual bead assay and assay devices of the present invention.

The present invention will prove particularly valuable in immunoassays screening for human immunodeficiency viruses, hepatitis a virus, hepatitis B virus, hepatitis C virus, and human herpes viruses.

It will further be appreciated that antibodies are exemplary of the broader concept of specific binding pairs, wherein the antibody may be considered the first member of the specific binding pair, and the antigen to which it binds the second member of the specific binding pair. In general, a specific binding pair may be defined as two molecules, the mutual affinity of which is of sufficient avidity and specificity to permit the practice of the present invention. Thus, the cleavable spacer of the present invention may include other specific binding pair members as side members. In such embodiments, the first side member of the cleavable signal element includes a first member of a first specific binding pair, the second side member of the cleavable spacer includes a first member of a second specific binding pair, wherein said second member of said first specific binding pair and said second member of said second specific binding pair are connectably attached to one another, permitting the formation of a tethering loop of the general formula: first member of first specific binding pair-second member of first specific binding pair-second member of second specific binding pair-first member of second specific binding pair.

Among the specific binding pairs well known in the art are biologic receptors and their natural agonist and antagonist ligands, proteins and cofactors, biotin and either avidin or streptavidin, alpha spectrin and beta spectrin monomers, and antibody Fc portions and Fc receptors.

Methods for DNA Conjugation onto Solid Phase

Successful conjugation of probes to a solid phase such as a bead or a bio-disc, is an important step for the dual bead assays of the invention. In certain embodiments of the invention, probes are attached covalently to the beads. Efficiency of the covalent conjugation depends on the type of bead utilized and the specific conjugation method employed.

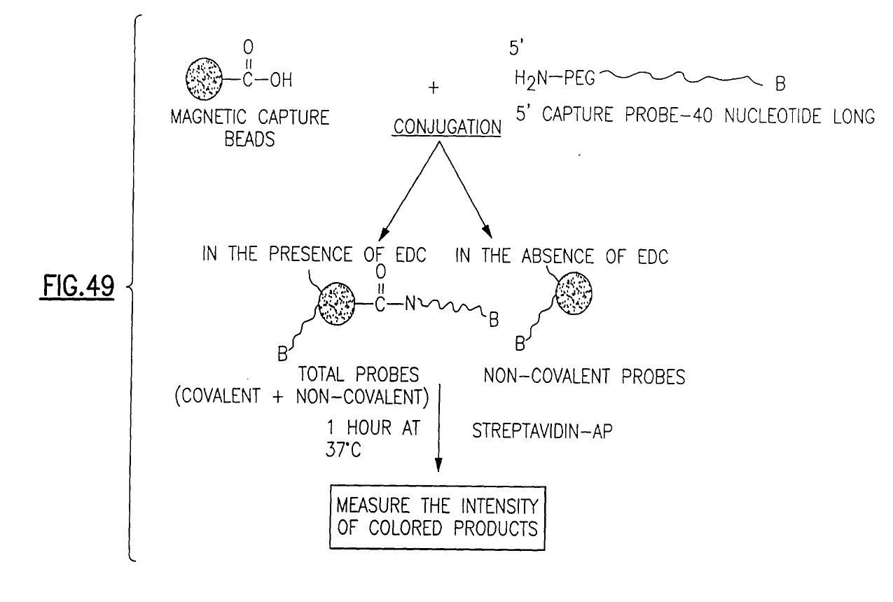

As illustrated in FIG. 49, a systematic method to evaluate the use of a solid phase for probe conjugation is presented. The methodology identifies covalent linkages that improve specificity of a dual bead assay. This approach can be used to evaluate treatment of solid phase (i.e., coating of a solid surface such as the surface of a bead or a surface on a biodisc) to see whether the treatment improves the solid phase conjugation efficiency. As a first step, probes are tagged with an appropriate molecule for detection and measurement of the amount of probe bound at a later time. By way of non-limiting example, a biotin moiety (B) can be attached at the 3′ end of a DNA probe. Next, the probe is conjugated in the presence or absence of a cross-linking agent, e.g., EDC (1-Ethyl 3-3 dimethylaminopropyl carbodiimide-HCl). In the presence of a cross-linking agent, a probe will be conjugated both covalently and non-covalently. Alternatively, in the absence of the cross-linking agent, a probe will only be absorbed to the bead non-covalently. After the appropriate washing steps are performed, a detection agent is added that binds specifically to the biotin molecule previously tagged to the probe. For example, streptavidin-alkaline phosphatase (S-AP) is added to the probe-bound beads, and the S-AP binds specifically to the biotinylated probes. Next, alkaline phosphatase substrate is added to the sample. This substrate develops color upon loss of a phosphate group, and the intensity of the color correlates with the amount of probes bound to the beads. After an appropriate incubation period, the solution is isolated and the optical density of the solution at an appropriate wavelength is determined with a spectrophotometer or microtiter plate reader.

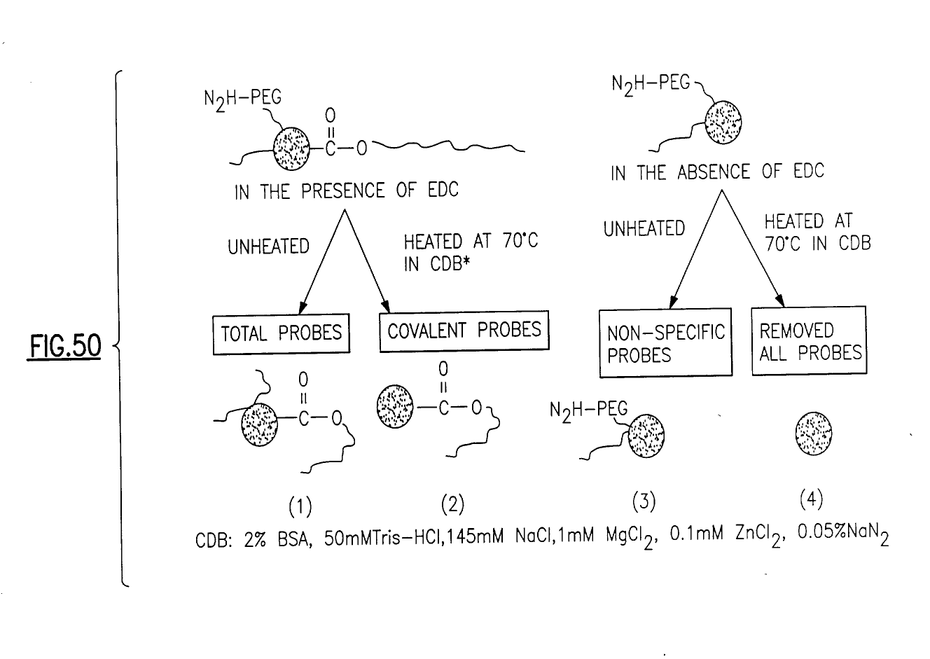

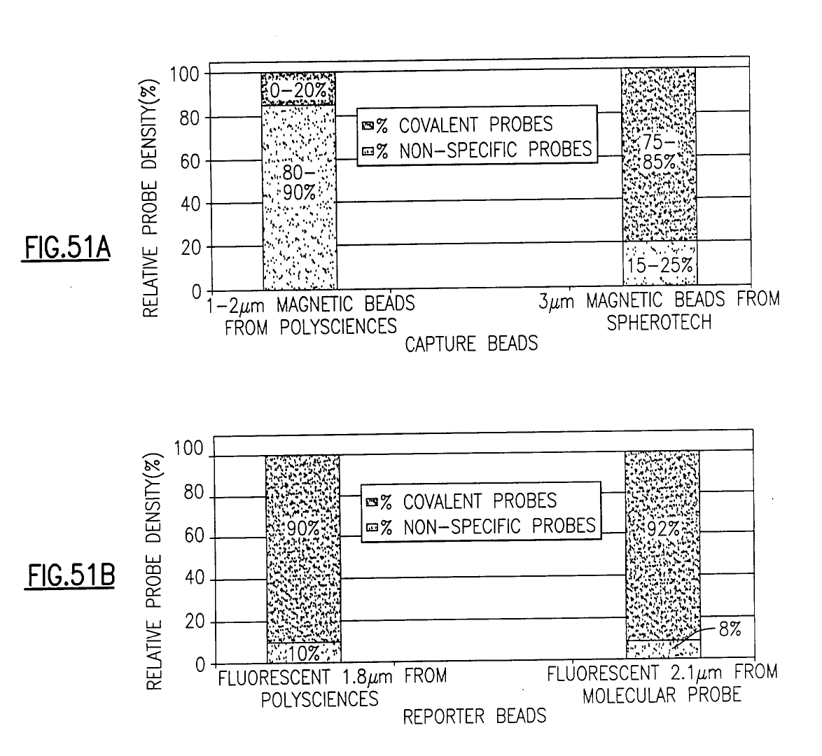

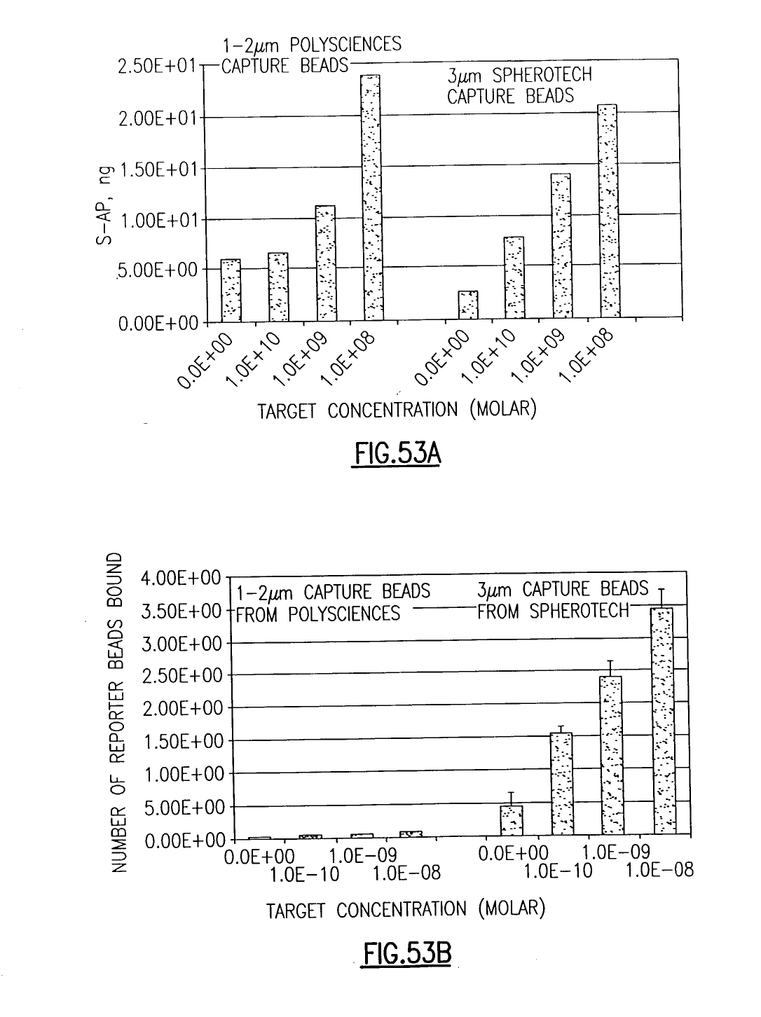

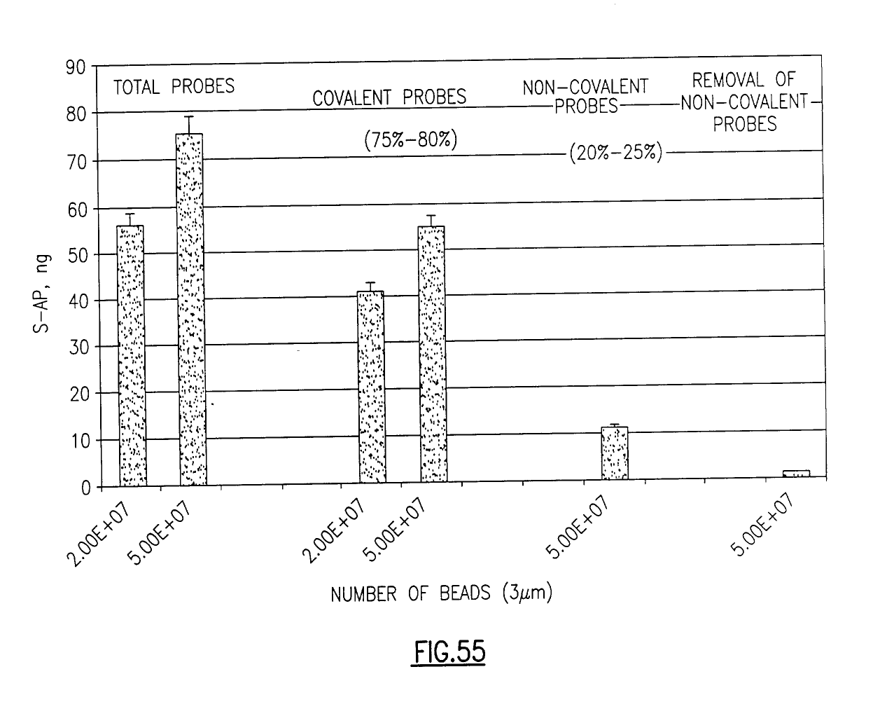

Referring to FIG. 50, there is illustrated conjugation of an oligonucleotide probe onto a carboxylated bead. Conjugation of probes may be carried out covalently or non-covalently. In a dual bead assay, covalent probe conjugation is preferred over non-covalent conjugation as discussed in further detail in connection with FIGS. 51A, 51B, 53A, and 53B. This conjugation process is performed prior to Step I of the dual bead assay as presented in FIGS. 11A, 11B, 12A, and 12B. The amount of probe covalently bound to the solid surface may be evaluated by determining the amount of probe that binds to the solid phase covalently and non-covalently, i.e., non-specifically, in the presence and absence of a crosslinking agent (e.g., EDC). The percentage of non-covalently bound probe can be determined according to the formula 100%*N/T, and the percentage of covalently bound probe can be determined by the formula 100%*(T-N)/T, wherein “T” represents the total amount of signal obtained in the presence of a cross-linking agent (i.e., the total amount of covalently and noncovalently bound probe) and “N” represents the total amount of signal obtained when no crosslinking agent is used. Alternatively, the amount of probes conjugated covalently can be obtained directly if all non-covalently bound probes are removed prior to the addition of the S-AP. This can be conveniently achieved by heating the beads to 70° C. prior to the step of adding the S-AP. If the percentage of non-covalently bound probe is less than 20%, the beads being tested can be used as solid phase for covalent conjugation. Results of an application of this methodology are presented in FIGS. 51A, 51B, and 55 (see Example 7 for details).

As depicted in FIGS. 51A and 51B, the 1.8 μm, 2.1 μm, and 3 μm beads provide suitable solid phase for covalent probe conjugation with at least 75% conjugation efficiency. The 1-2 μm beads, however, may not be suitable for covalent conjugation of probes due to their low covalent conjugation efficiency of less than 21%.

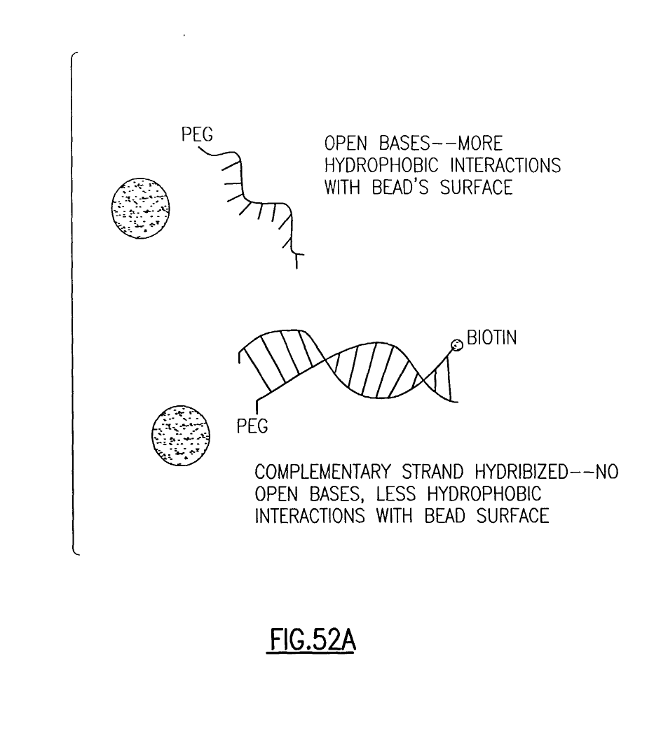

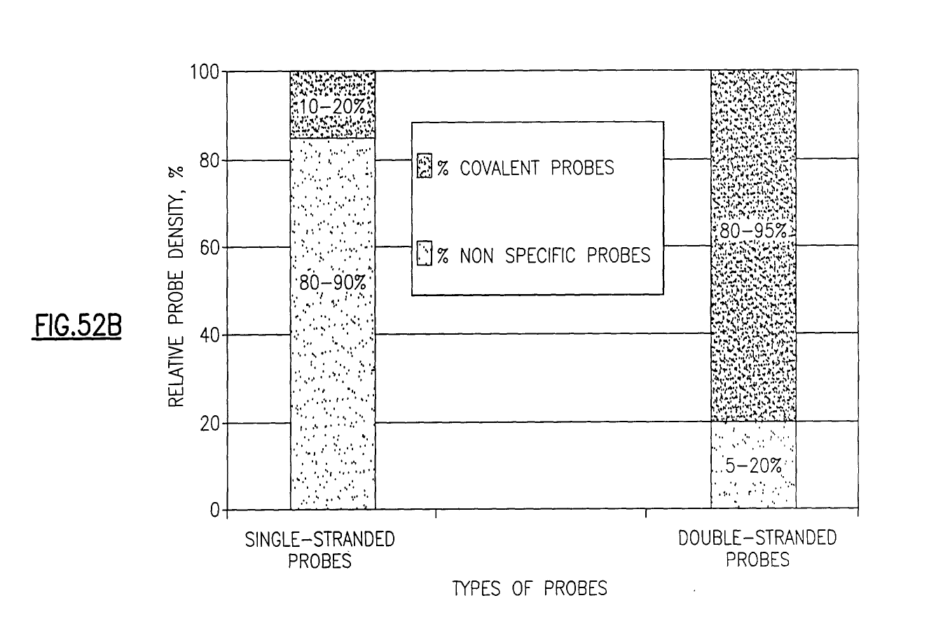

Various embodiments of the invention utilize nucleic acid molecules as probes. FIG. 52A shows the structural differences between single stranded and double stranded DNA in order to illustrate how the single stranded DNA can more readily bind non-covalently to a solid phase. Single-stranded DNA has hydrophobic base side chains that can readily absorb to a solid phase non-covalently. In contrast, with double-stranded DNA hydrophobic base interaction with a solid phase does not generally occur and non-covalent or non-specific binding is limited in comparison to a single-stranded DNA molecule. Thus, in various embodiments of the invention, double stranded DNA can be utilized in place of single-stranded DNA, thereby enhancing DNA binding to a solid phase by covalent linkage (FIG. 52B). After covalent binding of one of the strands of the double-stranded DNA probe to the solid phase, the non-covalently bound strand may be removed by heating the sample to 70° C. in the appropriate buffer. Under these conditions, the double stranded DNA are separated, and only single strand DNA probe that is covalently attached to the bead ramain and is used to capture the target. Experimental details regarding the use of double stranded DNA for covalent probe conjugation is described in further detail below in Example 8.

In various embodiments of the invention, heat treatment can be used to selectively remove non-covalently bound probe(s) from a solid phase. This method is useful when, for example, despite all optimizations with respect to the type of the solid phase, treatment of the solid phase, and the use of double stranded DNA, non-covalent binding to the solid phase is still problematic. The conditions for the heat treatment have been optimized; the optimal buffer consists of: 2% BSA, 50 mM Tris-HCl, 145 mM NaCl, 1 mM MgCl2, 0.1 mM ZnCl2. The treatment is done at a temperature less than or equal to approximately 70° C., since at higher temperatures, the magnetic beads can lose their magnetic properties.

In other embodiments of the invention, the methodology presented herein to determine optimal conditions to obtain covalent linkages that improve specificity of a dual bead assay can be applied to a disc surface that is used as a solid phase. Similarly, the invention provides in other embodiments analogous to those described herein above to evaluate solid surfaces for protein binding. For example, such an application would be useful where the probe utilized is an antigen or antibody.

Referring now to FIG. 53A, there is shown a bar graph of results collected from an enzyme assay detecting targets bound to probes on two different capture beads for use in a dual bead assay. As illustrated above in FIG. 51A, the 1-2 μm beads have a covalent binding efficiency up to 20% and the rest of the probes bind non-covalently and the covalent binding efficiency of the 3 μm beads is between 75-85%. The data shown in FIG. 53A indicates that both of the tested beads bind a similar amount of target regardless of whether the probe is bound covalently or non-covalently. This suggests that covalent binding is not necessary in an enzyme assay format.

In contrast to FIG. 53A, FIG. 53B represents results of a dual bead assay designed to examine the number of reporter beads captured by the same capture beads used in FIG. 53A. The results shown in FIG. 53B indicate that covalent binding of the probe to the capture bead is necessary to enhance the sensitivity of the assay. In this particular embodiment of the present invention, the 3 μm capture bead contains more covalently bound probes than the 1-2 μm beads, as mentioned above. This allows the retention of the reporter bead in the dual bead complex since covalently tethered probes on the capture bead have higher bond strength than non-covalently bound probes.

As mentioned in the summary of the invention above, the surface of the beads or solid phase may be uneven which limits the probe accessibility to the target in solution. Probe linkers may be used to extend the length of the probes to increase probe target accessibility as discussed with reference to FIG. 52A.

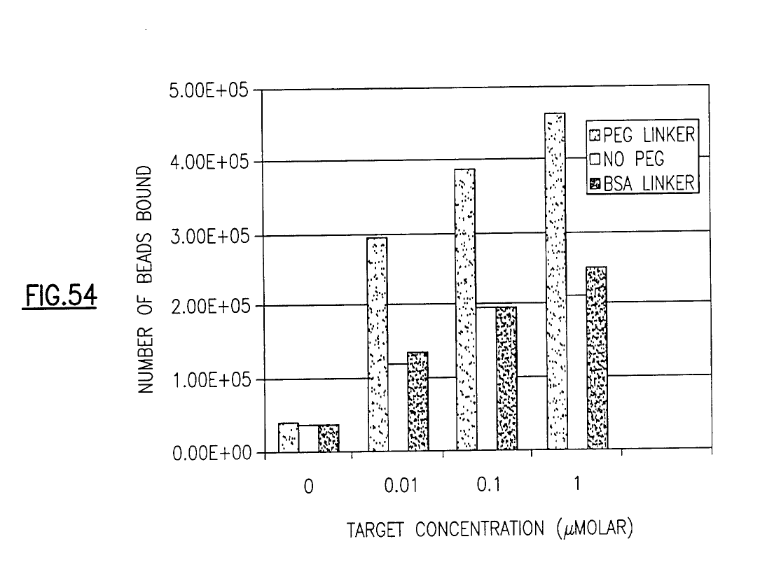

With reference now to FIG. 54, there is presented data collected from a dual bead assay showing enhanced target binding using PEG as a linker. Linkers may increase the assay sensitivity by approximately 50% or more. The use of linkers also decreases non-specific reporter bead binding to the capture beads. In this embodiment of the present invention, probes are attached to a solid phase by way of a linker molecule. The use of a linker molecule makes the probe longer and more rigid. These two properties increase the accessibility of the probe(s), and, therefore, maximize the efficiency of target capture and the sensitivity of the dual bead assay. As known to those skilled in the art, various linker molecules can be used that satisfy the criteria described herein. By way of non-limiting example, bovine serum albumin (BSA) or polyethylene glycol (PEG) can be used as linker molecules. In certain embodiments of the invention, the linker can be a series of 3 to 10 PEG molecules that are attached covalently to the 5′ end of a DNA probe. Details relating to the use of PEG as a linker molecule are described below in Example 9.

With reference now to FIG. 55, there is shown a bar graph demonstrating determination of percent covalent probe density on 3 μm Spherotech beads. These graphs represent signals generated from an enzyme assay using biotinylated probes and streptavidin-linked alkaline phosphatase enzyme reactions. As discussed with reference to FIG. 50, the covalent conjugation efficiency can be calculated by determining the total amount of probes bound to non heat-treated beads. A separate aliquot of the beads is then heated to remove the non-covalently bound probes and the amount of covalent probes is then determined using the enzyme assay as described in Example 7 below. With these data, the percentage of covalent probe binding can then be determined using the following formula: H/T*100 where H represents signal from heat treated beads and T is the total signal from the non-heat treated beads.

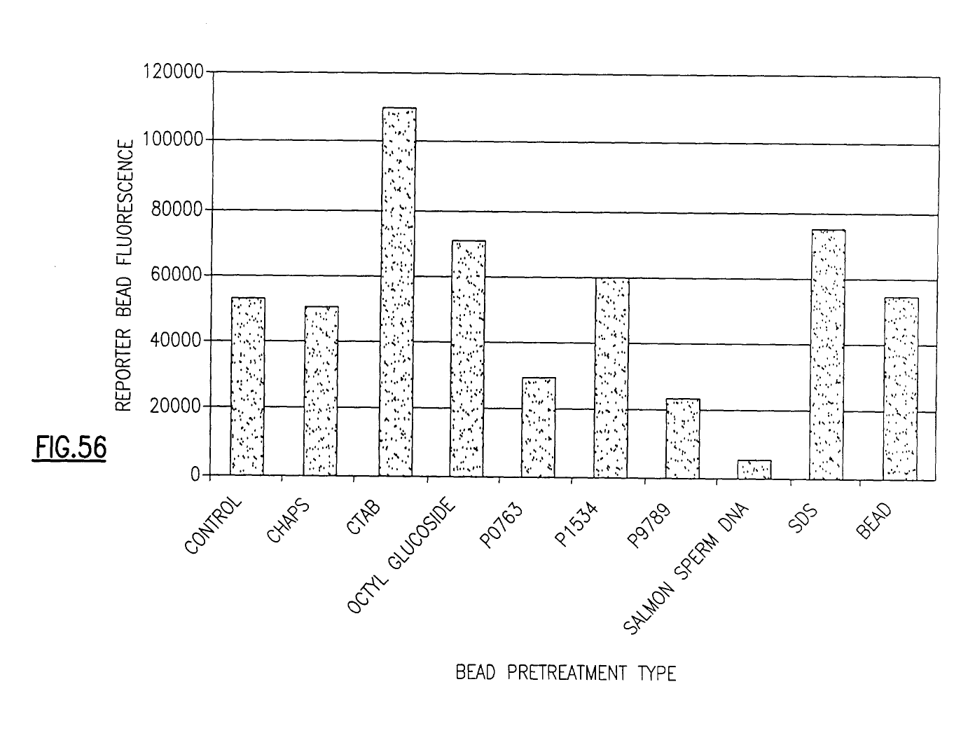

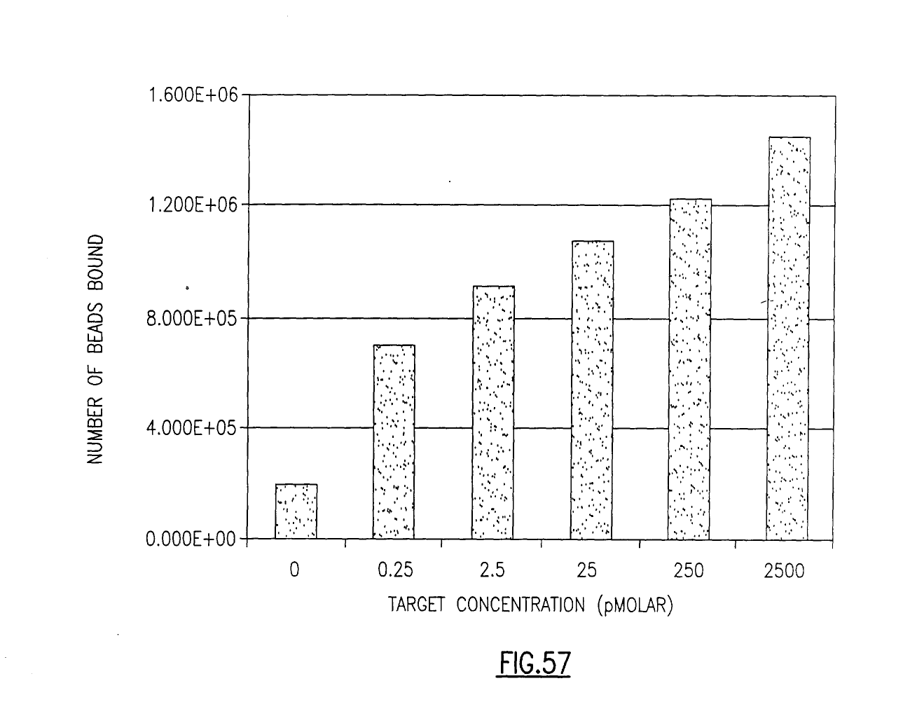

FIG. 56 is a bar graph presentation demonstrating the pretreatment of the beads with various blocking agents including detergents. Decreasing non-specific bead binding is critical in the dual bead assay since the assay sensitivity is inversely related to the baseline signal which is the non-specific binding of the reporter beads to the capture beads. Thus the lower the baseline, the more sensitive the assay becomes. As illustrated, the use of salmon sperm DNA worked best in reducing the nonspecific binding relative to the other blocking agents tested in this experiment. Salmon sperm DNA blocking reduced non-specific binding by approximately 10 fold. Salmon sperm DNA is, therefore, a preferred method for blocking non-specific bead binding in one aspect of the present invention. Other blocking agents may also be used including BSA, Denhardt's solution, and sucrose. Preferably, beads should be blocked by an appropriate blocking agent after conjugation and heat treatment as shown in FIG. 50 or prior to Step I in FIGS. 11A, 11B, 12A, and 12B above to increase the dual bead assay sensitivity.

Methods for Decreasing Non-Specific Bead Binding

As discussed above, FIG. 56 is a bar graph presentation demonstrating the pretreatment of the beads with various blocking agents including detergents to decrease non-specific binding of the beads. Decreasing non-specific bead binding is critical in the dual bead assay since the assay sensitivity is inversely related to the baseline signal which is the non-specific binding of the reporter beads to the capture beads. Thus the lower the baseline the more sensitive the assay becomes. As illustrated, the use of salmon sperm DNA worked best in reducing the nonspecific binding relative to the other blocking agents tested in this experiment. Salmon sperm DNA blocking reduced non-specific binding by approximately 10 fold. Salmon sperm DNA is, therefore, a preferred method for blocking non-specific bead binding in one aspect of the present invention. Other blocking agents may also be used including BSA, Denhardt's solution, and sucrose. Preferably, beads should be blocked by an appropriate blocking agent prior to Step I in FIGS. 11A, 11B, 12A, and 12B, above, to increase the dual bead assay sensitivity.

FIG. 57 is a bar graph of data generated using a fluorimeter showing a concentration dependent target detection using fluorescent reporter beads in a dual bead assay. This graph shows the picomolar concentration of target DNA versus number of beads bound in a dual bead complex. The dynamic range of target detection shown in this graph is 0.25 pM to 2500 pM (picomoles/liter). While this particular graph was generated using data from a fluorimeter, the results may also be generated using a fluorescent type optical disc drive. Experimental details from an experiment related to detection of a range of target concentrations is discussed in detail in Example 10.

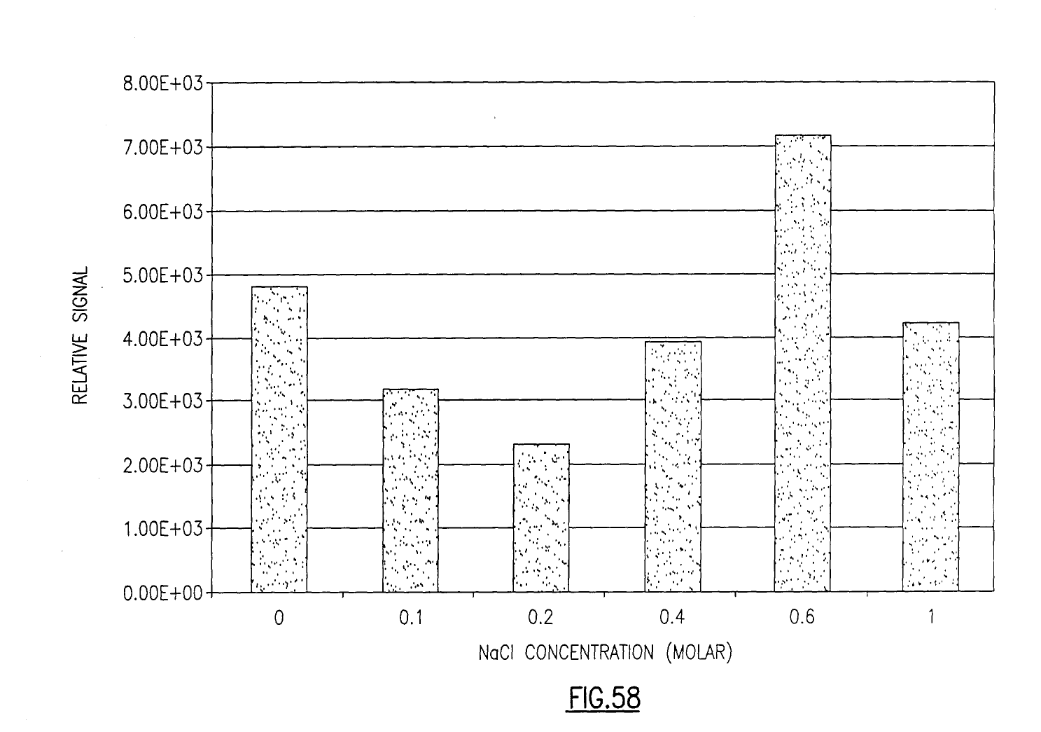

Referring now to FIGS. 58, 59, 60, 61A, and 61B, here are shown data from experiments performed to determine the optimal concentration or ionic strength of various salts in the hybridization buffer or assay buffer. The salt concentration in the assay buffer needs to be optimized in order to increase hybridization efficiency or binding efficiency and decrease non-specific bead binding between capture and reporter beads resulting in lower signal to noise ratio which increases the sensitivity of the assay. In general, the data presented in these figures show that 40 mM EDTA, 300 mM NaCl, 30 mM MgCl² are the optimal salt concentrations for use in one embodiment of the dual bead assay.

With specific reference to FIG. 58, there is a bar graph presentation showing data collected from an experiment using various concentrations of NaCl in the bead buffer and the related non-specific binding as a result of changes in the ionic strength of the buffer. Based on the results presented in FIG. 58, the optimal bead buffer concentration of sodium chloride for use in the dual bead assay is 0.2M since non-specific bead binding is minimal at this NaCl concentration.

Now referring to FIG. 59, there is shown a bar graph illustrating the effect of increasing EDTA concentration on the dual bead assay sensitivity using different target concentrations. FIG. 59 also shows the related non-specific binding as affected by the concentration of EDTA in the assay buffer. The optimal EDTA buffer concentration, based on the data presented, for use in the hybridization buffer is 40 mM since signal generated from the dual bead assay was highest at this concentration.

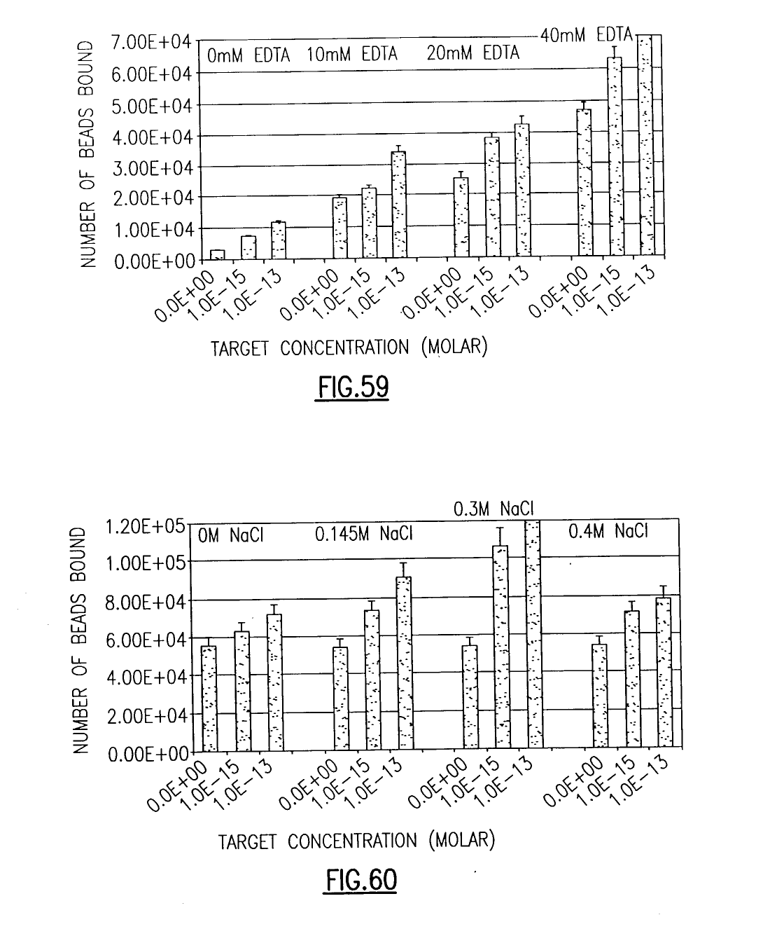

Similarly FIG. 60 presents a bar graph presentation showing the effect of increasing NaCl concentration on the dual bead assay sensitivity using different target concentrations. The non-specific bead binding data related to optimization of the buffer concentration of NaCl is represented in FIG. 58. As shown in FIG. 60, the optimal NaCl concentration for hybridization as implemented in a dual bead assay is 0.3M NaCl. A detailed description of the experimental procedure used to generate this data is discussed below in Example 11.

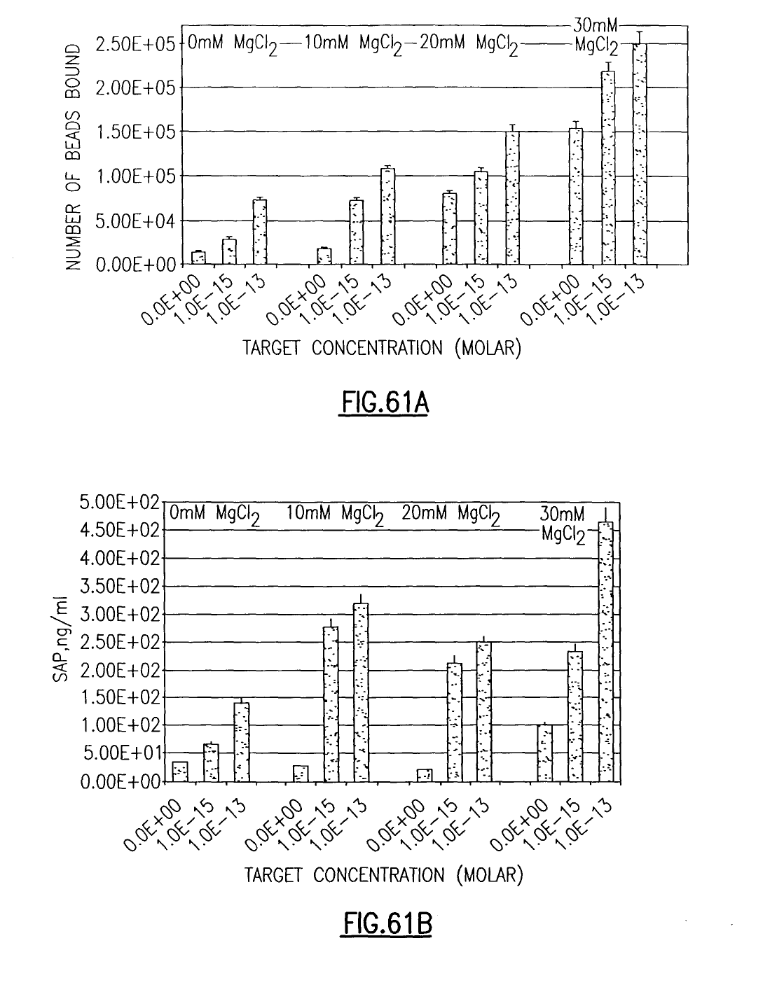

Turning now to FIGS. 61A and 61B, here are bar graph presentations showing the effect of increasing the concentration of MgCl₂ in the assay buffer on the dual bead assay sensitivity and an enzyme assay sensitivity, respectively. Data from these figures indicate that a concentration of 30 mM MgCl₂ in the hybridization buffer is optimal for increasing the signal generated and the assay sensitivity. According to the data shown in FIGS. 61A and 61B, the enzyme assay appears to be more sensitive than the dual bead assay in the 30 mM MgCl² treatment. This conclusion is based on the difference in signal within the treatment group from the various target concentrations. Thus as illustrated, the slope of the concentration curve in the 30 mM MgCl₂ group of the enzyme assay of FIG. 61B is steeper than the corresponding curve in FIG. 61A. Example 12 describes in detail the procedure for carrying out an experiment relating to FIG. 61A.

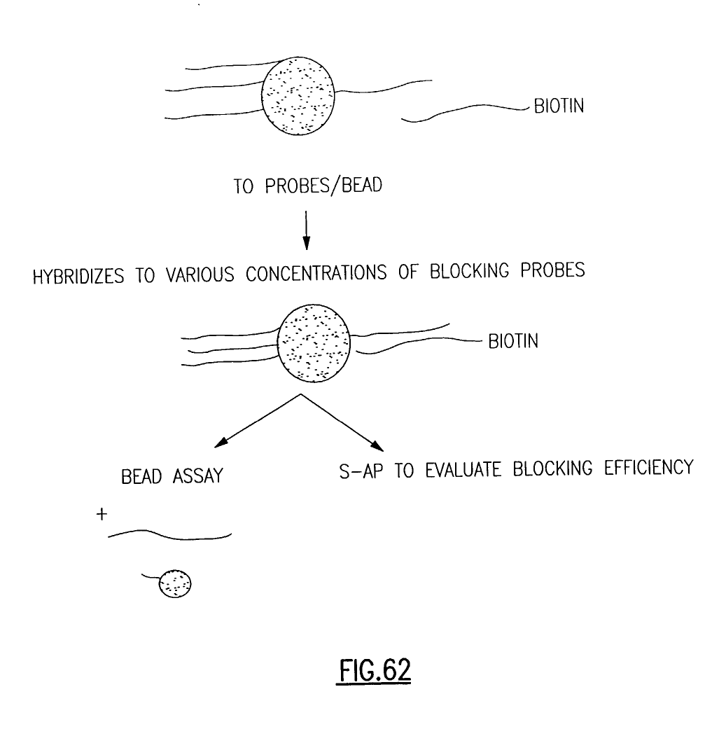

Referring next to FIG. 62, there is shown a pictorial representation of the use of probe blocking agents to increase the sensitivity of the bead assay. The probe blocking agent used in this particular example is a biotinylated DNA that is complimentary to the probe on the bead. The amount of probe blocking agent used to block excess probes on the bead is such that a pre-determined fraction of probes are blocked by the blocking agent. The use of the probe blocking agent in dual bead assay increases the sensitivity of the assay in that it enhances the probability of target binding to a single capture and reporter bead in a dual bead assay. This may increase the sensitivity of the dual bead assay up to one target per dual bead complex. The use of the biotinylated probe blocking agent allows for the quantitation of the blocking efficiency of the probe blocking agent for optimization of the assay. The amount of biotinylated probes bound to the beads may be quantitated by an enzyme assay using streptavidinated or neutravidinated enzymes including streptavidin-alkaline phosphatase (S-AP) and their appropriate substrates. The choice of enzyme and substrate for use in this test is dictated by the type of detection desired. In general, a colorimetric test is performed wherein the enzyme-substrate reaction produces color that is quantified by a spectrophotometer. Alternatively, streptavidinated or neutravidinated fluorescent tags may also be used which may be quantified using a fluorimeter or a Fluorimager. Both the colorimetric and fluorescent quantitation may also be carried out using the appropriate optical disc reader as shown in FIGS. 1 and 2.

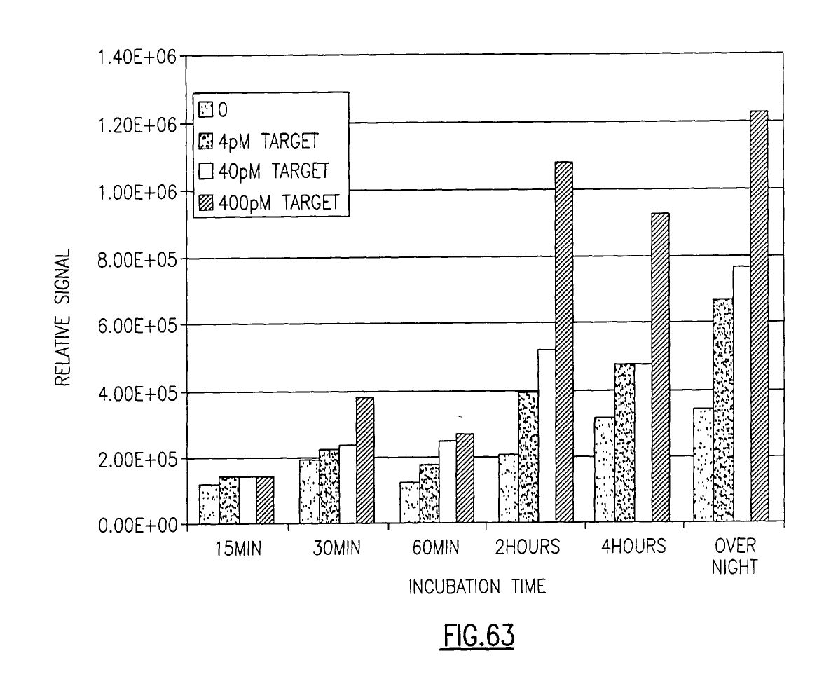

FIG. 63 shows a bar graph presentation of data illustrating the effect incubation time on the signal generated and the assay sensitivity using different target concentrations during a hybridization reaction in a dual bead assay. The data shows that 2 hours is the minimum incubation time required to generate the maximum signal and sensitivity for the dual bead assay and that a 4 hour or overnight hybridization is not necessary.

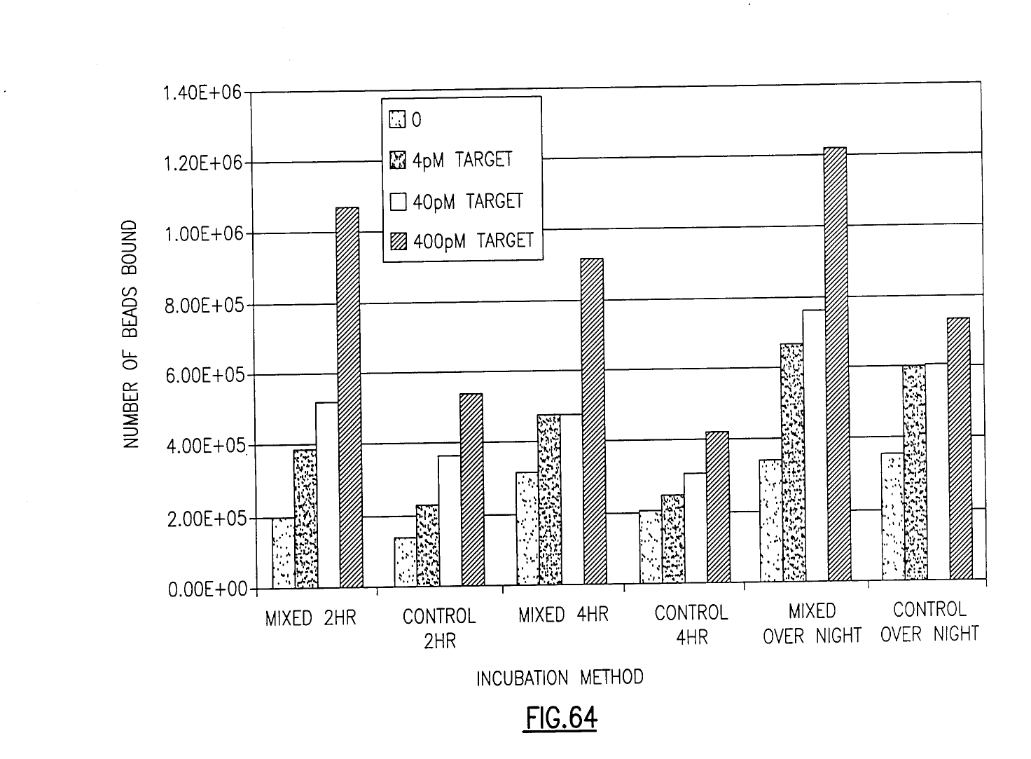

Similarly, FIG. 64 shows a bar graph of data collected illustrating the effect of incubation time and mixing on the hybridization efficiency and the assay sensitivity using different target concentrations in a hybridization reaction as implemented on a dual bead assay. As in FIG. 63, FIG. 64 also shows that 2 hours is an optimal time for hybridization and extending the hybridization time does not increase the signal generated. In addition, mixing significantly increased the hybridization efficiency after 2 hours of hybridization relative to control. Example 14, presented below, explains the details regarding the experiment performed to generate the data shown in FIGS. 63 and 64.

Dual Bead Preparation Methods Including Removal of Non-Specifically Bound Complexes

The following preparation methods are more particular alternative embodiments of the corresponding methods described above in connection with FIGS. 11A, 11B, 12A, and 12B.

With reference now to FIGS. 65A and 65B, there is illustrated a method of preparing a molecular assay using a “single-step hybridization” technique to create dual bead complex structures in a solution according to one aspect of the present invention. This method is similar to the method discussed above in connection with FIG. 11A. The present method includes eight principal steps identified consecutively as Steps I, II, III, IV, V, VI, VII, and VIII.

In Step I of this method, a number of capture beads 190 coated with oligonucleotide transport probes 198 are deposited into a test tube 212 containing a buffer solution 210. The number of capture beads 190 used in this method may be, for example, on the order of 10E+07 and each on the order of 1 um or greater in diameter. Capture beads 190 are suspended in hybridization solution and are loaded into the test tube 212 by injection with pipette 214. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius. In a preliminary step in this embodiment, transport probes 198 are conjugated to 3 um magnetic capture beads 190 by EDC conjugation. Further details regarding conjugation methods are disclosed in commonly assigned U.S. Provisional Application Ser. No. 60/271,922 entitled, “Methods for Attaching Capture DNA and Reporter DNA to Solid Phase Including Selection of Bead Types as Solid Phase” filed Feb. 27, 2001; and U.S. Provisional Application Ser. No. 60/277,854 entitled “Methods of Conjugation for Attaching Capture DNA and Reporter DNA to Solid Phase” filed Mar. 22, 2001, both of which are herein incorporated by reference in their entirety.

As shown in Step II, target DNA or RNA 202 is added to the solution. Oligonucleotide transport probes 198 are complementary to the DNA or RNA target agent 202. The target DNA or RNA 202 thus binds to the complementary sequences of transport probe 198 attached to the capture bead 190 as shown in FIG. 8A.

With reference now to Step II, there is added to the solution 210 reporter beads 192 coated with oligonucleotide signal probes 206. As also shown in FIGS. 9A and 10A, signal probes 206 are complementary to the target DNA or RNA 202. In one embodiment, signal probes 206, which are complementary to a portion of the target DNA or RNA 202, are conjugated to 2.1 um fluorescent reporter beads 192. Signal probes 206 and transport probes 198 each have sequences that are complementary to the target DNA 202, but not complementary to each other. After adding reporter beads 192, the dual bead complex 194 is formed such that the target DNA 202 links capture bead 190 and reporter beads 192. With specific and thorough washing, there should be minimal non-specific binding between reporter bead 192 and capture bead 190. The target agent 202 and signal probe 206 are preferably allowed to hybridize for three to four hours at 37 degrees Celsius.

In this embodiment and others, it was found that intermittent mixing (i.e., periodically mixing and then stopping) produced greater yield of dual bead complex than continuous mixing during hybridization. Thus when this step is performed on-disc, the disc drive motor 140 and controller 142, FIG. 2, may be advantageously employed to periodically rotate the disc to achieve the desired intermittent mixing. This may be implemented in mixing protocols encoded on the disc that rotate the disc in one direction, then stop the disc, and thereafter rotate the disc again in the same direction in a prescribed manner with a preferred duty cycle of rotation and stop sessions. Alternatively, the encoded mixing protocol may rotate the disc in a first direction, then stop the disc, and thereafter rotate the disc again in the opposite direction with a preferred duty cycle of rotation, stop, and reverse rotation sessions. These features of the present invention are discussed in further detail in connection with FIGS. 33A and 35.

As next shown in Step IV of FIG. 65A, after hybridization, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated. Alternatively, this magnetic removal step may be performed on-disc as shown in FIGS. 33A, 35, and 36A-36C.

The purification process illustrated in Step IV includes the removal of supernatant containing free-floating particles. Wash buffer is added into the test tube and the bead solution is mixed well. The preferred wash buffer for the one step assay consists of 145 mM NaCl, 50 mM Tris, pH 7.5, 0.1% SDS, 0.05% Tween, 0.25% NFDM, and 10 mM EDTA. Most of the unbound reporter beads 182, free-floating DNA, and non-specifically bound particles are agitated and removed from the supernatant. The dual bead complex can form a matrix of capture beads, target sequences, and reporter beads, wherein the wash process can further assist in the extraction of free floating particles trapped in the lattice structure of overlapping dual bead particles. Further details relating to other aspects associated with methods of decreasing non-specific binding of reporter beads to capture beads are disclosed in, for example, commonly assigned U.S. Provisional Application Ser. No. 60/272,134 entitled “Reduction of Non-Specific Binding in Dual Bead Assays by Selection of Bead Type and Bead Treatment” filed Feb. 28, 2001; and U.S. Provisional Application Ser. No. 60/275,006 entitled “Reduction of Non-Specific Binding in Dual Bead Assays by Selection of Buffer Conditions and Wash Conditions” filed Mar. 12, 2001. Both of these applications are herein incorporated by reference in their entirety.

The next step in FIG. 65A is Step V. In this step, once the dual bead complex has been washed approximately 3-5 times with wash buffer solution, restriction enzymes, urea, acids (preferably strong acids), or bases (preferably strong bases) may be added to the dual bead solution, as illustrated. The dual bead complexes are thus dissociated by the actions of these dissociation agents thereby releasing the reporter beads 192 from the capture beads 190 as shown in FIG. 65B, Step VI.

After the dissociation of the dual bead structure, the capture beads 190 are separated from the, now unbound, reporter beads 192 in the solution, as shown in Step VII. The solution can be exposed to a magnetic field to capture the magnetic capture beads 190. The magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VI will also be removed from the solution. During step VI, the supernatant containing the released reporter beads are collected using a pipette 214. The assay mixture may then be loaded into the disc 144 or 180 and analyzed using an optical bio-disc or medical CD reader, as illustrated in Step VIII. Either a transmissive bio-disc 180 or a reflective bio-disc 144 may be used to analyze the reporter beads. Details relating to the reflective and transmissive optical bio-discs are discussed above in conjunction with FIGS. 3A-3C and 4A-4C, respectively. The optical bio-disc reader and other alternative disc readers that may be used to analyze optical bio-discs are described in detail above in connection with FIGS. 1 and 2. If the reporter beads are fluorescent, the reporter beads isolated in Step VI may also be quantified using a fluorimeter or any similar fluorescent type analyzer including fluorescent optical disc readers.

FIGS. 66A and 66B taken together illustrate an immunoassay using a “single-step antigen binding” method, similar to that in FIGS. 65A and 65B, to create dual bead complex structures in a solution. This method similarly includes eight principal steps and is related to the method discussed above in connection with FIG. 11B. The eight steps of this method are respectively identified as Steps I, II, III, IV, V, VI, VII, and VIII in FIGS. 66A and 66B.

As shown in Step I, FIG. 66A, capture beads 190, e.g., on the order of 10E+07 in number and each on the order of 1 um or above in diameter, which are coated with antibody transport probes 196 are added to a buffer solution 210. This solution may be the same as that employed in the method shown in FIGS. 65A and 65B or alternatively may be specifically prepared for use with immunochemical assays. The antibody transport probes 196 have a specific affinity for the target antigen 204. The transport probes 196 bind specifically to epitopes within the target antigen 204 as also shown in FIG. 8B. In one embodiment, antibody transport probes 196 which have an affinity for a portion of the target antigen may be conjugated to 3 um magnetic capture beads 190 via EDC conjugation. Alternatively, conjugation of the transport probes 196 to the capture bead 190 may be achieved by passive adsorption.

With reference now to Step II shown in FIG. 66A, the target antigen 204 is added to the solution. The target antigen 204 binds to the antibody transport probe 196 attached to the capture bead 190 as also shown in FIG. 8B.

As illustrated in Step III, reporter beads 192 coated with antibody signal probes 208 are added to the solution. Antibody signal probes 208 specifically binds to the epitopes on target antigen 204 as also represented in FIGS. 9B and 10B. In one embodiment, signal probes 208 are conjugated to 2.1 um fluorescent reporter beads 192. Signal probes 208 and transport probes 196 each bind to specific epitopes on the target antigen, but not to each other. After adding reporter beads 192, the dual bead complex 194 is formed such that the target antigen 204 links capture bead 190 and reporter bead 192. With specific and thorough washing, there should be minimal non-specific binding between reporter bead 192 and capture bead 190.

In Step IV, after the binding in Step III, the dual bead complex 194 is separated from unbound reporter beads in the solution. The solution can be exposed to a magnetic field to capture the dual bead complex structures 194 using the magnetic properties of capture bead 190. The magnetic field can be encapsulated in a magnetic test tube rack 216 with a built-in magnet 218, which can be permanent or electromagnetic to draw out the magnetic beads and remove any unbound reporter beads in the suspension. Note that capture beads not bound to reporter beads will also be isolated. Alternatively, as indicated above, this magnetic removal step may also be performed on-disc as shown in FIGS. 33A, 35, and 36A-36C.

The purification process of Step IV includes the removal of supernatant containing free-floating particles. Wash buffer is added into the test tube and the bead solution is mixed well. Most of the unbound reporter beads 182, free-floating protein samples, and non-specifically bound particles are agitated and removed from the supernatant. The dual bead complex can form a matrix of capture beads, target antigen, and reporter beads, wherein the wash process can further assist in the extraction of free floating particles trapped in the lattice structure of overlapping dual bead particles.

The next step in FIG. 66A is Step V. In this step, once the dual bead complex has been washed approximately 3-5 times with wash buffer solution. Urea, acids, or bases may be added to the dual bead solution as dissociation agents, using a pipette 214 as illustrated. The acids or bases employed herein are preferably strong acids or bases, respectively. The dual bead complexes are thus dissociated by the actions of these dissociation agents thereby releasing the reporter beads 192 from the capture beads 190 as shown in FIG. 66B, Step VI.

After the dissociation of the dual bead structure, the capture beads 190 are separated from the, now unbound, reporter beads 192 in the solution, as shown in Step VII. The solution can be exposed to a magnetic field, either on-disc or off-disc, to capture the magnetic capture beads 190. In the preparatory off-disc method shown here, the magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VI will also be removed from the solution. In Step VII, the supernatant containing the released reporter beads are collected using a pipette 214. The assay mixture may then be loaded directly into the disc and analyzed using an optical bio-disc reader, as illustrated in Step VIII. Either a transmissive bio-disc 180 or a reflective bio-disc 144 may be used to analyze the reporter beads. Details relating to the reflective and transmissive optical bio-discs are discussed in detail in connection with FIGS. 3A-3C and 4A-4C, respectively. The optical bio-disc reader and other alternative disc readers that may be used to analyze optical bio-discs are described in detail above with reference to FIGS. 1 and 2. If the reporter beads are fluorescent, the reporter beads isolated in Step VI may also be quantified using a fluorimeter or any similar fluorescent type analyzer including fluorescent optical disc readers.

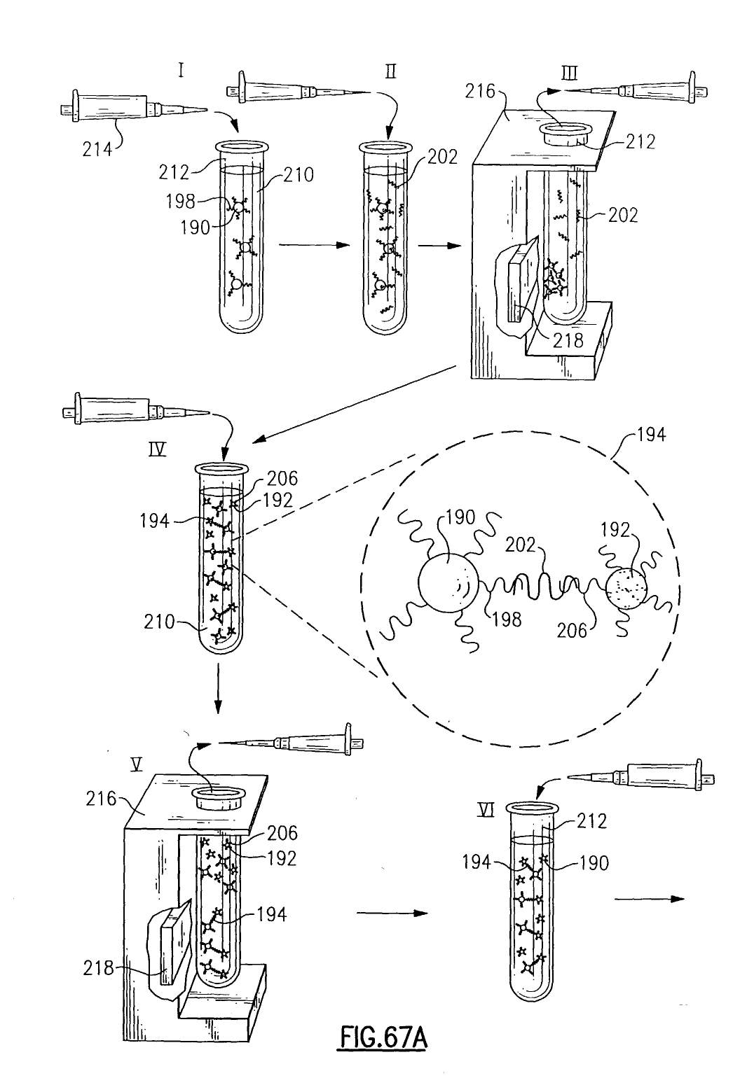

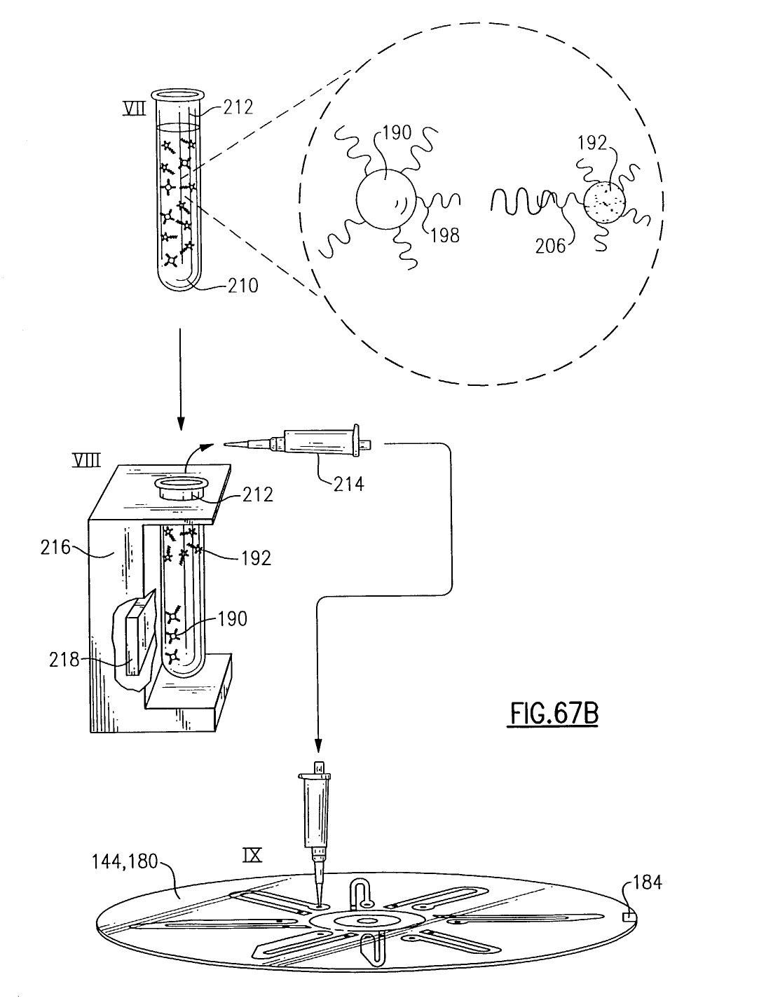

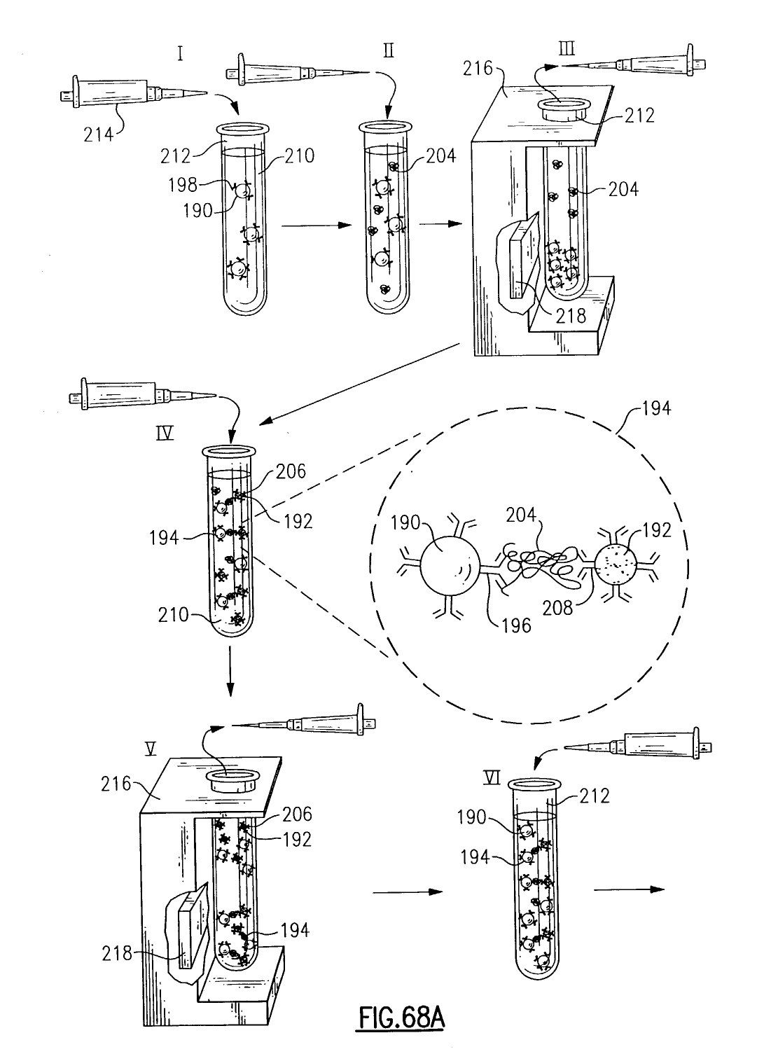

FIGS. 67A and 67B taken together show an alternative genetic assay method referred to here as a “two-step hybridization”. This method is a modified embodiment of the method discussed above in connection with FIG. 12A. The present method has nine principal steps directed to creating the dual bead complex. Generally, capture beads are coated with oligonucleotide transport probes 198 complementary to DNA or RNA target agent and placed into a buffer solution. In this embodiment, transport probes which are complementary to a portion of target agent are conjugated to 3 um magnetic capture beads via EDC conjugation. Other type of conjugation of the oligonucleotide transport probes to a solid phase may be utilized. These include, for example, passive adsorption or use of streptavidin-biotin interactions. The nine main steps according to this method of the present invention are consecutively identified as Steps I, II, III, IV, V, and VI in FIG. 67A, and Steps VII, VIII, and IX in FIG. 67B.

More specifically now with reference to Step I shown in FIG. 67A, capture beads 190, suspended in hybridization solution, are loaded from the pipette 214 into the test tube 212. The preferred hybridization solution is composed of 0.2M NaCl, 10 mM MgCl₂, 1 mM EDTA, 50 mM Tris-HCl, pH 7.5, and 5× Denhart's mix. A desirable hybridization temperature is 37 degrees Celsius.

In Step II, target DNA or RNA 202 is added to the solution and binds to the complementary sequences of transport probe 198 attached to capture bead 190. In one specific embodiment of this method, target agent 202 and the transport probe 198 are allowed to hybridize for 2 to 3 hours at 37 degrees Celsius. Sufficient hybridization, however, may be achieved within 30 minutes at room temperature. At higher temperatures, hybridization may be achieved substantially instantaneously.

As next shown in Step III, target agents 202 bound to the capture beads are separated from unbound species in solution by exposing the solution to a magnetic field to isolate bound target sequences by using the magnetic properties of the capture bead 190. The magnetic field can be enclosed in a magnetic test tube rack 216 with a built-in magnet permanent 218 or electromagnet to draw out the magnetic beads and remove any unbound target DNA 202 free-floating in the suspension via pipette extraction of the solution. As with the above methods, in the on-disc counterpart hereto, this magnetic removal step may be performed as shown in FIGS. 33A, 35, and 36A-36C. A wash buffer is added and the separation process can be repeated. The preferred wash buffer after the transport probes 198 and target DNA 202 hybridize, consists of 145 mM NaCl, 50 mM Tris, pH 7.5, and 0.05% Tween. Hybridization methods and techniques for decreasing non-specific binding of target agents to beads are further disclosed in commonly assigned and co-pending U.S. Provisional Application Ser. No. 60/278,691 entitled “Reduction of Non-Specific Binding of Dual Bead Assays by Use of Blocking Agents” filed Mar. 26, 2001. This application is herein incorporated by reference in its entirety.

Referring now to Step IV illustrated in FIG. 67A, reporter beads 192 are added to the solution as discussed in conjunction with the method shown in FIG. 65A. Reporter beads 192 are coated with signal probes 206 that are complementary to target agent 202. In one particular embodiment of this method, signal probes 206, which are complementary to a portion of target agent 202, are conjugated to 2.1 um fluorescent reporter beads 192. Signal probes 206 and transport probes 198 each have sequences that are complementary to target agent 202, but not complementary to each other. After the addition of reporter beads 192, the dual bead complex structures 190 are formed. As would be readily apparent to one of skill in the art, the dual bead complex structures are formed only if the target agent of interest is present. In this formation, target agent 202 links magnetic capture bead 190 and reporter bead 192. Using the preferred buffer solution, with specific and thorough washing, there is minimal non-specific binding between the reporter beads and the capture beads. Target agent 202 and signal probe 206 are preferably allowed to hybridize for 2-3 hours at 37 degrees Celsius. As with Step II discussed above, sufficient hybridization may be achieved within 30 minutes at room temperature. At higher temperatures, the hybridization taking place in this step may also be achieved substantially instantaneously.

With reference now to Step V shown in FIG. 67A, after the hybridization in Step IV, the dual bead complex 194 is separated from unbound species in solution. The solution is again exposed to a magnetic field to isolate the dual bead complex 194 using the magnetic properties of the capture bead 190. Note again that the isolate will include capture beads not bound to reporter beads. As with Step III above in the on-disc counterpart hereto, this magnetic separation step may be performed as shown in FIGS. 33A, 35, and 36A-36C.

A purification process to remove supernatant containing free-floating particles includes adding wash buffer into the test tube and mixing the bead solution well. The preferred wash buffer for the two-step assay consists of 145 mM NaCl, 50 mM Tris, pH 7.5, 0.1% SDS, 0.05% Tween, 0.25% NFDM, and 10 mM EDTA. Most unbound reporter beads, free-floating DNA, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles. Other related aspects directed to reduction of non-specific binding between reporter bead, target agent, and capture bead are disclosed in, for example, commonly assigned U.S. Provisional Application Ser. No. 60/272,243 entitled “Mixing Methods to Reduce Non-Specific Binding in Dual Bead Assays” filed Feb. 28, 2001; and U.S. Provisional Application Ser. No. 60/272,485 entitled “Dual Bead Assays Including Linkers to Reduce Non-Specific Binding” filed Mar. 1, 2001, which are incorporated herein in their entirety.

The next step shown in FIG. 67A is Step VI. In this step, once the dual bead complex 194 has been washed approximately 3-5 times with wash buffer solution, the assay mixture is loaded into the disc and analyzed. Alternatively, during this step, the oligonucleotide signal and transport probes may be ligated to prevent breakdown of the dual bead complex during the disc analysis and signal detection processes. Further details regarding probe ligation methods are disclosed in commonly assigned and co-pending U.S. Provisional Application Ser. No. 60/278,694 entitled “Improved Dual Bead Assays Using Ligation” filed Mar. 26, 2001, which is herein incorporated in its entirety by reference. In yet another embodiment of the present invention, restriction enzymes, urea, acids, or bases may be added using a pipette 214 to the dual bead solution, as illustrated in Step VI of FIG. 67A. The dual bead complexes are dissociated by the actions of these dissociation agents thus releasing the reporter beads 192 from the capture beads 190 as shown in FIG. 67B, Step VII. The acids or bases utilized herein are preferably strong acids or bases, respectively.

After the dissociation of the dual bead structure, the capture beads 190 are separated from the, now unbound, reporter beads 192 in the solution, as shown in Step VIII. The solution can be exposed to a magnetic field to capture the magnetic capture beads 190. The magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VIII will also be removed from the solution. During Step VIII, the supernatant containing the released reporter beads are collected using a pipette 214. The assay mixture may then be loaded into the disc and analyzed using an optical bio-disc reader, as illustrated in Step 1×. Either a transmissive bio-disc 180 or a reflective bio-disc 144 may be used to analyze the reporter beads. Details relating to the reflective and transmissive optical bio-discs are discussed in detail in conjunction with FIGS. 3A-3C and 4A-4C, respectively. The optical bio-disc reader and other alternative disc readers that may be used to analyze optical bio-discs are described in detail above with reference to FIGS. 1 and 2. If the reporter beads are fluorescent, the reporter beads isolated in Step VIII may also be quantified using a fluorimeter or any similar fluorescent type analyzer including fluorescent optical disc readers. Experiments performed using dissociation agents to release the reporter beads from the dual bead complex are described in detail below in Examples 15 and 16.

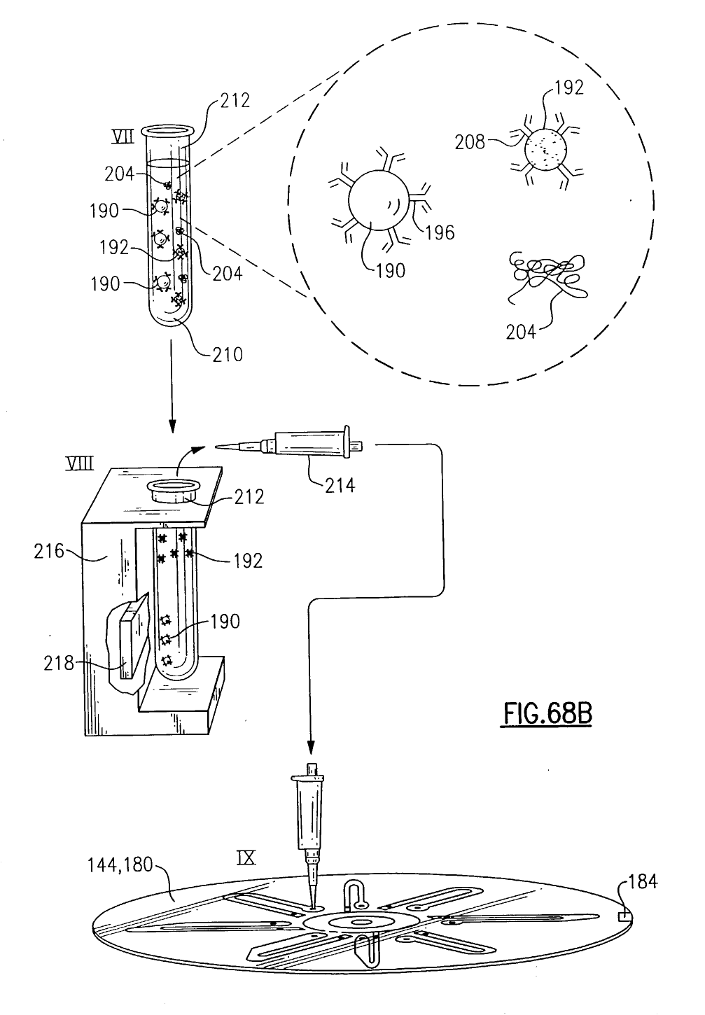

In accordance with another aspect of this invention, FIGS. 68A and 68B taken together show an immunoassay method, similar to those discussed in connection with FIGS. 66A and 66B, and following the techniques of the genetic assay in FIGS. 67A and 67B. This method is also referred to here as a “two-step binding” to create the dual bead complex in an immunochemical assay. This method is a related and more specific embodiment of the method illustrated above in FIG. 12B. As with the method shown in FIGS. 67A and 67B, this method includes nine main steps. In general, capture beads coated with antibody transport probes which specifically binds to epitopes on target antigen are placed into a buffer solution. In one specific embodiment, antibody transport probes are conjugated to 3 um magnetic capture beads. Different sized magnetic capture beads may be employed depending on the type of disc drive and disc assembly utilized to perform the assay. The nine main steps according to this alternative method of the invention are respectively identified as Steps I, II, III, IV, V and VI in FIG. 68A and Steps VI, VII, and IX in FIG. 68B.

With specific reference now to Step I shown in FIG. 68A, capture beads 190, suspended in buffer 210 solution, are loaded into a test tube 212 via injection from pipette 214.

In Step II, target antigen 204 is added to the solution and binds to the antibody transport probe 196 attached to capture bead 190. Target antigen 204 and the transport probe 196 are preferably allowed to bind for 2 to 3 hours at 37 degrees Celsius. Shorter binding times are also possible.

As shown in Step III, target antigen 204 bound to the capture beads 190 are separated from unbound species in solution by exposing the solution to a magnetic field to isolate bound target proteins or glycoproteins by using the magnetic properties of the capture bead 190. The magnetic field can be enclosed in a magnetic test tube rack 216 with a built-in magnet permanent 218 or electromagnet to draw out the magnetic beads and remove any unbound target antigen 204 free-floating in the suspension via pipette extraction of the solution. A wash buffer is added and the separation process can be repeated.

As next illustrated in Step IV, reporter beads 192 are added to the solution as discussed in conjunction with the method shown in FIG. 66A. Reporter beads 192 are coated with signal probes 208 that have an affinity for the target antigen 204. In one particular embodiment of this two-step immunochemical assay, signal probes 208, which bind specifically to a portion of target agent 204, are conjugated to 2.1 um fluorescent reporter beads 192. Signal probes 208 and transport probes 196 each bind to specific epitopes on the target agent 204, but do not bind to each other. After the addition of reporter beads 192, the dual bead complex structures 190 are formed. As would be readily apparent to those skilled in the art, these dual bead complex structures are formed only if the target antigen of interest is present. In this formation, target antigen 204 links magnetic capture bead 190 and reporter bead 192. Using the preferred buffer solution, with specific and thorough washing, there is minimal non-specific binding between the reporter beads and the capture beads. Target antigen 204 and signal probe 208 are allowed to hybridize for 2-3 hours at 37 degrees Celsius. As with Step II discussed above, sufficient binding may be achieved within 30 minutes at room temperature. In the case of immunoassays temperatures higher than 37 degrees Celsius are not preferred because the proteins will denature.

Turning next to Step V as illustrated in FIG. 68A, after the binding shown in Step IV, the dual bead complex 194 is separated from unbound species in solution. This is achieved by exposing the solution to a magnetic field to isolate the dual bead complex 194 using the magnetic properties of the capture bead 190 as shown. Note again that the isolate will include capture beads not bound to reporter beads.

A purification process to remove supernatant containing free-floating particles includes adding wash buffer into the test tube and mixing the bead solution well. Most unbound reporter beads, free-floating proteins, and non-specifically bound particles are agitated and removed from the supernatant. The dual bead complex can form a matrix of capture beads, target agents, and reporter beads, wherein the wash process can further assist in the extraction of free floating particles trapped in the lattice structure of overlapping dual bead particles.

The last step shown in FIG. 68A is Step VI. In this step, once the dual bead complex 194 has been washed approximately 3-5 times with wash buffer solution, urea, acids (preferably strong acids), or bases (preferably strong bases) may be added to the dual bead solution using a pipette 214, as illustrated in Step VI of FIG. 68A. The dual bead complexes are dissociated by the actions of these dissociation agents thus releasing the reporter beads 192 from the capture beads 190 as shown next in Step VII of FIG. 68B.

After the dissociation of the dual bead structure, the capture beads 190 are separated from the, now unbound, reporter beads 192 in the solution, as shown in Step VIII of FIG. 68B. The solution can be exposed to a magnetic field to capture the magnetic capture beads 190. The magnetic field can be encapsulated in a magnetic test tube rack 216 with a built-in magnet 218, which can be permanent or electromagnetic to draw out the magnetic beads and remove any unbound reporter beads in the suspension. Note that non-dissociated dual bead complexes not separated during Step VII will also be removed from the solution. During Step VIII in this method, the supernatant containing the released reporter beads 192 are collected using a pipette 214. The assay mixture may then be loaded into the disc and analyzed using an optical bio-disc reader, as illustrated in Step IX. Either a transmissive bio-disc 180 or a reflective bio-disc 144 may be used to analyze the reporter beads. Details relating to the reflective and transmissive optical bio-discs are discussed in detail with reference to FIGS. 3A-3C and 4A-4C, respectively. The optical bio-disc reader and other alternative disc readers that may be used to analyze optical bio-discs or medical CDs are described in detail above in conjunction with FIGS. 1 and 2. The reporter beads isolated in Step VIII may also be quantified using a fluorimeter or any similar fluorescent type analyzer including fluorescent optical disc readers.

As with any of the other methods discussed above, the magnetic removal or separation steps in the method shown in FIGS. 68A and 68B may be alternatively performed on-disc using the disc, fluidic circuits, and apparatus illustrated in FIGS. 33A-33D, 34A-34C, 35, 36A-36C, and 37.

Use of Dissociation Agents to Increase Assay Sensitivity and Decrease Non-specific Bead Binding

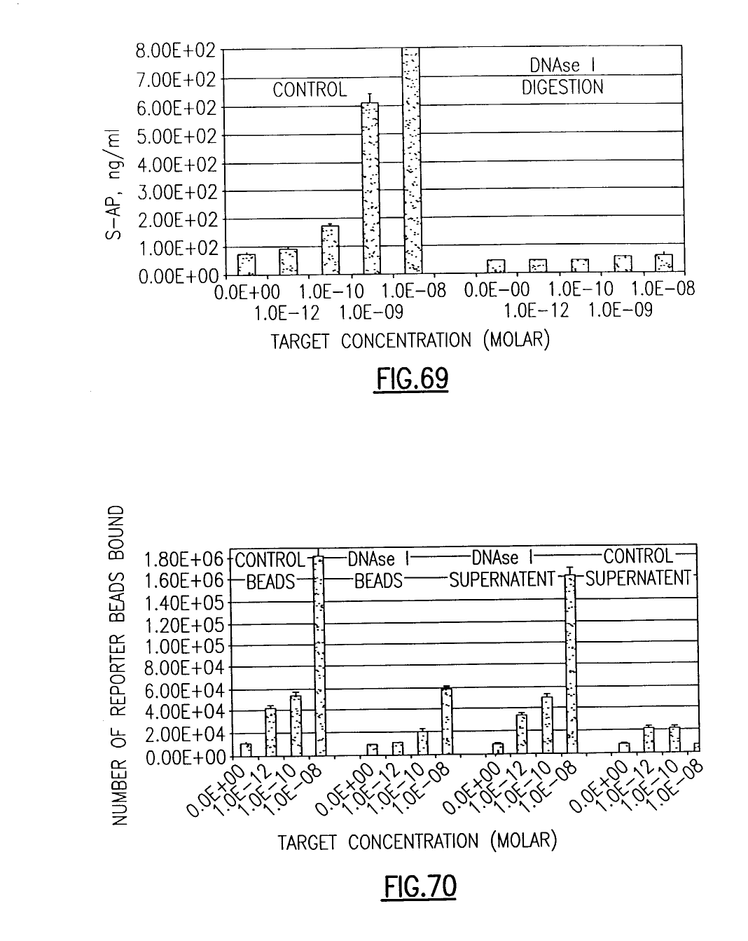

Moving along now to FIG. 69, there is shown a bar graph presentation demonstrating the DNAseI digestion efficiency in the absence of reporter beads. In this experiment, biotinylated target DNA 202 was hybridized to transport probes 198 on the magnetic capture bead 190 as illustrated above in FIG. 8A. After hybridization, streptavidinated alkaline phosphatase (S-AP) was added to the assay mix and allowed to bind with the biotin on the target DNA. Following a series of wash steps, an S-AP chromagen substrate was added to an aliquot of the assay solution and the amount of bound target was quantified colorimetrically using a spectrophotometer. At the same time, an aliquot of equal volume to that taken above was incubated in buffer containing DNAseI. After incubation, the assay mix was washed and S-AP was added to the solution and allowed to bind to residual targets that were not digested by DNAseI. The observations showed a high DNAse digestion activity as manifested in the difference in signal between the control and the DNAseI digestion treatments. Details regarding an experiment similar to the one discussed here is described in detail below in Example 15.

Referring now to FIG. 70, there is shown a bar graph illustration of data collected from an experiment similar to that discussed in FIG. 69. In this experiment, dual bead complexes were used instead of the magnetic capture bead alone as described in FIG. 69. In this case, the target 202 is situated between the capture bead 190 and reporter bead 192 as shown above in FIG. 10A. After formation of the dual bead complex, as described in FIG. 65A, Steps I-V, the amount of reporter beads in the supernatant collected in Step IV (FIG. 65A) and the reporter beads bound to the capture beads in the assay mix shown in Step V (FIG. 65A) were quantitated using a fluorimeter. DNAseI was then added to the assay mix and allowed to cleave the target bound to the probes in the dual bead complex, as represented in Step VI in FIG. 65B, thereby releasing the reporter bead from the magnetic capture beads. The next step involved the isolation of the capture beads and collection of the supernatant containing newly released fluorescent reporter beads as shown in Step VI in FIG. 65B. The signal from the reporter beads in the supernatant was quantified using a fluorimeter. The undissociated reporter beads were also quantified by a fluorimeter. Data collected from this experiment shows the DNAseI enzyme digestion was not as efficient in a dual bead complex relative to that in a single bead set-up as described in FIG. 69. The decrease in DNAse digestion activity may be due to steric hindrance from the beads in the dual bead complex blocking DNAse access to the target. Experimental details regarding the use of restriction enzyme digestion as implemented in a dual bead assay is discussed in further detail below in Example 15. The fluorescent reporter beads collected in this assay may also be quantified using an optical bio-disc or medical CD with a fluorescent type optical disc reader or any similar device as discussed above.

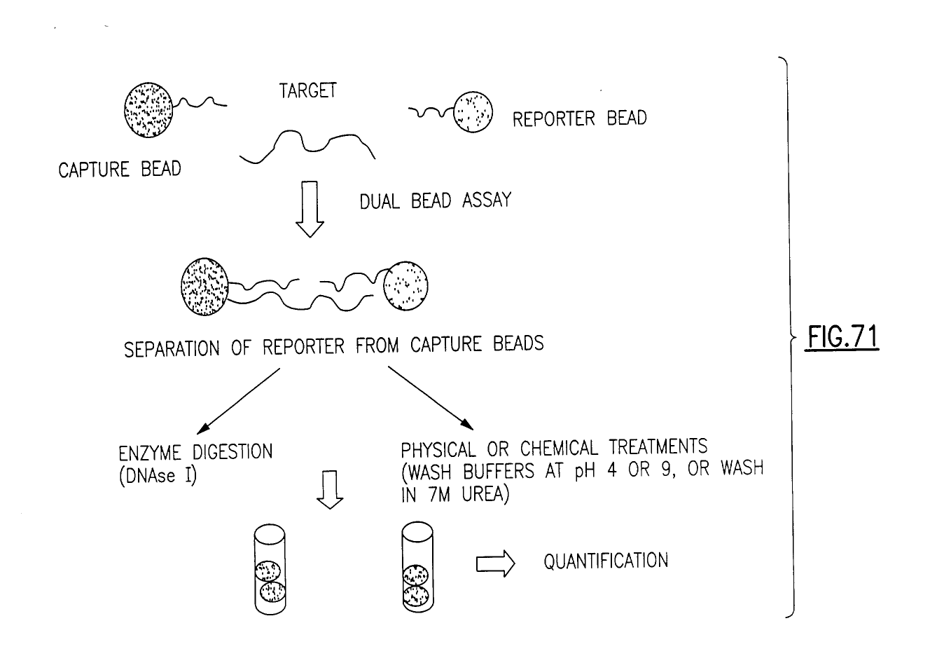

The sensitivity of any assay depends on the ratio of signal over noise. The sensitivity of the dual bead assay relies on the minimization of non-specific binding between capture beads and reporter beads. The non-specific interactions between the dual beads in the absence of targets are so stable that stringent washing cannot eliminate them. The contribution of the non-specific dual beads, however, can be negated by the exclusive detection and quantification of target-mediated dual beads. As shown in FIG. 71, the dual bead complexes can be separated by enzyme digestion (DNAse, restriction enzymes) or by chemical and physical treatments (heat, urea, base, or acid treatment). Furthermore, the quantification of reporter beads in the presence of a large excess of unbound capture beads may reduce the sensitivity of the detection assay. Therefore, the separation of reporter beads from capture beads will facilitate the quantification of reporter beads by the bio-disc reader and thus increase the sensitivity of the assay.

With more particular reference now to FIG. 71, there is a schematic representation of separation of reporter beads from capture beads in a dual bead complex by enzyme digestion and physical or chemical treatments. FIG. 71 shows a summary of the dual bead formation and dissociation using various dissociation agents as discussed in conjunction with FIGS. 65A, 65B, 67A, and 67B. After the released reporter beads are collected, the beads are quantified by any one of several methods including a fluorimeter, a fluorimager, a fluorescent type optical disc reader system, a CD-R type optical disc system or any device capable of detecting micro-spheres or fluorescence. The current preferred method of detection is the use of an optical bio-disc or medical CD system as discussed in detail in connection with FIGS. 1, 2, 3A, 3B, 3C, 4A, 4B, and 4C.

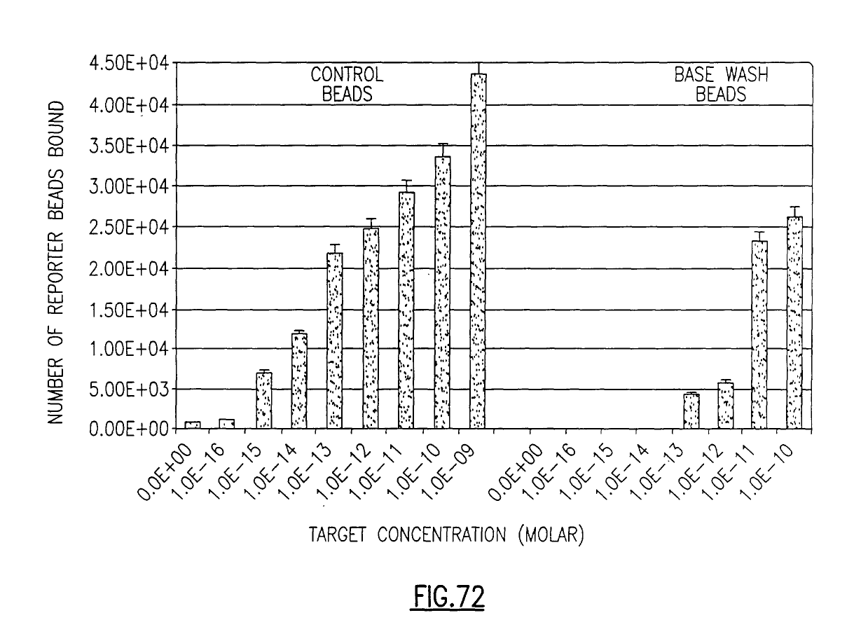

With reference next to FIG. 72, there is a bar graph presentation showing separation of reporter beads from capture beads using high pH washes at various target concentrations similar to that described in FIG. 70. The dual bead complex is formed as described in Steps I-VI in FIG. 67A. Once the dual beads are formed and isolated, a strong base is added to the dual bead solution as shown in Step VI (FIG. 67A). After a brief incubation, the dual beads are dissociated by the actions of the base that disrupts the hydrogen bonding between the target and the probes thereby releasing the fluorescent reporter beads and the magnetic capture beads from the dual bead complex as shown in Step VI (FIG. 67B). The next step is to isolate the capture beads as described in Step VIII of FIG. 67B. The isolated capture beads will also contain non-dissociated dual bead complexes. This isolate was quantified by a fluorimeter in this particular experiment. The data shown in FIG. 72 illustrate 100% dissociation of dual bead complexes at target concentrations from 1×10⁻¹⁶ M to 1×10⁻¹⁴M. Details of an experiment performed using base as a dissociation agent is described in detail below in Example 16. The reporter beads may also be quantified by any one of several methods including a fluorimager, a fluorescent type optical disc reader system, a CD-R type optical disc system or any device capable of detecting microspheres or fluorescence. The preferred method of detection is the use of an optical bio-disc or medical CD system as discussed in detail in conjunction with FIGS. 1, 2, 3A, 3B, 3C, 4A, 4B, and 4C.

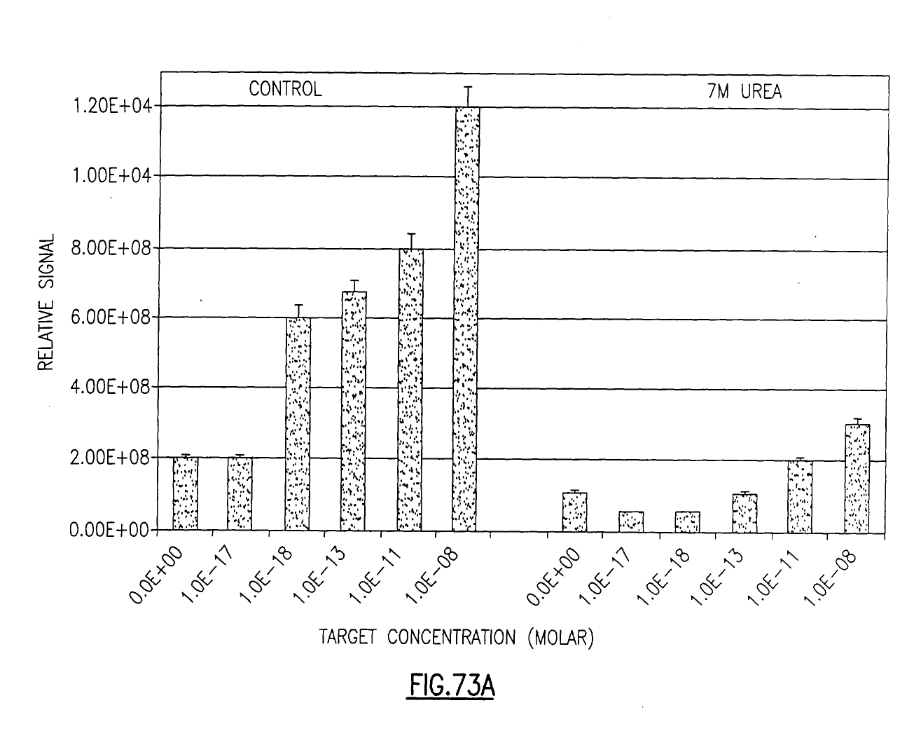

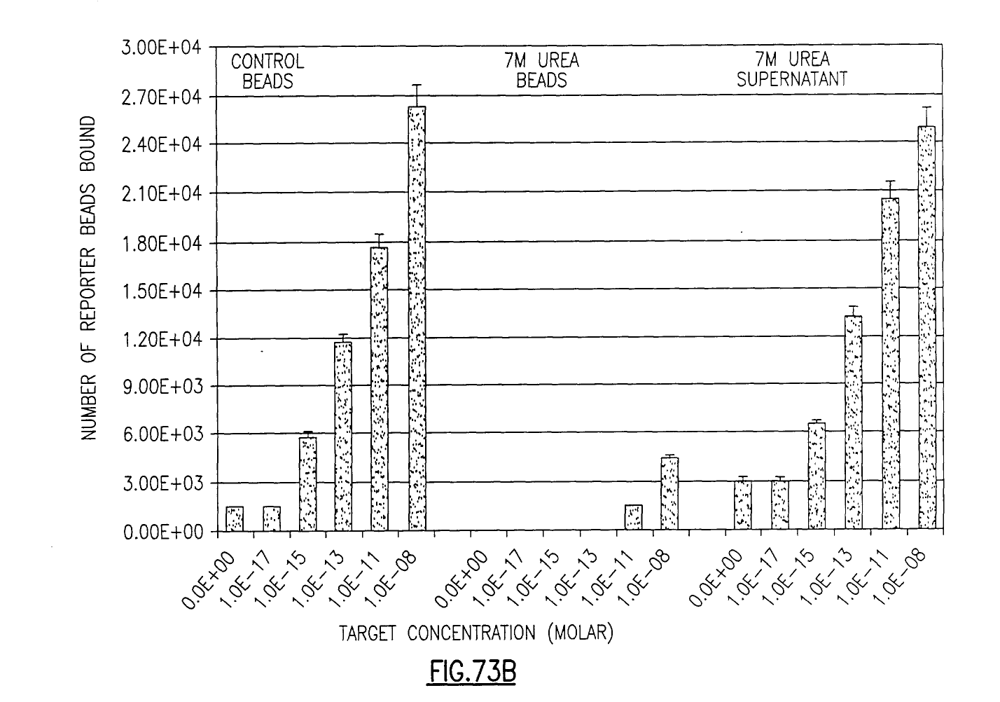

Referring now to FIG. 73A, there is a bar graph illustration of data collected from an experiment using urea as a denaturing or dissociation agent. Details of this experiment are similar to those discussed in conjunction with FIG. 69. As in FIG. 69, biotinylated target DNA 202 was hybridized to transport probes 198 on the magnetic capture bead 190 as illustrated above in FIG. 8A. After hybridization, streptavidinated alkaline phosphatase (S-AP) was added to the assay mix and allowed to bind with the biotin on the target DNA 202. Following a series of wash steps, an S-AP chromagen substrate was added to an aliquot of the assay solution and the amount of bound target was quantified colorimetrically using a spectrophotometer. At the same time, an aliquot of equal volume to that taken above was incubated in buffer containing 7M urea. After incubation, the assay mix was washed and S-AP was added to the solution and allowed to bind to residual targets, still bound to transport probes on the capture bead, that were not denatured by the 7M urea. The results thereof showed a relatively efficient denaturation activity as revealed by the difference in signal between the control and the 7M urea treatments. Details regarding an experiment similar to the one discussed here is described in detail below in Example 16.

Referring next to FIG. 73B, there is a bar graph illustrating data collected from an experiment using urea as a dissociation agent in a dual bead assay. Details of this experiment are similar to that discussed in conjunction with FIG. 70. As in FIG. 70; dual bead complexes were used instead of the magnetic capture bead alone as described in FIG. 73A. In this case, the target is situated between the capture and reporter beads as shown above in FIG. 10A. After formation of the dual bead complex, as described in FIG. 67A, Steps I-VI, the amount of reporter beads 192 in the supernatant collected in Step V (FIG. 67A) and the reporter beads 192 bound to the capture beads 190 in the assay mix shown in Step VI (FIG. 67A) were quantitated using a fluorimeter. A predetermined amount of urea was then added to make the final urea concentration in the assay solution 7M. This resulted in denaturation of the target bound to the probes in the dual bead complex, as represented in Step VII in FIG. 67B, thus releasing the reporter bead from the magnetic capture beads. The next step involved the isolation of the capture beads 190 and collection of the supernatant containing newly released fluorescent reporter beads 192 as shown in Step VIII in FIG. 67B. The signal from the reporter beads in the supernatant was quantified using a fluorimeter. The undissociated reporter beads were also quantified by a fluorimeter. Data collected from this experiment shows the 7M urea denaturation was more efficient in a dual bead complex assay relative to using DNAseI as a dissociation agent as described in FIG. 70. The increase in dissocaition may be due to the lack of steric hindrance from the beads in the dual bead complex since urea is a significantly smaller molecule than DNAse. Experimental details regarding the use of 7M urea denaturation as implemented in a dual bead assay is discussed in further detail in Example 16. The fluorescent reporter beads collected in this assay may also be quantified using an optical bio-disc or medical CD with a fluorescent type optical disc reader or any similar device as discussed above.

Use of DNA Denaturing Agents to Improve DNA Target Detection

It is a principal aspect of the invention to further modify the dual bead assays to detect medical targets. In real samples, the DNA targets are double-stranded and very long. The ability of the dual bead assay, as well as for any other DNA diagnostic assays, to detect sequences of clinical interest within the whole genome relies first on the specificity of the probes for the sequence of interest and second on the use of very strong detergent to keep the DNA target in the denatured, single-stranded, form for capture.

The success of the dual bead assays in detecting sequences of clinical interest relies primarily on the design of the probes. Given the complexity and degeneracy of the human genome, the probes designed to detect sequences of clinical interest have to be unique to the diagnostic sequence and yet common enough to recognize mutants of the sequences. The design of the probes using computer software allows comparison of sequences to existing sequences in the data bank such as Blast search. Once probes specific for the sequence of interest have been designed, the major modification introduced to the dual bead assays includes the use of a denaturing agent in the hybridization buffer to prevent re-annealing of complementary sequences of the target DNA. This allows hybridization between the target and probes.

The present invention is also addressed at implementing the methods recited above on to an analysis disc, modified optical disc, medical CD, or a bio-disc. A bio-disc or medical CD drive assembly, such as those discussed above with reference to FIGS. 1 and 2, may be employed to rotate the disc, read and process any encoded information stored on the disc, and analyze the DNA samples in the flow channel of the bio-disc or medical CD. The bio-disc drive is thus provided with a motor for rotating the bio-disc, a controller for controlling the rate of rotation of the disc, a processor for processing return signals form the disc, and an analyzer for analyzing the processed signals. The rotation rate of the motor is controlled to achieve the desired rotation of the disc. The bio-disc drive assembly may also be utilized to write information to the bio-disc either before, during, or after the medical test material in the flow channel and target zones is interrogated by the read beam of the drive and medically diagnosed by the analyzer. In this embodiment of the present invention, the analyzer may advantageously include specialized diagnostic software to thereby provide a medical expert system. The bio-disc may similarly include corresponding medical expert system software and encoded information for controlling the rotation rate of the disc, providing processing information specific to the type of DNA test to be conducted, and for displaying the results on a monitor associated with the bio-drive.

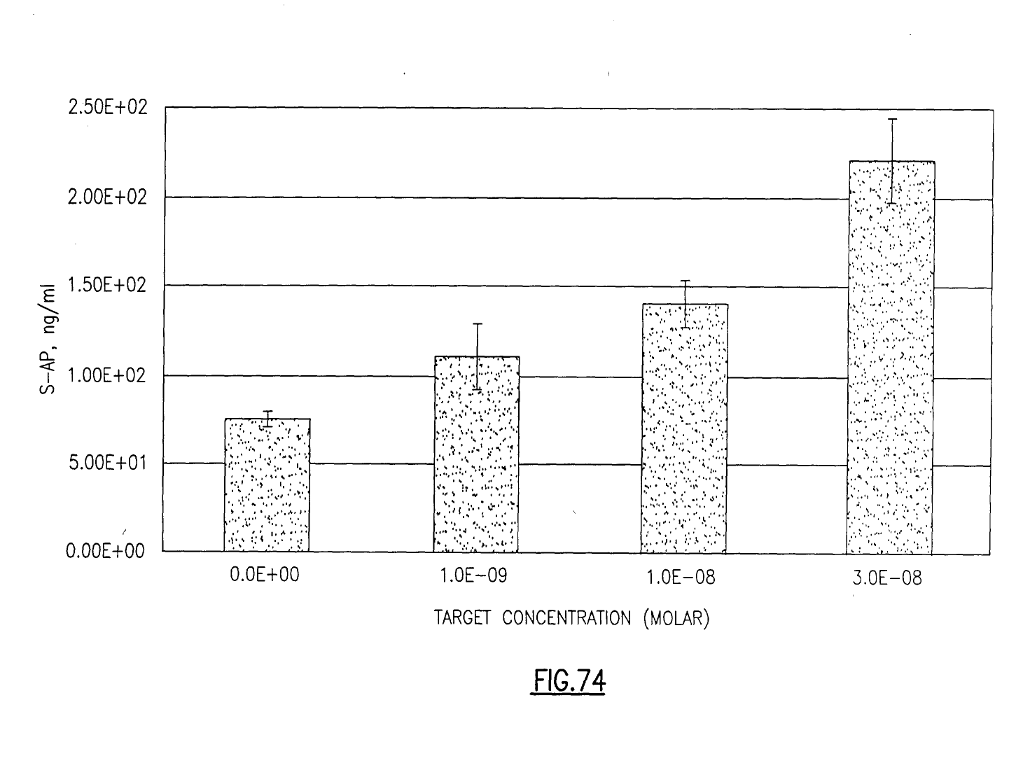

In a preferred embodiment of this invention, guanidine isothiocyanate is used as a typical denaturing agent. Data collected from an experiment using 1.5M guanidine isothiocyanate as denaturing agent is illustrated in FIG. 74. The experimental procedure followed for this experiment is described in detail below in Example 17. FIG. 74 further illustrates that the assay sensitivity is significantly increased if an appropriate amount of denaturing agent is used in a hybridization experiment. In this particular assay, biotinylated target DNA 202 was hybridized to transport probes 198, in the presence of 1.5M guanidine isothiocyanate, on the magnetic capture bead 190 as illustrated above in FIG. 8A. After hybridization, streptavidinated alkaline phosphatase (S-AP) was added to the assay mix and allowed to bind with the biotin on the target DNA 202. Following a series of wash steps, an S-AP chromagen substrate was added to the assay solution, ample time was allotted for color development, and the amount of bound target was quantified calorimetrically using a spectrophotometer.

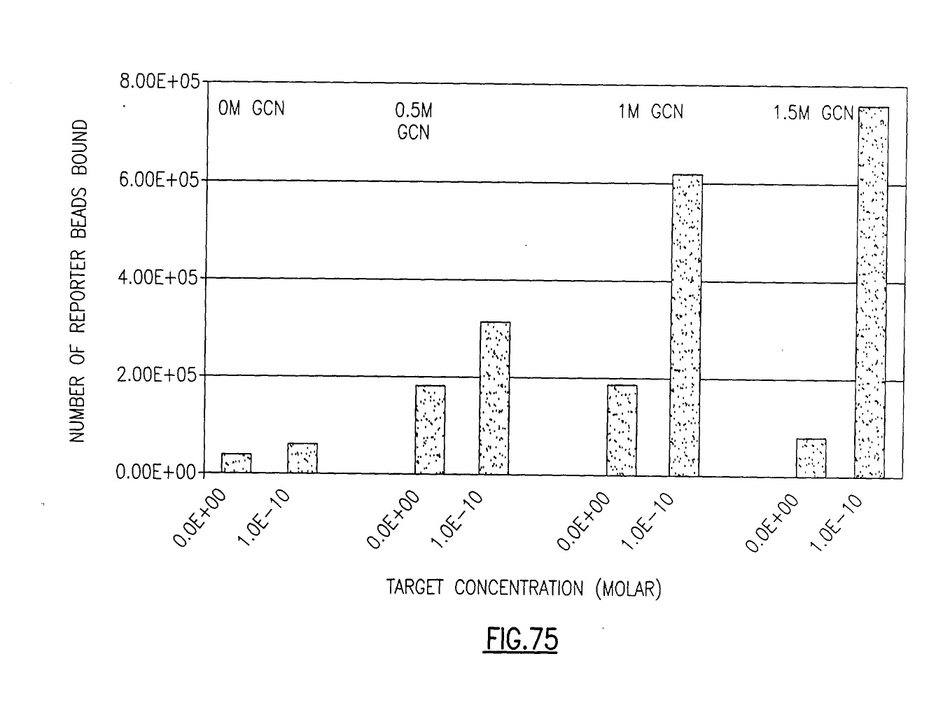

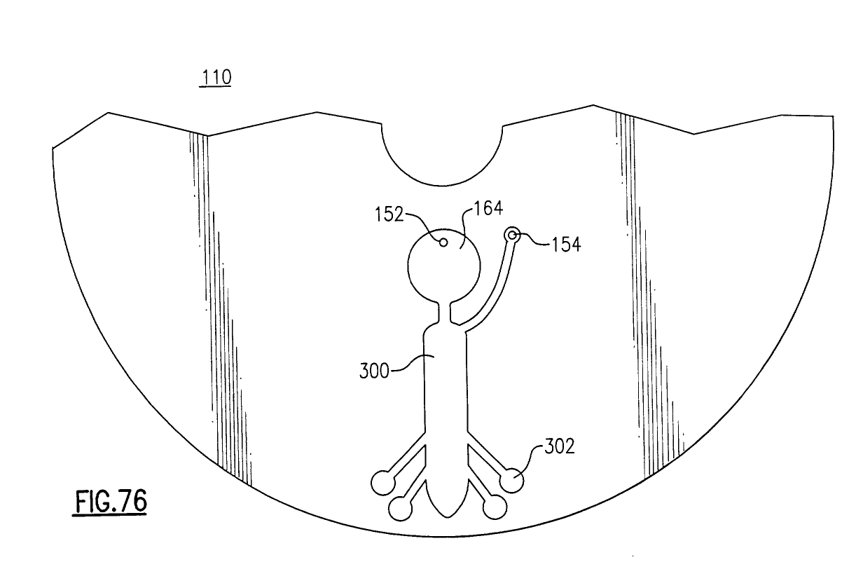

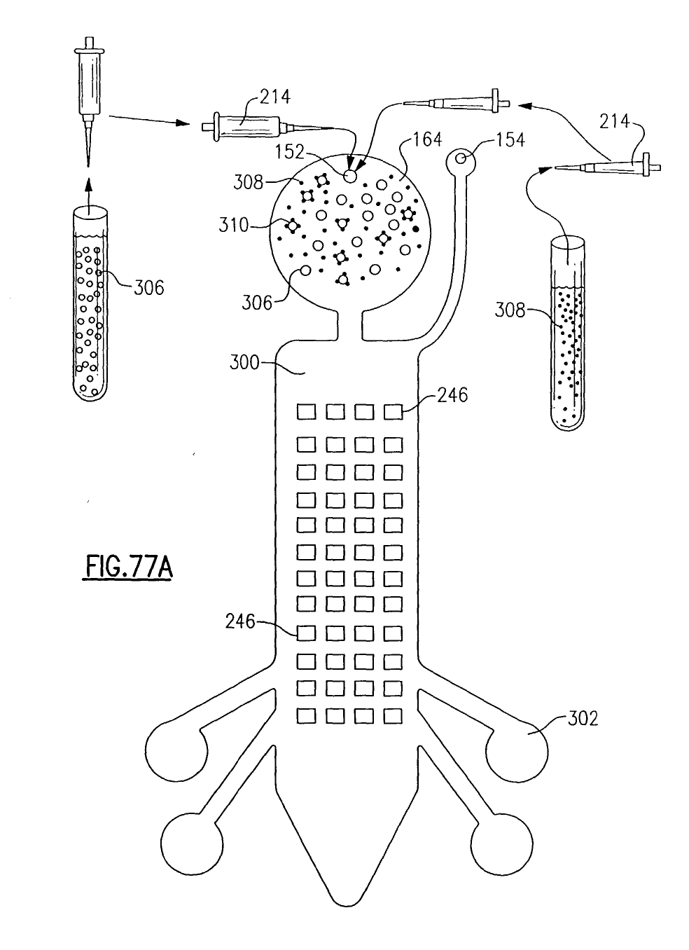

An appropriate amount of guanidine isothiocyanate is necessary to prevent re-annealing of the complementary sequences of the target DNA while allowing hybridization between the target and the probes. At high concentrations, however, guanidine isothiocyanate prevents any hybridization. To determine the appropriate buffer concentration of guanidine isothiocyanate for use in a dual bead assay, a titration of guanidine isothiocyanate was performed. The data from this titration experiment is shown in FIG. 75. As illustrated in FIG. 75, the optimal hybridization buffer concentration of guanidine isothiocyanate is 1.5M since the addition of 1.5M guanidine isothiocyanate showed the highest difference in signal between the 0.0M and 1.0×10⁻¹⁰M target concentration.

MO Bio-Magnetic Assays (MOBMA) for Selecting, Detecting, and Manipulating Specific Cell Populations using the MO Bio-Disc System (MOBDS)

A further aspect of the present invention relates to a magnetic method for detection of specific target cells in cell populations and solutions of cell populations, using magnetic particles or beads as described above and referred to as the MOBMA. In one embodiment of the MOBMA aspect of the current invention, the magnetic beads are coated with one or more binding agents or capture probes to thereby form bio-magnetic particles or beads. Further details related to attaching binding agents onto a solid support including magnetic particles is disclosed in commonly assigned and co-pending U.S. patent application Ser. No. 10/194,396 entitled “Multi-Purpose Optical Analysis Disc For Conducting Assays and Various Reporting Agents for use Therewith” filed Jul. 12, 2002, which is incorporated by reference in its entirety herein. The binding agents may include, for example, antibodies recognizing Fc portions of target cell associating antibodies directed to specific antigen determinants on cell membranes. Capture, detection, manipulation, and quantitation of target cells are carried out using the MOBDS of the present invention. The captured cells may also be magnetically manipulated or moved from one analysis, separation, or testing chamber to another, on the MO bio-disc, to facilitate cellular testing. For example, captured target cancer cells may be magnetically aliquoted into equal cell numbers to different analysis chambers containing cancer therapeutic agents to determine the effect of these agents on a cellular level. Specific examples of applications of this method are described below.

Incubation of the cell suspension with a mild detergent and/or second set of antibodies or antibody fragments, pre-labeled or not with fluorescent agents, metallocolloids, bio-magnetic particles, radioisotopes, biotin complexes or certain enzymes allowing visualization, may dramatically increase the specificity and sensitivity of said method. The method can further be used to dynamically isolate, purify, manipulate, and quantify target cells magnetically captured on magnetic capture zones on the MO bio-disc. Further details relating to a method for detection of specific target cells in specialized or mixed cell population and solutions containing mixed cell populations is disclosed in, for example, U.S. Pat. No. 6,184,043 to Fodstad and Kvalheim, which is herein incorporated by reference in its entirety.

The binding agents may bind one or more chemical/biochemical entities together by affinity. In affinity binding, a pair of binding partners, which for example are attached to the substances to be linked, bind to each other when brought in contact. A molecule on a cell surface may represent one of the binding partners, in such a linkage. Several such binding partner systems are known, such as, for example, antigen-antibody, enzyme-receptor, ligand-receptor interactions on cells, biotin-avidin binding, hapten/anti-hapten binding pairs, and oligonucleotide complementary sequence binding, of which antigen-antibody binding is most frequently used.

Methods are known in which one of the binding partners is attached to an insoluble support, such as magnetic particles or beads, and by which isolation of target cells in a mixed cell population is performed as negative isolation or positive isolation. In a negative isolation procedure the unwanted cells can be removed from the cell preparation by incubating the cells with antibody-coated magnetic beads (bio-magnetic beads or particles) specific for the unwanted cells. Following the incubation the resulting cell-bead complex can be removed or isolated magnetically, leaving the wanted target cells behind. In a positive isolation procedure, on the other hand, the wanted target cells are removed from the mixed cell population using magnetic beads coated with antibodies (bio-magnetic beads) directed to the cells of interest.

An object of the MOBMA aspect of the present invention is to detect and study specific target cells for diagnostic purposes. The test samples may include blood, bone marrow, ascites fluid, and cells from various tissues or tumors. The present invention represents a sensitive detection system and method for detecting a variety of cell types, such that a high number of cells can be readily screened using the MOBDS and the procedure is rapid and simple. Furthermore, the present MOBDS may be used for isolation of cells for biochemical, biological and immunological examination, and for studying of specific genes at the nucleotide or protein level. In addition, the isolated or captured cell-bead complexes may be released by disrupting or removing the magnetic field within the magnetic capture zone, on the MO bio-disc, where cells of interest are located, using the associated MO drive and the cells cultured or used for in vitro cytotoxicity studies without the need for cleaving the cell-bead complex.

Another embodiment of the MOBMA aspect of the present invention includes immunomagnetic positive isolation of target cells, normal or pathogenic, in a mixed cell population or physiological solution. In this embodiment of the present invention, a linkage is created between a specific target cell and an insoluble support such as a magnetic bead or particle. The particle is either coated with an anti-cell antibody directed to specific antigen determinants on the membranes of the target cells, or the particles are coated with a polyclonal anti-mouse or anti-human antibody capable of binding to the Fc-portions of a specific anti-cell antibody (primary antibody) directed to the antigen determinants on the membrane of the target cell. Instead of using the polyclonal anti-mouse/anti-human antibody for coating the particles, a monoclonal rat anti-mouse/anti-human antibody may be used. The use of a monoclonal antibody may reduce the risk for possible cross-reactions with non-target cells in solutions. Furthermore, incubation of the cell suspension with a mild detergent and/or second set of antibodies or antibody fragments that bind to antigenic determinants on one or more cell types among the captured cells, in a mixed cell capture, pre-labeled with fluorescent agents, magnetic particles, metallocolloids, bio-magnetic particles, radioisotopes, biotin-complexes or certain enzymes allowing visualization, will dramatically increase the specificity of the method of this embodiment.

A detailed application of the immunomagnetic positive isolation embodiment described above is presented below using cancer cells as the target cells for detection and isolation. This embodiment is, however, not limited to cancer cells and the disclosure shall not limit the method to this particular field of use since the method is suitable for use with a range of cytological applications including assays directed to, for example, cells of the immune system such as natural killer cells, monocytes, T-cells, B-cells, and subtypes thereof including CD4+ and CD8+ cells; bacterial cells (Chandler, et al., Int. J of Food Micr., 70: 143-154, 2001, and Yu, J. of Immuno. Mthds., 218: 1-8, 1998); and peroxisomes (Luers, et al., Electrophoresis, 19: 1205-1210, 1998).

In the management of cancer patients, the staging of the disease with regards to whether it is localized or if metastatic spread has occurred to other tissues is of utmost importance for the choice of therapeutic regimens for the individual patient. Malignant cells spread by direct invasion into the surrounding tissue, through the lymphatics, or by the distribution of tumor cells in the blood to distant organs, including the bone marrow the central nervous system, and the cerebrospinal fluid. Detection of metastatic tumor cells has, until recently, relied on morphological methods using light and electron microscopy on biopsied tumor specimens, on smears of bone marrow and peripheral blood, and on slides prepared after cytocentrifugation of various body fluids. Since the advent of monoclonal antibodies recognizing antigens predominantly expressed on the surface of different types of malignant cells, the identification of metastatic cells has, to an increasing extent, also involved immunocytochemistry and immunofluorescence. Thus, slides prepared from biopsied tumors or cytocentrifugates are treated with monoclonal antibodies, and the binding of these to the tumor cells is visualized calorimetrically or by fluorescence. The latter method requires the use of a fluorescence microscope, alternatively preparing a cell suspension and use of a flow cytometer.

The previous methods suffer from limited sensitivity and/or specificity, and is usually laborious and time consuming, also requiring a high degree of expertise. Flowcytometric examinations also involve expensive equipment.

The morphological methods for the detection of tumor cells in blood and bone marrow are much less sensitive than methods involving immunocytochemistry and immunofluorescence (Beiske, et al., Am. J. Pathology 141 (3), September 1992). Also, the latter methods are, however, inadequate in cases where the tumor cells represent less than 1% of the total number of cells in a test sample. Flow cytometry may provide better sensitivity than the methods involving the use of a microscope, but requires the availability of a high number of cells, and also involves several technical difficulties. Furthermore, aggregation of cells may cause problems with flow cytometry, and this method does not provide possibilities to distinguish between labeled tumor cells and non-specifically fluorescing normal cells.

The present invention allows for a very sensitive detection of, for example, metastatic tumor cells, since a high number of cells can readily be screened and the cell-bead complexes are easily detected and quantitated by the MOBDS. The monoclonal antibodies used for the detection of target cells in the present invention are selected such that these antibodies bind with sufficient specificity to, for example, tumor cells and not to non-target cells present in mixed cell suspensions or test sample. The test sample may include, for example blood, bone marrow, and other solutions containing tumor manifestations, such that all cells with attached beads represent the target cells. The target cells are then captured on specific magnetized areas or magnetic domains in a magneto-optical (MO) bio-disc and quantitated using the associated magneto-optical drive and software. In addition, the procedure is rapid and simple, and can be performed by any investigator without the need for expensive and sophisticated equipment such as a flow cytometer. Further details relating to the MOBDS are disclosed in, for example, commonly assigned co-pending U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; and the above referenced U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” filed Mar. 14, 2002. Details relating to magneto-optical recording, precise creation of magnetic regions on a magneto-optical disc, and magneto-optical detection methods are described above in conjunction with FIG. 37 and in, for example, Tsunashima, Magneto Optical Recording, J. Phys. D: Appl. Phys. 34, R87-R102, 2001, and in Coombs, Differential Phase Contrast and Magneto-optic Edge Detection, Applied Optics, 34(29), 6723-6729, 1995, which are both incorporated by reference in their entireties as if fully repeated herein.

As mentioned above, the immunomagnetic embodiment of the MOBMA aspect of the present invention involves the binding of capture antibodies including monoclonal antibodies, e.g. of murine or human origin, that specifically recognize antigens present on tumor cells, but not antigens on normal cells, or for other purposes to specified sub-populations of normal cells, to magnetic particles or beads to thereby form bio-magnetic particles. The capture antibodies may bind directly to antigens on the target cell or the Fc-portion of primary antibodies or cell binding antibodies that bind to the tumor cells. The cell binding antibodies may be of the IgG or IgM type or a fragment of IgG or IgM antibodies. The capture or primary antibodies may be antibodies directed against groups of antigen determinants including, for example, tumor associated glycoprotein 72 (TAG 72) antigen (CC49 antibody) (Barjer et al., Gynec. Onco. 82: 57-63, 2001), CD56/NCAM antigen (MOC-1 antibody)(Speirs et al., J. Histochem. Cytochem., 41(9):1303-10, 1993), epithelial cell surface antigen (BER-EP4 antibody) (Borgen et al., J. Hematother., 6(2):103-114, 1997, and Ziggeuner et al. J. Urol., 164: 1834-1837, 2000), Cluster 2 epithelial antigen (MOC-31 antibody) (Rye et al., Am J. Patho., 150(1): 99-106, 1997, and Ree et al., Int. J. Cancer, 97: 28-33, 2002), Cluster 2 (MW 40 kD) antigen (NrLu10 antibody) (Myklebust et al., Br. J. Cancer Suppl. 14: 49-53, 1991), HMW-melanoma-associated antigen (225.28S antibody) (Dell'Erba et al., Anticancer Res., 21(2A):925-930, 2001), 80 kD, Sarcoma-associated antigen (TP-1 & TP-3 antibodies) (Bruland, et al., Cancer Res., 48: 5302-5309, 1988), cytokeratin antigens (pan-anti-CK antibody) (Bilkenroth et al., Int. J. Cancer, 92: 577-582, 2001), mucin antigens TAG 12 (2E11) (Diel et al., J. Natl. Cancer Inst., 88(22):1652-8, 1996), and EGF-receptor antigen (425.3 antibody) (Merck). The 425.3 antibody is directed towards antigens in both normal and malignant cells. The capture antibodies can furthermore be directed against growth factor receptors, for example EGF-receptor, PDGF (A and B) receptor, insuline receptor, insuline-like receptor, transferrin receptor, NGF and FGF receptors, group of integrins, other adhesion membrane molecules and MDR proteins in both normal cells and abnormal cells, and antigens present on subpopulations of normal cells, in addition to oncogenic products, expressed on the membranes of normal and malignant cells and on malignant cells alone, for example Neu/erb B2/HER2. As for the malignant cells, these may be breast, ovarian and lung carcinoma cells, melanoma, sarcoma, glioblastoma, cancer cells of the gastrointestinal tract and the reticuloendothelial system, or the target cells may be associated with non-neoplastic diseases, such as cardiovascular, neurological, pulmonary, autoimmune, gastrointestial, genitourinary, reticuloendothelial and other disorders. Furthermore, the malignant cell population may be located in bone marrow, peripheral blood, come from pleural and peritoneal effusions and other body fluid compartments, such as urine, cerebrospinal fluid, semen, lymph or from solid tumors in normal tissues and organs, for example liver, lymph nodes, spleen, lung, pancreas, bone tissue, the central nervous system, prostatic gland, skin and mucous membranes. A partial list of the antigen determinants and the corresponding antibodies or antibody fragments that may be used in conjunction with the present improved invention is presented below in Table 1.

In cases where the density of the target cells is low, for example, malignant cells or the target cells representing a very low fraction of the total number of cells (≦1%), the target cells can be positively separated from non-target cells and pre-concentrated using a magnet prior to analysis in the MOBDS. The isolated target cells, can then be enumerated using the MOBDS and the fraction of target cells relative to the total number of cells in the initial cell suspension can be calculated.

Drug Sensitivity Assays on the MOBDS

As mentioned above, the MOBDS may be used to isolate, manipulate, and study target cells in vitro. One assay that can be performed on target cells is a cytotoxicity, chemosensitivity, drug sensitivity, or drug resistance assay to measure drug-induced cell death following drug exposure. In this method, the target cells may be, for example, collected from a malignant tumor biopsy, bacteria, plant, virus, and unicellular organisms. The target cells are immunomagnetically isolated in one or more chambers in the MO bio-disc, as described above, to thereby constitute an isolate. Once the target cells are isolated, a pre-determined number of cells, from the isolate, are magnetically moved or manipulated to various chambers within the MO bio-disc including test chambers. These chambers may contain drugs including, but not limited to, chemotherapeutic agents, antibiotics, or antiviral, individually or in combination, at various pre-determined dosages. The chemotherapeutic agents may include, for example, breast cancer, lung cancer, brain cancer, liver cancer, and ovarian cancer drugs such as Cisplatin, Topotecan, Taxol, Gemcitabine (1), Mitomycin-C, Navelbine, Nitrogen Mustard, 5-Fluorouracil, Doxorubicin, Etoposide, Trimetrexate, and various combinations thereof including, but not limited to, Cisplatin and Topotecan, Cisplatin and Taxol, Cisplatin and Gemcitabine (1), Cisplatin and Nitrogen Mustard, and Cisplatin and 5-Fluorouracil. The isolated cells are then incubated at pre-determined conditions in the various concentrations and combinations of drugs. After incubation, the number of live cells are counted, using the MOBDS, and the IC50 (drug concentration in which 50% of the cells die as a result of exposure a drug or a combination of drugs) from all the drugs are calculated to determine the best therapy regimen for the patient. This test is very important since drugs found active in vitro are approximately 10 times more likely to be clinically effective relative to drugs found inactive in vitro. In vivo drug sensitivity assays are well know in the art (Bosanquet et al., Leukemia 16(6):1035-44, 2002; Nagourney et al., J. Clin. Onco. 18:11, 2245-49, 2000; Bosanquet et al., Br. J. Haem., 106:71-77, 1999; and Cortazar et al., J. Clin. Onco., 17:5, 1625-31, 1999). The assays of the prior art are mostly performed using a microtiter plate and the cell counting and analysis done using a microscope or calorimetrically using a microtiter plate reader by preferential staining of the live or dead cells, to determine the number of live cells. The present invention automates the process in that the cell selection, manipulation, incubation, analysis, and IC50 determination is performed using the MOBDS without the need for a separate analysis device such as a microscope or a microtiter plate reader. The automated process increases the efficiency and the accuracy of these drug sensitivity assays.

Sample Preparation

Steps for performing the above described immunomagnetic embodiment of the present invention may differ depending on type of tissues to be examined including, for example:

a) cells from solid tissue or needle tumor biopsies are separated mechanically or with mild enzymatic treatment to generate a suspension of single cells, to which the primary, specific antibodies or antibody fragments are added directly or after washing the cell suspension in phosphate buffered saline or culture medium with or without serum, such as fetal calf serum, bovine, horse, pig, goat, or human serum;

b) cells in pleural or ascitic effusion, cerebrospinal fluid, urine, lymph or body fluids such as effusions in the joints of patients with various forms of arthritis, the specific capture antibodies or antibody fragments are either added to the samples directly, or after centrifugation with or without washings before or after the cells in the samples are spun down and brought back into suspension; and

c) mononuclear cells from blood or bone marrow aspirate are isolated by gradient centrifugation and the capture antibodies are added to the isolated mononuclear cells on or before washing and resuspension.

TABLE 1
LIST OF RELEVANT ANTIGENS AND
EXAMPLES OF ASSOCIATED ANTIGEN-BINDING ANTIBODIES

| ANTIGENS | MONOCLONAL ANTIBODIES |
|---|---|
| Adhesion molecules | |
| Fibronectin receptor (α5β1 integrin) | Pierce 36114, BTC 21/22 |
| | Calbiochem 341649 |
| Integrin α3β 1 | M-Kiol 2 |
| Vitronectin receptor (αγβ3 integrin) | TP36.1, BTC 41/42 |
| Integrin α2 | Calbiochem 407277 |
| Integrin α3 | Calbiochem 407278 |
| Integrin α4 | Calbiochem 407279 |
| Integrin α5 | Calbiochem 407280 |
| Integrin αV | Calbiochem 407281 |
| Integrin β2 | Calbiochem 407283 |
| Integrin β4 | Calbiochem 407284 |
| GpIIβIIIα | 8221 |
| ICAM-I (CD54) | C57-60, CL203.4, RR 1/1 |
| VCAM-1 | Genzyme 2137-01 |
| ELAM-1 | Genzyme 2138-01 |
| E-selectin | BBA 8 |
| P-selectin/GMP-140 | BTC 71/72 |
| LFA-3 (CD58) | TS 2/9 |
| CD44 | BM 1441 272, 25.32 |
| CD44-variants | 11.24, 11.31, 11.10 |
| N-CAM(CD56) | MOC-1 |
| H-CAM | BCA9 |
| L-CAM | BM 1441 892 |
| N-CAM | TURA-27 |
| MACAM-1 | NRI-M9 |
| E-cadherin | BTC 111, HECD-1, 6F9 |
| P-cadherin | NCC-CAD-299 |
| Tenascin | BM 1452 193, Calbiochem 580664 |
| Thrombospondin receptor (CD36) | BM 1441 264 |
| VLA-2 | A1.43 |
| Laminin receptor | |
| HNK-1 epitope | HNK-1 |
| Carbohydrate antigens | |
| T-antigen | HH8, HT-8 |
| Tn-antigen | TKH6, BaGs2 |
| Sialyl Tn | TKH-2 |
| Gastrointestinal cancer associated antigen (MW 200 kD) | CA 19-9 |
| Carcinoma associated antigen | C-50 |
| Le^{y} | MLuC1, BR96, BR64 |
| di-Le^{z}, tri-Le¹ | B3 |
| Dimeric Le¹ epitope | NCC-ST-421 |
| H-type 2 | B1 |
| CA15-3 epitope | CA15-3 |
| CEA | I-9, I-14, I-27, II-10, I-46, |
| | Calbiochem 250729 |
| Galb1-4GlcNac (nL4,6,8) | 1B2 |
| H-II | BE2 |
| A type 3 | HH8 |
| Lacto-N-fucopentanose III (CD15) | PM-81 |
| Glycolipids | |
| GD₃ | ME 36.1, R24 |
| GD₂ | ME36.1, 3F8, 14.18 |
| Gb₃ | 38-13 |
| GM₃ | M2590 |
| GM₂ | MKI-8, MKI-16, |
| FucGM₁ | 1D7, F12 |
| Growth factor receptors | |
| EGF receptor | 425.3, 2.E9, 225 |
| c-erbB-2 (HER2) | BM 1378 988, 800 E6 |
| PDGFα receptor | Genzyme 1264-00 |
| PDGFβ receptor | Sigma P 7679 |
| Transferrin receptor | OKT 9, D65.30 |
| NGF receptor | BM 1198 637 |
| IL-2 receptor (CD25) | BM 1295 802, BM 1361 937 |
| c-kit | BM 428 616, 14 A3, ID9.3D6 |
| TNF-receptor | GEnzyme 1995-01, PAL-M1 |
| NGF receptor | |
| Melanoma antigens | |
| High molecular weight antigen (HMW 250.000) | 9.2.27, NrML5, 225.28, |
| | 763.74, TP41.2, IND1 |
| MW105 melanoma-associated glycoprotein | ME20 |
| 100 kDa antigen (melanoma/carcinoma) | 376.96 |
| gp 113 | MUC 18 |
| p95-100 | PAL-M2 |
| Sp75 | 15.75 |
| gr 100-107 | NKI-bereb |
| MAA | K9.2 |
| MW 125 kD (gp125) | Mab 436 |
| Sarcoma antigens | |
| TP-1 and TP-3 epitope | TP-1, TP-3 |
| MW 200 kD | 29-13, 29.2 |
| MW 160 kD | 35-16, 30-40 |
| Carcinoma markers | |
| MOC-31 epitope (cluster 2 epithelial antigen) | MOC-31, NrLu10 |
| MUC-1 antigens (such as DF3-epitope (gp290 kD)) | MUC-1, DF3, BCP-7 to -10 |
| MUC-2 and MUC-3 | PMH1 |
| LUBCRU-G7 epitope (gp 230 kD) | LUBCRU-G7 |
| Prostate specific antigen | BM 1276 972 |
| Prostate cancer antigen | E4-SF |
| Protate high molecular antigen MW > 400 kD | PD41 |
| Polymorphic epithelial mucins | BM-2, BM-7, 12-H-12 |
| Prostate specific membran antigen (Cyt-356) | 7E11-C5 |
| Human milk fat globulin | Immunotech HMFG-1, 27.1 |
| 42 kD breast carcinoma epitope | B/9189 |
| MW > 10⁶ mucin | TAG-72, CC-49, CC-83 |
| Ovarian carcinoma OC125 epitope (MW 750 kD) | OC125 |
| Pancreatic HMW glycoprotein | DU-PAN-2 |
| Colon antigen Co17-1A (MW 37000) | 17-1A |
| G9-epitope (colon carcinoma) | G9 |
| Human colonic sulfomucin | 91.9H |
| MW 300 kD pancreas antigen | MUSE11 |
| GA 733.2 | GA733, KS1.4 |
| TAG 72 | B72.3, CC49, CC83 |
| Undefined | Oat1, SM1 |
| Pancreatic cancer-associated | MUSE 11 |
| Pancarcinoma | CC49 |
| Prostate adenocarcinoma-antigen | PD 41 |
| MW 150-130 kD adenocarcinoma of the lung | AF-10 |
| gp160 lung cancer antigen (Cancer Res. 48, 2768, 1988) | anti gp160 |
| MW 92 kD bladder carcinoma antigen | 3G2-C6 |
| MW 600 kD bladder carcinoma antigen | C3 |
| Bladder carcinoma antigen (Cancer Res. 49, 6720, 1989) | AN43, BB369 |
| CAR-3 epitop MW > 400 kD | AR-3 |
| MAM-6 epitope (C15.3) | 115D8 |
| High molecular ovarian cancer antigen | OVX1, OVX2 |
| Mucin epitope la3 | la3 |
| Hepatocellular carcinoma antigen MW 900 kD | KM-2 |
| Hepernal epitope (gp43) Hepatocellular carc. ag | Hepema-1 |
| O-linked mucin containing N-glycolylneuraminic acid | 3E1.2 |
| MW 48 kD colorectal carcinoma antigen | D612 |
| MW 71 kD breast carcinoma antigen | BCA 227 |
| 16.88 epitope (colorectal carcinoma antigen) | 16.88 |
| CAK1 (ovarian cancers) | K1 |
| Colon specific antigen p | Mu-1, Mu-2 |
| Lung carcinoma antigen MW 350-420 kD | DF-L1, DF-L2 |
| gp54 bladder carcinoma antigen | T16 |
| gp85 bladder carcinoma antigen | T43 |
| gp25 bladder carcinoma antigen | T138 |
| Neuroblastoma antigens | |
| Neuroblastoma-associated, such as UJ13A epitope | UJ13A |
| Glioma antigens | |
| Mel-14 epitope | Mel-14 |
| Head and neck cancer antigens | |
| MW 18-22 kD antigen | E48 |
| HLA-antigens | |
| HLA Class 1 | TP25.99 |
| HLA-A | VF19LL67 |
| HLA-B | H2-149.1 |
| HLA-A2 | KS1 |
| HLA-ABC | W6.32 |
| HLA-DR, DQ, DP | Q 5/13, B 8.11.2 |
| β₂-microglobulin | NAMB-1 |
| Apoptosis receptor | |
| Apo-1 epitope | Apo 1 |
| Various | |
| Plasminogen activator antigens & receptors | Rabbit polyclonal |
| p-glycoprotein | C219, MRK16, JSB-1, 265/F4 |
| cathepsin D | CIS-Diagnostici, Italy |
| biliary epithelial antigen | HEA 125 |
| neuroglandular antigen (CD63) | ME491, NKI-C3, LS62 |
| CD9 | TAPA-1, R2, SM23 |
| pan-human cell antigen | pan-H |

In another embodiment of the MOBDS aspect of the present invention, target cells may be separated by a two dimensional or dual parameter immunomagnetic cell separation method such as that described in, for example, Partington, et al., A Novel Method of Cell Separation Based on Dual Parameter Immunomagnetic Cell Selection, J. of Immuno. Mthds., 223: 195-205, 1999, which is incorporated by reference in its entirety herein. In this embodiment, the tagging and separation of target cells with bio-magnetic particles may be performed within the fluidic circuits in the MO bio-disc. This approach takes advantage of size differences between commercially available immunomagnetic beads and/or particles and their differing properties in terms of attraction to magnetic fields of various strengths. For example, the first step of separation may be the positive selection of cells utilising 50 nm beads. Cells isolated in the first step—still rosetted with the 50 nm beads—can then be subjected to further positive or negative selection using larger beads, for example, M280 or M450 Dynabeads, without the need for prior bead removal. The strength of the magnetic field during the second separation step is modulated such that the magnetic force generated on the MO disc magnetic domain is sufficient to attract only the larger Dynabeads but not the 50 nm beads. This dual parameter embodiment thus provides a better method for isolating and purifying cells of interest.

In yet another embodiment of the MOBDS aspect of the present invention, target cells may be labeled or tagged with magnetic particles by ballistic transfer. The ballistic transfer technology employs a cold gas shock wave to accelerate microprojectiles that carry matter into cells by mechanical force. This embodiment of the present invention relates to a method that enables separation of target cells rendered magnetically susceptible and can thus be separated by retaining them in a strong magnetic field. The tagged cells may then be captured, analyzed, manipulated, and quantitated using the MOBDS. Further details relating to ballistic tagging of cells with magnetic particles is disclosed in, for example, U.S. Pat. No. 6,348,338 to Wittig, which is herein incorporated in its entirety by reference.

A further embodiment of the current invention is using the MOBDS to perform cellular analysis, manipulation, and quantitation by immunomagnetic cell selection, as described above, and aligning the target cells in various pre-determined configurations on the MO bio-disc such as alignment of cells on the tracks of the MO bio-disc. In this embodiment, cells are magnetically sorted and aligned on the MO bio-disc, and analyzed using the MO disc drive and associated software. Related methods and systems for immunomagnetic cellular analysis are described in, for example, Tibbe et al., Cytometry, 43:31-37, 2001, and Tibbe et al., Cytometry, 47:163-172, 2002, both of which are herein incorporated by reference in their entireties. These methods of the prior art require specialized devices and systems including fluorescence detection. The current invention circumvents the need for these specialized devices in that a standard MO disc drive may be used to perform the immunomagnetic cell selection, manipulation, detection, quantitation, and analyses described above.

Molecular Applications of the MOBDS Biomagnetic Assay

The isolated target cells may be characterized for the presence of specific biochemical and biological features. Of particular importance will be the use of such cells for studies in molecular biology. In contrast to the above-cited methods of the prior art, the present method allows studies and growth of the target cells without performing a cleavage of the magnetic particle-target cell linkage. For several purposes it may be of interest to examine specific genes in a pure population of target cells at the DNA, mRNA, and protein level, both in tumor biopsies as well as in tumor cells present in blood, bone marrow and other body fluids, for example urine, cerebrospinal fluid, semen, lymph, or from otherwise normal tissues and organs, for example liver, lymph nodes, spleen, lung, pancreas, bone tissues, central nervous system, prostatic gland, skin and mucous membranes, and in other areas of cytological assays.

With the methods of prior art, signals obtained on Southern, Northern and Western blots represent the normal cells as well as the tumor cells in the biopsy. If a single cell suspension is first prepared from the tumor material, and the tumor cells are then positively immunomagnetically detected and separated using the MOBDS of the present invention, any gene studies performed on this material would represent the target cells only. This also relates to, for example, malignant cells present in mammalian tissues including bone marrow, peripheral blood, pleural and peritoneal effusions, and other body fluids, for example urine, cerebrospinal fluid, semen and lymph. Studies involving polymerase chain reaction (PCR) and microarray methodologies will also gain increased specificity and reliability when performed on pure target cell populations obtained using the methods and apparatus of the present invention.

Neurobiological Assays on the MO Bio-Disc System (MOBDS)

In still another embodiment of the MOBDS aspect of the present invention, neurons may be isolated and stimulated to grow and regenerate in biological matrix or solution in a MO bio-disc. The biological matrix may be formed from a gel or any biologically compatible material that provides a solid support for the cells while allowing passage of essential nutrients and gases to enable survival of the cells. The biological matrix also allows growth and movement of the cells and its components such as axon and dendrite movement within the matrix. The direction and rate of growth of the axons, dendrites, and cells may be controlled by a magnetic field on the MO bio-disc, generated by the MO drive. In this embodiment, neurites, for example, may be provided with magnetic nanoparticles or beads that are absorbed or actively incorporated into the neurons and their axons. Arrays of these neurons are then aligned in predetermined locations within the biological matrix on the MO bio-disc. The aligned neurons or neurites are then exposed to a magnetic field that is displaced to thereby physically move the magnetic particle-loaded neurons and their axons along a desired axis for bridging the gap or gaps between the neurons in the array. Since magnetic regions generated on the MO disc are relatively small and can be precisely controlled, use of the magnetically directed neurite growth embodiment of the present invention allows a more precise control of neurite growth relative to methods of the prior art. Magnetically directed neurite growth in vitro and nerve regeneration in vivo are well known in the art (Moorman et al., Brain Res. Bulletin, 35(5-6):419-422, 1994, Dubey et al., Exp. Neuro., 158:338-350, 1999, Macias et al., Bioelectromagnetics, 21:272-286, 2000, Shah et al., Bioelectromagnetics, 22:267-271, 2001, and U.S. Pat. No. 6,132,360 to Halpern, all of which are herein incorporated by reference in their entireties). This embodiment of the present invention is directed at using the MOBDS to study precise magnetic control of neurite growth in vitro and at creating neural networks in vitro.

Referring now to FIG. 76, there is illustrated a top plan view of a portion of a magneto-optical bio-disc having fluidic circuits including an inlet port 152, a mixing chamber 164, a separation or analysis chamber 300, and testing chambers 302. The magneto-optical bio-disc may include one or more components of the bio-discs described above in conjunction with FIGS. 3A to 3C, 4A to 4C, 33A to 33C, 35, 36A-36C, and 37.

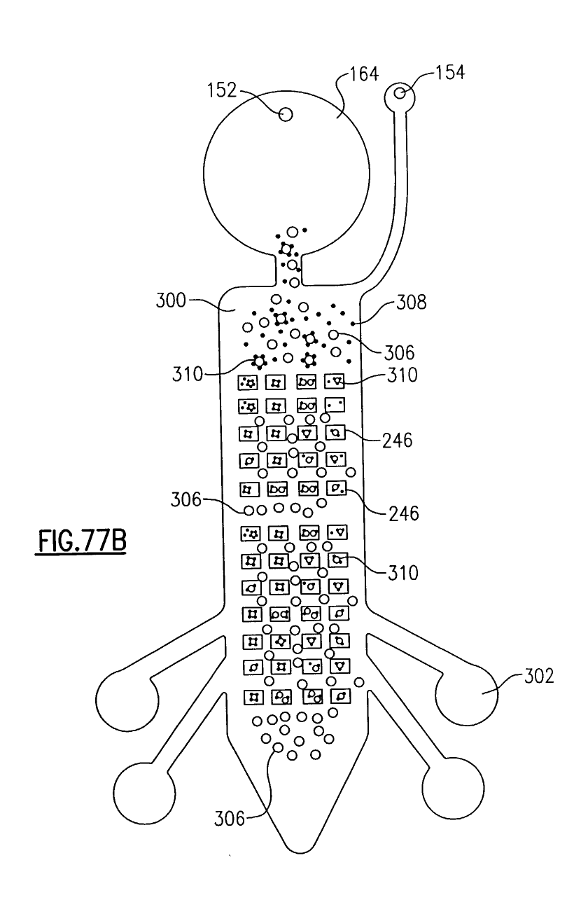

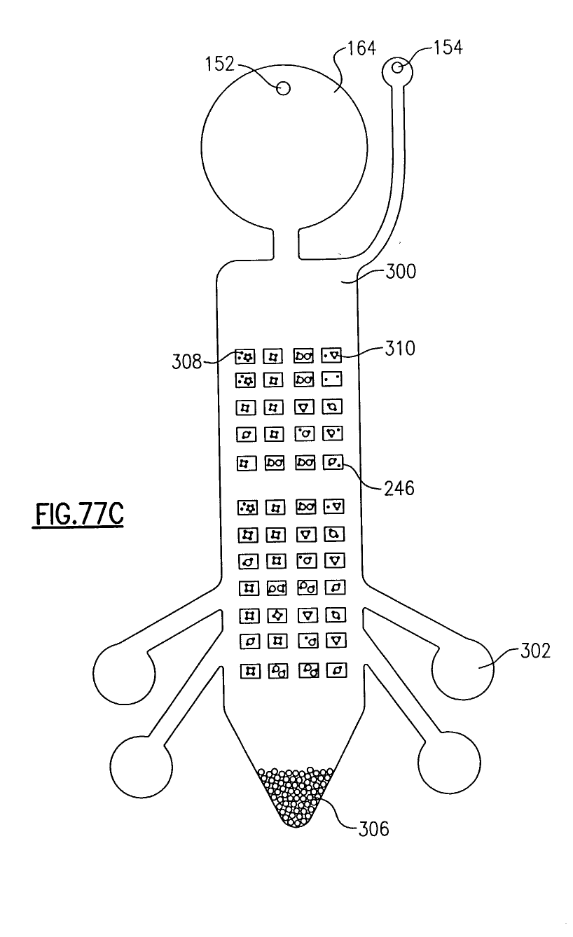

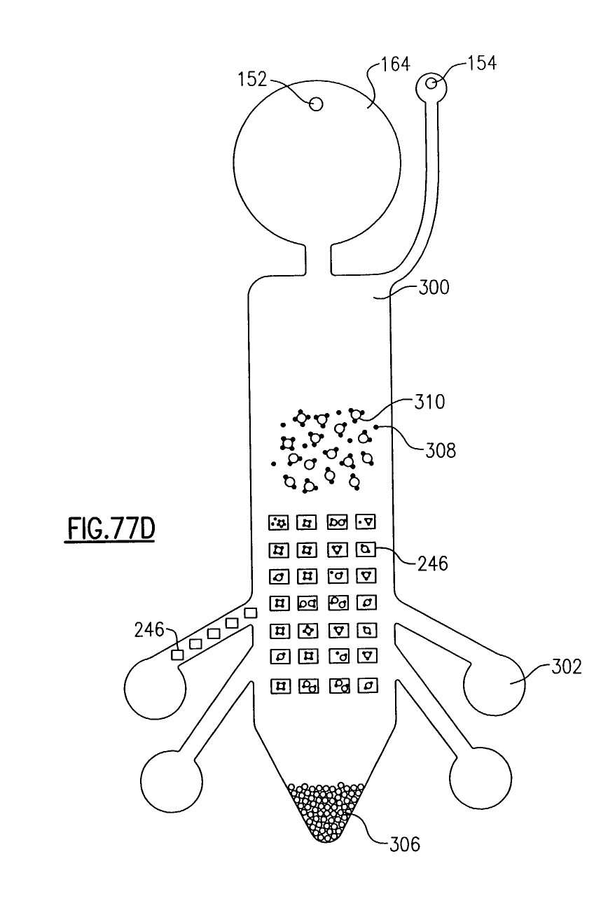

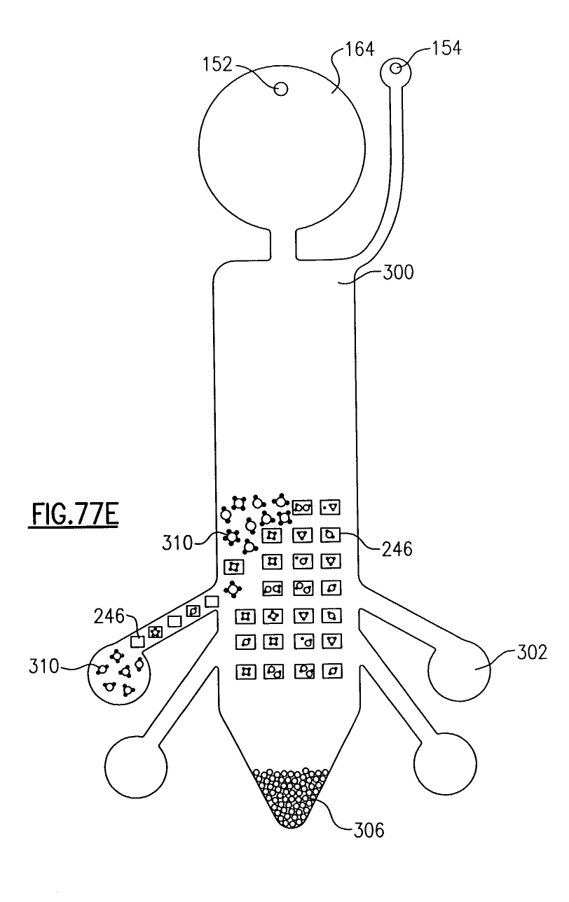

With reference next to FIGS. 77A-77E, there are shown plan views illustrating a method of separation and testing of cells in the fluidic circuit shown in FIG. 76. In this particular method of the present invention, test cells 306 are loaded into the mixing chamber 164 through the inlet port 152 using a pipette 214 (FIG. 77A). Bio-magnetic particles 308 are then loaded into the mixing chamber 164 using a pipette 214. The bio-magnetic particles are coated with binding agents specific for surface markers on the cells of interest in the sample. The binding agents may include, for example, antibodies outlined above in Table 1. The cells 306 and the bio-magnetic particles are then incubated for a sufficient time to allow binding of the bio-magnetic particles 308 to the cells of interest through binding of the binding agents and the cell surface markers. After incubation, cells of interest or target cells are thus labeled with bio-magnetic particles resulting in a labeled cell 310. This renders the labeled cells 310 susceptible to magnetic manipulation. Magnetic domains or regions 246 may also be formed in the separation chamber 300 before of after the incubation using a magneto-optical drive. Details relating to the formation of magnetic domains 246 on magneto-optical bio-discs are discussed above in conjunction with FIG. 37. Once the incubation is complete, the disc is rotated at a pre-determined speed and duration, using the magneto-optical drive, to move the suspension containing unlabeled cells 306, bio-magnetic particles 308, and labeled cells 310 into the separation chamber 300 (FIG. 77B). As the suspension moves through the separation chamber, the labeled cells 310 and free or unattached bio-magnetic particles 308 magnetically bind to the magnetic domains 243 within separation chamber 300. The disc is then rotated at another pre-determined speed and time to move unbound and unlabeled cells 306 in the suspension to the bottom of the separation chamber 300 (FIG. 77C). The magnetic domains 246 are then sequentially erased and formed to selectively release the magnetically bound labeled cells 310 (FIG. 77D). The labeled cells 310 are then guided magnetically to one or more testing chambers 302 by sequentially erasing and forming the magnetic domains 246 (FIG. 77E). The testing chamber 302 may be pre-loaded with a test solution having a test agent including, but not limited to, chemotherapeutic agents, antibiotics, and anti-viral medications. The labeled cells 310 may then be incubated With the test agent. A beam of electromagnetic radiation may then be scanned through the testing chamber 302 to quantify the live and apototic cells and thereby determine the sensitivity of the cells when exposed to the test agent. Apototic cells are characterized by membrane blebbing, cell shrinkage, protein fragmentation, chromatin condensation and DNA degradation, all of which alter the optical properties of the cell rendering it distinguishable from non-apototic cells using an optical disc reader.

Other Implementations of the Current Invention

This invention or different aspects thereof may be readily implemented in or adapted to many of the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional patent application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Bio-discs with Reflective Layers” filed on Nov. 15, 2001; U.S. patent application Ser. No. 09/988,728 entitled “Methods And Apparatus For Detecting And Quantifying Lymphocytes With Optical Biodiscs” filed on Nov. 20, 2001; U.S. patent application Ser. No. 09/988,850 entitled “Methods and Apparatus for Blood Typing with Optical Bio-discs” filed on Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disc-Based Laboratory And Improved Optical Bio-Disc Including Same” filed Dec. 14, 2001; U.S. patent application Ser. No. 10/035,836 entitled “Surface Assembly For Immobilizing DNA Capture Probes And Bead-Based Assay Including Optical Bio-Discs And Methods Relating Thereto” filed Dec. 21, 2001; U.S. patent application Ser. No. 10/038,297 entitled “Dual Bead Assays Including Covalent Linkages For Improved Specificity And Related Optical Analysis Discs” filed Jan. 4, 2002; U.S. patent application Ser. No. 10/043,688 entitled “Optical Disc Analysis System Including Related Methods For Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002; U.S. patent application Ser. No. 10/086,941 entitled “Methods For DNA Conjugation Onto Solid Phase Including Related Optical Biodiscs and Disc Drive Systems” filed Feb. 26, 2002; U.S. Provisional Application Ser. No. 60/363,949 entitled “Methods for Differential Cell Counts Including Leukocytes and Use of Optical Bio-Disc for Performing Same” filed Mar. 12, 2002; U.S. Provisional Application Ser. No. 60/382,944 entitled “Methods and Apparatus for Use in Detection and Quantitation of Cell Populations and Use of Optical Bio-Disc for Performing Same” filed May 24, 2002; and U.S. Provisional Application Ser. No. 60/384,205 entitled “Optical Disc Systems For Determining The Concentration Of Cells Or Particles In A Sample And Methods Relating Thereto” filed May 30, 2002. All of these applications are herein incorporated by reference in their entireties. They thus provide background and related disclosure as support hereof as if fully repeated herein.

Experimental Details

While this invention has been described in detail with reference to the drawing figures, certain examples and further illustrations of the invention are presented below.

### EXAMPLE 1

The two-step hybridization method demonstrated in FIG. 12A was used in performing the dual bead assay of this example.

A. Dual Bead Assay

In this example, the dual assay in carried out to detect the gene sequence DYS that is present in male but not in female. The assay is comprised of 3μ magnetic and capture beads coated with covalently attached capture probe; 2.1μ fluorescent reporter beads coated with a covalently attached sequence specific for the DYS gene, and target DNA molecule containing DYS sequences. The target DNA is a synthetic 80 oligonucleotide sequence. The capture probe and reporter probes are 40 nucleotides in length and are complementary to DYS sequence but not to each other.

The specific methodology employed to prepare the assay involved treating 1×10⁷ capture beads and 2×10⁷ reporter beads in 100 microgram per milliliter Salmon Sperm DNA for 1 hr. at room temperature. This pretreatment will reduce non-covalent binding between the capture and reporter beads in the absence of target DNA as shown in FIG. 38. The capture beads were concentrated magnetically with the supernatant being removed. A 100 microliter volume of the hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were added to the capture beads and the beads were re-suspended. Various concentrations of target DNA ranging from 1, 10, 100, 1000 femtomoles were added while mixing at 37° C. for 2 hours. The beads were magnetically concentrated and the supernatant containing target DNA was removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

A 2×10⁷ amount of reporter beads in 100 microliter hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were then added to washed capture beads. The beads were re-suspended and incubated while mixing at 37° C. for an additional 2 hours. After incubation the capture beads were concentrated magnetically, and the supernatant containing unbound reporter beads were removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

After the final wash, the beads were re-suspended in 20 microliters of binding buffer (50 mM Tris, 200 mM NaCl, 10 mM MgCl₂, 0.05% Tween 20, 1% BSA). A 10 microliter volume was-loaded on to the disc that was prepared as described below in Part B of this example.

B. Preparation of the Disc

A gold disc was coated with maleic anhydride polystyrene. An amine DNA sequence complementary to the reporter probes (or capture agent) was immobilized on to the discrete reaction zones on the disc. Prior to sample injection, the channels were blocked with a blocking buffer (50 mM Tris, 200 mM NaCl, 10 mM MgCl₂, 0.05% Tween 20, 1% BSA, 1% sucrose) to prevent non-covalent binding of the dual bead complex to the disc surface. A perspective view of the disc assembly showing capture agents 220, the capture zones 170, and fluidic circuits as employed in the present invention is illustrated in detail in FIGS. 25A-25D. Alternatively, if the reporter beads are coated with Streptavidin, a capture zone could be created with the capture agent such as BSA Biotin which could be immobilized on to the disc (pretreated with Polystyrene) by passive absorption. A perspective view of the disc assembly showing the use of biotin capture agents is presented in FIGS. 26A-26D. Various methods for use in this type of anchoring of beads onto the disc are also shown in FIGS. 15A-15B, 17, 19A-19C, and 23A-23B.

C. Capture of Dual Bead Complex Structure on the Disc

A 10 microliter volume of the dual bead mixture prepared as described in Part A above was loaded in to the disc chamber and the injection ports were sealed. To facilitate hybridization between the reporter probes on the reporter beads and the capture agents, the disc was centrifuged at low speed (less than 800 rpm) upto 15 minutes. The disc was read in the CD reader at the speed 4× (approx. 1600 rpm) for 5 minutes. Under these conditions, the unbound magnetic capture beads were centrifuged away from the capture zone. The magnetic capture beads that were in the dual bead complex remained bound to the reporter beads in the capture zone. The steps involved in using the disc to capture and analyze dual bead complexes are presented in detail in FIGS. 25A-25D, 26A-26D, and 27A-27D.

D. Quantification of Dual Bead Complex Structures

The amount of target DNA captured could be enumerated by quantifying the number of capture magnetic beads and the number of reporter beads since each type of bead has a distinct signature.

### EXAMPLE 2

The following example is directed to dual bead multiplexing and related assays as discussed above with reference to, for example, FIG. 32.

A. Dual Bead Assay Multiplexing

In this example, the dual bead assay is carried out to detect two DNA targets simultaneously. The assay is comprised of 3 μm magnetic capture bead. One population of the magnetic capture bead is coated with capture probes 1 which are complementary to the DNA target 1, another population of magnetic capture beads is coated with capture probes 2 which are complementary to the DNA target 2. Alternatively two different types of magnetic capture beads may be used. There are two distinct types of reporter beads in the assay. The two types may differ by chemical composition (for example Silica and Polystyrene) and/or by size. Various combinations of beads that may be used in a multiplex dual bead assay format are depicted in FIG. 32. One type of reporter bead is coated with reporter probes 1, which are complementary to the DNA target 1. The other reporter beads are coated with reporter probes 2, which are complementary to the DNA target 2. Again the capture probes and the reporter probes are complementary to the respective targets but not to each other.

The specific methodology employed to prepare the dual bead assay multiplexing involved treating 1×10⁷ capture beads and 2×10⁷ reporter beads in 100 μg/ml salmon sperm DNA for 1 hour at room temperature. This pretreatment will reduce non-covalent binding between the capture and reporter beads in the absence of target DNA. The capture beads were concentrated magnetically with the supernatant being removed. A 100 microliter volume of the hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were added and the beads were re-suspended. Various concentrations of target DNA ranging from 1, 10, 100, 1000 femto moles were added to the capture beads suspension. The suspension was incubated while mixing at 37° C. for 2 hours. The beads were magnetically concentrated and the supernatant containing target DNA was removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

A 2×10⁷ amount of reporter beads in 100 microliter hybridization buffer (0.2 M NaCl, 1 mM EDTA, 10 mM MgCl₂, 50 mM Tris HCl, pH 7.5, and 5× Denhart's mixture, 10 microgram per milliliter denatured salmon sperm DNA) were then added to washed capture beads. The beads were re-suspended and incubated while mixing at 37° C. for an additional 2 hours. After incubation the capture beads were concentrated magnetically, and the supernatant containing unbound reporter beads were removed. A 100 microliter volume of wash buffer (145 mM NaCl, 50 mM Tris pH 7.5, 0.1% SDS, 0.05% Tween, 0.25% NFDM, 10 mM EDTA) was added and the beads were re-suspended. The beads were magnetically concentrated and the supernatant was again removed. The wash procedure was repeated twice.

After the final wash, the beads were re-suspended in 20 microliters of binding buffer (50 mM Tris, 200 mM NaCl, 10 mM MgCl₂, 0.05% Tween 20, 1% BSA). A 10 microliter volume of this solution was loaded on to the disc that was prepared as described in below in Part B of this example.

B. Disc Preparation

A gold disc was coated with maleic anhydride polystrene as described. Distinct reaction zones were created for two types of reporter beads. Each reaction zone consisted of amine DNA sequences complementary to the respective reporter probes (or capture agents). Prior to sample injection, the channel were blocked with a blocking buffer (50 mM Tris, 200 mM NaCl, 10 mM MgCl₂, 0.05% Tween 20, 1% BSA, 1% sucrose) to prevent non-covalent binding of the dual bead complex to the disc surface. Alternatively, magnetic beads employed in a multiplexing dual bead assay format may be detected using a magneto-optical disc and drive. The chemical reaction zones, in the magnetic disc format, are replaced by distinctly spaced magnetic capture zones as discussed in conjunction with FIG. 37, see below Examples 5 and 6.

C. Capture of Dual Bead Complex Structures on the Disc

A 10 microlitre volume of the dual bead mixture prepared as described above in Part A of this example, was loaded in to the disc chamber and the injection ports were sealed. To facilitate hybridization between the reporter probes on the reporter beads and the capture agents, the disc was centrifuged at low speed (less than 800 rpm) for up to 15 minutes. The disc was read in the CD reader at the speed 4×(approx. 1600 rpm) for 5 minutes. Under these conditions, the unbound magnetic capture beads were centrifuged to the bottom of the channels. The reporter beads bound to the capture zone via hybridization between the reporter probes and their complementary agent.

D. Quantification of Dual Bead Complex Structures

The amount of target DNA 1 and 2 captured could be enumerated by quantifying the number of the respective reporter beads in the respective reaction zones.

### EXAMPLE 3

The sensitivity of the dual bead assay depends on the strength of the target mediated-bonds holding the dual beads together. The dual beads are held together by hydrogen bonds. The strength of the bond would increase significantly if the bond holding the dual beads is covalent. For this purpose, after target capture, a ligation reaction is carried out to create a covalent bond between the capture and reporter probes as illustrated above in FIG. 38. The 5′ end of the reporter probe carries a phosphate group which is required in the ligation reaction.

Ligation Experiment: The assay is comprised of 3 μm magnetic capture beads (Spherotech, Libertyville, Ill.) coated with covalently attached capture probes; 2.1 μm fluorescent reporter beads (Molecular Probes, Eugene, Oreg.) coated with a covalently attached sequence specific for the DYS gene, and target DNA molecules containing DYS sequences. The target DNA is a synthetic 80 oligonucleotide long. The capture probes and reporter probes are 40 nucleotides in length and are complementary to the DYS sequence but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA. The capture beads were concentrated magnetically with the supernatant being removed. Then 100 μl of the hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) was added and the beads were resuspended. Various concentration of target DNA ranging from 1, 10, 100, and 1000 femtomoles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

A 2×10⁷ amount of reporter beads in 100 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) was then added to washed capture beads. The beads were resuspended and incubated while mixing at 37 degrees Centigrade for an additional 2 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

After the final wash, the beads were resuspended in 20 μl of binding buffer (50 mM Tris, 200 mM NaCl, 10 mM MgCl₂, 0.05% T-20, 1% BSA). Then 10 μl was loading onto the bio-disc which was prepared as described above in Example 2, Part B.

A. Preparation of Capture Beads

The specific methodology employed to prepare the above assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA. The capture beads were concentrated magnetically with the supernatant being removed. Then 100 μl of the hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) was added and the beads were resuspended. Various concentrations of target DNA ranging from 1, 10, 100, and 1000 femtomoles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

B. Hybridization to Target DNA or Bridging Sequence

Various concentration of target DNA at concentrations 0 mole, 1E-14, 1E-13, 1 E-12, and 1E-11 moles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice. The capture beads were re-suspended in 50 μL of 40 mM NaCl solution.

C. Hybridization to Reporter Probes or Reporter Beads

A 2×10⁷ amount of reporter beads or 100 pmoles of reporter probes in 100 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) was then added to washed capture beads. The beads were resuspended and incubated while mixing at 37 degrees Centigrade for an additional 2 hours. After incubation, the capture beads were concentrated magnetically, and the supernatant containing unbound reporter beads or unbound reporter probes were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

D. Ligation Reactions

A 10 μL volume of the 10× ligation buffer (final concentration 66 mM Tris, pH 7.6, 6.6 mM MgCl₂, 100 mM DTT, 66 μM ATP) and 4 units ligase (concentrations 10 units per μL) was added to the bead suspensions. The ligation reaction was carried out for 2 hours at room temperature. The bead suspensions were washed 3 times with wash buffer (145 mM NaCl, 50 mM Tris, pH 7.5, 0.2% SDS, 0.05% Tween 20, 0.25% NFDM). In the control tube, no ligase was added.

E. Enzyme Assays

The amount of reporter probe was directly correlated with the amount of target DNA captured. Therefore, one way to quantify the target captured was to quantify the amount of reporter probe. The rationale for this assay is that the reporter probe was biotinylated. The concentrations of the reporter probe therefore could be determined by an enzyme assay wherein the enzyme Streptavidin-Alkaline phosphatase binds to the biotin moiety. A chromogenic substrate for Alkaline phosphatase, p-nitrophenyl phosphate, was used as reporter. This colorless substrate is hydrolyzed by alkaline phosphatase to a yellow product which has an absorbance at 405 nm. The beads were washed with 100 μl of CDB (2% BSA, 50 mM Tris-HCl, pH 7.5, 145 mM NaCl, 1.0 mM MgCl₂, 0,1 mM ZnCl₂, 0.05% NaN₃) and incubated with 100 μl of 250 ng/ml Streptavidin-Phosphatase for 1 hour at 37° C. The beads were washed 3 times with wash buffer (145 mM NaCl, 50 mM Tris, pH 7.5, 0.05% Tween) to get rid of unbound S-AP. The beads were incubated with 100 μl of the S-AP substrate p-nitrophosphate at 3.7 mg/ml in 0.1M Tris, pH 10, 2 mM MgCl₂ for 5-15 minutes at room temperature. The color development of the supernatant was monitored at 405 nm. The intensity of the color is directly correlated with the amount of the biotinylated reporter probe and thus the amount of target captured.

F. Dual Bead Assays

The amount of reporter beads was directly correlated with the amount of target captured. Therefore, one way to quantify the target captured was to quantify the amount of reporter beads. After hybridization and ligation, the beads were re-suspended in 200 μL PBS and the amount of reporter beads was quantified by the fluorimeter Fluoromax-2 at Ex=500 nm, Slit=2.0; Em=530 nm, Slit=2.0. Alternatively, the number of fluorescent reporter beads can be quantified by the bio-CD reader as described above.

### EXAMPLE 4

The use of cleavable spacers in dual bead assay increases the specificity of the assay. The following example is directed to a dual bead assay using cleavable spacers.

A. Design of Capture and Reporter Probes

The design of capture probes and reporter probes is critical in the success of the dual bead assay using cleavable spacers. The capture probes and reporter probes contain 3 branches as illustrated above in Fig. One branch of the reporter or capture probes participates in the target capture. Several linkers (PEG groups) are introduced into the capture or reporter probes to minimize coiling of the probe and to increase target capture efficiency. The second branch of the capture or reporter probes contains 3 linkers followed by a biotin at the end. Other functional groups such as carboxyl or amine could also be used. The biotin participates in the conjugation of the capture or reporter probes onto the solid phase. The third branch of the capture probe hybridizes to the reporter probe.

When restriction enzyme digestion is the method of choice for cleaving the capture and reporter probes, a restriction site is introduced into the sequences of the probes. The choice of restriction site is important in that it has to be unique (not common) so that only the sequence holding the capture and reporter probes (and not the target DNA) is cleaved. The formation of the capture and reporter probes in the presence of the target is shown above in FIG. 42C.

When displacement of the reporter probe is the method of choice for cleaving the capture and reporter probes, the sequence on the reporter probes that participates in the hybridization with the capture probe is relatively short (about 10 nucleotides). The remaining sequence is not complementary to the capture probe and therefore will be available for the displacement probe to hybridize. This is generally illustrated above in FIGS. 43A and 43B to show hybridization of capture probe (Probe 1) to reporter probe (Probe 2B). In this example, the probes used were synthesized by Biosource of Camarillo, Calif.

B. Immobilization of Capture Probe onto Streptavidin Beads

1. Preparation of capture beads: The first step in the assay is the conjugation of the capture probe onto a solid phase. In this example, 2.8 μm magnetic beads coated with streptavidin from DynaI were used as the solid phase. Typically, 6.7×10⁷ DynaI beads were used per conjugation. The beads were resuspended in 200 μl of binding and washing buffer (10 mM Tris-HCl, pH 7.5, 1 mM EDTA, 2M NaCl). The beads were magnetically concentrated and the supernatant was removed. The wash procedure was repeated twice.

2. Conjugation of capture probes onto capture beads: The magnetic beads were resuspended in 400 μl binding and washing buffer (10 mM Tris-HCl, pH 7.5, 1 mM EDTA, 2M NaCl) to a final concentration of 5 μg of beads/μl. Then 600 picomoles of capture probes in water was added to the bead suspension. The final salt concentration in the mixture is 1M NaCl. It should be noted that high salt is required for efficient conjugation. The mixture was incubated at 37 degrees Centigrade for 2 to 4 hours with occasional mixing. The beads were then magnetically concentrated and the supernatant was removed. The beads were washed 3 times with binding and washing buffer (10 mM Tris-HCl, pH 7.5, 1 mM EDTA, 2M NaCl).

3. Determination of conjugation efficiency: The optical density of the supernatant before and after conjugation was measured at 260 nm to quantify the amount of capture probes conjugated. Typically, over 50% of the capture probes were conjugated onto the streptaividin beads. The density of probes was from 0.5×10⁶ to 1×10⁶ probes/bead. Table 2 below presents a listing of an example for the determination of conjugation efficiency of biotinylated probe onto Streptavidin coated magnetic beads.

4. Blocking of remaining streptavidin sites on the bead: The beads were incubated in 400 μl of PBS containing 2 mg/ml biotin for 1 hour on a rotating mixer to block all remaining streptavidin sites on the DynaI magnetic beads. The magnetic beads were washed 3 times with binding and washing buffer (10 mM Tris-HCl, pH 7.5, 1 mM EDTA, 2M NaCl) and resuspended in 1000 μl hybridization buffer (0.2M NaCl, 1 mM MgCl₂, 1 mM EDTA, 50 mM Tris, pH 7.5).

TABLE 2
Conjugation of Biotinylated Capture Probe onto
Streptavidin Coated Magnetic Beads

| | |
|---|---|
| 1. | Number of beads used: 1.2 × 10⁸ beads |
| 2. | Number of streptavidin molecules per bead: 7 × 10⁵ molecules/bead |
| 3. | Amount of biotinylated capture probe 1 bound to 1 mg of bead: 127 |
| | pmoles or 8 × 10¹³ molecule |
| 4. | Number of biotin probes/bead: 8 × 10⁶ molecules/bead |
| 5. | All free streptavidin binding sites were saturated with biotin |

C. Hybridization of Capture Probe to Reporter Probes

1. Hybridization: Out of the 1000 μl bead suspension, 400 μl was mixed with 400 μl TE buffer containing 1 nanomole of reporter probe 2A, 400 μl was mixed with 400 μl TE buffer containing 1 nanomole of reporter probe 2B, 200 μl was mixed with 200 μl TE (Tris-EDTA) as a negative control. The hybridization was carried out at 37° C. for 2 hours.

2. Washing: Following hybridization, the magnetic beads were washed 3× with wash buffer (145 mM NaCl, 50 mM Tris, pH 7.5, 0.05% Tween).

3. Determination of hybridization efficiency: Here 50 μl out of 800 μl was assayed for the hybridization efficiency. The rationale for this assay is that the reporter probes 2A and 2B were biotinylated. The concentrations of these probes therefore could be determined by an enzyme assay wherein the enzyme Streptavidin-Alkaline phosphatase binds to the biotin moiety. A chromogenic substrate for Alkaline phosphatase, p-nitrophenyl phosphate, was used as reporter. This colorless substrate is hydrolyzed by alkaline phosphatase to a yellow product which has an absorbance at 405 nm. The beads were washed with 100 μl of CDB (2% BSA, 50 mM Tris-HCl, pH 7.5, 145 mM NaCl, 1.0 mM MgCl₂, 0,1 mM ZnCl₂, 0.05% NaN₃) and incubated with 100 μl of 250 ng/ml Streptavidin-Phosphatase for 1 hour at 37° C. The beads were washed 3 times with wash buffer (145 mM NaCl, 50 mM Tris, pH 7.5, 0.05% Tween) to get rid of unbound S-AP. The beads were incubated with 100 μl of the S-AP substrate p-nitrophosphate at 3.7 mg/ml in 0.1M Tris, pH 10, 2 mM MgCl₂ for 5-15 minutes at room temperature. The color development of the supernatant was monitored at 405 nm. The intensity of the color is directly correlated with the amount of the biotinylated reporter probe 2A or 2B hybridized.

At this point, the reporter probes could be attached to another solid phase via their biotin moiety. For this alternate dual bead assay, a different type of streptavidin coated beads, i.e. polystyrene or fluorescent, is added to the bead suspension, resulting in the formation of the dual bead complexes. If the solid phase is the surface of the bio-disc, then the mixture of capture and reporter probes is incubated on a streptavidin coated disc surface.

D. Hybridization of Probes to Target DNA

1. Hybridization: In this example, the target DNA was a single stranded 80mer oligonucleotide. Various concentrations of target DNA ranging from 0, 1, and 1000 picomoles were added to the bead suspensions. The beads suspensions were incubated while mixing at 37 degrees Centigrade for 2 hours.

2. Washing: The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

E. Distinction of Target-Mediated Capture by Restriction Enzyme Digestion or by Probe Displacement

1. Restriction enzyme digestion: The restriction enzyme site that was introduced in the capture and reporter probes was NOT1. This restriction enzyme site is rare and in this model system is not found in any other sites. The beads were resuspended in 400 μl CDB (2% BSA, 50 mM Tris-HCl, pH 7.5, 145 mM NaCl, 1.0 mM MgCl₂, 0,1 mM ZnCl₂, 0.05% NaN₃). The bead suspension was aliquoted into seven tubes, one control and 6 digestion tubes. The enzyme NOT1 was prepared according to the manufacturer's specifications. Then 5 units of enzyme were added to the each digestion tubes in a total volume of 100 μl. Water was added to the control tube. The digestion was carried out for 3-4 hours at 37° C.

2. Displacement of the reporter probe by the displacement probe: The beads were resuspended in 400 μl CDB (2% BSA, 50 mM Tris-HCl, pH 7.5, 145 mM NaCl, 1.0 mM MgCl₂, 0,1 mM ZnCl₂, 0.05% NaN₃). The bead suspension was aliquoted into two tubes, one control and one displacement tube. The beads were heated for 5 minutes at 55° C. in 200 μl of 6×SSC, 1 mM EDTA. The heat treatment was used to induce the melting of the reporter probe 2B from the capture probe. At this point, a 10 fold excess of displacement probe was added to the bead suspension and the mixture was incubated at 37° C. for several hours Water was added to the control tube.

F. Quantification of Target Captured by Enzyme Assay

The amount of reporter probe remaining after the restriction enzyme digestion or probe displacement was directly correlated with the amount of target DNA captured. Therefore, one way to quantify the target captured was to quantify the amount of remaining reporter probe. The rationale for this assay is that the reporter probes 2A and 2B were biotinylated. The concentrations of these probes therefore could be determined by an enzyme assay wherein the enzyme Streptavidin-Alkaline phosphatase binds to the biotin moiety.

A chromogenic substrate for Alkaline phosphatase, p-nitrophenyl phosphate, was used as reporter. This colorless substrate is hydrolyzed by alkaline phosphatase to a yellow product which has an absorbance at 405 nm. The beads were washed with 100 μl of CDB (2% BSA, 50 mM Tris-HCl, pH 7.5, 145 mM NaCl, 1.0 mM MgCl₂, 0,1 mM ZnCl₂, 0.05% NaN₃) and incubated with 100 μl of 250 ng/ml Streptavidin-Phosphatase for 1 hour at 37° C. The beads were washed 3 times with wash buffer (145 mM NaCl, 50 mM Tris, pH 7.5, 0.05% Tween) to get rid of unbound S-AP. The beads were incubated with 100 μl of the S-AP substrate p-nitrophosphate at 3.7 mg/ml in 0.1M Tris, pH 10, 2 mM MgCl₂ for 5-15 minutes at room temperature. The color development of the supernatant was monitored at 405 nm. The intensity of the color is directly correlated with the amount of the biotinylated reporter probe 2A or 28 hybridized.

G. Quantification of Target Captured by Dual Bead Assay

In the case when the reporter probes are immobilized on another solid phase such as fluorescent or polystyrene streptavidin coated beads, the amount of target captured could be quantified by dual bead assay. The number of reporter beads remaining following restriction enzyme digestion or probe displacement could be enumerated by the fluorimeter (for fluorescent beads) or by the bio-CD reader since each type of bead has a distinct signal signature.

### EXAMPLE 5

The following example illustrates a dual bead assay carried out on a magnetically writable and erasable analysis disc such as the magneto-optical bio-disc 110 discussed in conjunction with FIG. 37.

In this example, the dual bead assay is carried out to detect the gene sequence DYS which is present in male but not female. The assay is comprised of 3 μm magnetic capture beads (Spherotech, Libertyville, Ill.) coated with covalently attached transport probes; 2.1 μm fluorescent reporter beads (Molecular Probes, Eugene, Oreg.) coated with a covalently attached sequence specific for the DYS gene, and target DNA molecules containing DYS sequences. The target DNA is a synthetic 80 oligonucleotides long. The transport probes and reporter probes are 40 nucleotides in length and are complementary to the DYS sequence but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA.

After pretreatment with salmon sperm DNA, the capture beads are loaded inside the MO bio-disc via the injection port. The MO bio-disc contains magnetic regions created by the magneto optical drive. The capture beads thus are held within specific magnetic regions on the MO bio-disc.

The sample containing target DNA and reporter beads in 200 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) is then added to the MO bio-disc via the injection port. The injection port is then sealed. The magnetic field is released. The disc is rotated at very low speed (less than 800 rpm) in the drive to facilitate hybridization of target DNA and reporter beads to the capture beads. The temperature of the drive is kept constant at 33 degrees Centigrade. After 2 hours of hybridization, the magnetic field is created by the magneto optical drive. At this stage, only magnetic capture beads, unbound or as part of a dual bead complex, remain on the MO bio-disc. Unbound target and reporter beads are directed to a waste chamber by any of the mechanisms described above. Two hundred microliters of wash buffer (145 mM NaCl, 50 mM Tris, pH 7.5, 0.1% SDS, 0.05% Tween, 0.25% NFDM (Non Fat Dried Milk), 10 mM EDTA) is then added. The magnetic field is released and the disc is rotated at low speed (less than 800 rpm) for 5 minutes to remove any non-specific binding between the capture beads and reporter beads. The magnetic field is then reapplied. The wash buffer is directed to the waste chamber by any of the mechanisms described above. The wash procedure is repeated twice.

At this stage, only magnetic capture beads, unbound or as part of a dual bead complex, remain. The magnetic field is released and the dual bead complexes are directed to a detection chamber. The amount of target DNA captured is then enumerated by quantifying the number of capture magnetic beads and the number of reporter beads since each type of bead has a distinct signature as illustrated above in FIGS. 28A, 28B, 29A, and 29B.

### EXAMPLE 6

In this example, a dual bead assay using the multiplexing techniques described above in connection with FIGS. 32 and 37 is carried out on a magnetically writable and erasable analysis disc such as the MO bio-disc 110 discussed with reference to FIG. 37.

The dual bead assay is carried out to detect 2 or more DNA targets simultaneously. The assay is comprised of 3 μm magnetic capture beads (Spherotech, Libertyville, Ill.). One population of the magnetic capture beads is coated with transport probes 1 which are complementary to the DNA target 1. Another population of the magnetic capture beads is coated with transport probes 2 which are complementary to the DNA target 2. Alternatively, 2 or more different types of magnetic capture beads may be used. There are two or more distinct types of reporter beads in the assay. The reporter beads may differ by chemical composition (for example silica and polystyrene) and/or by size. One type of reporter beads is coated with reporter probes 1, which are complementary to the DNA target 1. The other reporter beads are coated with reporter probes 2, which are complementary to the DNA target 2. Again, the transport probes and reporter probes are complementary to the respective targets but not to each other.

The specific methodology employed to prepare the dual bead assay multiplexing involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA.

After pretreatment with salmon sperm DNA, the capture beads are loaded in the MO bio-disc. The magnetic field is applied to create distinct magnetic zones for specific capture beads. The capture beads can be held on the MO bio-disc at a density of 1 capture bead per 10 μm². The surface area usable for bead deposition on the MO bio-disc is approximately 3×10⁹ μm². The capacity of the MO bio-disc for 3 μm beads at the given density is about 3×10⁸ beads.

The sample containing the targets DNA of interest is mixed with different types of reporter beads in 200 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) and added to the MO bio-disc via the injection port. The injection port is then sealed. The magnetic field is released. The disc is rotated at very low speed (less than 800 rpm) in the drive to facilitate hybridization of targets DNA and reporter beads to the different types of capture beads. The temperature of the drive is kept constant at 33 degrees Centigrade. After 2 to 3 hours of hybridization, the magnetic field is regenerated by the magneto optical drive. At this stage, only magnetic capture beads, unbound or as part of dual bead complexes, remain on the MO bio-disc. Unbound targets and reporter beads are directed to a waste chamber by any of the mechanisms described above. Two hundred microliters of wash buffer (145 mM NaCl, 50 mM Tris, pH 7.5, 0.1% SDS, 0.05% Tween, 0.25% NFDM (Non Fat Dried Milk), 10 mM EDTA) is then added. The magnetic field is released and the disc is rotated at low speed (less than 800 rpm) for 5 minutes to remove any non-specific binding between the capture beads and reporter beads. The magnetic field is then reapplied. The wash buffer is directed to the waste chamber by any of the mechanisms described above. The wash procedure is repeated twice.

At this stage, the magnetic field is released and the dual bead complexes are directed to a detection chamber. The amount of different types of target DNA can be enumerated by quantifying the number of corresponding capture magnetic beads and reporter beads since each type of bead has a distinct signature as shown above in FIGS. 28A, 28B, 29A, and 29B.

### EXAMPLE 7

This experiment was performed to determine the amount of covalently conjugated probe on different beads to determine which bead type is best for covalent probe linking.

A. Conjugation

Magnetic beads (1-2 μm) from Polysciences, magnetic beads (3 μm) from Spherotech, fluorescent beads (1.8 μm) from Polysciences and fluorescent beads (2.1 μm) from Molecular Probes were evaluated in this example. Approximately 5×10⁸ beads were used per conjugation reaction. The beads were washed and resuspended in 0.05 M MES buffer (2-N-morpholeno-ethanesulphonic acid), pH 6.0 and activated for 15 minutes by the addition of 0.1M EDC (1-ethyl 3-3 dimethylaminopropyl carbodimide-HCl). After activation, the pH of the bead solution was adjusted to ˜7.5 with NaOH. Then 0.5 nanomoles of biotinylated probes were added to the solution. The probes were allowed to conjugate for 2-3 hours at room temperature on a rotating mixer. The beads were then magnetically concentrated and the supernatant was collected. To estimate the amount of biotinylated probes bound to the beads, the optical density (at 260 nm) of the supernatant was measured before and after the conjugation.

B. Determination of Covalent Conjugation Efficiency

Typically 1 to 5×10⁷ beads, conjugated with biotinylated probes as discussed above, were used in the determination of covalent conjugation efficiency of the probes. These beads were washed three times in wash buffer and were resuspended in 200 μl CDB (145 mM NaCl, 50 mM Tris HCl, 2% BSA, 1 mg/ml MgCl₂, 0.1 mM ZnCl₂, 0.05% NaN₂). The beads were then magnetically concentrated, and the supernatant was removed. The beads were resuspended in 100 μl CDB containing 550 ng/ml streptavidin-alkaline phosphatase (S-AP) and incubated for 1 hour at 37° C. to allow sufficient time for the streptavidin to bind to the biotin on the probe. Following incubation with S-AP, the beads were magnetically concentrated, and the supernatant containing unbound S-AP was removed. The beads were washed three times in wash buffer. Next 100 μl of p-nitrophenylphosphate (pNPP), a substrate for alkaline phosphatase at a concentration of 3.7 mg/ml in 0.1M Tris-HCl, pH 10 was added to the beads at fixed time intervals to minimize the variation due to difference in incubation time. The incubation time with the substrate was varied (from 2 min upto 30 min) as needed to obtain reliable OD at 405 nm since time for color development varies depending upon the concentration of probe. The optical density obtained from a spectrophotometer at 405 nm wavelength was proportional to the amount of probes bound to the beads.

The results of the experiment are presented in FIGS. 51A and 51B. As indicated, 87% of the probes that bound to the 1-2 μm magnetic beads from Polysciences were non covalently bound, as compared to 15% of non-covalently bound probes on the 3 μm Spherotech beads.

Referring to FIGS. 53A and 53B, data showing a correlation between the covalent conjugation efficiency and the sensitivity of the dual bead assay is presented. These results indicate that with higher covalent conjugation efficiency, the more sensitive and specific the dual bead assay is. The amount of covalenty bound probes may be calculated by repeating steps in this Part B after performing the steps in Part C below. The calculation of the amount of covalent binding is presented in FIG. 55.

C. Heat Treatment in Removal of Non-Covalently Bound Probes

After determining which bead type has the desired covalent conjugation efficiency, the steps in Parts A and B above may be repeated using non-biotinylated probes and the appropriate bead type for use in a dual bead assay.

Following conjugation, the non-covalently bound probes could be selectively removed by heat treatment of the beads. For this purpose, up to 3×10⁷ beads were resuspended in 100 μl of CDB solution heated at 70° C. for 10 minutes. The beads were then immediately magnetically concentrated and the supernatant was removed. The beads were washed twice in wash buffer and once in CDB and re-suspended in 100 μl CDB. At this point the beads are now ready for use in a dual beads test.

### EXAMPLE 8

Experiments were also done to evaluate the use of double stranded DNA during probe conjugation to increase the covalent conjugation efficiency of the DNA probe on the solid phase.

A. Formation of Double Stranded DNA

The capture probe utilized was 40 nucleotides in length and contained an aminogroup (NH₂) at the 5′ end and several chains of PEG (polyethylene glycol) linker. The strand complimentary to the aminated probe used in this experiment was 40 nucleotides in length and contained a biotin group at the 5′ end. A hybridization reaction was carried out with an excess of complementary probes under stringent conditions at 37° C.

B. Conjugation of Double-Stranded DNA Probe onto Beads

Magnetic beads (1-2 μm) from Polysciences, magnetic beads (3 μm) from Spherotech, fluorescent beads (1.8 μm) from Polysciences and fluorescent beads (2.1 μm) from Molecular Probes were evaluated in this example. Approximately 5×10⁸ were used per conjugation reaction. The beads were washed and resuspended in 1 ml of 0.05 M MES buffer (2-N-morpholeno-ethanesulphonic acid), pH 6.0 and activated for 15 minutes by the addition of 0.1M EDC (1-ethyl 3-3 dimethylaminopropyl carbodimide-HCl). After activation, the pH was adjusted to ˜7.5 with NaOH. A volume of 0.5 nanomoles of probes were then added to the solution. The probe conjugation was carried out for 2-3 hours at room temperature on a rotating mixer. The beads were then magnetically concentrated and the supernatant was removed. To estimate the amount of probes bound to the beads, the optical density at 260 nm of the supernatant was measured before and after the conjugation.

After the conjugation, all unreacted carboxyl groups on the beads were blocked with 1 ml 0.1M Tris-HCl pH 7.5 for 1 hour at room temperature on a mixer. The beads were then blocked for 30 minutes in 1 ml of 10 mg/ml BSA in PBS at room temperature on the mixer to block any unspecific protein binding sites. After blocking, the beads were washed three times with PBS and resuspended in storage buffer (PBS with 10 mg/ml BSA, 5% glycerol, 0.1% sodium azide).

C. Determination of Covalent Conjugation Efficiency

An aliquot of 2×10⁸ magnetic beads was taken out from the above conjugated beads and pre-treated with 0.1 mg/ml salmon sperm DNA for 1 hour at room temperature. The beads were then washed 3 times in wash buffer and resuspended in 200 μl CDB. Then 200 picomoles of blocking probes and 100 μl of hybridization buffer were added to the bead solution. The blocking probes were allowed to hybridize for two hours at 37° C. After hybridization, the beads were magnetically concentrated and the supernatant was removed. The beads were then washed three times in wash buffer using by magnetic concentration. The beads were resuspended with 100 μl of buffer containing 550 ng/ml streptavidin-alkaline phosphatase (S-AP) and incubated for 1 hour at 37° C. Following incubation with S-AP, the beads were magnetically concentrated, and the supernatant containing unbound S-AP was removed. The beads were washed three times in wash buffer. Next 100 μl of p-nitrophenylphosphate (pNPP), a substrate for alkaline phosphatase at a concentration of 3.7 mg/ml, was added to the beads at fixed time intervals to minimize the variation due to difference in incubation time. The time for color development varies depending upon the concentration of probe. The incubation time with the substrate was varied from 2 min up to 30 min as needed to obtain reliable OD at 405 nm. The optical density at 405 nm was proportional to the amount of probes bound to the beads. The results from one of these double stranded conjugation experiments are presented in FIGS. 52A and 52B above.

D. Use of Heat Treatment to Separate Complimentary Strands from Capture Probes

An aliquot of 100 μl of beads were heated for 10 min. at 70° C. Magnetically concentrate the beads and take out the supernatant promptly. Wash once in hot wash buffer and once in CDB. Then resuspend in CDB.

### EXAMPLE 9

Experiments were also conducted to test the use of linkers of longer spacers to increase the efficiency of conjugation and the accessibility and rigidity of the probes attached to a solid phase. In these experiments, the capture and reporter probes were 40 nucleotides in length. These synthetic nucleotide sequences were specific to the analyte of interest. In this example, the 5′ end of the capture probe and 3′ end of the reporter probe contained conjugated 3 polyethylene glycol moieties. These covalently bound linkers were introduced to the probes during probe synthesis. Data collected from one of these experiments are depicted in FIG. 54 above. As shown in FIG. 54, the use of linkers significantly increases the sensitivity of the dual bead assay.

The beads used in this particular assay were 3 μm magnetic beads from Spherotech and 2.1 μm reporter beads from Molecular Probes. The probes were covalently conjugated to the beads as described above. An aliquot of 2×10⁷ of probe conjugated capture beads and 6×10⁷ of reporter beads per assay were washed three times with PBS. After washing, the beads were pretreated with 100 μg/ml of salmon sperm DNA in water for one hour at room temperature. The beads were washed three times in wash buffer (0.145M NaCl, 50 mM Tris-HCl pH 7.5, 0.5% Tween-20), once with hybridization buffer (50 mM Tris-HCl pH 7.5, 0.1M NaCl, 10 mM MgCl, 1 mM EDTA pH 7.5) and re-suspended in hybridization buffer containing 100 μg/ml DNA, and 5× Denhart's mixture.

The two-step hybridization method, as presented in FIG. 12A, was employed in performing the dual bead assay of this example. Different concentrations of a single target were used including Control (0 femtomole), 10 femtomole, 1 femtomole, 0.1 femtomole, 0.01 femtomole, 0.001 femtomole, 0.0001 femtomole diluted in hybridization buffer containing 100 μg/ml of salmon sperm DNA and 5× Denhart's solution. The various target solutions were then mixed with the capture beads and incubated at 37° C. for 2 hours to allow ample time for target hybridization to the capture probe on the beads. After hybridization the hybridized capture beads were washed three times with wash buffer, once with hybridization buffer, and re-suspended in 100 μl hybridization buffer including 100 μg/ml DNA, and 5× Denhart's mixture. The capture bead solution, containing hybridized target, was then mixed with 100 μl of reporter beads and incubated at 37° C. for 2 hours while continuously mixing. Then washed 6 times with new wash buffer (145 mM NaCl, 50 mM Tris-HCl pH 7.5, 05% Tween 20, 0.1% SDS, 0.25% NFDM) and once with PBS. The washed solution containing the dual bead complexes was then re-suspended with 250 μl PBS. The fluorescent signal from the reporter beads were then quantified using a fluorimeter.

Results showed that when 3 PEG linkers were introduced into the capture probe, it lowered the background in dual bead assays and improved the assay sensitivity significantly as compared to probes without linkers.

### EXAMPLE 10

This study was performed to elucidate the assay sensitivity and range of detection of a genetic dual bead assay; the results are shown in FIG. 57.

A. Preparation of Capture and Reporter Beads

The beads used in this experiment were magnetic capture beads (3 um Spherotech) and yellow-fluorescent reporter beads (1 um Polysciences) each covalently conjugated with DNA transport probes and DNA signal probes, respectively. Approximately 1×10⁷ capture beads and 2×10⁷ reporter beads were used for this experiment. These beads were washed 3× with PBS and resuspended in 1 ml water containing 100 μg/ml digested salmon sperm DNA. The bead solutions in the salmon sperm DNA mixture were then incubated for 1 hour at room temperature. After incubation the beads were washed 3× with wash buffer (145 mM NaCl, 50 mM Tris, pH 7.5, 0.05% Tween) and 1× with hybridization buffer (0.1M NaCl, 10 mM MgCl₂, 1 mM EDTA, 50 mM Tris, pH 7.5). The beads were then resuspended in hybridization buffer (containing 100 μg/ml of DNA).

B. Dual Bead Assay

A serial dilution of DNA target agents were prepared containing: 100 femtomole, 10 femtomole, 1 femtomole, 0.1 femtomole, 0.01 femtomole, and 0 femtomoles (negative control). Equal amounts of capture beads were then mixed with the various solutions of target and incubated at 37° C. for 2 hours to let target hybridized to the 5′ capture probe on the beads. After incubation, the bead solutions were washed 3× with the wash buffer (145 mM NaCl, 50 mM Tris, pH 7.5, 0.05% Tween) and 1× with hybridization buffer (0.1M NaCl, 10 mM MgCl₂, 1 mM EDTA, 50 mM Tris, pH 7.5), then resuspended in hybridization buffer (containing 100 μg/ml salmon sperm DNA).

Reporter beads were mixed with the capture bead solution and incubated at 37° C. for 2 hours in a rotating mixer. After incubation the bead solutions were washed 6× with 0.5 ml of wash buffer (145 mM NaCl, 50 mM Tris, pH 7.5, 0.05% Tween, 0.1% SDS, 0.25% NFDM) and resuspended in 250 μl water. The number of reporter beads bound were then quantified by a fluorimeter at Excitation=500 nm, Emission=530 nm, Slit=Ex−2, Em−2, and integration time of 0.1 second.

### EXAMPLE 11

This study was performed to determine the optimal salt concentration in the hybridization buffer for use in a genetic dual bead type assay.

A. Preparation of Capture and Reporter Beads

The beads used in this experiment were magnetic capture beads (3 um Spherotech) and yellow-fluorescent reporter beads beads (2.1 um from Molecular Probes) each covalently conjugated with DNA transport probes and DNA signal probes, respectively. The beads were washed 1× with hybridization buffer (0.1M NaCl, 10 mM MgCl₂, 1 mM EDTA, 50 mM tris, pH 7.5). The beads were pretreated with 0.1% CHAPS and salmon sperm DNA for 1 hour at room temp. The beads were then washed 3× with wash buffer (145 mM NaCl, 50 mM Tris, pH 7.5, 0.1% SDS, 0.05% Tween, 0.25% NFDM), 1× with respective hybridization buffer (145 mM NaCl, 10 mM MgCl₂, 1 mMEDTA, 100 μg/ml salmon sperm DNA, 50 mM Tris, pH 7.5). After washing, the beads were resuspended in hybridization buffer.

B. Dual Bead Assay

The magnetic capture beads, prepared in section A, were divided into 24 10 ul aliquots. Six sets of aliquots were diluted in hybridization solution containing various concentrations of NaCl (0 mM, 145 mM, 300 mM, and 400 mM). The target DNA mixture was made in the hybridization buffer, of varying salt concentrations, in the following concentrations: 100 fmole, 10 fmole, 1 fmole, 0.1 fmole, 0.01 fmole, 0 femtomole (negative control). The various target solutions were then mixed with their respective bead solutions according to the salt concentration of the hybridization buffer and incubated at 37° C. for 2 hours to let target to hybridize to the 5′ transport probe on the capture beads. After hybridization, the assay solutions were washed 3× with wash buffer containing the appropriate amount of NaCl required for each treatment group (0 mM, 0.145 mM, 0.3 mM, 0.4 mM NaCl), 1× with the appropriate hybridization buffer. The beads were then resuspended in their respective hybridization buffers containing the appropriate amount of NaCl.

A 100 μl volume of reporter beads in hybridization buffer containing various NaCl concentrations (0 mM, 0.145 mM, 0.3 mM, 0.4 mM NaCl), were added to the appropriate assay solution, so that the same concentration of NaCl was maintained within the different treatment groups. These assay solutions were then incubated at 37° C. for 2 hours in a rotating mixer. After incubation, the various solutions were then washed 6× with 0.5 ml of wash buffer containing the appropriate amount of NaCl and once with water also containing NaCl at concentrations equal to that in each respective hybridization buffer (0 mM, 0.145 mM, 0.3 mM, 0.4 mM NaCl). The various dual bead solutions were then resuspended in 250 μl water. The number of reporter beads bound were then quantified using a fluorimeter at Excitation=500 nm, Emission=530 nm, Slit=Ex−2, Em−2 and an integration time of 0.1 second. The results from this assay are shown in above FIG. 60. Detection of the dual bead complex may be carried out using the optical disc system described as described in conjunction with FIG. 2., a magneto-optical disc system, a fluorescent disc system, or any similar device. Unique signature traces of a dual bead complex collected from an optical disc reader are shown above in FIG. 29B.

### EXAMPLE 12

In this case, magnetic capture beads (3 μm) from Spherotech and yellow-fluorescent (2.1 μm) reporter beads from Molecular Probes were evaluated. This study was performed to determine the optimal MgCl₂ concentration in the hybridization buffer for use in a genetic dual bead type assay.

A. Preparation of Capture and Reporter Beads

The magnetic capture beads and yellow-fluorescent reporter beads were each covalently conjugated with DNA transport probes and DNA signal probes, respectively. After conjugation, the beads were washed 1× with hybridization buffer (0.1M NaCl, 10 mM MgCl₂, 1 mM EDTA, 50 mM Tris, pH 7.5). The beads were pretreated with 100 μg/ml salmon sperm DNA for 1 hour at room temp. The beads were then washed 3× with wash buffer (145 mM NaCl, 50 mM Tris, pH 7.5, 0.1% SDS, 0.05% Tween, 0.25% NFDM), 1× with hybridization buffer (145 mM NaCl, 10 mM MgCl₂, 1 mM EDTA, 100 μg/ml salmon sperm DNA, 50 mM Tris, pH 7.5). After washing, the beads were resuspended in hybridization buffer.

B. Dual Bead Assay

The magnetic capture beads, as prepared in Part A, were divided into 24 10 ul aliquots. Six sets of aliquots were diluted in hybridization solution containing various concentrations of MgCl₂ (0 mM, 10 mM, 20 mM, and 30 mM). The target DNA mixture was made in hybridization buffer containing varying amounts MgCl₂, in the following concentrations: 100 fmole, 10 fmole, 1 fmole, 0.1 fmole, 0.01 fmole, 0 fmole (negative control). The various target solutions were then mixed with their respective bead solutions according to the salt concentration of the hybridization buffer and incubated at 37° C. for 2 hours to let target to hybridize to the 5′ transport probe on the capture beads. After hybridization, the assay solutions were washed 3× with wash buffer containing the appropriate amount of MgCl₂ required for each treatment group (0 mM, 10 mM, 20 mM, 30 mM MgCl₂), 1× with the appropriate hybridization buffer. The beads were then resuspended in their respective hybridization buffers containing the appropriate amount of MgCl₂.

A 100 μl volume of reporter beads in hybridization buffer containing various MgCl₂ concentrations (0 mM, 10 mM, 20 mM, 30 mM MgCl₂), were added to the appropriate assay solution, so that the same concentration of MgCl₂ was maintained within the different treatment groups. These assay solutions were then incubated at 37° C. for 2 hours in rotating mixer. After incubation, the various solutions were then washed 6× with 0.5 ml of wash buffer containing the appropriate amount of NaCl and once with water also containing MgCl₂ at concentrations equal to that in each respective hybridization. The various dual bead solutions were then resuspended in 250 μl water. The number of reporter beads bound were then quantified by the fluorimeter at Excitation=500 nm, Emission=530 nm, Slit=Ex−2, Em−2, and an integration of 0.1 second. The results from this assay are shown in above FIG. 61A. The dual bead complex may also be quantifed using using an optical disc reader as shown in FIGS. 1 and 2.

### EXAMPLE 13

The following experiment was performed to determine the effect of using a probe blocking agent to reduce the density of probes on beads on the sensitivity of the dual bead assay.

A. Preparation of Capture and Reporter Beads

The magnetic capture beads (3 um Spherotech) and yellow-fluorescent reporter beads (2.1 um Polysciences) were each covalently conjugated with DNA transport probes and DNA signal probes, respectively. After conjugation, the beads were washed 1× with hybridization buffer (0.1M NaCl, 10 mM MgCl₂, 1 mM EDTA, 50 mM tris, pH 7.5). The beads were pretreated with 100 μg/ml salmon sperm DNA for 1 hour at room temp. The beads were then washed 3× with wash buffer (145 mM NaCl, 50 mM Tris, pH 7.5, 0.1% SDS, 0.05% Tween, 0.25% NFDM), 1× with hybridization buffer (100 mM NaCl, 1 mM MgCl₂, 1 mMEDTA, 100 g/ml salmon sperm DNA, 50 mM Tris, pH 7.5). After washing, the beads were resuspended in hybridization buffer.

B. Probe Blocking

A biotinylated transport blocking probe was diluted to the following final concentrations: 500 pmole, 50 pmole, 35 pmole, 30 pmoles. A 13 μl (2×10⁷) volume of magnetic beads were used for each tube (5 tubes total). A 5 μl amount of blocking probe, as prepared above, and 32 μl hybridization buffer was added to each tube. The blocking probes and the transport probes were then hybridized for 2 hours at 37° C. After hybridization, the beads were washed 3× with wash buffer (145 nM NaCl, 50 nM Tris, pH 7.5, 0.05% Tween) and resuspended in 100 μl CDB (2% BSA, 50 mM Tris-HCl, pH7.5, 145 mM NaCl, 1.0 mM MgCl₂, 0.1 mM ZnCl₂, 0.05% NaN₃). The reporter beads were prepared in a similar fashion using a biotinylated reporter blocking probe.

C. Probe Density Determination on Beads after Blocking Agent Treatment

An aliquot of each set of bead solution prepared in Part B was incubated with 100 μl of S-AP (1420 ng/ml) for one hour at 37° C. then washed 3× with wash buffer. After washing, a 100 μl volume of S-AP substrate, p-nitrophenyl phosphate, at a concentration of 3.7 mg/ml in 0.1M Tris, 2 mM MgCl₂, pH 10 was added to the bead solution. After allowing sufficient time for color development, the solution was analyzed using a spectrophotometer (OD@405 nm). The amount of blocking probes on the beads was calculated from the absorbance at 405 nm.

D. Dual Bead Assay

The beads, as prepared in Part B, were washed and resuspended in hybridization buffer containing 100 μg/ml salmon sperm DNA and 5× Denhart's solution. A solution target DNA mixture was prepared in the hybridization buffer with the following concentrations: 0 fmole-control, 10 fmole, 1 fmole, 0.1 fmole, 0.01 fmole, 0.001 fmole, 0.0001 fmole. The target solutions were then mixed with equal amounts of capture and reporter beads and incubated at 37° C. for 2 hours. The capture and reporter beads having been blocked with the same amount of probe blocking agent for each set of assay mixture, i.e., add 10 ul 10 fmole target to 100 ul reporter and capture bead solution each having been blocked with 50 pmole blocking probe. After hybridization, the assay solution was washed 3× with wash buffer and 1× with hybridization buffer and resuspended in hybridization buffer (containing 100 μg/ml salmon sperm DNA and 5× Denhart's Mix). The assay solution was concentrated and resuspended in 250 μl water. The number of reporter beads bound were then quantified by the fluorimeter at Excitation=500 nm, Emission=530 nm, Slit=Ex−2, Em−2, and an integration time of 0.1 seconds.

### EXAMPLE 14

The following experiment was performed to determine the optimal hybridization incubation time of a genetic dual bead assay. The results from this experiment are shown above in FIGS. 63 and 64.

A. Preparation of Capture and Reporter Beads

The beads used in this experiment were 25 μl of capture beads (3 μm carboxylated magnetic particles at a concentration of 1.5×10⁷ beads/μl) with 5′ transport probes attached by covalent conjugation and 400 μl of reporter beads (2 μm YF beads at a concentration of 6.6×10⁶ beads/μl). These beads were washed 3× with PBS and pretreated with 100 μg/ml salmon sperm DNA and 0.1% CHAPS for 1 hour at room temperature. The beads were then washed 3× with wash buffer (145 mM NaCl, 50 mM Tris, pH 7.5, 0.05% Tween) and 1× with hybridization buffer (50 mM Tris-HCl pH 7.5, 10 mM MgCl₂, 0.1M NaCl, 1 mM EDTA). The capture beads were then resuspended in 250 μl hybridization buffer, and the reporter beads in 400 μl hybridization buffer.

B. Dual Bead Assay

A set of target DNA solutions are made in hybridization solution with the following concentrations: 0 picomole, 1 picomole, 10 picomole, 100 picomole target. A test sample was prepared containing 10 μl capture beads, 15 μl of reporter beads, 1 μl salmon sperm DNA and 74 μl target solution all in hybridization buffer. Aliquots of this test sample were analyzed at various incubation times 30 min, 1 hr, 2 hr, 3 hr, 4 hr, and overnight. One set was incubated at 37° C. without mixing and the other set was mixed on a rotating mixer.

The sample aliquots were washed 6× with 0.5 ml wash buffer (145 mM NaCl, 50 mM Tris, pH 7.5, 0.05% Tween, 0.1% SDS, 0.25% NFDM) and then resuspended in 202 μl PBS. The number of reporter beads bound were then quantified by the fluorimeter at Excitation=450 nm, Emission=480 nm, Slit=Ex−1.365, Em−1.05, integration time=0.1 second.

### EXAMPLE 15

After formation of the dual bead complexes, as discussed in connection with FIG. 67A the reporter beads can be separated from the capture beads in a DNA dependent procedure. The dual bead complexes are subjected to DNAases (enzymes that specifically cut DNA). This treatment separates the reporter beads from the capture beads by cutting the DNA that holds them together. Thus, the non-target mediated dual beads will not be affected. The reporter beads that are released after the DNAse treatment are indicative of the amount of target DNA present in the sample. In this experiment, the DNAseI effect in a dual bead assay was evaluated. The results from this experiment is shown in FIGS. 69 and 70.

A. Dual Bead Assay

The dual bead assay was carried out as described previously in Example 1, Part A. Briefly, the assay is comprised of 3 μm magnetic capture beads (Spherotech, Libertyville, Ill.) coated with covalently attached transport probes; 2.1 μm fluorescent reporter beads (Molecular Probes, Eugene, Oreg.) coated with a covalently attached reporter probes, and target DNA molecules of interest. In this example, the target DNA is a synthetic 80 oligonucleotides long. The transport probes and reporter probes are 40 nucleotides in length and are complementary to the target DNA but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA. The capture beads were concentrated magnetically with the supernatant being removed. A 100 μl volume of the hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) were added and the beads were resuspended. Various concentration of target DNA ranging from 1, 10, 100 and 1000 femtomoles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

A 2×10⁷ amount of reporter beads in 100 μl hybridization buffer (0.2M NaCl, 1 mM EDTA, 10 mM MgCl₂, 50 mM Tris-HCl, pH 7.5 and 5× Denhart's mix, 10 g/ml denatured salmon sperm DNA) were then added to washed capture beads. The beads were resuspended and incubated while mixing at 37 degrees Centigrade for an additional 2 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

B. DNAseI Assays

DNAseI was selected for this purpose because it is not sequence specific. Following washing, the dual bead complexes were resuspended in 87.5 μL water. Ten (10) units of DNAseI (2.5 μL) and 10 μL of DNAseI reaction buffer (40 mM Tris-HCl, 10 mM MgSO₄, 1 mM CaCl₂) were added to the re-suspended beads. The digestion reaction was carried out for 1 hour at 37° C. After digestion, the capture beads were concentrated magnetically and the supernatant containing reporter beads was removed. The magnetic capture beads were washed 2 times with 100 μl water. The washed water was combined with the supernatant. The number of reporter beads was quantified by the fluorimeter Fluoromax-2 at excitation 500 nm, emission 530 nm and slit sizes 2.0. See FIGS. 69 and 70 for results from this experiment. Alternatively, the number of fluorescent reporter beads can be quantified by the bio-disc reader as described above.

### EXAMPLE 16

In this example, the dual bead complexes are separated by physical or chemical treatments. The dual bead assay was carried out as described above in Example 15. Following washing, the bead products were washed 5 times with the wash buffer (145 mM NaCl, 50 mM Tris, pH 7.5, 0.1% SDS, 0.05% Tween, 0.25% NFDM (Non Fat Dried Milk), 10 mM EDTA) and divided into four sets.

1. Control: No treatment, the beads were washed twice with 200 μL wash buffer.

2. Acid Wash: The beads were washed twice with 200 μL wash buffer containing 0.1M acetic acid (pH 4).

3. Basic Wash: (The beads were washed twice with 200 μL wash buffer containing 0.1M sodium bicarbonate (pH 9).

4. Urea: The beads were washed twice with 200 μL wash buffer containing 7M urea.

After the physical or chemical treatment, the capture beads were concentrated magnetically, and the supernatant containing released reporter beads was saved. The beads were washed 3 times with wash buffer. The wash was also saved. The magnetic capture beads were re-suspended in 400 μL wash buffer. The amount of reporter beads in the supernatants and in the solution of capture beads were quantified by the fluorimeter Fluoromax-2 at Ex 500 nm, Slit=2.0; Em=530 nm, Slit=2.0. Alternatively, the number of fluorescent reporter beads can be quantified by the bio-disc reader as described above.

As evident by this example, high pH washes can dissociate the reporter beads from the capture beads at low target concentrations. As shown by the experimental results in FIG. 72, the basic wash completely dissociated the reporter beads from capture beads at low target concentrations.

The results of this experiment also established that a 7M urea treatment efficiently dissociates reporter beads from capture beads without significantly compromising the sensitivity. As illustrated by the experimental results present in the bar graphs of FIGS. 73A and 73B, urea treatment efficiently dissociates reporter beads from capture beads.

### EXAMPLE 17

In Examples 15 and 16 discussed above, the target DNA is single stranded. When clinical samples are used, the DNA is double stranded and therefore the hybridization buffer requires a denaturing reagent such as guanidine isothiocyanate. The concentration of the denaturing reagent used in the assay is a major contributor in the specificity and sensitivity of the dual bead assay. In this example, the dual bead assay to detect HSV was carried out in the presence of 1.5M guanidine isothiocyanate.

A. Preparation of Capture Beads

The dual bead assay is comprised of 3 μm magnetic capture beads (Spherotech, Libertyville, Ill.) coated with covalently attached 5′ HSV transport probes and 2.1 μm fluorescent reporter beads from Molecular Probes (Eugene, Oreg.) conjugated to the 3′ HSV reporter probes and target DNA molecules of interest. In this example, the target was a double-strand PCR product containing the HSV gene sequence, amplified for 30 cycles and Qiagen column purified. The transport probes and reporter probes are 40 nucleotides in length and are complementary to the target DNA but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA. The capture beads were concentrated magnetically with the supernatant being removed. The capture beads were resuspended in 600 μL of hybridization buffer (1.5 GuSCN, 8 mM EDTA, 40 mM Tris, pH7.5) containing 5× Denhart's mix, and 10 μg/ml salmon sperm DNA.

B. Preparation of Target DNA

The target was a double-strand PCR product, amplified for 30 cycles and Qiagen column purified. The target was diluted to appropriate concentrations, and heated at 95° C. for 5 minutes to denature the double strand then quickly chilled on ice.

C. Hybridization with the Target DNA

A total of 12.5 μL of the chilled target was added to the 100 μL of the pretreated capture beads. Various concentrations of target DNA ranging from 0, 10^{-16, 10}^{-15, 10}^{-14, 10}⁻¹³, and 10⁻¹² moles were added to the capture bead suspensions. The beads suspension was incubated while mixing at 37 degrees Centigrade for 2 hours. The beads were magnetically concentrated and the supernatant containing unbound target DNA was removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

D. Dual Bead Assay

A 2×10⁷ amount of reporter beads in 100 μl hybridization buffer (1.5 GuSCN, 8 mM EDTA, 40 mM Tris, pH7.4) containing 5× Denhart's mix and 10 μg/ml denatured salmon sperm DNA) were then added to washed capture beads. The beads were resuspended and incubated while mixing at 37 degrees Centigrade for an additional 3 hours. After incubation, the capture beads were concentrated magnetically, and the supernatant containing unbound reporter beads were removed. One hundred microliters of wash buffer (145 mM NaCl, 50 mM Tris, pH 7.5, 0.1% SDS, 0.05% Tween, 0.25% NFDM (Non Fat Dried Milk), 10 mM EDTA) was added and the beads were resuspended. The beads were magnetically concentrated and the supernatant was again removed. The wash procedure was repeated twice.

E. Quantification of Target DNA

The dual bead complexes were resuspended in 250 μl PBS and the amount of target was quantified by fluorescence measurement of the reporter beads using the fluorimeter Fluoromax-2 at Ex=500 nm, Slit=2.0; Em=530 nm, Slit=2.0. Alternatively, the number of fluorescent reporter beads can be quantified by use of the optical bio-disc reader as described above.

### EXAMPLE 18

The following example illustrates a dual bead assay carried out on a magnetically writable and erasable analysis disc such as the magneto-optical bio-disc 110 discussed in conjunction with FIG. 37.

In this example, the dual bead assay is carried out to detect the gene sequence DYS which is present in male but not female. The assay is comprised of 3 μm magnetic capture beads (Spherotech, Libertyville, Ill.) coated with covalently attached transport probes; 2.1 μm fluorescent reporter beads (Molecular Probes, Eugene, Oreg.) coated with a covalently attached sequence specific for the DYS gene, and target DNA molecules containing DYS sequences. The target DNA is a synthetic 80 oligonucleotides long. The transport probes and reporter probes are 40 nucleotides in length and are complementary to the DYS sequence but not to each other.

The specific methodology employed to prepare the assay involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA.

After pretreatment with salmon sperm DNA, the capture beads are loaded inside the MO bio-disc via the injection port. The MO bio-disc contains magnetic regions created by the magneto optical drive. The capture beads thus are held within specific magnetic regions on the MO bio-disc.

The sample containing target DNA and reporter beads in 200 μl hybridization buffer (0.2M NaCl, 1 mM EDTA, 10 mM MgCl₂, 50 mM Tris-HCl, pH 7.5 and 5× Denhart's mix, 10 μg/ml denatured salmon sperm DNA) is then added to the MO bio-disc via the injection port. The injection port is then sealed. The magnetic field is released. The disc is rotated at very low speed (less than 800 rpm) in the drive to facilitate hybridization of target DNA and reporter beads to the capture beads. The temperature of the drive is kept constant at 33 degrees Centigrade. After 2 hours of hybridization, the magnetic field is created by the magneto optical drive. At this stage, only magnetic capture beads, unbound or as part of a dual bead complex, remain on the MO bio-disc. Unbound target and reporter beads are directed to a waste chamber by any of the mechanisms described above. Two hundred microliters of wash buffer (145 mM NaCl, 50 mM Tris, pH 7.5, 0.1% SDS, 0.05% Tween, 0.25% NFDM (Non Fat Dried Milk), 10 mM EDTA) is then added. The magnetic field is released and the disc is rotated at low speed (less than 800 rpm) for 5 minutes to remove any non-specific binding between the capture beads and reporter beads. The magnetic field is then reapplied. The wash buffer is directed to the waste chamber by any of the mechanisms described above. The wash procedure is repeated twice.

At this stage, only magnetic capture beads, unbound or as part of a dual bead complex, remain. The magnetic field is released and the dual bead complexes are directed to a detection chamber. The amount of target DNA captured is then enumerated by quantifying the number of capture magnetic beads and the number of reporter beads since each type of bead has a distinct signature as illustrated above in FIGS. 28A, 28B, 29A, and 29B.

### EXAMPLE 19

In this example, a dual bead assay using the multiplexing techniques described above in connection with FIGS. 32 and 37 is carried out on a magnetically writable and erasable analysis disc such as the MO bio-disc 110 discussed with reference to FIG. 37.

The dual bead assay is carried out to detect 2 or more DNA targets simultaneously. The assay is comprised of 3 μm magnetic capture beads (Spherotech, Libertyville, Ill.). One population of the magnetic capture beads is coated with transport probes 1 which are complementary to the DNA target 1. Another population of the magnetic capture beads is coated with transport probes 2 which are complementary to the DNA target 2. Alternatively, 2 or more different types of magnetic capture beads may be used. There are two or more distinct types of reporter beads in the assay. The reporter beads may differ by chemical composition (for example silica and polystyrene) and/or by size. One type of reporter beads is coated with reporter probes 1, which are complementary to the DNA target 1. The other reporter beads are coated with reporter probes 2, which are complementary to the DNA target 2. Again, the transport probes and reporter probes are complementary to the respective targets but not to each other.

The specific methodology employed to prepare the dual bead assay multiplexing involved treating 1×10⁷ capture beads and 2×10⁷ reporter beads in 100 μg/ml salmon sperm DNA for 1 hour at room temperature. This pre-treatment will reduce the non-specific binding between the capture and reporter beads in the absence of target DNA.

After pretreatment with salmon sperm DNA, the capture beads are loaded in the MO bio-disc. The magnetic field is applied to create distinct magnetic zones for specific capture beads. The capture beads can be held on the MO bio-disc at a density of 1 capture bead per 10 μm². The surface area usable for bead deposition on the MO bio-disc is approximately 3×10⁹ μm². The capacity of the MO bio-disc for 3 μm beads at the given density is about 3×10⁸ beads.

The sample containing the targets DNA of interest is mixed with different types of reporter beads in 200 μl hybridization buffer (0.2M NaCl, 1 mM EDTA, 1 mM MgCl₂, 50 mM Tris-HCl, pH 7.5 and 5× Denhart's mix, 10 μg/ml denatured salmon sperm DNA) and added to the MO bio-disc via the injection port. The injection port is then sealed. The magnetic field is released. The disc is rotated at very low speed (less than 800 rpm) in the drive to facilitate hybridization of targets DNA and reporter beads to the different types of capture beads. The temperature of the drive is kept constant at 33 degrees Centigrade. After 2 to 3 hours of hybridization, the magnetic field is regenerated by the magneto optical drive. At this stage, only magnetic capture beads, unbound or as part of dual bead complexes, remain on the MO bio-disc. Unbound targets and reporter beads are directed to a waste chamber by any of the mechanisms described above. Two hundred microliters of wash buffer (145 mM NaCl, 50 mM Tris, pH 7.5, 0.1% SDS, 0.05% Tween, 0.25% NFDM (Non Fat Dried Milk), 10 mM EDTA) is then added. The magnetic field is released and the disc is rotated at low speed (less than 800 rpm) for 5 minutes to remove any non-specific binding between the capture beads and reporter beads. The magnetic field is then reapplied. The wash buffer is directed to the waste chamber by any of the mechanisms described above. The wash procedure is repeated twice.

At this stage, the magnetic field is released and the dual bead complexes are directed to a detection chamber. The amount of different types of target DNA can be enumerated by quantifying the number of corresponding capture magnetic beads and reporter beads since each type of bead has a distinct signature as shown above in FIGS. 28A, 28B, 29A, and 29B.

### EXAMPLE 20

A. Separation of T-Helper Cells in AIDS Patients using Monoclonal CD4 Antibodies Attached to Paramagnetic Beads

A sample (whole blood or mononuclear cells) is injected into the mixing/loading chamber where it is mixed with anti-CD4 coated paramagnetic beads (bio-magnetic particles). After a 15-minute incubation, to allow binding of the paramagnetic beads to CD4+ cells, magnetic domains are created within the flow channel or analysis chamber using the laser in the MO drive. The tagged CD4+ cells will then bind to these magnetic domains such that when the disc is rotated at a pre-determined speed and time untagged cells and cellular components will move down the flow channel toward a waste chamber. The number of CD4+ cells bound to the magnetic domains is then quantified by the MO reader. The number of CD4+ cells thus determined will be indicative of the heath status of the patient.

B. Manipulation of the Selected T-helper Cells

After the CD4+ cells have been separated from other cellular components, the magnetic domains are then erased. The disc is then rotated at a pre-determined speed and direction to cause the newly released CD4+ cells to move to a flow channel into different testing chambers where the cells could be exposed to different drug treatments that could reduce their susceptibility to HIV destruction. The design of the fluidic circuits and the speed and direction of rotation of the disc can be determined by one of skill in the art by no more than routine experimentation.

### EXAMPLE 21

Detection of Cancer Cells using Carcinoma Marker Antibodies such as MOC-31 or NrLu10 Attached to Paramagnetic Beads

A sample (single cell suspension prepared for example from the biopsied tissue) is loaded into the mixing chamber of the MO bio-disc or MO analysis disc. Paramagnetic beads are then coated with MOC-31 capture antibodies to thereby form bio-magnetic beads. These bio-magnetic beads are then loaded in to the mixing chamber containing the sample. After a 15-minute incubation, magnetic domains are created within the mixing chamber in the MO disc to bind magnetically tagged or labeled cells. The disc is then rotated at a pre-determined speed and duration to remove unlabelled cells. The labeled or tagged carcinoma cells, which are bound to the paramagnetic fields, will be immobilized in the magnetic field whereas other cells will move down the flow channel toward a waste chamber. The number of cancer cells can be quantified by the reader. Further details related to quantification of particles and cells within fluidic circuits are disclosed in, for example, commonly assigned and co-pending U.S. patent application Ser. No. 10/241,512 entitled “Methods for Differential Cell Counts Including Related Apparatus and Software Performing Same” filed Sep. 11, 2002; and U.S. patent application Ser. No. 10/279,677 entitled “Segmented Area Detector for Biodrive and Methods Relating Thereto” filed Oct. 24, 2002. Both of which are herein incorporated by reference in their entireties.

The magnetic field is turned off and the rotation of the disc will cause the identified cancels cells to move to various testing chambers on the MO disc where the cancer cells will be exposed to different anticancer drugs. The efficacy of the drug can be determined by quantifying the number of live cells or dead cells since apoptotic cells have a different signature trace than live cells.

### EXAMPLE 22

Detection of Tuberculosis (TB) using Magnetic Beads Coated with Specific TB Probes

A sample containing DNA fragments is injected into the mixing/loading chamber where it is mixed with paramagnetic beads or bio-magnetic particles coated with DNA probes for one or more species of TB. The sample and the probes are then allowed to hybridize for about 1 hour in the disc with intermittent mixing by rotating the disc clockwise then counter clockwise in the reader. Magnetic domains are then created in the mixing chamber where the magnetic beads are captured. The mixing chamber is then washed to remove unbound DNA fragments. The magnetic domains are then erased and the disc is rotated to cause the magnetic particles to be released and move to an analysis chamber where the captured specific TB DNA sequence could be amplified for downstream applications.

### EXAMPLE 23

The interactions of different neurons in the brain to generate specific action potentials are still unclear. Interactions of specific neurons in the brain can be studied on the MO disc. In various incubation chambers, neurons carrying different cellular markers can be isolated using paramagnetic beads coated with antibodies directed against specific cellular surface markers.

The neurons isolated in one chamber can then be manipulated, moved and mixed with the neurons isolated in a different chamber using magnetic field on the disc. To determine whether there is “communication” between the 2 types of neurons, the production of an action potential (K⁺ or Ca²⁺) could be monitored.

Concluding Summary

All patents and other publications mentioned in this specification are incorporated herein in their entireties by reference.

While this invention has been described in detail with reference to certain preferred embodiments and technical examples, it should be appreciated that the present invention is not limited to those precise embodiments or examples. Rather, in view of the present disclosure, which describes the current best mode for practicing the invention, many modifications and variations would present themselves to those of skill in the art without departing from the scope and spirit of this invention.

For example, any of the off-disc preparation procedures may be readily performed on-disc by use of suitable fluidic circuits employing the methods described herein. Also, any of the fluidic circuits discussed in connection with the reflective and transmissive discs may be readily adapted to the MO bio-disc. In addition, the scope of the present invention is not solely limited to the formation of only dual bead complexes. The methods and apparatus hereof may be readily applied to the creation of multi-bead assays. For example, a single capture bead may bind multiple reporter beads. Similarly, a single reporter bead may bind multiple capture beads. Furthermore, linked chains of multi-bead or dual bead complexes may be formed by target mediated binding between capture and reporter beads. The linked chains may further agglutinate to thereby increase detectability of a target agent of interest.

The scope of the invention is, therefore, indicated by the following claims rather than by the foregoing description. All changes, modifications, and variations coming within the meaning and range of equivalency of the claims are to be considered within their scope.

What is claimed is:

1. A magneto-optical bio-disc for use in bio-magnetic assays, said bio-disc comprising:
a substrate having a center and an outer edge;
a magneto-optic stack associated with said substrate; and
one or more magnetic domains formed on said magneto-optic stack, said one or more magnetic domains employed to bind and release bio-magnetic particles.

2. The magneto-optical bio-disc according to claim 1 wherein said bio-magnetic particles are paramagnetic beads having attached thereto a binding agent.

3. The magneto-optical bio-disc according to claim 2 wherein said binding agent is selected from the group comprising an antigen, an antibody, a ligand, a receptor, biotin, streptavidin, DNA fragments, and RNA fragments.

4. The magneto-optical bio-disc according to claim 1 wherein said one or more magnetic domains are selectively formed within said center and outer edge of said substrate using a magneto-optical disc drive.

5. The magneto-optical bio-disc according to claim 4 wherein said one or more magnetic domains are selectively erased using said magneto-optical disc drive.

6. The magneto-optical bio-disc according to either claim 4 or 5 wherein said one or more magnetic domains are formed and erased to thereby allow said bio-magnetic particles to be selectively bound and released.

7. The magneto-optical bio-disc according to claim 6 wherein said magneto-optical disc drive is used to read and detect features located in said magnetic domains.

8. The magneto-optical bio-disc according to claim 7 further comprising a channel layer associated with said substrate, said channel layer having cut-out portions to form fluidic circuits.

9. The magneto-optical bio-disc according to claim 8 further comprising a cap portion associated with said channel layer.

10. The magneto-optical bio-disc according to claim 9 wherein said fluidic circuits include an inlet port, a mixing chamber, a separation chamber, one or more testing chambers, and a vent port, all of which being in fluid communication with each other.

11. The magneto-optical bio-disc according to claim 10 wherein said one or more testing chambers are pre-loaded with a test solution having a test agent.

12. A method of using the magneto-optical bio-disc of claim 11 to detect, quantify, and test target cells for drug sensitivity, said method of using comprising the steps of:
providing a sample of cells;
loading said sample into said mixing chamber through said inlet port;
providing bio-magnetic particles having attached thereto binding agents specific for surface markers on the surface of target cells in the sample;
loading said bio-magnetic particles into said mixing chamber;
incubating said sample and bio-magnetic particles for a sufficient time to allow binding of the binding agents with the surface markers on the target cells in the sample to thereby create labeled cells;
loading said magneto-optical bio-disc into said magneto-optical disc drive;
forming magnetic domains in pre-determined locations within said separation chamber;
rotating said magneto-optical bio-disc to move said sample and bio-magnetic particles into said separation chamber;
allowing said bio-magnetic particles and labeled cells to bind to said magnetic domains within said separation chamber; and
scanning said separation chamber with a beam of electromagnetic radiation to determine whether magnetic domains contain labeled cells.

13. The method according to claim 12 further comprising the steps of:
quantitating the number of labeled cells bound in said magnetic domains;
erasing selectively said magnetic domains having labeled cells bound thereto, thereby selectively releasing the labeled cells;
guiding magnetically said labeled cells into said one or more test chambers by sequentially erasing and forming said magnetic domains to move a pre-determined number of labeled cells into the one or more test chambers;
incubating said labeled cells with said test agent; and
scanning said test chamber with beam of electromagnetic radiation to determine the number of live and apototic cells and thereby determine the sensitivity of the cells to said test agent.

14. A method of using the magneto-optical bio-disc of claim 9 to create neural networks within a biological matrix in said fluidic circuits, said method of using comprising the steps of:
forming said biological matrix in said fluidic circuits;
providing neurons having dendrites and axons into said biological matrix, said neurons having magnetic particles incorporated therein;
loading said magneto-optical bio-disc into said magneto-optical disc drive;
writing and erasing magnetic domains within said fluidic circuits to cause said dendrites and axons of said neurons to grow toward each other; and
allowing interaction between said neurons through said dendrites and axons thereby forming said neural networks magnetically controlled by said magnetic domains.

15. The magneto-optical bio-disc of claim 11.

16. A magneto-optical bio-disc system for use in magneto-optical bio-magnetic assays, said system comprising:
a magneto-optical bio-disc comprising:
a substrate having a magneto-optic stack; and
one or more magnetic domains formed within in said substrate, said one or more magnetic domains employed to bind and release bio-magnetic particles; and
a magneto-optical disc drive comprising:
a light source for directing light to the disc, said light source further used to form or erase said one or more magnetic domains; and
a detector for detecting light reflected from or transmitted through the disc and providing a signal.

17. The magneto-optical bio-disc system according claim 16 further comprising a processor for utilizing said signal to count items bound to said one or more magnetic domains.

18. The magneto-optical bio-disc system according to claim 17.

19. A method of making a magneto-optical bio-disc, said method comprising the steps of:
providing a substrate having a center, an outer edge, and one or more magneto optical stacks;
forming one or more magnetic domains within said center and outer edge; and
forming an analysis chamber in fluid communication with said one or more magnetic domains.

20. The method according to claim 19 further comprising the step of forming a mixing chamber in fluid communication with the analysis chamber.

21. The method according to claim 19 further comprising the step of forming a biological matrix within said analysis chamber.

22. The method according to claim 20 further comprising the step of depositing a plurality of bio-magnetic particles in said mixing chamber, each of said bio-magnetic particles including a binding agent.

23. The method according to claim 22 further comprising the step of designating an input site associated with said mixing chamber, said input site implemented to receive a sample of cells to be tested for the presence of any target cells.

24. The method according to either claim 21 or 23 further comprising the step of encoding information on an information layer associated with the substrate, said encoded information being readable by a disc drive assembly to control rotation of the disc.

25. The method according to claim 24 further comprising the step of forming a test chamber in fluid communication with said analysis chamber.

26. The method according to claim 25 further comprising the step of filling said test chamber with a test agent.

27. A method of using the magneto-optical bio-disc made according to claim 21 to create neural networks within the biological matrix in said fluidic circuits, said method of using comprising the steps of:
providing neurons having dendrites and axons into said biological matrix;
incorporating magnetic particles into said neurons;
loading said magneto-optical bio-disc into a magneto-optical disc drive;
erasing and forming said one or more magnetic domains within said analysis chamber to cause said dendrites and axons of said neurons to grow toward each other; and
allowing interaction between said neurons through said dendrites and axons thereby forming said neural networks magnetically controlled by said magnetic domains.

28. A method of using the magneto-optical bio-disc made according to claim 26 to detect, quantify, and test said any target cells in said sample of cells for drug sensitivity, said method of using comprising the steps of:
loading said sample into said mixing chamber through said input site;
incubating said sample and bio-magnetic particles for a sufficient time to allow binding of the binding agents with surface markers on the target cells in the sample to thereby create labeled cells;
loading said magneto-optical bio-disc into a magneto-optical disc drive;
forming magnetic domains in pre-determined locations within said analysis chamber;
rotating said magneto-optical bio-disc to move said sample and bio-magnetic particles into said analysis chamber;
allowing said bio-magnetic particles and labeled cells to bind to said magnetic domains within said analysis chamber; and
scanning said analysis chamber with a beam of electromagnetic radiation to determine whether magnetic domains contain labeled cells.

29. The method according to claim 28 further comprising the steps of:
quantitating the number of labeled cells contained in said magnetic domains;
erasing selectively said magnetic domains having labeled cells bound thereto, thereby selectively releasing the labeled cells;
guiding magnetically said labeled cells into said test chamber by sequentially erasing and forming said magnetic domains to move a pre-determined number of labeled cells into the test chamber;
incubating said labeled cells with said test agent; and
scanning said test chamber with beam of electromagnetic radiation to determine the number of live and apototic cells and thereby determine the sensitivity of the cells to said test agent.
